SEQUENCE LISTING (11 sequences)

SEQ ID NO: 1 - protein, 635 aa, Homo sapiens
MASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCQKIGTQGNVNFGGRPQLPGSHPATWSAGGIISRIKSYSFPKPGHRKSSPAQGNRQESTVSFNPYEPELAPWAADKGPQREALPMDTEVYESPYADPEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYYDVVN

SEQ ID NO: 2 - protein, 8 aa, Artificial Sequence
  Decsription of Artificial Sequence Synthetic peptide
  MOD_RES (1): NAc-Glu
  c-term amidation
EEDDYESP

SEQ ID NO: 3 - DNA, 39 bp, Artificial Sequence
  Decsription of Artificial Sequence Primer
cgcggatccgccaccatggacacagaggtgtacgagagc

SEQ ID NO: 4 - DNA, 58 bp, Artificial Sequence
  Decsription of Artificial Sequence Primer
cggcggatccttaatgatgatgatgatgatggttcaccacgtcatagtagtaattgcg

SEQ ID NO: 5 - protein, 8 aa, Artificial Sequence
  Decsription of Artificial Sequence Synthetic peptide
EEDDYESP

SEQ ID NO: 6 - protein, 5 aa, Artificial Sequence
  Decsription of Artificial Sequence Synthetic peptide
DEEDY

SEQ ID NO: 7 - protein, 5 aa, Artificial Sequence
  Decsription of Artificial Sequence Synthetic peptide
DEEYD

SEQ ID NO: 8 - protein, 5 aa, Artificial Sequence
  Decsription of Artificial Sequence Synthetic peptide
DEYED

SEQ ID NO: 9 - protein, 5 aa, Artificial Sequence
  Decsription of Artificial Sequence Synthetic peptide
DYEEV

SEQ ID NO: 10 - protein, 5 aa, Artificial Sequence
  Decsription of Artificial Sequence Synthetic peptide
  MOD_RES (5): Nle
YSIIX

SEQ ID NO: 11 - protein, 26 aa, Artificial Sequence
  Description of Artificial Sequence Consensus sequence
  MOD_RES (6)..(12): Variable amino acid
  MOD_RES (14): Variable amino acid
  MOD_RES (16): Variable amino acid
  MOD_RES (20): Variable amino acid
DFGWSXXXXXXXRXTXCGTXDYLPPE

(19) United States
(12) Patent Application Publication
(10) Pub. No.: US 2007/0020253 A1
(43) Pub. Date: Jan. 25, 2007

(54) Spleen tyrosine kinase catalytic domain:crystal structure and binding pockets thereof

(76) Inventors: David Williams, Melbourn (GB); Maria Bac Lamers, Linton (GB); Michael Cherry, Newmarket (GB); Stephanie Smith, Norwich (GB)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/531,532

(22) PCT Filed: Oct. 16, 2003
(86) PCT No.: PCT/US03/32812

§ 371 Date: Aug. 9, 2006

Related U.S. Application Data
(60) Provisional application No. 60/419,382, filed on Oct. 16, 2002.

(51) Int. Cl. A61K 38/48; A61K 38/08; G06F 19/00; C12N 9/12

(52) U.S. Cl. 424/94.5; 435/194; 702/20; 514/17

(57) ABSTRACT

The present invention provides crystalline molecules and molecular complexes that comprise binding pockets of Sykcat and its homologues. The invention also provides crystals comprising the catalytic domain of Syk protein. The invention further provides a computer comprising a data storage medium encoded with the structure coordinates of Sykcat binding pockets and methods for using a computer to evaluate the ability of a chemical entity or compounds to bind to a crystalline molecule or molecular complex of the invention. This invention also provides methods of using the structure coordinates to solve the structure homologous proteins or protein complexes. The invention further provides methods of using the structure coordinates to screen for, design and optimize chemical entities or compounds, including inhibitory compounds, that bind to the catalytic domain of Syk or homologues thereof.

This application claims benefit of U.S. Provisional Application No. 60/419,382, filed Oct. 16, 2002, the disclosure of which is incorporated herein by reference.

### TECHNICAL FIELD OF THE INVENTION

The present invention relates to molecules or molecular complexes which comprise binding pockets of the catalytic domain of Spleen Tyrosine Kinase protein (Syk_{Cat}) and its homologues. The present invention provides a computer comprising a data storage medium encoded with the structure coordinates of such binding pockets. This invention also relates to methods of using the structure coordinates to solve the structure of homologous proteins or protein complexes. In addition, this invention relates to methods of using the structure coordinates to screen for and design compounds, including inhibitory compounds, that bind to Syk_{Cat} or homologues thereof. The invention also relates to crystallizable compositions and crystals comprising Syk_{Cat} protein or Syk_{Cat} protein complexes.

### BACKGROUND OF THE INVENTION

Syk (Spleen tyrosine kinase) is a cytoplasmic 72 kDa (˜630 amino acids) protein-tyrosine kinase (PTK) that was originally found to be expressed in the spleen and thymus (Zioncheck et al., J. Appl. Cryst. 263, pp. 19195-19202 (1988)). It is widely expressed in a variety of hematopoietic cells, including B- and T-cells at various stages of development. Syk belongs to a family of non-receptor PTKs which also includes Zap70 protein-tyrosine kinase (ZAP-70), a PTK implicated in T-cell receptor signaling (Chan et al., J. Immunology, 152, pp. 4758-4766 (1994)). Human Syk has 93% amino acid homology to porcine Syk, greater than 90% amino acid sequence identity with murine Syk, and 73% identity to human ZAP-70 (Law et al., J. Biol. Chem., 269, pp. 12310-12319 (1994); Furlong et al., Biochim. Biophys. Acta, 1355, pp. 177-190 (1997)). Syk is more abundantly expressed than ZAP-70, whose expression is restricted to T- and natural killer (NK-) cells (Chan et al., J. Immunology, 152, pp. 4758-4766 (1994)).

Immune receptors, including the T- and B-cell receptors (TCR and BCR, respectively), control lymphocyte development through the combination of the responses of native and acquired immunity (Alberola-Ila et al., Annu. Rev. Immunol., 15, pp. 317-404 (1997); Reth & Wienands Annu. Rev. Immunol., 15, pp. 453-479 (1997); Turner et al., Immunology Today, 3, pp. 148-154 (2000)). The activation of various PTKs is an early and essential event in the transduction of signals from immune receptors.

Gene-targeting studies have demonstrated an essential role for Syk in signal transduction via immune receptors (Turner et al., Immunology Today, 3, pp. 148-154 (2000)). Src family kinases phosphorylate Immunoreceptor tyrosine-based activation motifs (ITAMs) on the cytoplasmic tail of cell-surface receptors, creating docking sites for the SH2 domains of Syk. Binding of the tandem SH2 domains in Syk to the phosphorylated ITAMs leads to activation of Syk and thereby to signal transduction (Bolen & Brugge, Annu. Rev. Immunol., 15, pp. 371-404, (1997); Steele et al., Gene, 239, pp. 91-97 (1999)).

Directed disruption of the Syk gene in knockout mice leads to embryonic hemorrhage and death (Cheng et al., Nature, 378, pp. 303-306 (1995); Turner et al., Nature, 378, pp. 298-302 (1995)). In addition, Syk is commonly expressed in normal human breast tissue, benign breast lesions, and low-tumorigenic breast cancer cell lines (Coopman et al., Nature, 406, pp. 742-747 (2000)). In cancerous breast tissue or cell lines, Syk mRNA and protein levels are low or undetectable, suggesting that loss of Syk expression may be associated with the development of a malignant phenotype in breast cancer (Coopman et al., Nature, 406, pp. 742-747 (2000)). Introduction of wild type Syk into a Syk-knockout breast cancer cell line potently inhibited tumor growth and metastasis in athymic mice. Conversely, overexpression of a Syk protein that has been mutated to have no kinase activity in a Syk-positive breast cancer cell line markedly increased its tumor incidence and growth. Tumor incidence and growth was retarded by reintroduction of wildtype Syk. Thus, Syk appears to be an important feature of epithelial cell growth control and a potential tumor suppressor in human breast cancers (Coopman et al., Nature, 406, pp. 742-747 (2000)).

Syk family tyrosine kinases contain tandem N-terminal SH2 domains and a C-terminal catalytic kinase domain. These domains are separated by a “linker region”, designated inter-domain B. As discussed above, the SH2 domains bind phosphotyrosines in ITAMs. The linker region contains multiple tyrosine residues that, upon phosphorylation, act as docking sites for other proteins such as phospholipase Cγ1 (PLCγ1), VAV and CBL, all of which are possible Syk substrates (Sillman and Monroe, J. Biol. Chem., 270, pp. 11806-11811 (1995); Furlong et al., Biochim. Biophys. Acta, 1355, pp. 177-190 (1997); Law et al., Mol. Cell. Biol., 16, pp. 1305-1315 1996)). Syk does not contain an SH3 domain or a membrane-spanning region. The kinase (catalytic) and SH2 domains show 25-40% identity in sequence to PTKs in other families, but the intervening sequences, including linker regions, are unique.

The crystal structure of the regulatory SH2 domains of Syk bound to a phosphorylated ITAM peptide was solved by multiple isomorphous replacement at 3.0 Å (Fütterer et al., J. Mol. Biol., 281, pp. 523-537 (1998)). The two SH2 domains and an intervening region which connects them together form a Y-shaped molecule. Both SH2 domains fold in a similar manner to other SH2 domains, each of which contain a large β-sheet flanked by two α-helices (Kuriyan & Cowburn, Curr. Opin. Struct. Biol., 3, pp. 828-837 (1993)).

Regulation of Syk protein expression has been implicated as a strategy for treatment of breast cancer (Stewart & Pietenpol, Breast Cancer Res., 3, pp. 5-7 (2001)), leukemia (Goodman, et al., Oncogene, 20, pp. 3969-78 (2001)), asthma (Yamada, et al., J. Immunol., 167, pp. 283-8 (2001)), Systemic Lupus Erythematosus (SLE)(Liossis, et al., J. Investig. Med., 49, pp. 157-65 (2001)), and other inflammatory diseases (Malaviya, et al., Am. J. Ther., 8, pp. 417-24 (2001)).

As no structural information on the catalytic domain of Syk or ZAP-70 has been available, detailed information about the catalytic ATP-binding site and the substrate binding site has been absent. It would be desirable to have detailed structural models of the catalytic domain of Syk to screen for, design and optimize drugs to modulate Syk and treat diseases including cancer, asthma, Systemic Lupus Erythematosus (SLE) and other inflammatory diseases. Additional information about the kinase mechanism of Syk also would be revealed by a structure of the catalytic domains of the enzyme with a substrate or inhibitor.

### SUMMARY OF THE INVENTION

The present invention provides, for the first time, the crystal structures of complexes of the catalytic domain of Syk (Syk_{Cat}) and methods of using these crystal structures for drug design and discovery.

The present invention also provides crystalline molecules or molecular complexes comprising Syk_{Cat} binding pockets, or Syk_{Cat}-like binding pockets that have similar three-dimensional shapes. In one embodiment, the crystalline molecules or molecular complexes are Syk proteins, Syk_{Cat} proteins, Syk or Syk_{Cat} protein complexes or homologues thereof. The invention provides crystal compositions comprising Syk_{Cat} protein, Syk_{Cat} protein complex, or homologues thereof in the presence or absence of a chemical entity. The invention also provides a method of crystallizing Syk_{Cat} protein, Syk_{Cat} protein complex, or homologues thereof.

The invention further provides a computer comprising a data storage medium that comprises the structure coordinates of molecules and molecular complexes comprising all or part of the Syk_{Cat} or Syk_{Cat}-like binding pocket. Such storage medium, when read and utilized by a computer programmed with appropriate software, displays on a computer screen or similar viewing device, a three-dimensional graphical representation of the molecule or molecular complex comprising such binding pockets.

The invention provides methods for screening, designing, optimizing, evaluating and identifying compounds or chemical entities that bind to the molecules or molecular complexes or their binding pockets. The methods can be used to identify agonists and antagonists of Syk and its homologues.

The invention also provides a method for determining at least a portion of the three-dimensional structure of molecules or molecular complexes which contain some structurally similar features to Syk, particularly Syk_{Cat} homologues. This is achieved by using at least some of the structure coordinates obtained from the Syk_{Cat} complexes.

### BRIEF DESCRIPTION OF THE DRAWINGS

The following abbreviations are used in FIGS. 1 and 2:

“Atom type” refers to the element whose coordinates are measured. The first letter in the column defines the element.

“Resid” refers to the amino acid residue identity.

“X, Y, Z” define the atomic position of the element measured.

“B” is a thermal factor that measures movement of the atom around its atomic center.

“Occ” is an occupancy factor that refers to the fraction of the molecules in which each atom occupies the position specified by the coordinates. A value of “1” indicates that each atom has the same conformation, i.e., the same position, in all molecules.

“Mol” refers to the molecule in the asymmetric unit.

FIG. 1A (1A-1 to 1A-88) lists the atomic structure coordinates for the Syk_{Cat} (amino acid residues 358-405 and 411-635 of full-length human Syk protein (SEQ ID NO: 1)) complexed with staurosporine at a resolution of 1.65 Å as derived by X-ray diffraction from the crystal. The second line for each atom gives the values from anisotropic B factor refinement for that atom. The coordinates are listed in Protein Data Bank (PDB) format. Residues STU B, HOH W represent staurosporine and water, respectively. Amino acid residues identified as C residues are part of N-terminal end of the neighboring Syk_{Cat} molecule

FIG. 2A (2A-1 to 2A-39) lists the atomic structure coordinates for the Syk_{Cat}-PT426-adenylyl imidodiphosphate (AMP-PNP) complex at 2.4 Å as derived by X-ray diffraction from the crystal. The Syk_{Cat} model contains amino acid residues 364-380, 383-404, and 412-634 of full-length Syk protein (SEQ ID NO: 1). The coordinates are listed in Protein Data Bank (PDB) format. Residues PTR, ANP B, HOH W, and MG M represent phosphorylated tyrosine, adenylyl imidodiphosphate, water, and magnesium ion, respectively. Amino acid residues identified as “C” are from the peptide PT426 (SEQ ID NO: 2).

FIG. 3 depicts a ribbon diagram of the overall fold of the Syk_{Cat}-staurosporine complex. Staurosporine is shown in stick representation. The α-helices and β-strands are labeled as αC-αI and β1-β11, respectively.

FIG. 4 depicts a ribbon diagram of the overall fold of the Syk_{Cat}-PT426-AMP-PNP complex. PT426 and AMP-PNP are shown in stick representation. The α-helices and β-strands are labeled as αC-αI and β1-β11, respectively.

FIG. 5 shows a detailed representation of pockets in the Syk_{Cat}-staurosporine complex. Staurosporine is shown in stick representation and Syk_{Cat} is shown as a ribbon. Contacts between Syk_{Cat} and staurosporine are represented by dashed lines.

FIG. 6 shows a detailed representation of the substrate binding pocket in the Syk_{Cat}-PT426-AMP-PNP complex. PT426 is shown in stick representation and Syk_{Cat} is shown as a ribbon. Contacts between Syk_{Cat} and staurosporine are represented by dashed lines.

FIG. 7 shows a diagram of a system used to carry out the instructions encoded by the storage medium of FIGS. 8 and 9.

FIG. 8 shows a cross section of a magnetic storage medium.

FIG. 9 shows a cross section of an optically-readable data storage medium.

### DETAILED DESCRIPTION OF THE INVENTION

In order that the invention described herein may be more fully understood, the following detailed description is set forth.

Throughout the specification, the word “comprise”, or variations such as “comprises” or “comprising” will be understood to imply the inclusion of a stated integer or groups of integers but not exclusion of any other integer or groups of integers.

The following abbreviations are used throughout the application:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

As used herein, the following definitions shall apply unless otherwise indicated.

The term “about” when used in the context of root mean square deviation or RMSD values takes into consideration the standard error of the RMSD value, which is ±0.1 Å.

The term “associating with” refers to a condition of proximity between a chemical entity or compound, or portions thereof, and a binding pocket or binding site on a protein. The association may be non-covalent—wherein the juxtaposition is energetically favored by hydrogen bonding, hydrophobic, van der Waals or electrostatic interactions—or it may be covalent.

The term “ATP analogue” refers to a compound derived from adenosine-5′-triphosphate (ATP). The compound can be adenosine, AMP, ADP, or a non-hydrolyzable analogue, such as, but not limited to adenylyl imidodiphosphate (AMP-PNP). The analogue may be in complex with magnesium or manganese ions.

The term “binding pocket” refers to a region of a molecule or molecular complex, that, as a result of its shape, favorably associates with another chemical entity or compound. The term “pocket” includes, but is not limited to, a cleft, channel or site or some combination thereof. Syk_{Cat} or Syk_{Cat}-like molecules may have binding pockets which include, but are not limited to, peptide or substrate binding sites, and ATP-binding sites.

The term “catalytic active site” or “active site” refers to the portion of the protein kinase to which nucleotide substrates bind. For example, the catalytic active site of Syk_{Cat} is at the interface between the N-terminal, β-strand lobe or sub-domain and the C-terminal, α-helical lobe or sub-domain.

The term “catalytic domain of Syk” or “Syk catalytic domain” refers to the kinase domain of the human Syk molecule. This domain is located at the C-terminal end of the Syk protein. (See, Latour et al., EMBO J., 17, pp. 2584-2595 (1998)). The domain includes, for example, the catalytic active site comprising the catalytic residues. The domain in the Syk protein comprises amino acid residues from about 343 to 639.

The term “chemical entity” refers to chemical compounds, complexes of at least two chemical compounds, and fragments of such compounds or complexes. The chemical entity can be, for example, a ligand, substrate, nucleotide triphosphate, nucleotide diphosphate, phosphate, nucleotide, agonist, antagonist, inhibitor, antibody, peptide, protein or drug. In one embodiment, the chemical entity is an inhibitor or substrate for the active site.

The term “complex” or “molecular complex” refers to a protein associated with a chemical entity.

The term “conservative substitutions” refers to residues that are physically or functionally similar to the corresponding reference residues. That is, a conservative substitution and its reference residue have similar size, shape, electric charge, and chemical properties, including the ability to form covalent or hydrogen bonds, or the like. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

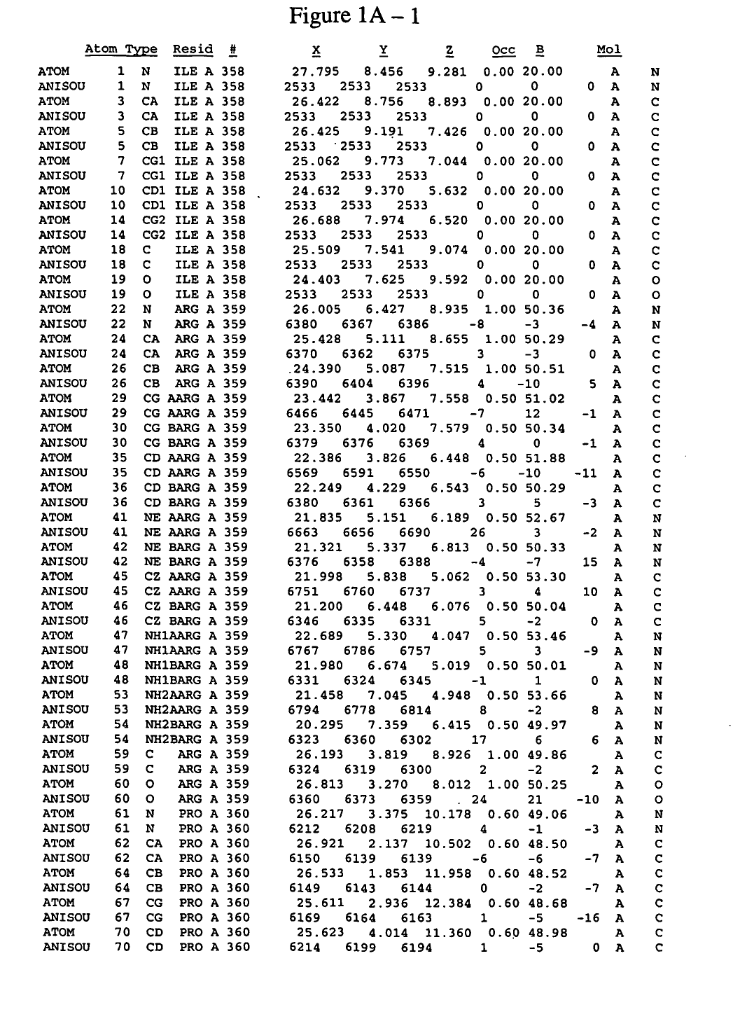

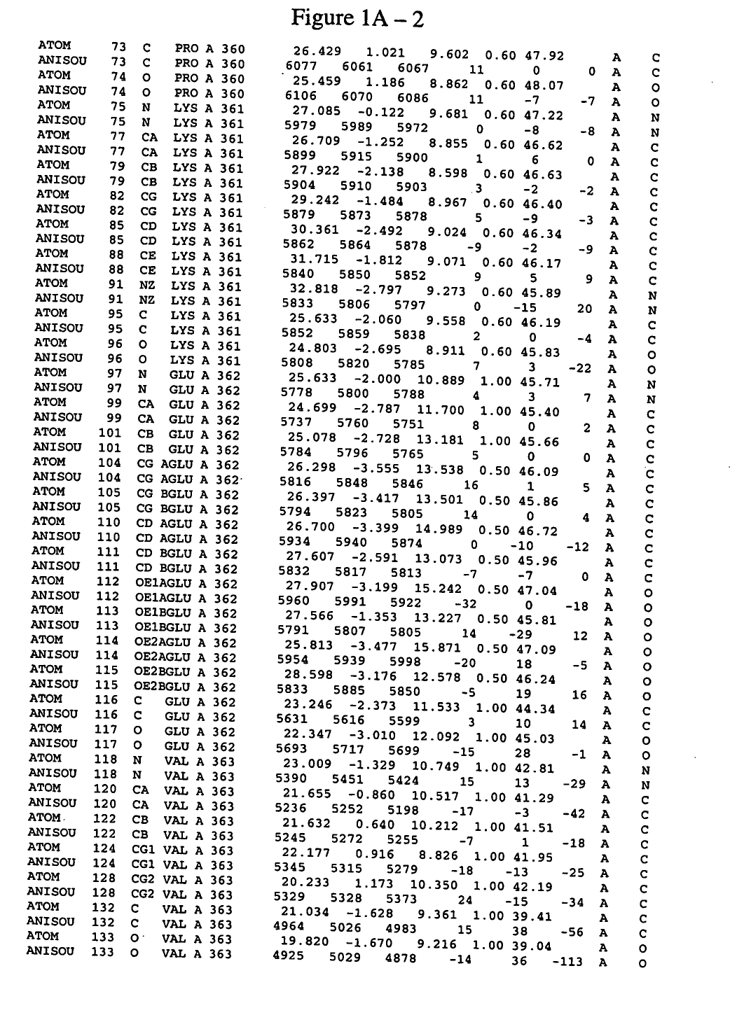

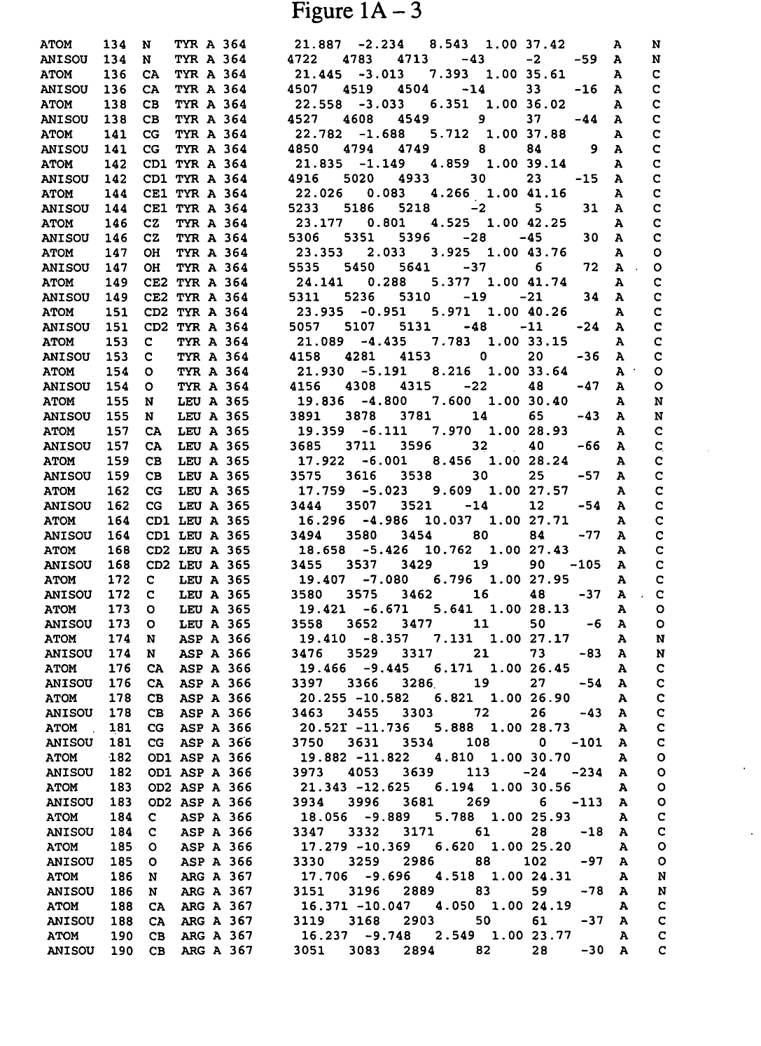

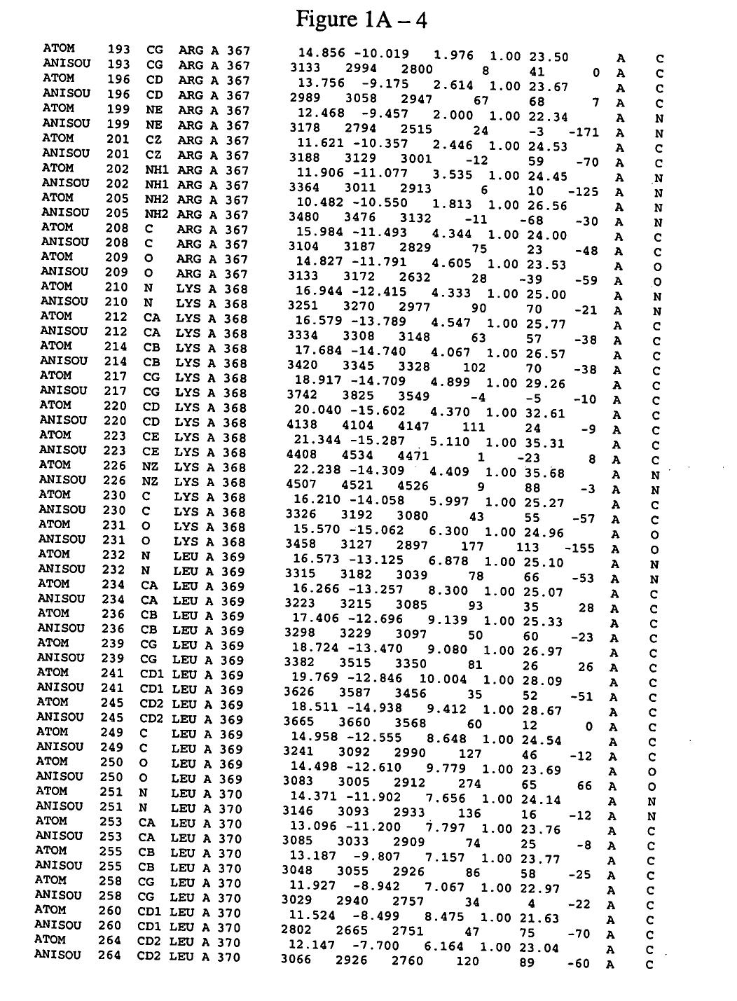

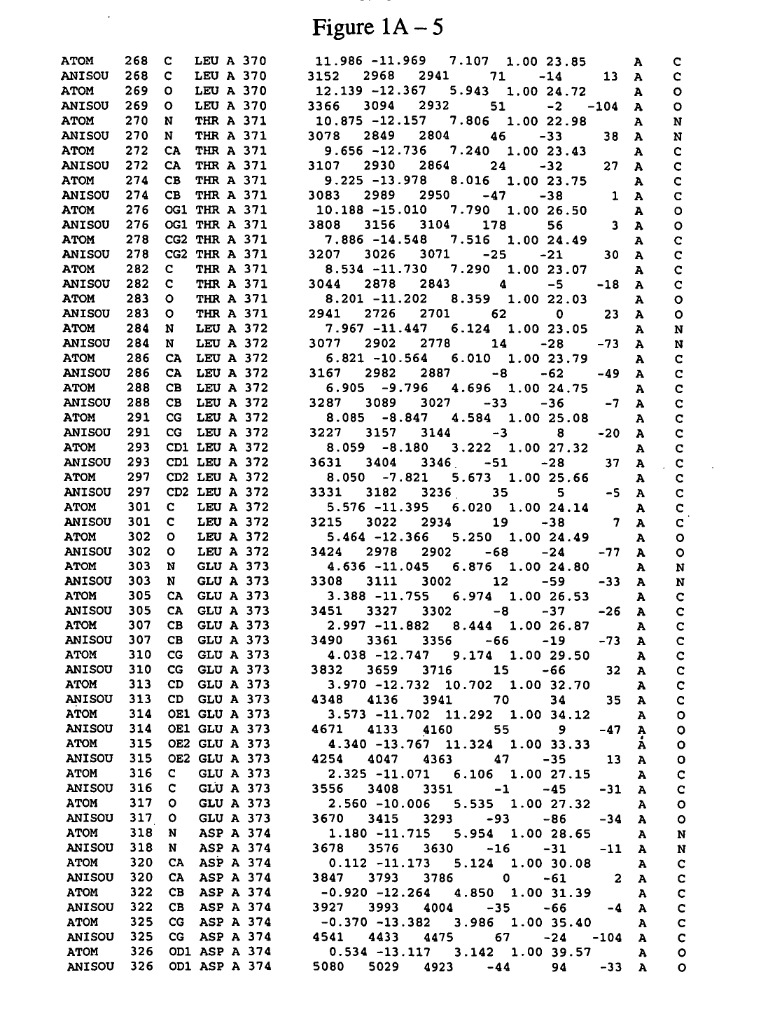

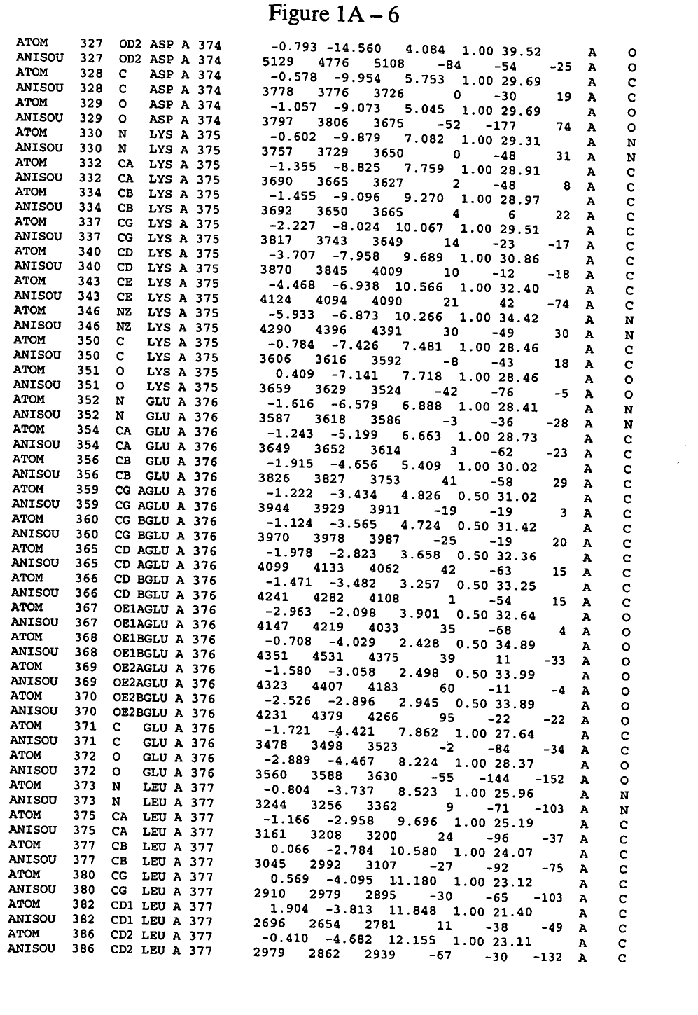

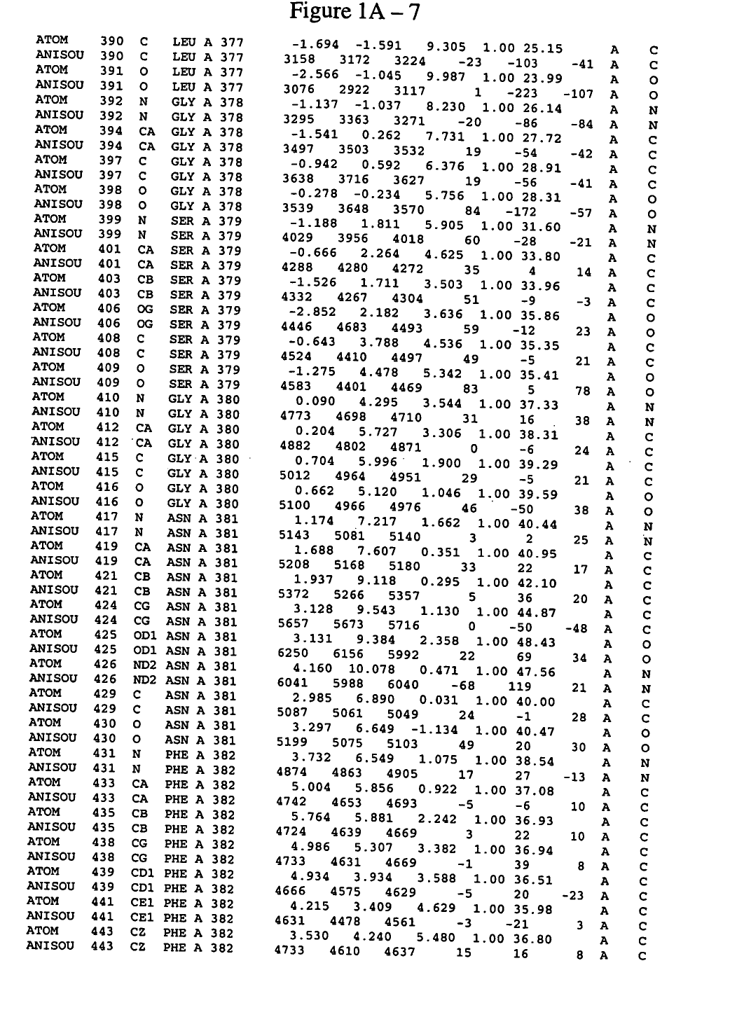

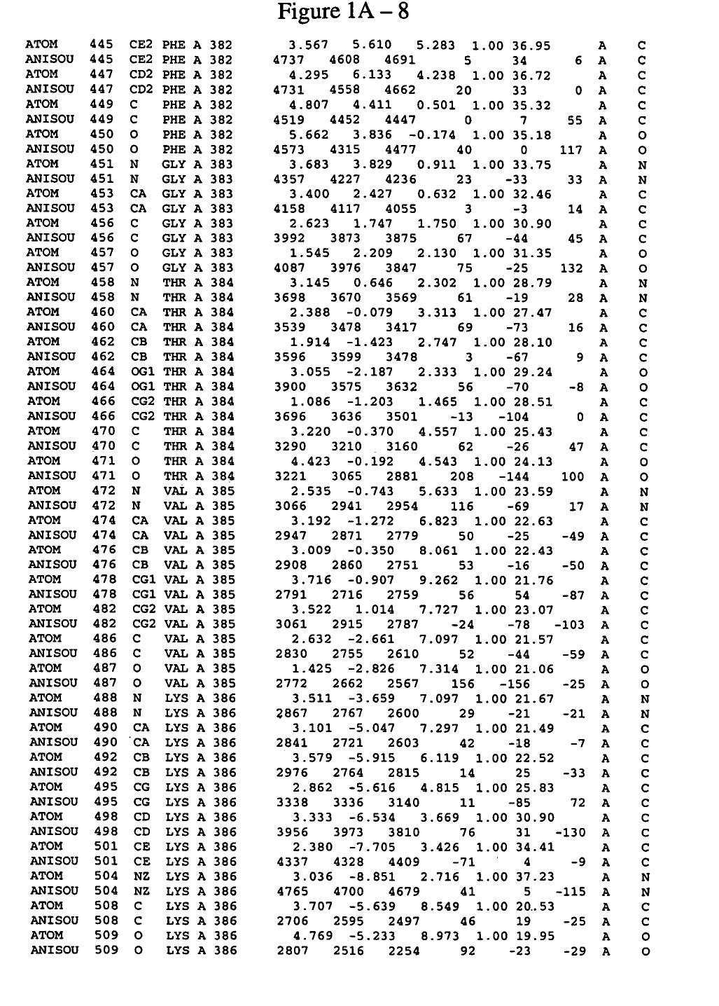

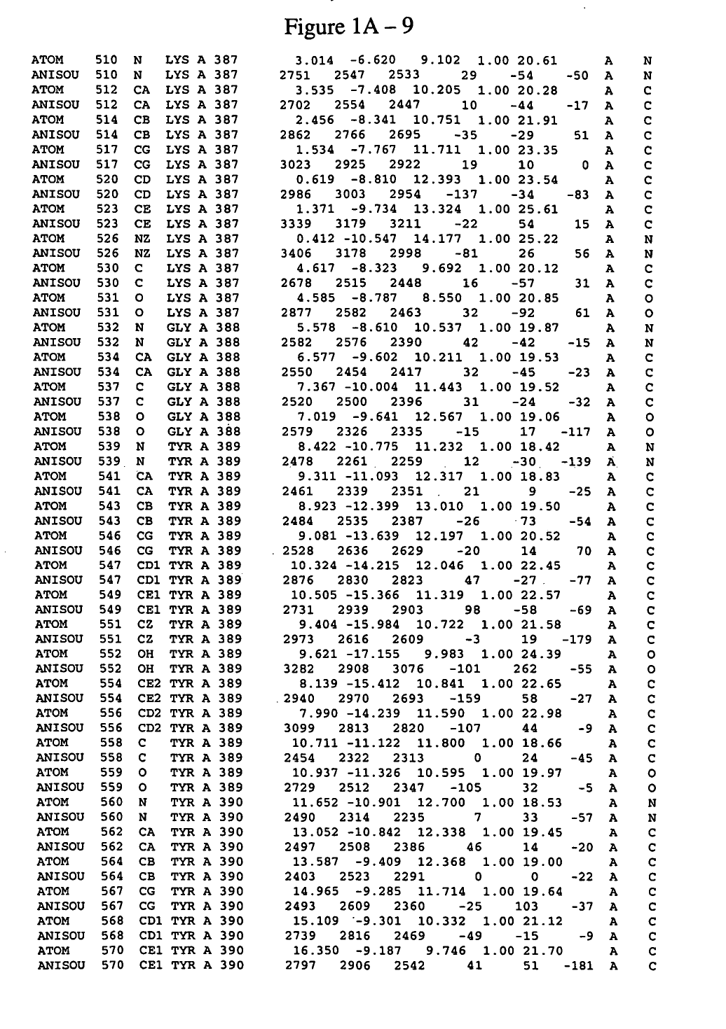

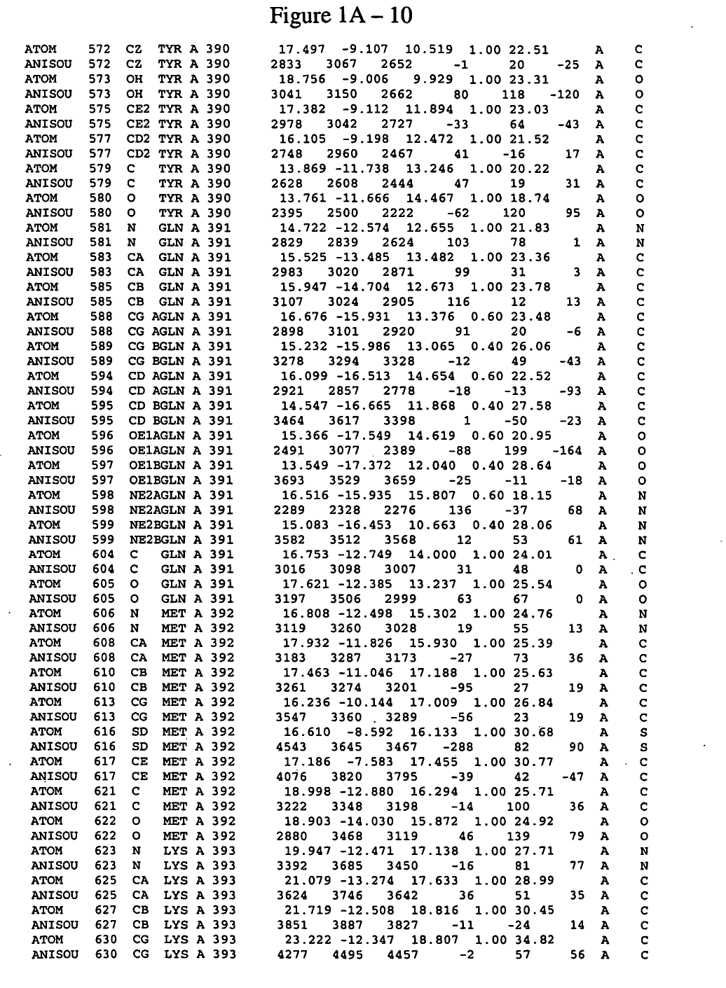

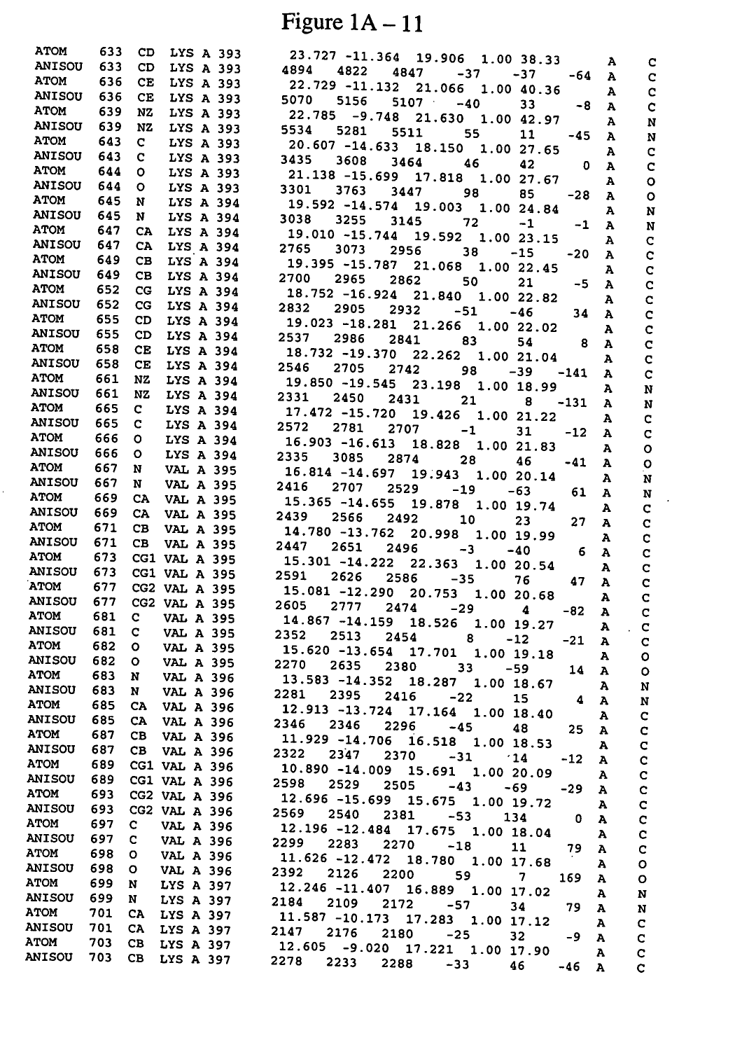

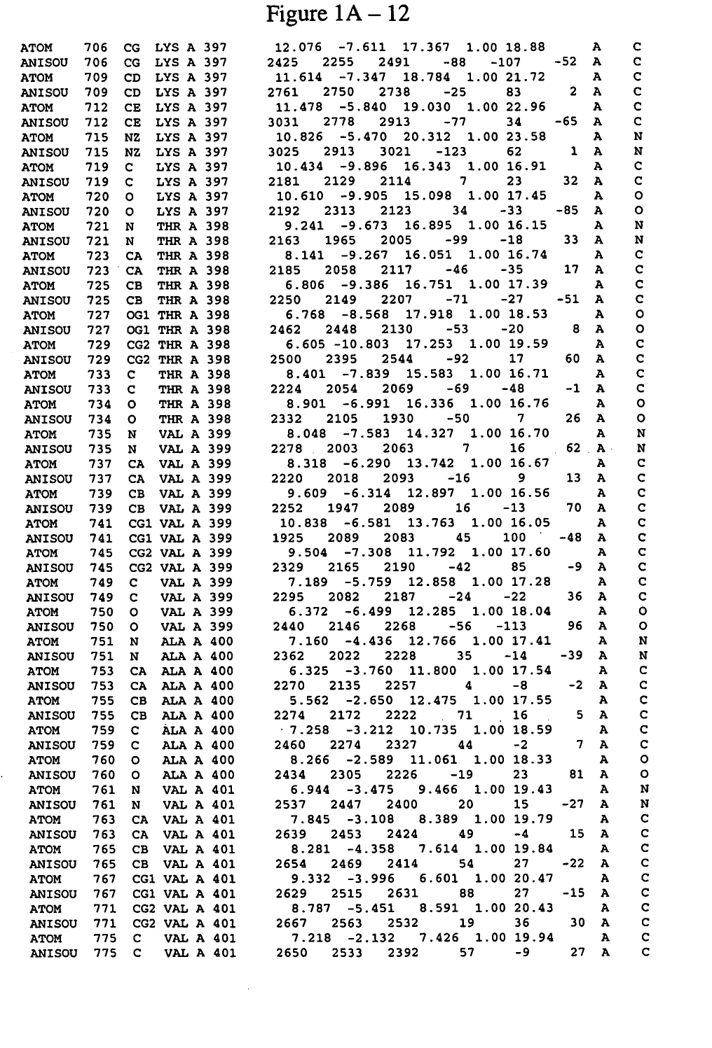

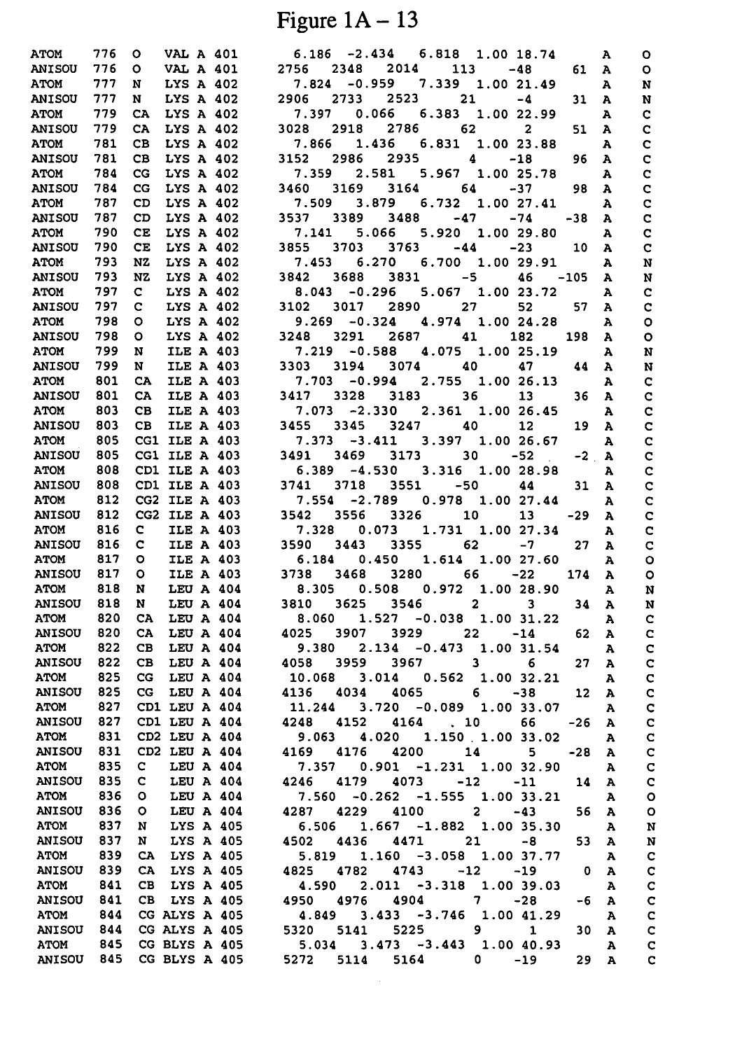

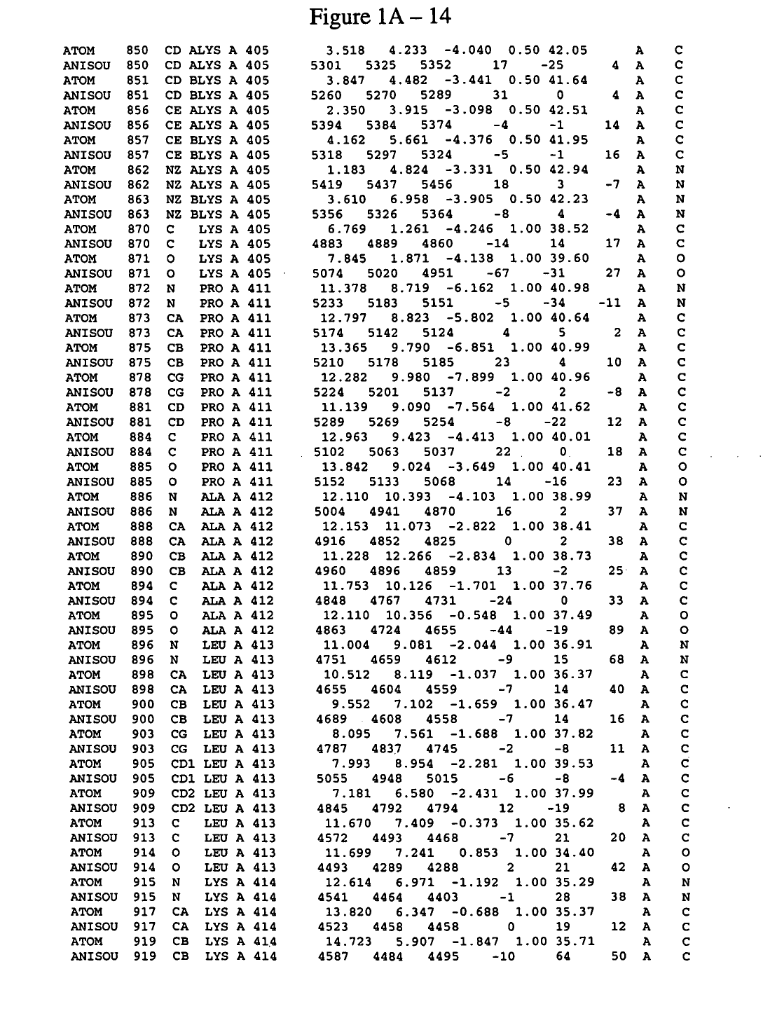

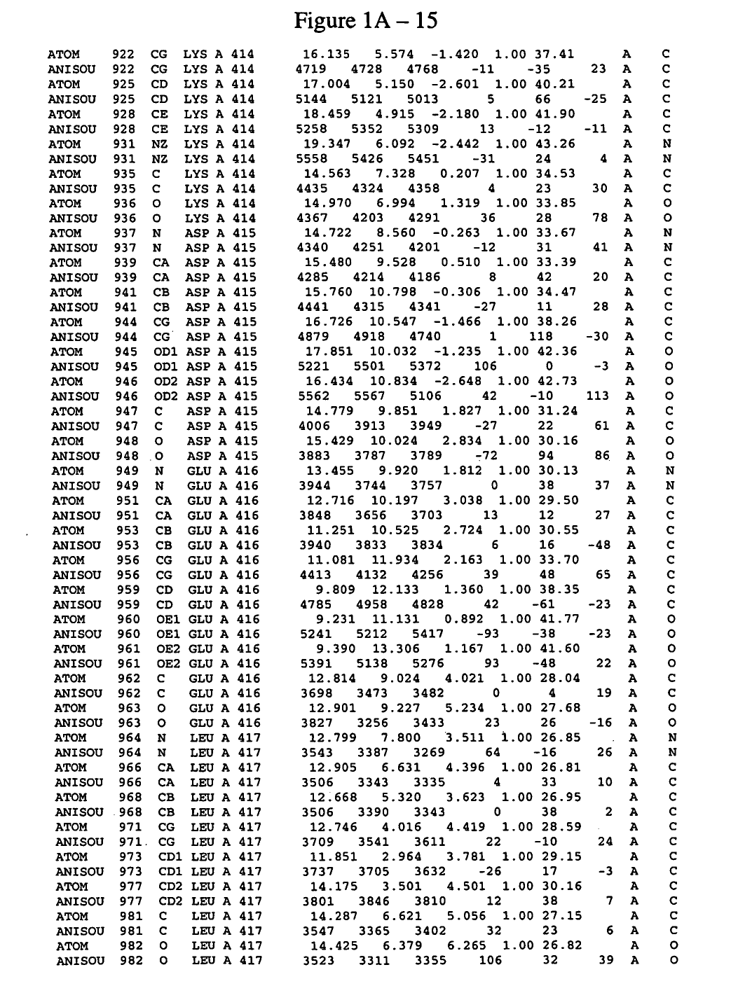

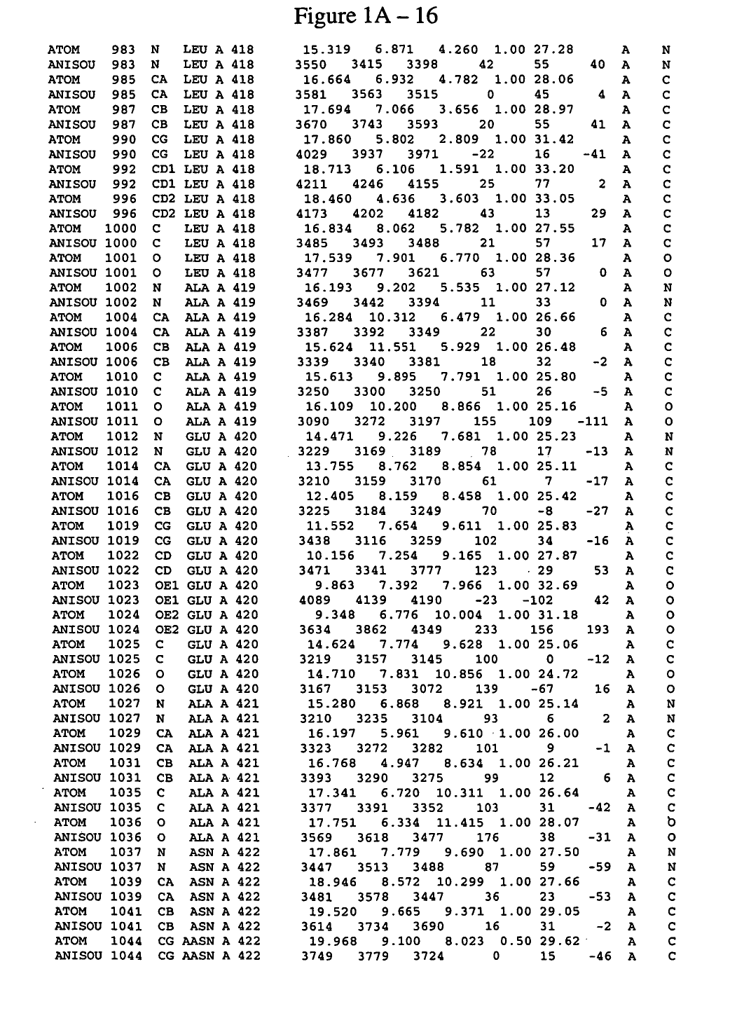

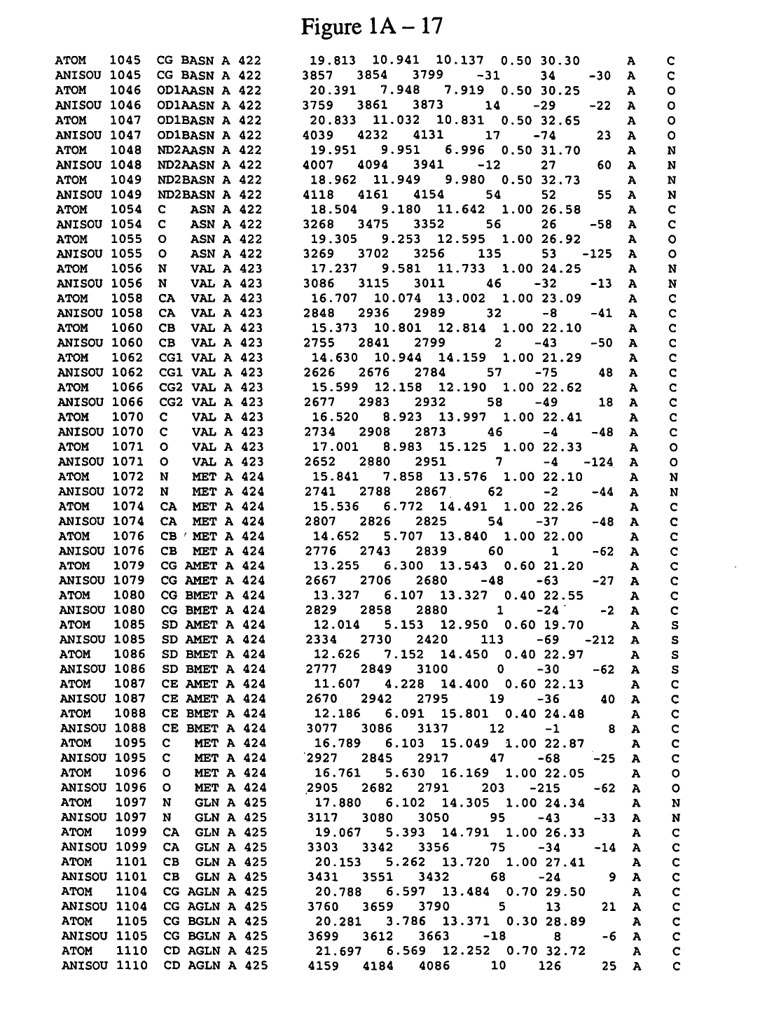

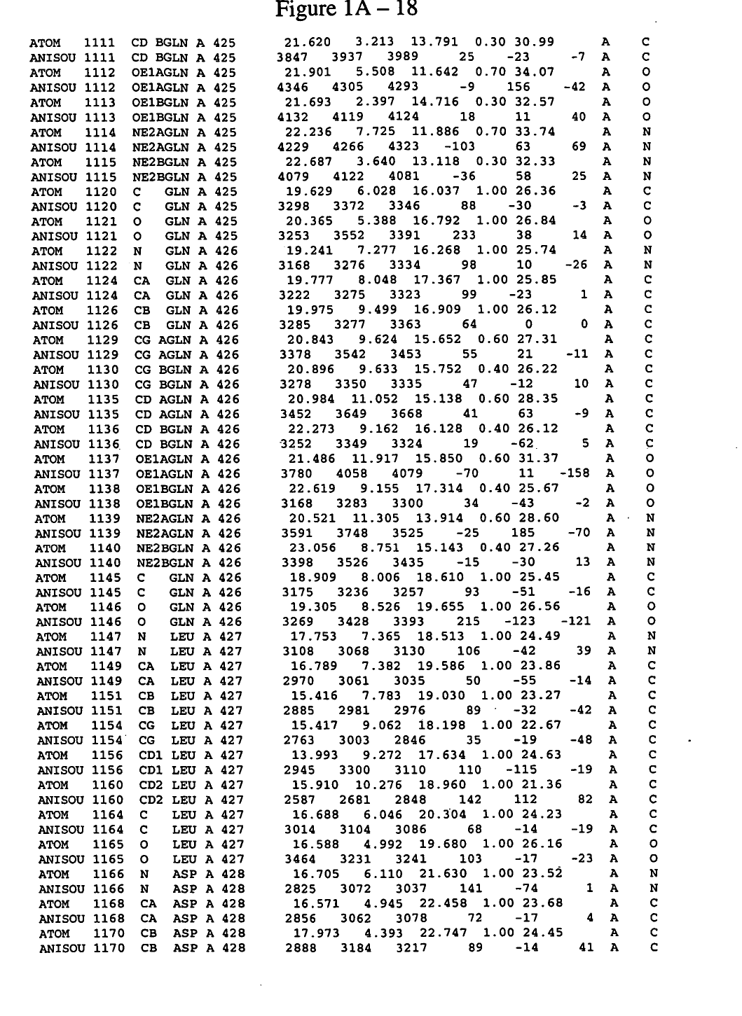

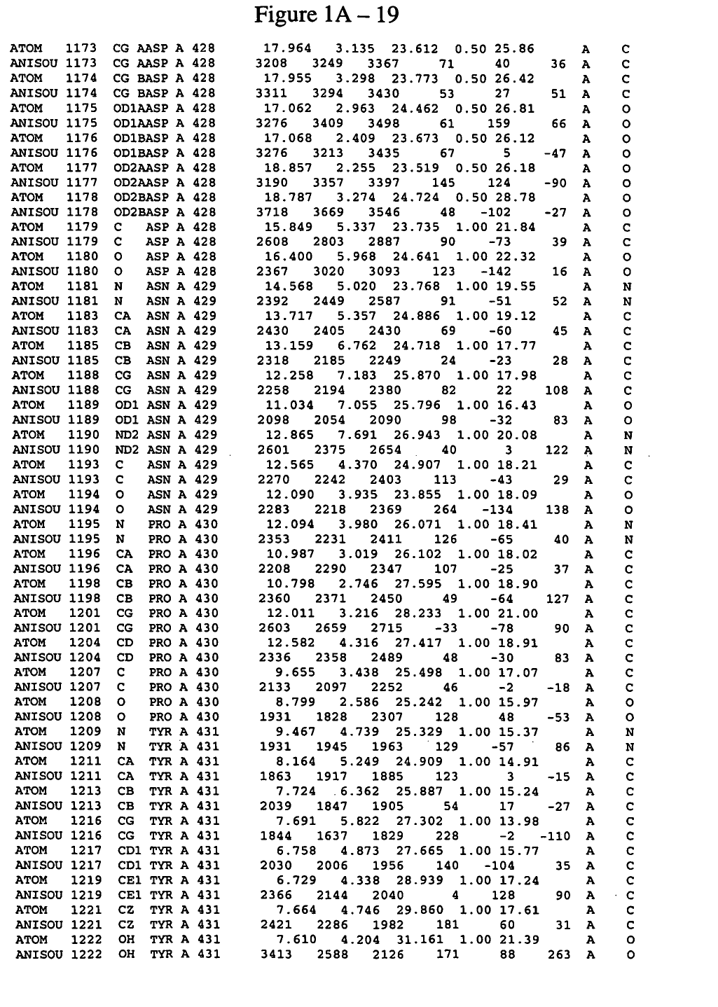

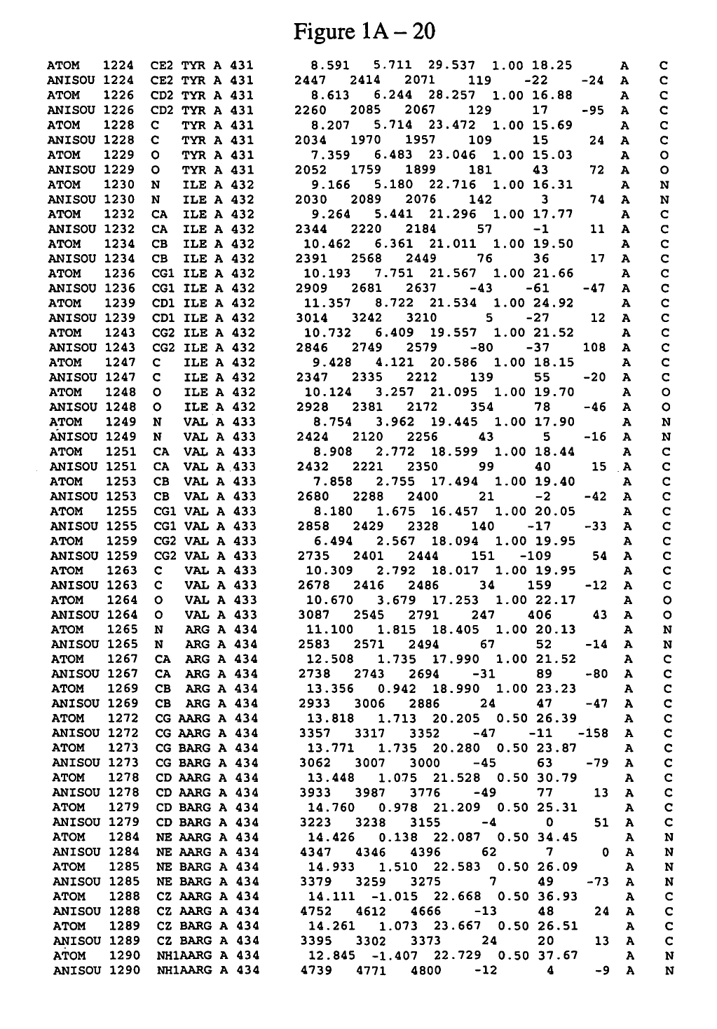

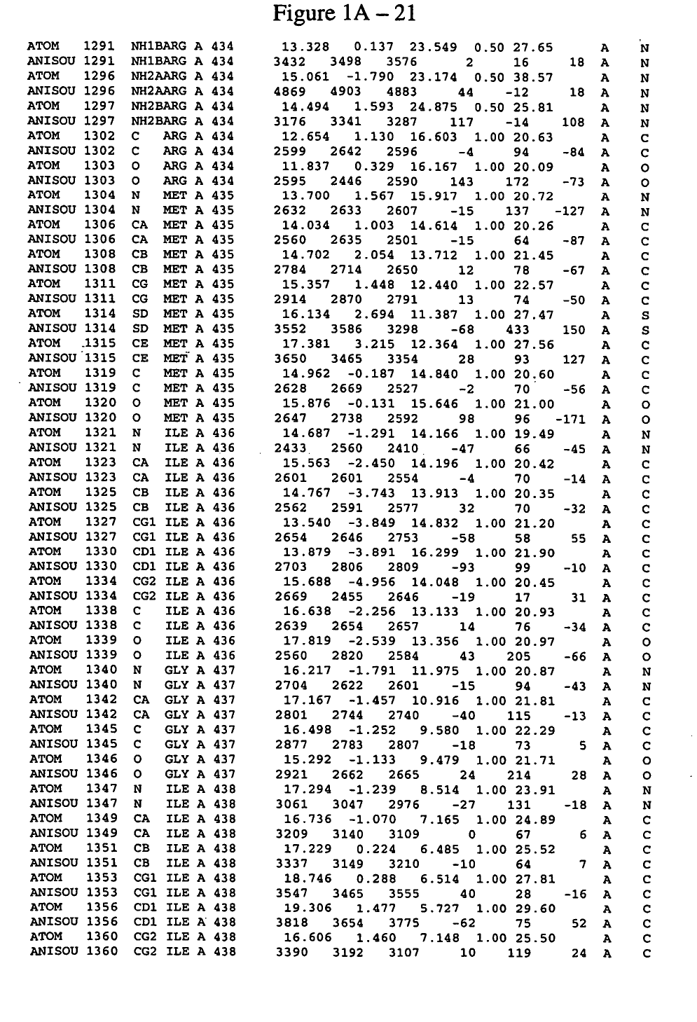

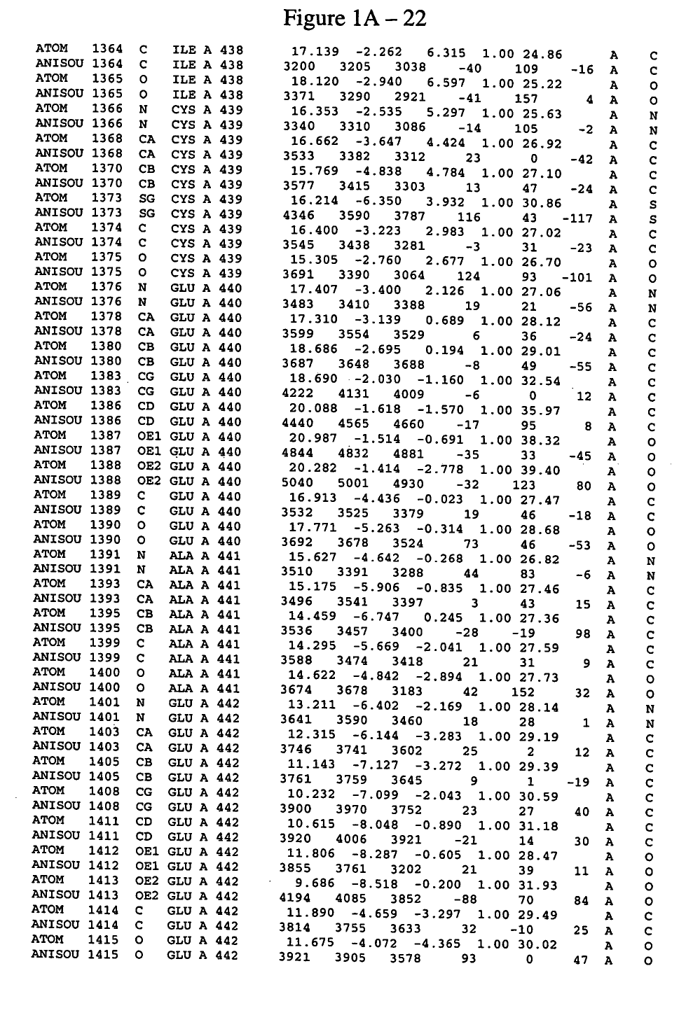

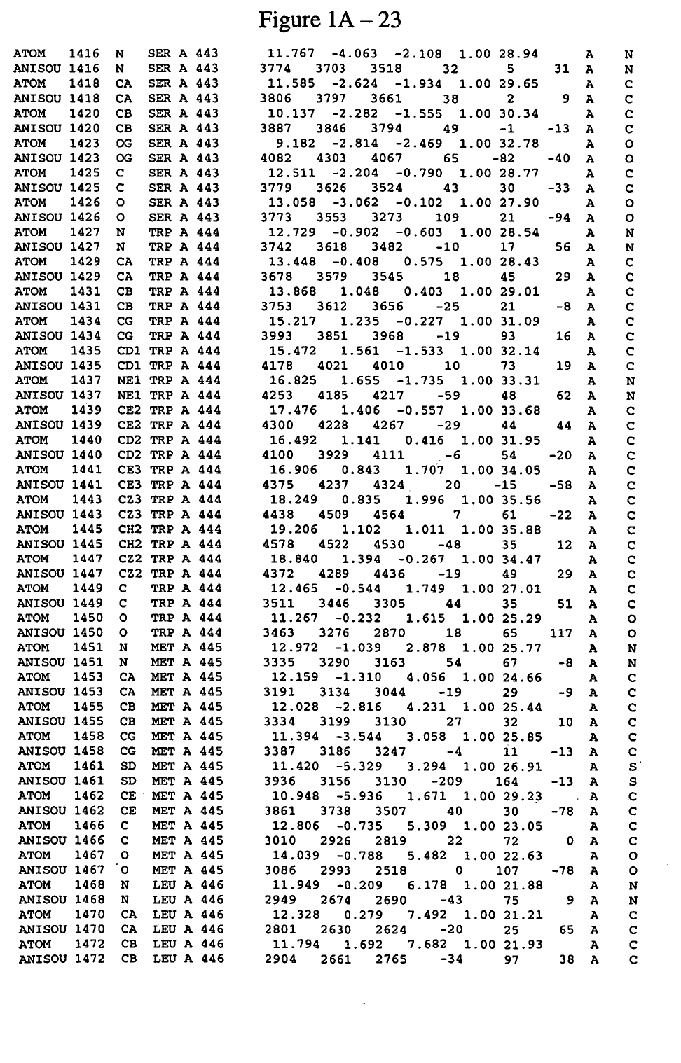

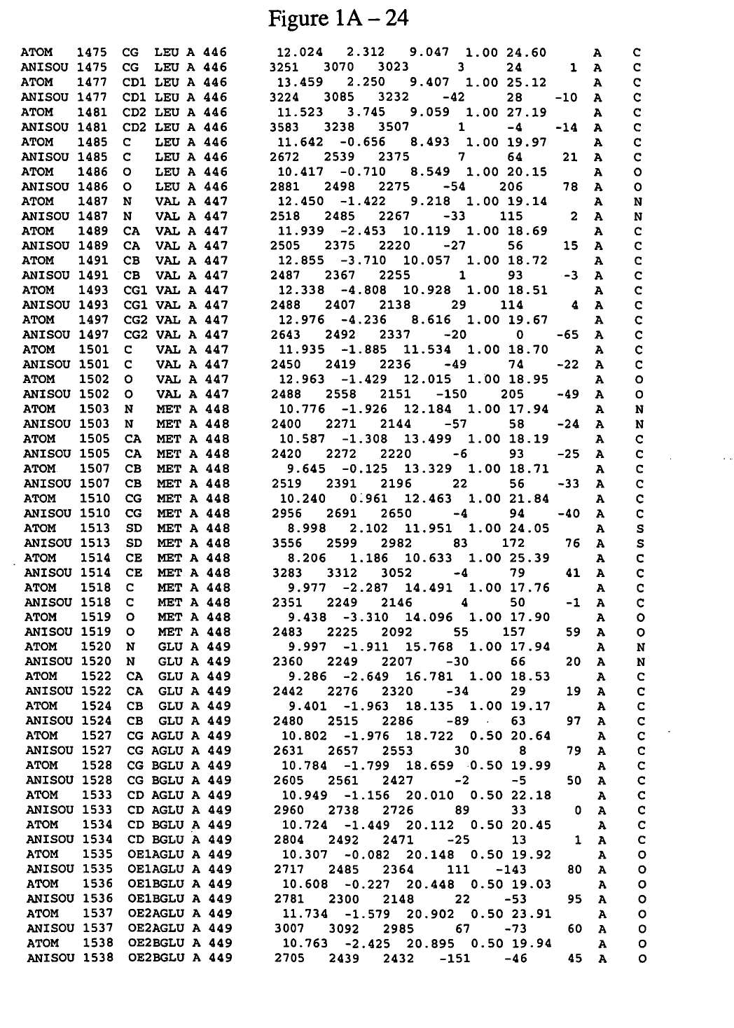

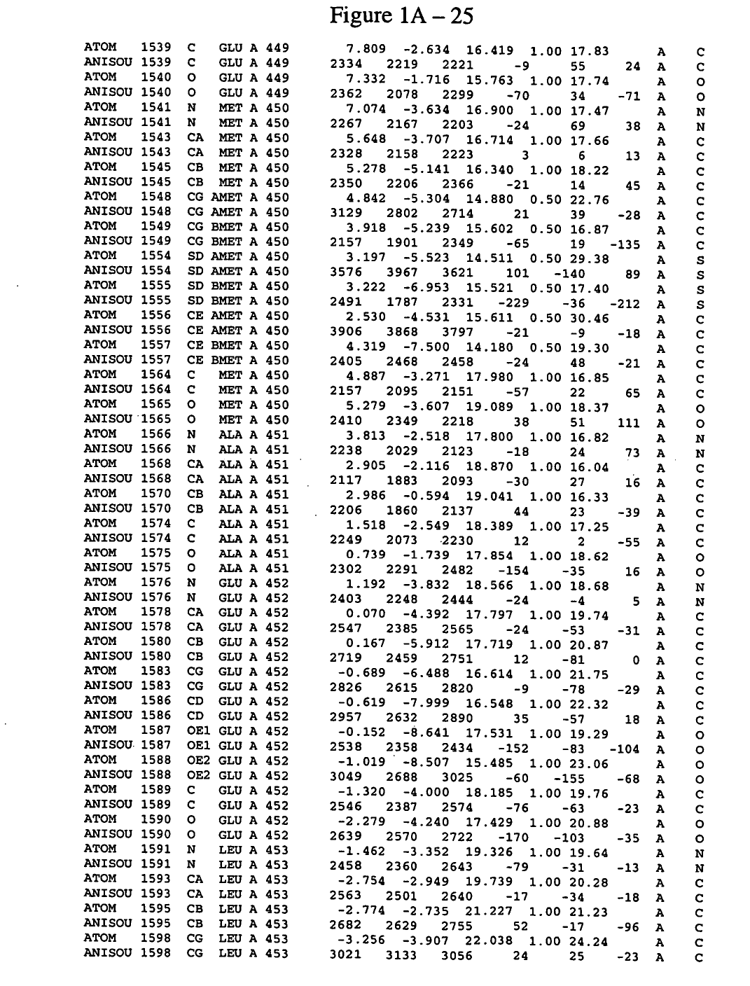

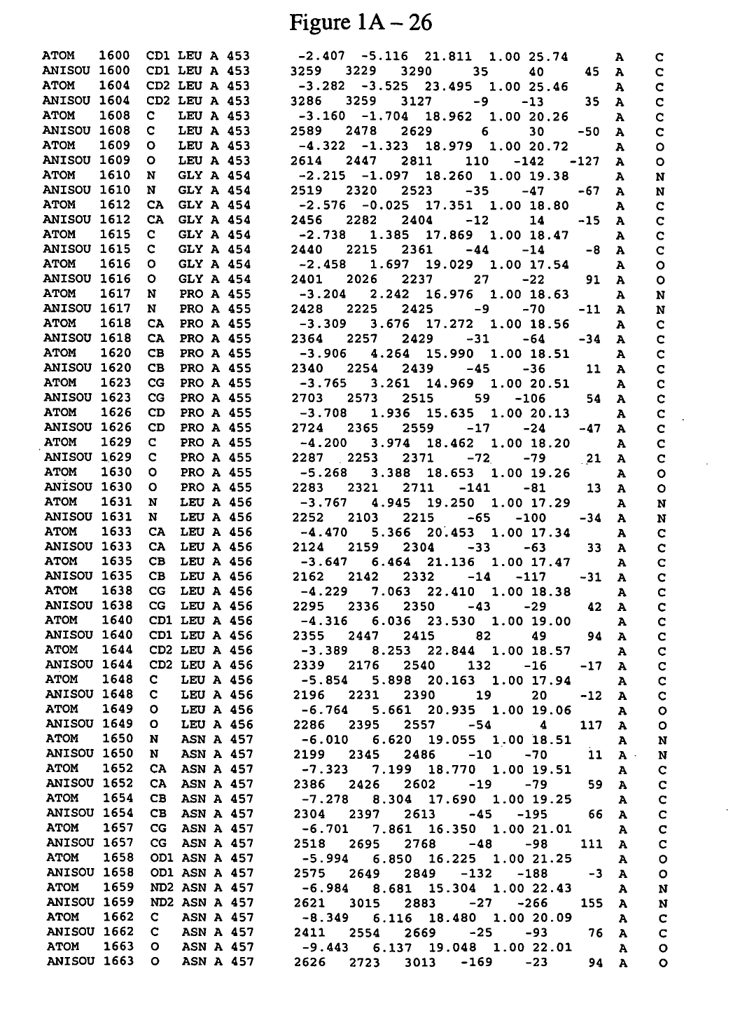

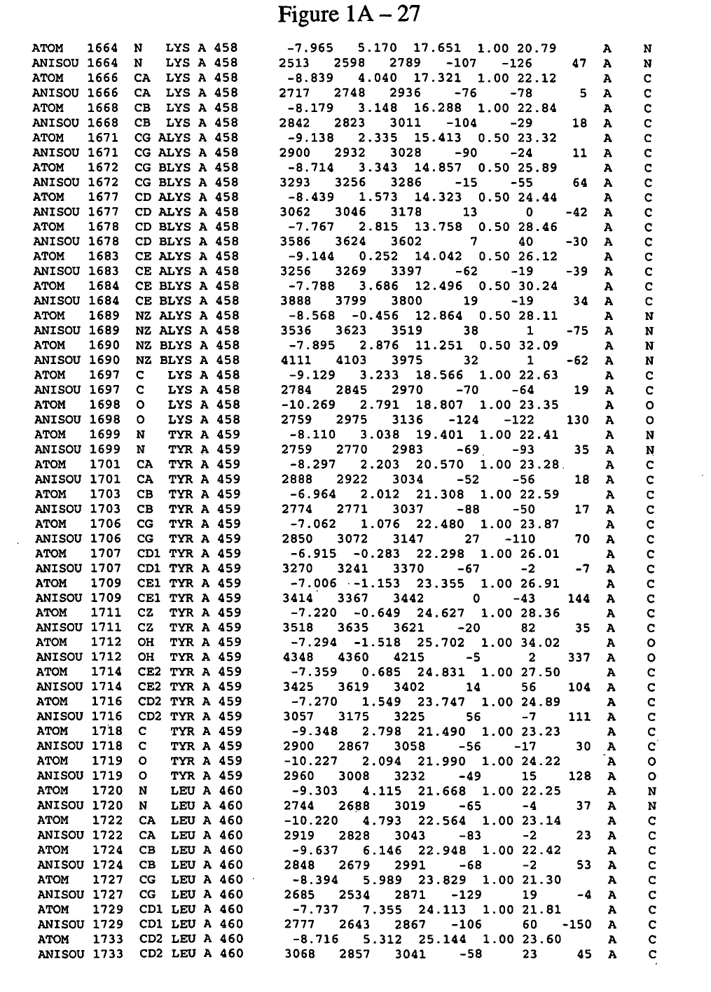

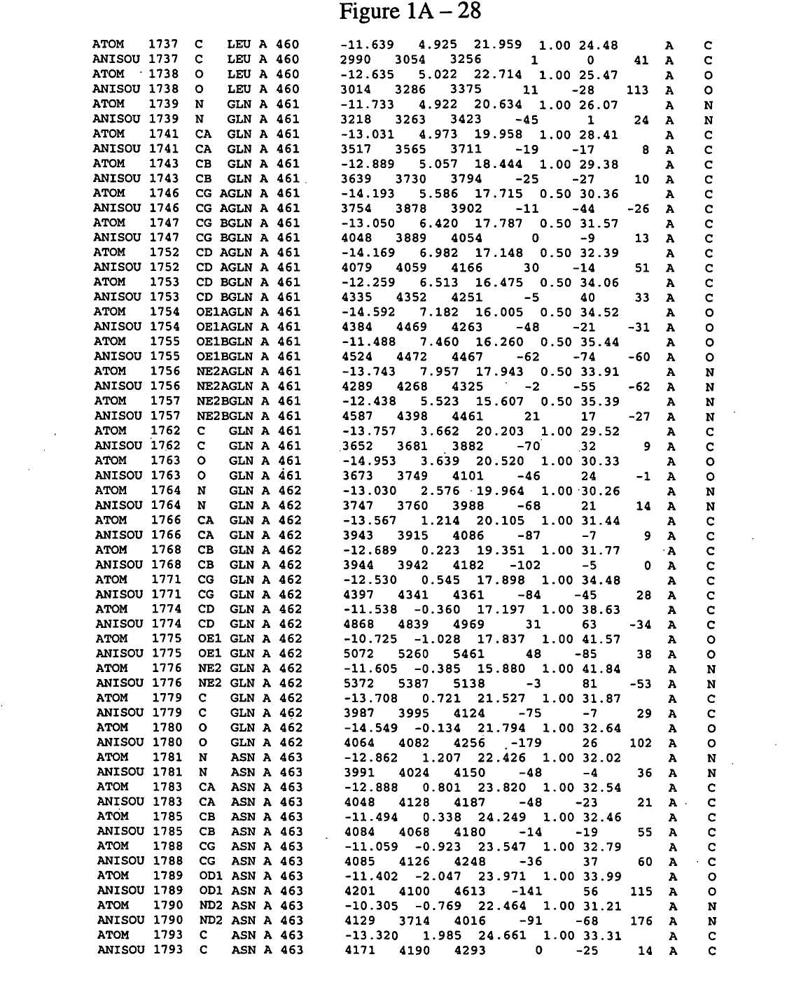

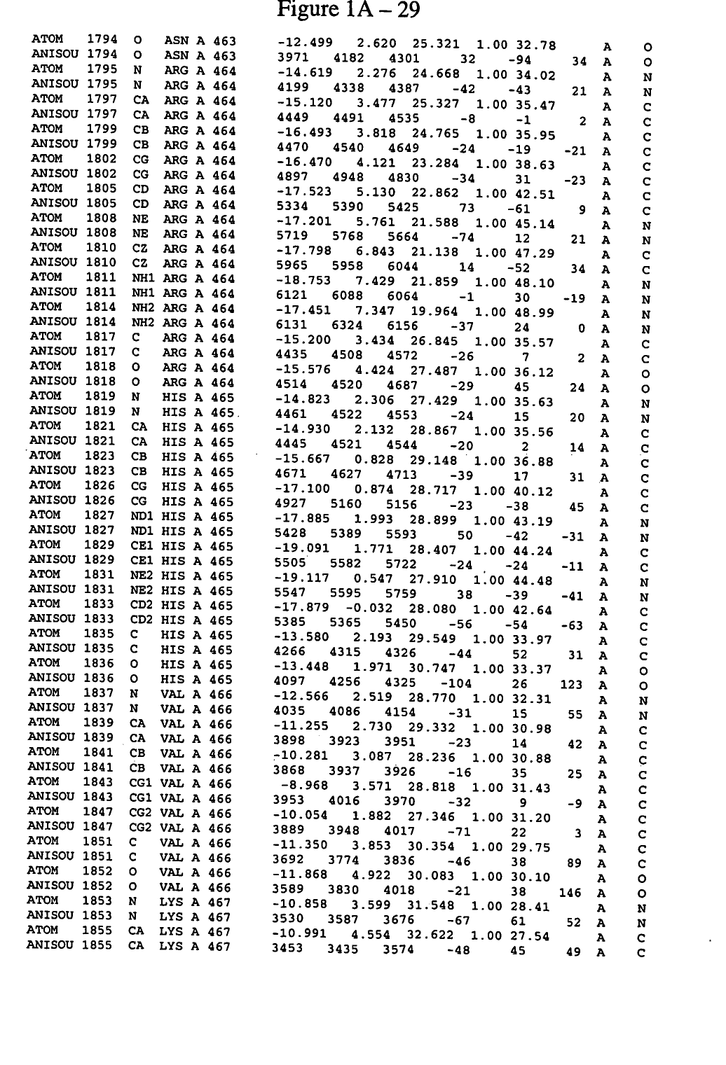

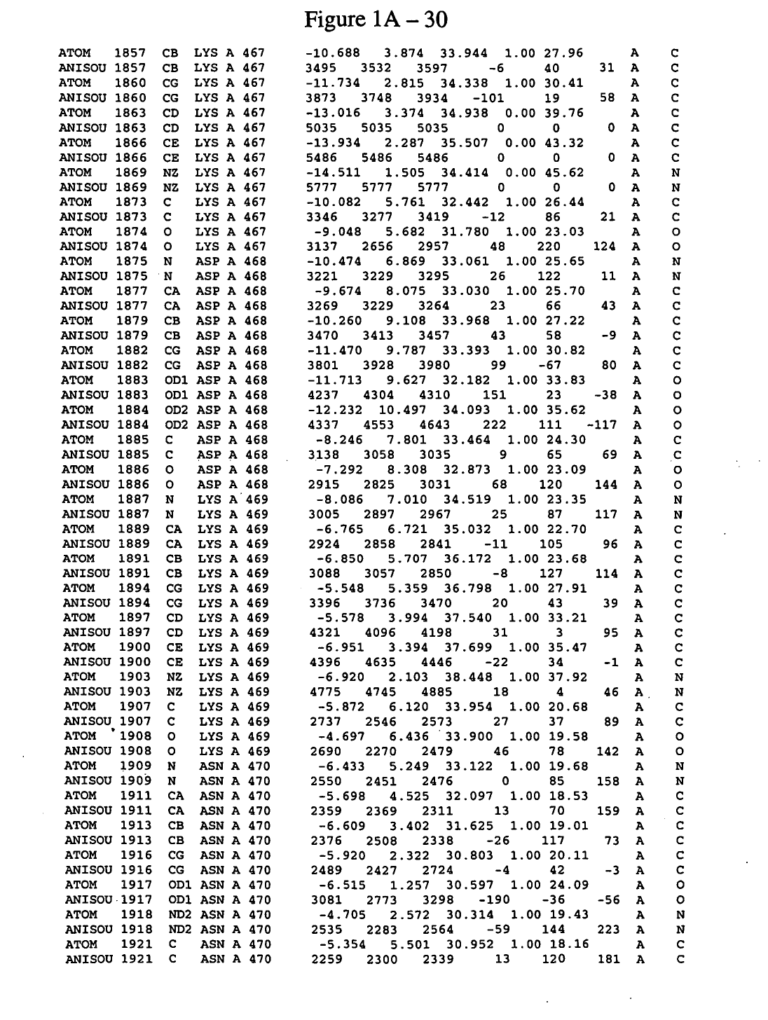

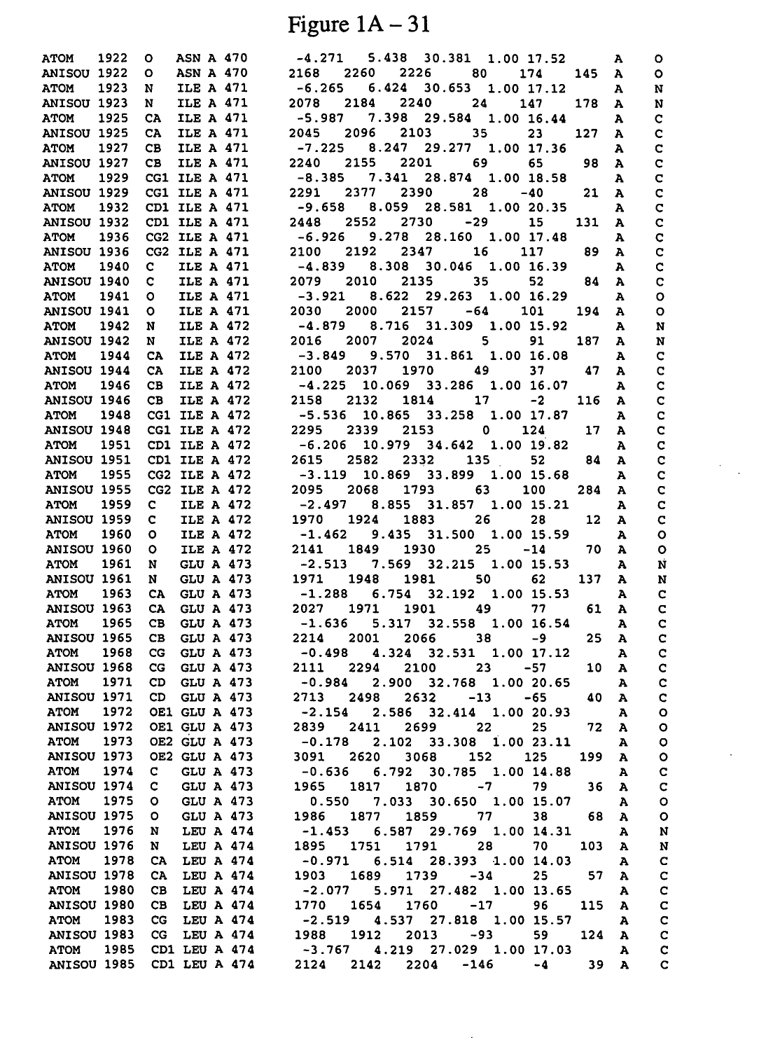

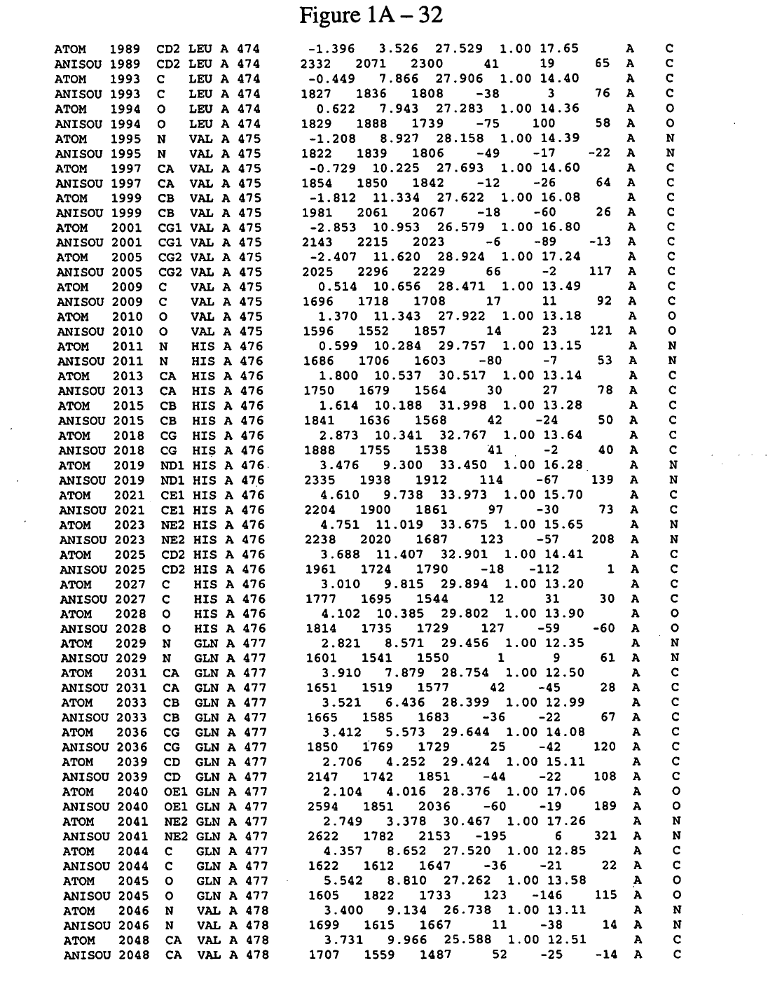

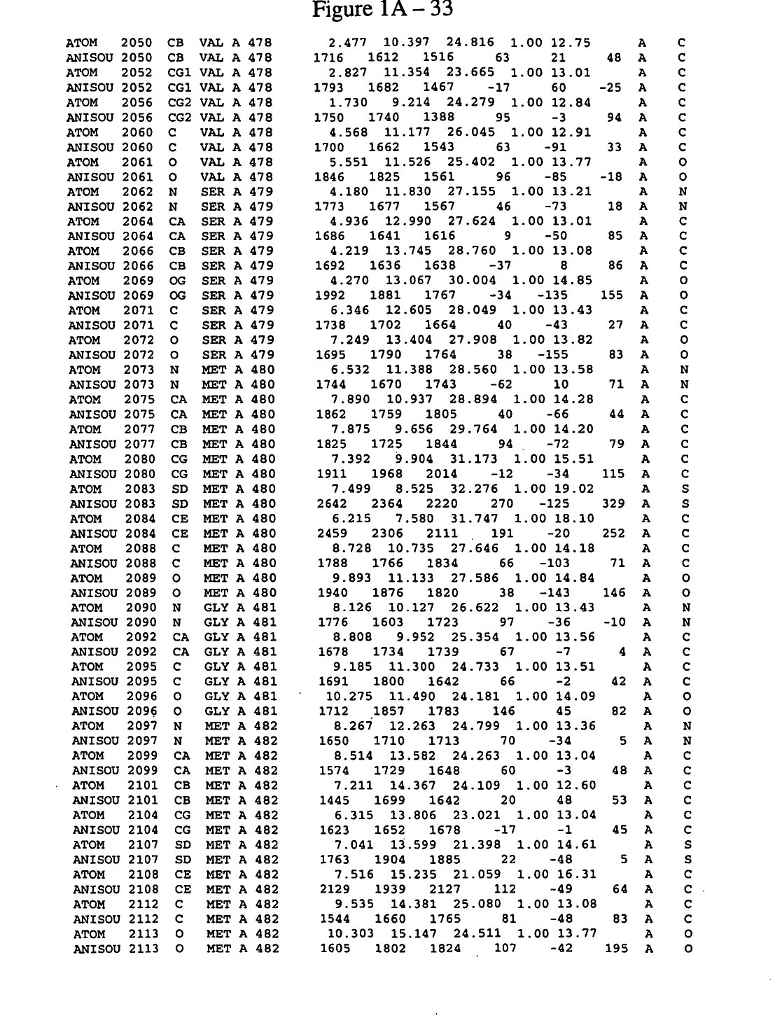

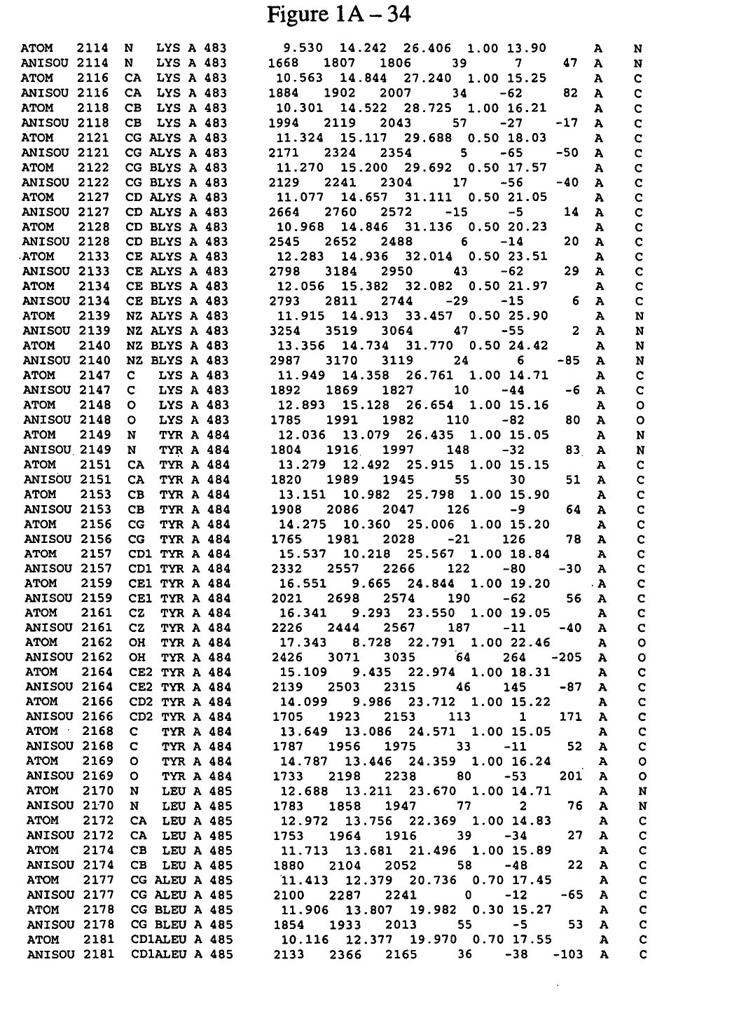

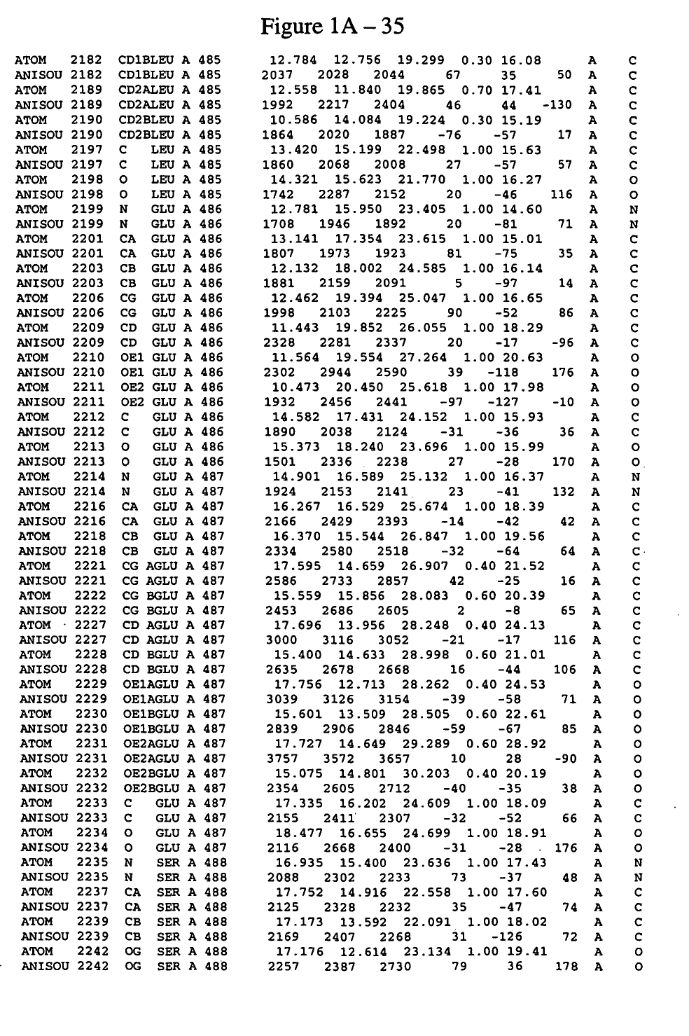

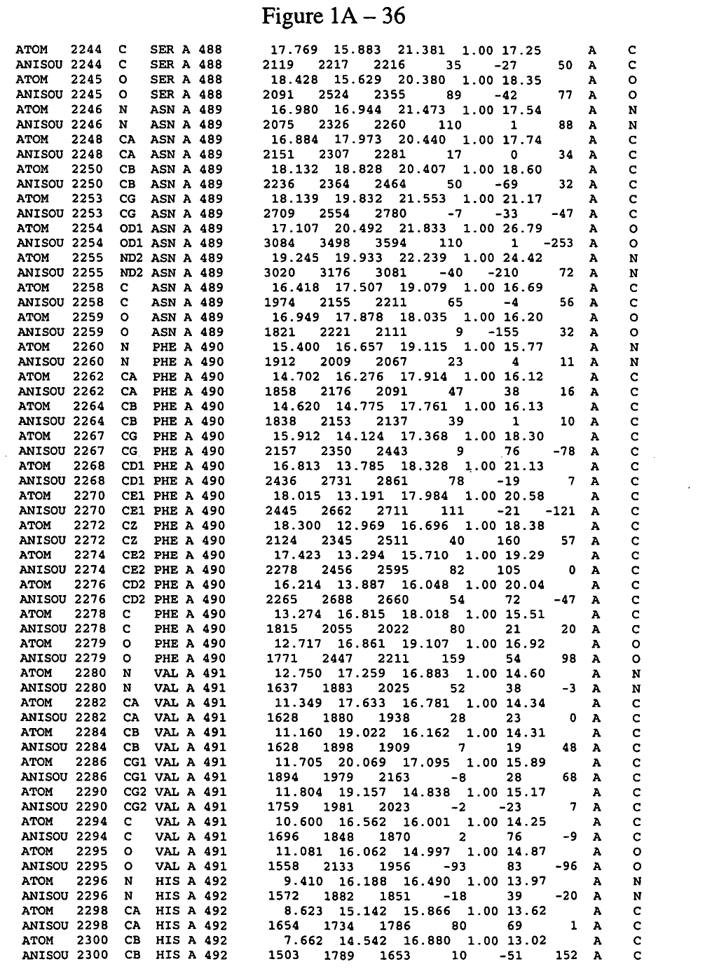

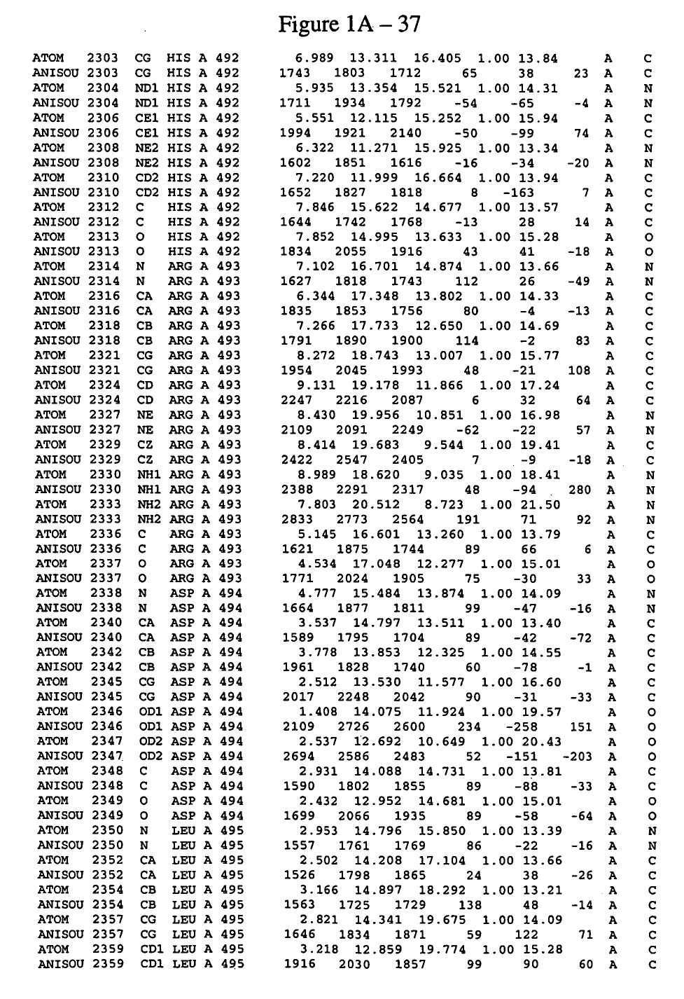

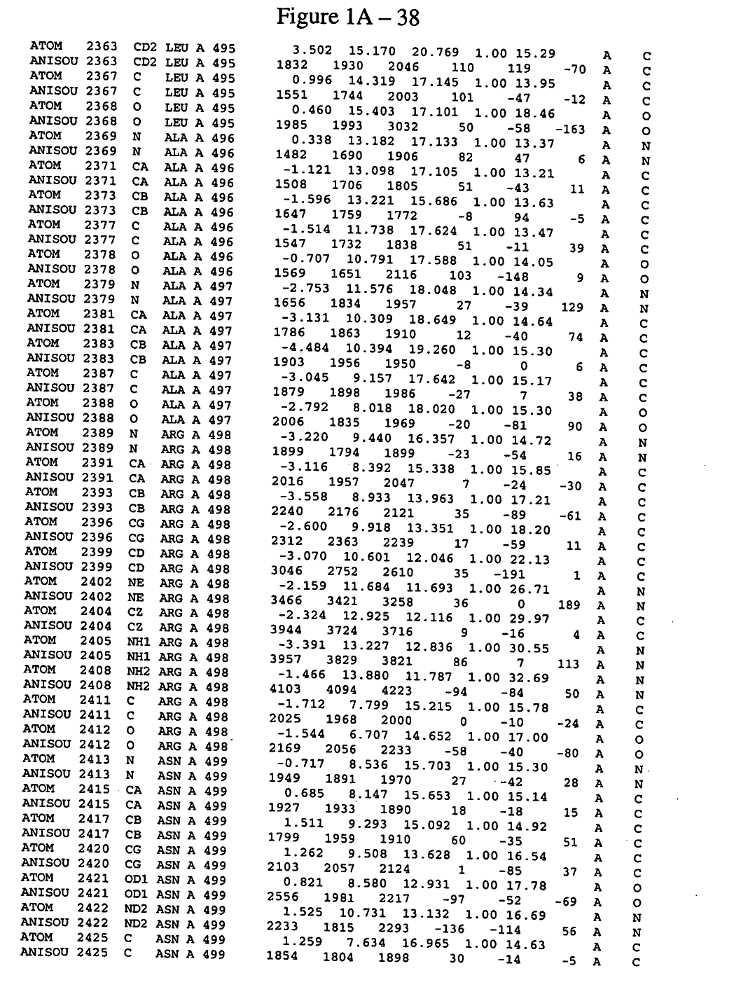

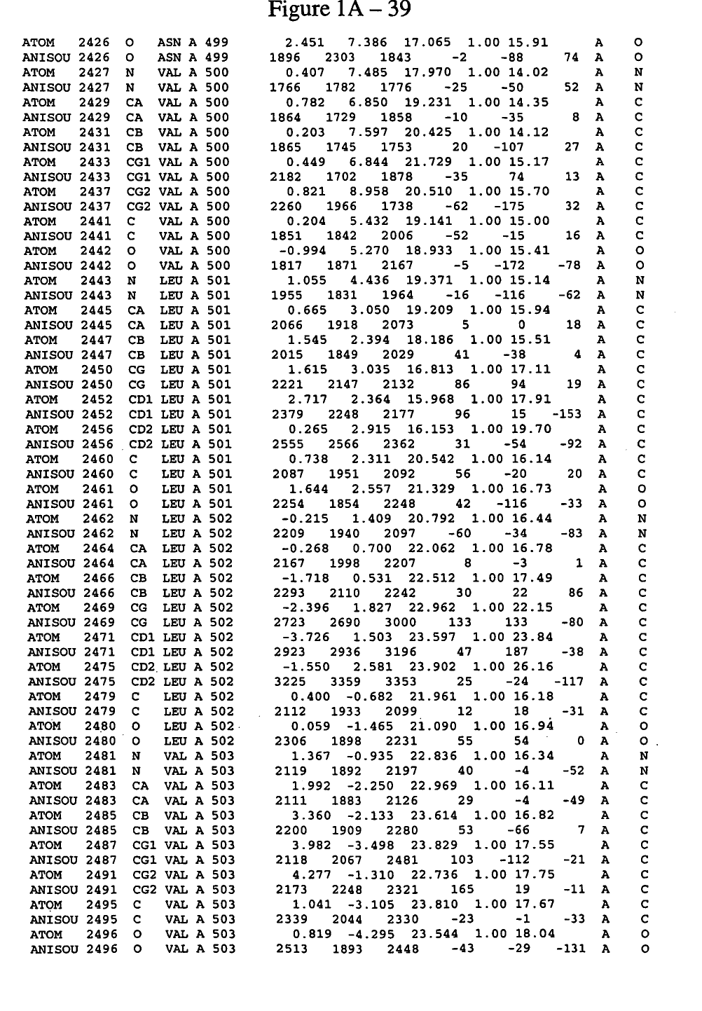

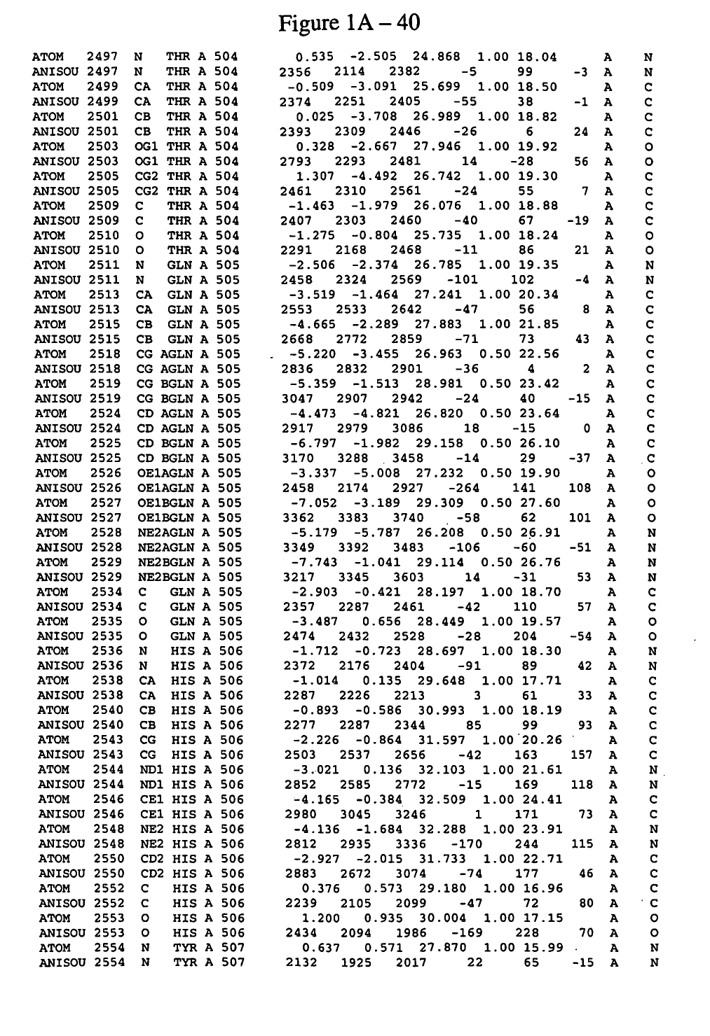

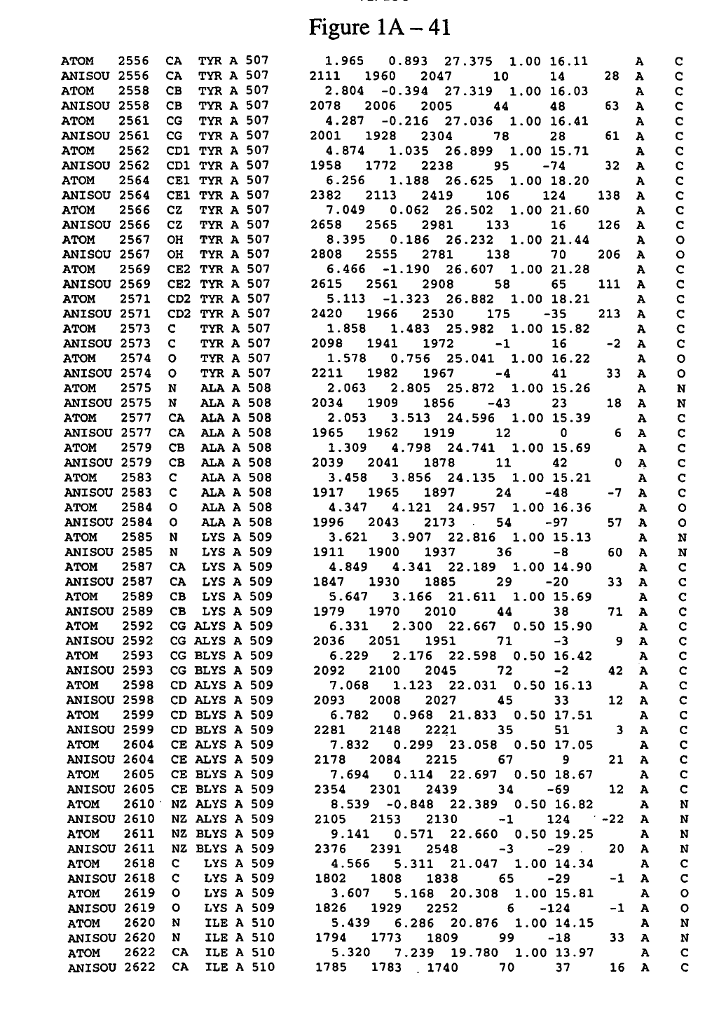

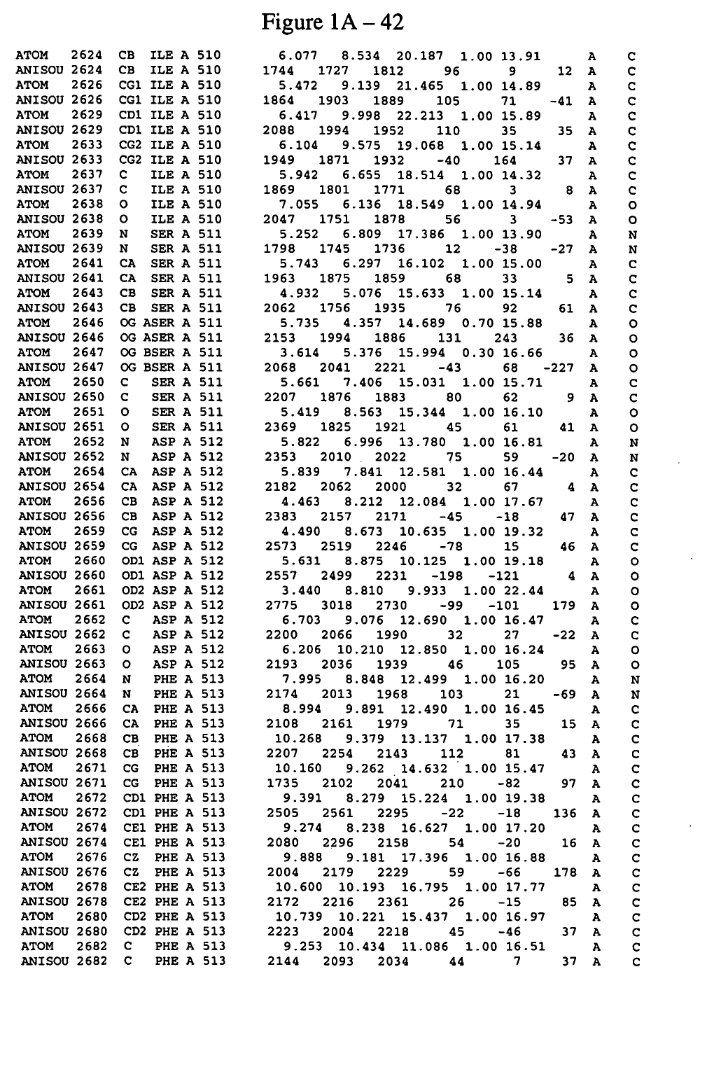

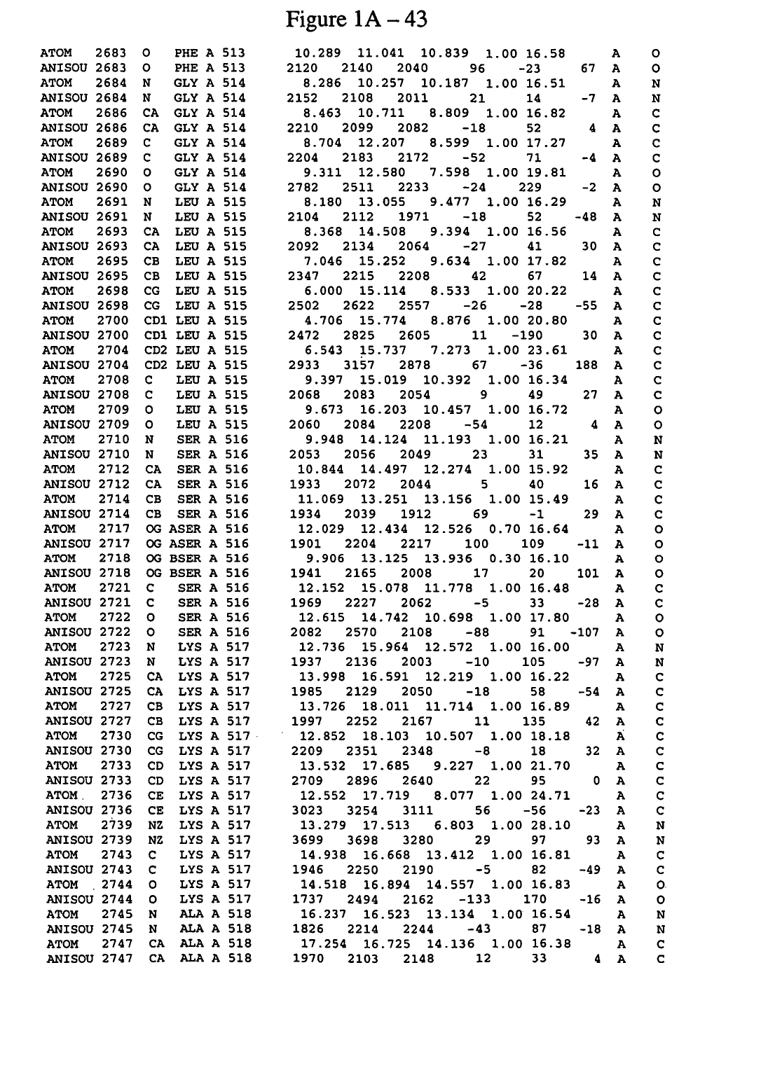

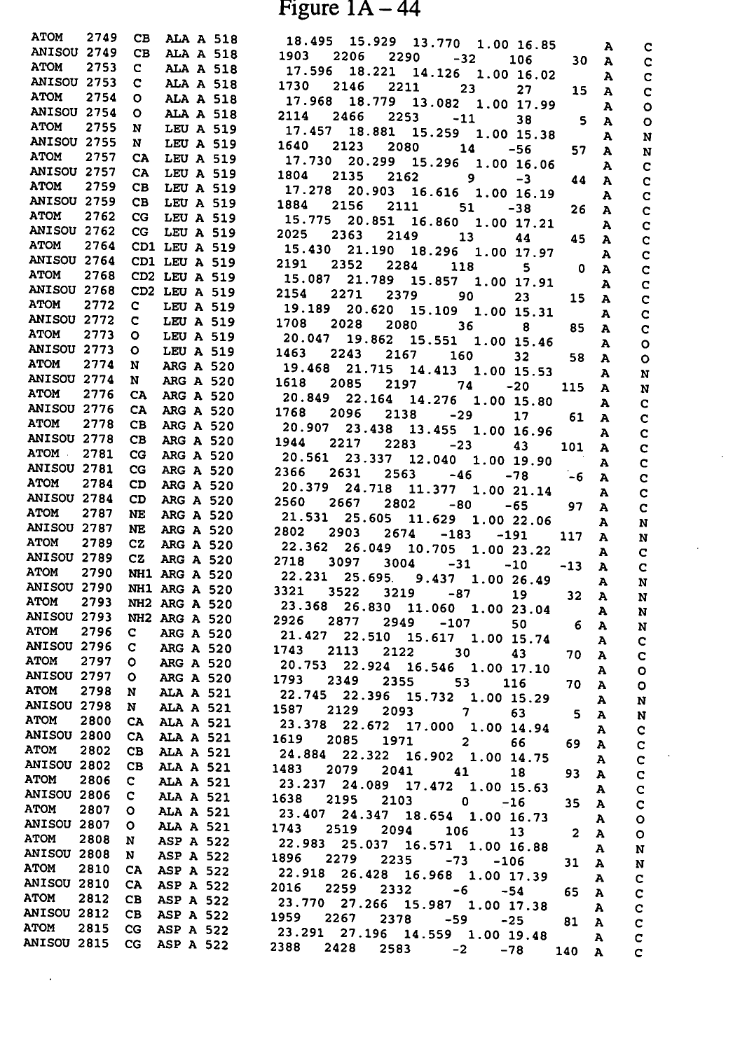

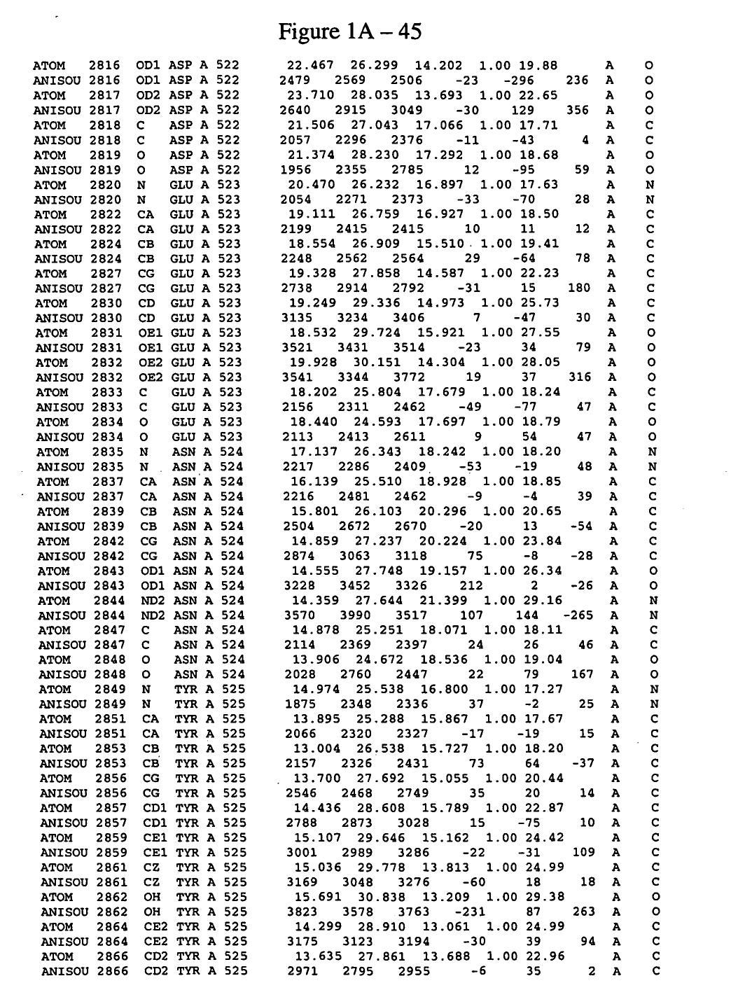

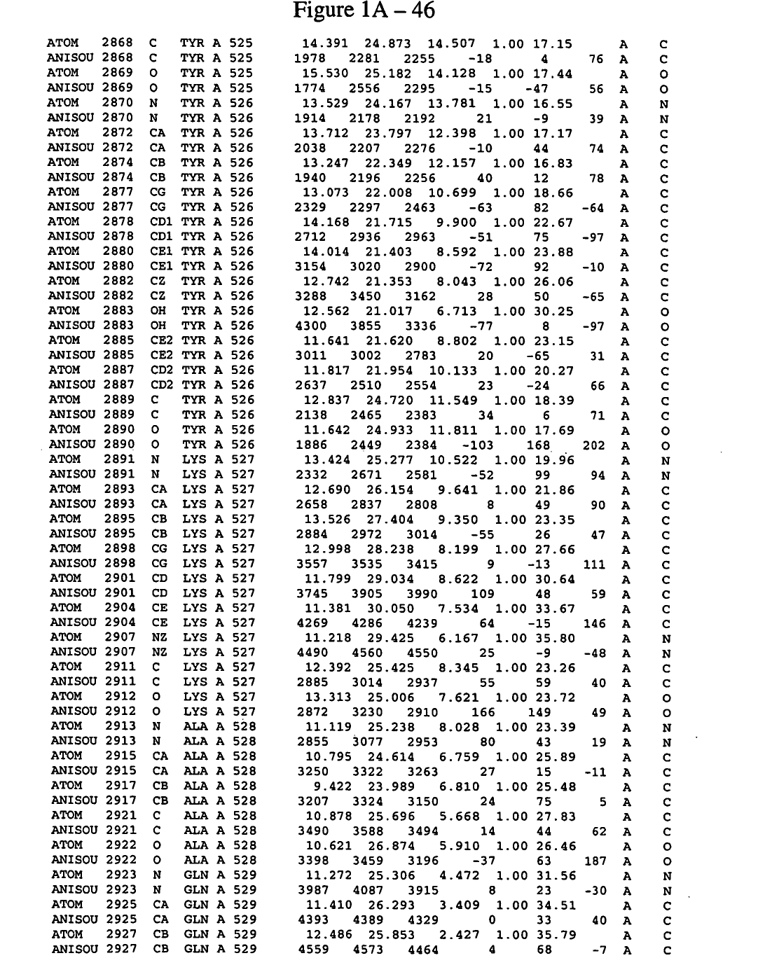

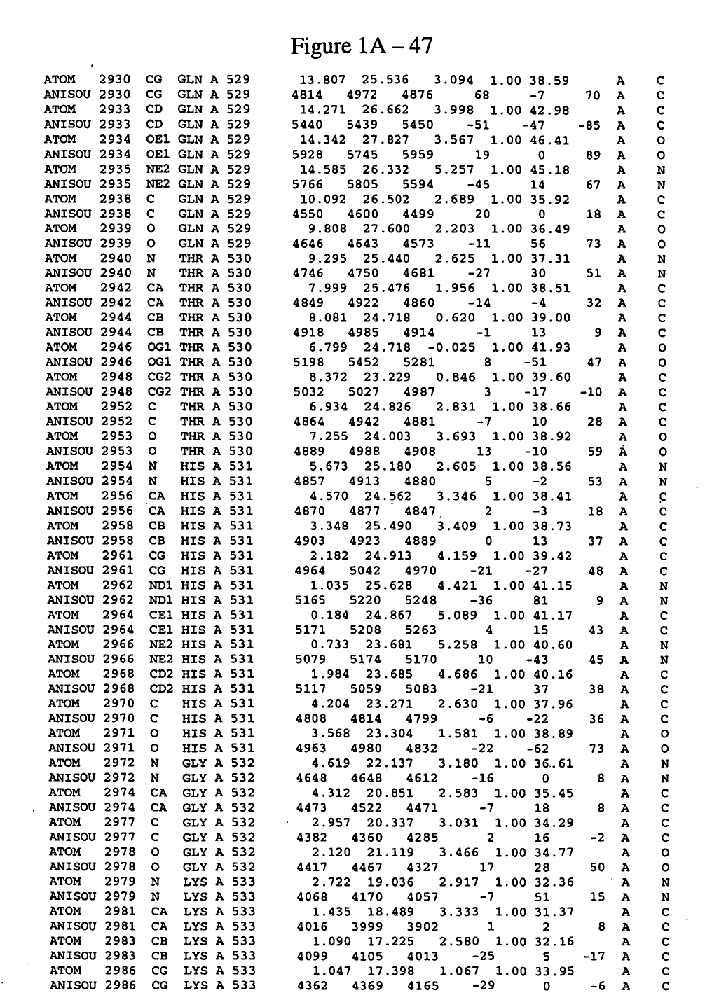

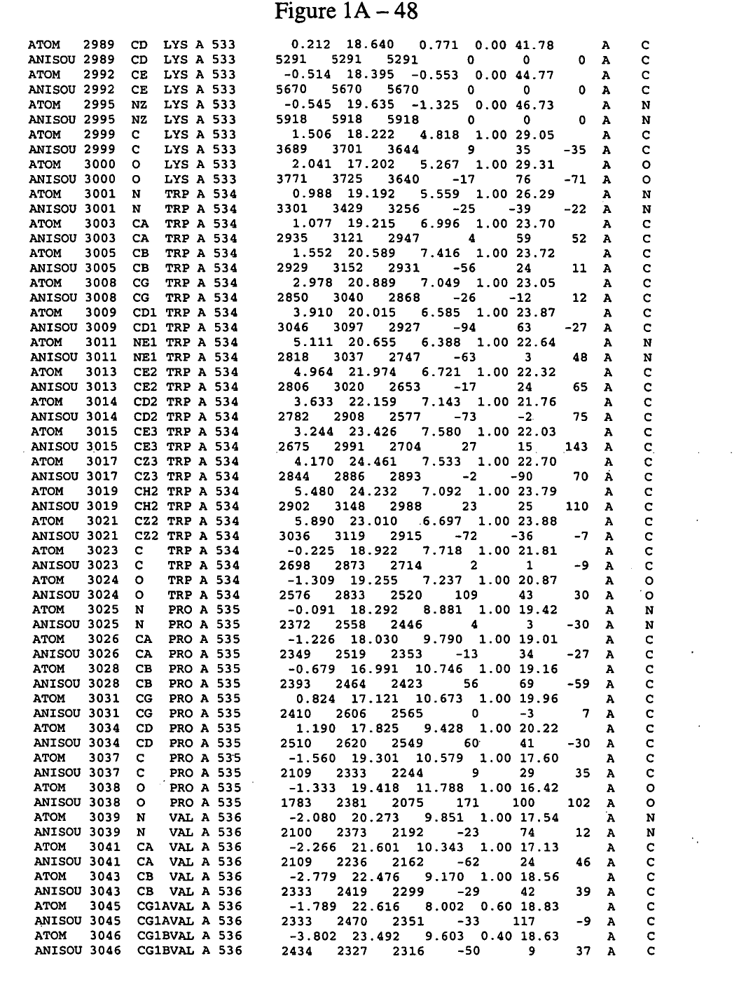

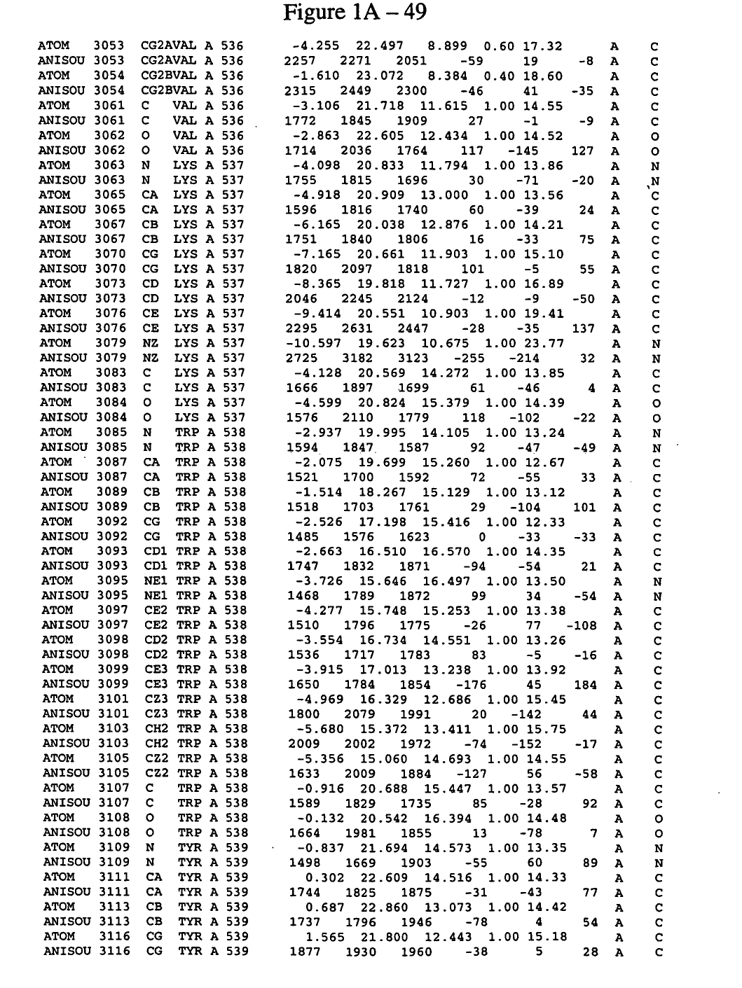

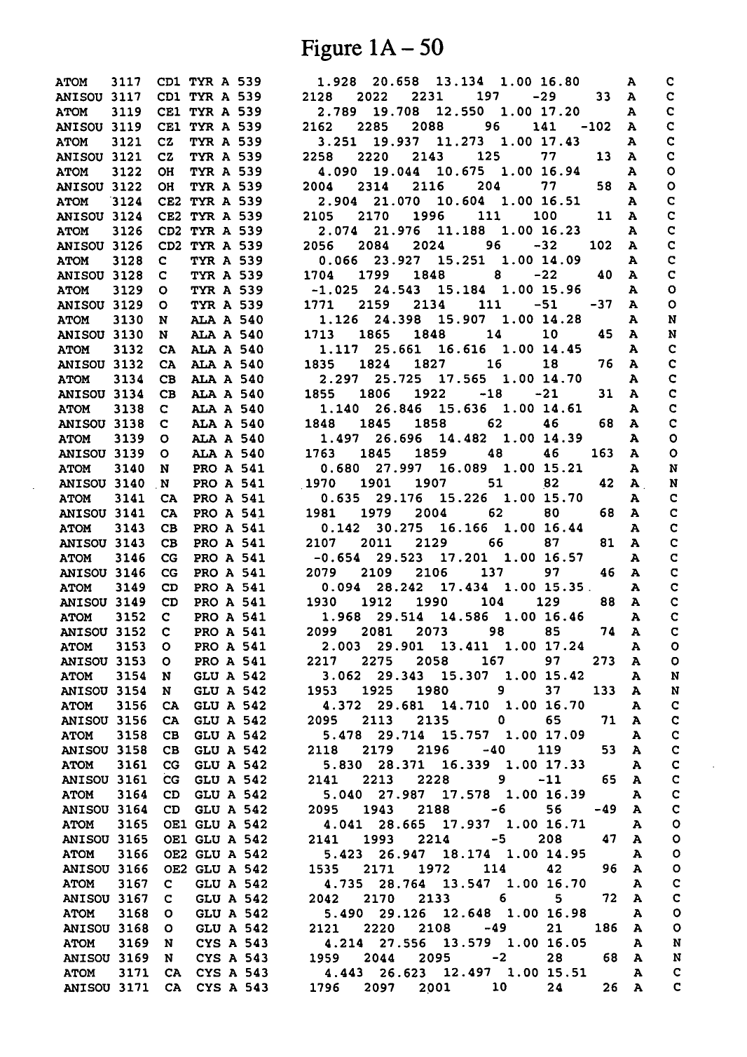

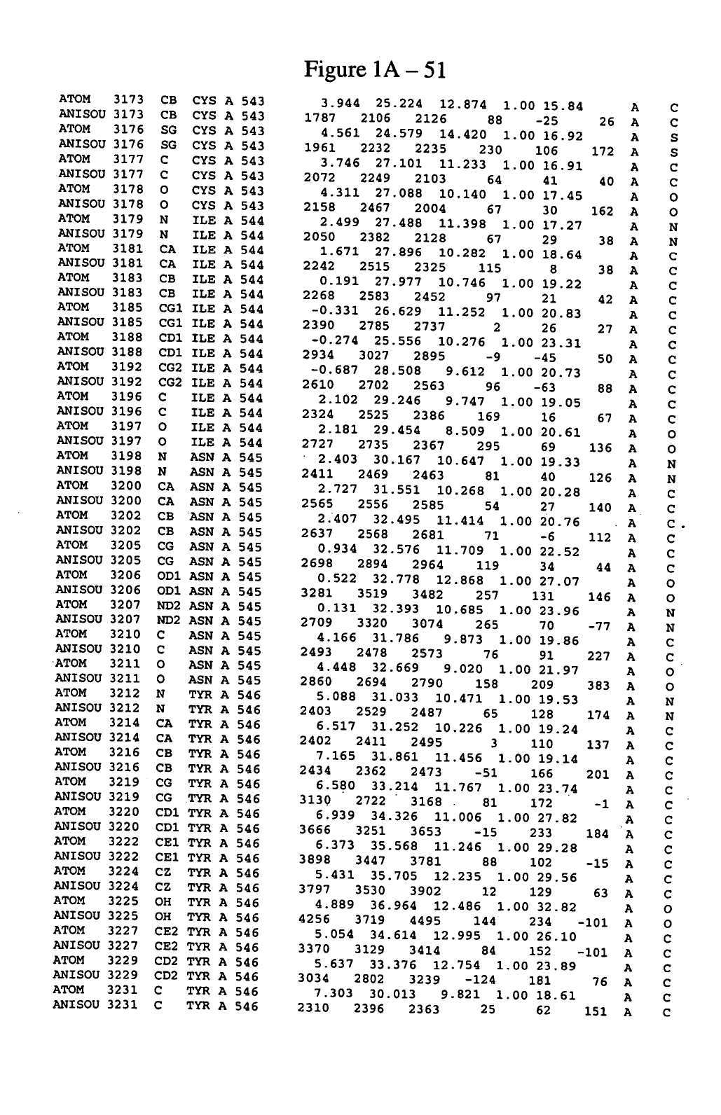

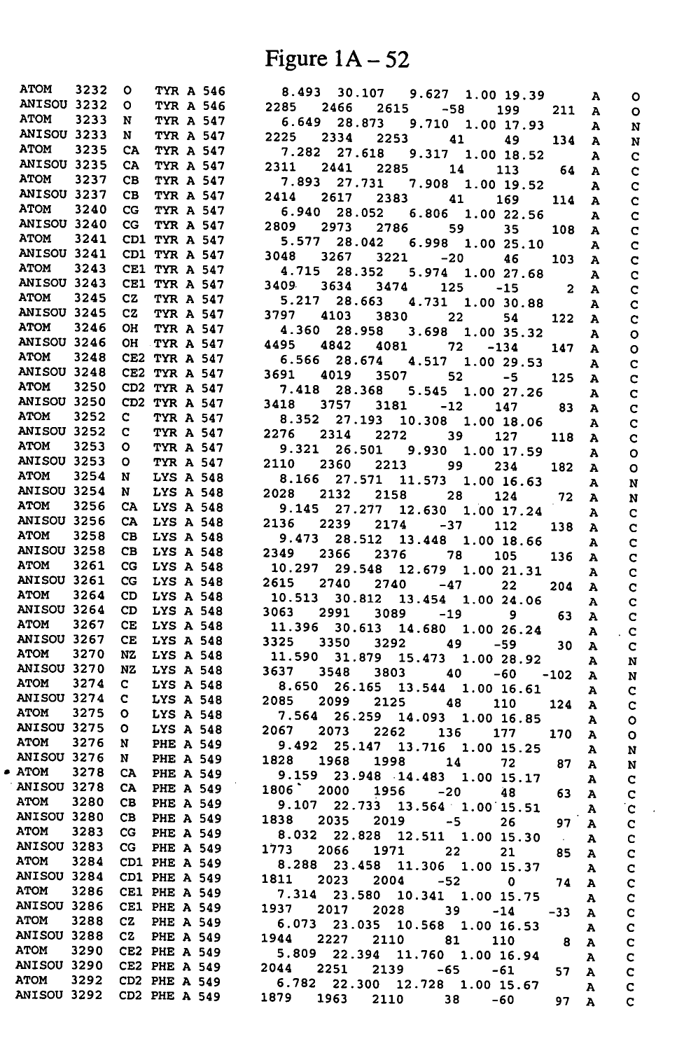

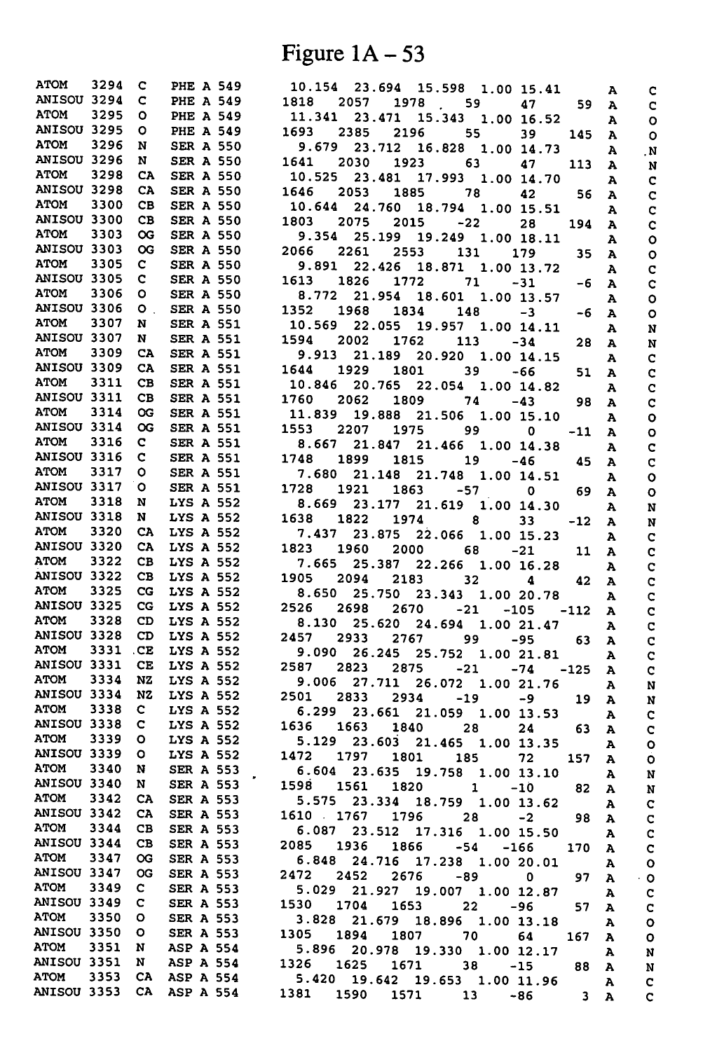

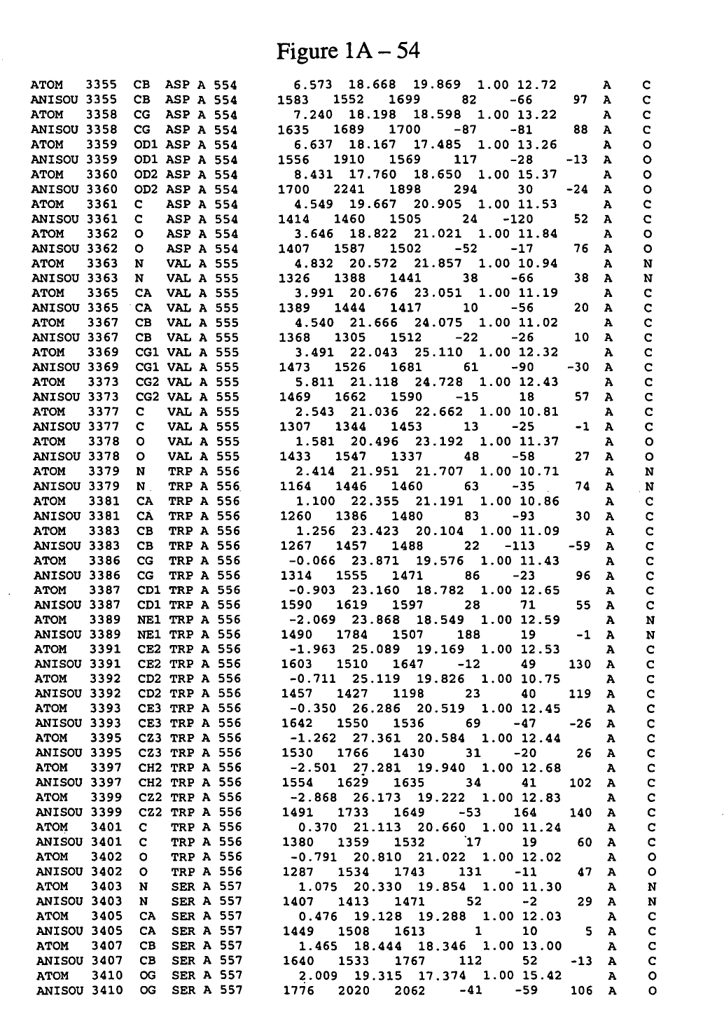

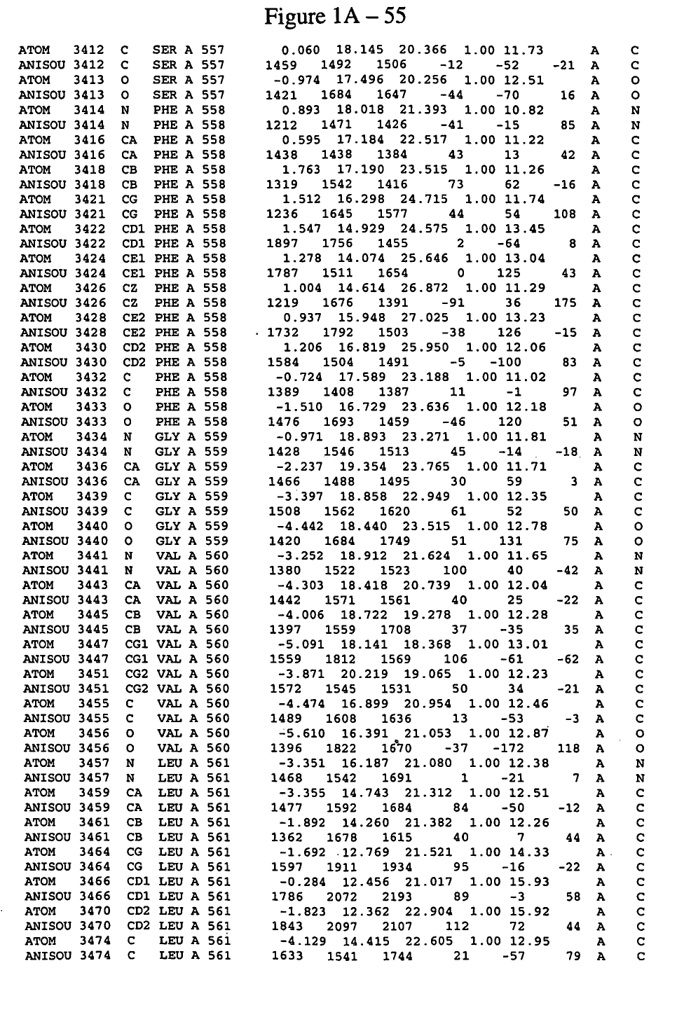

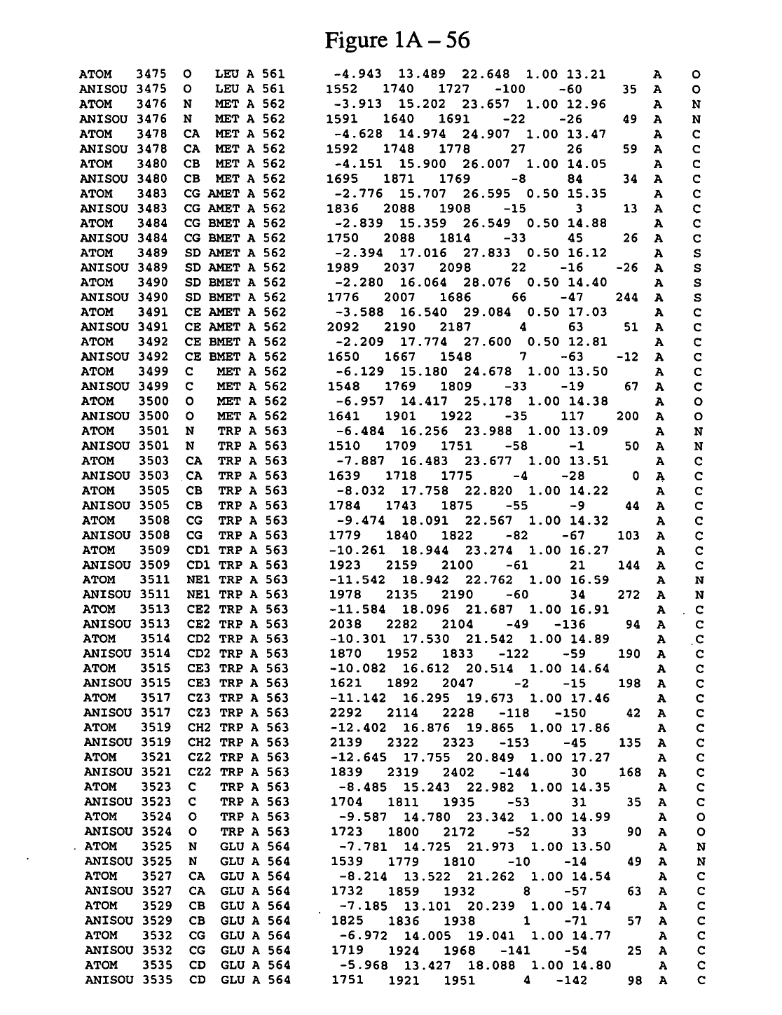

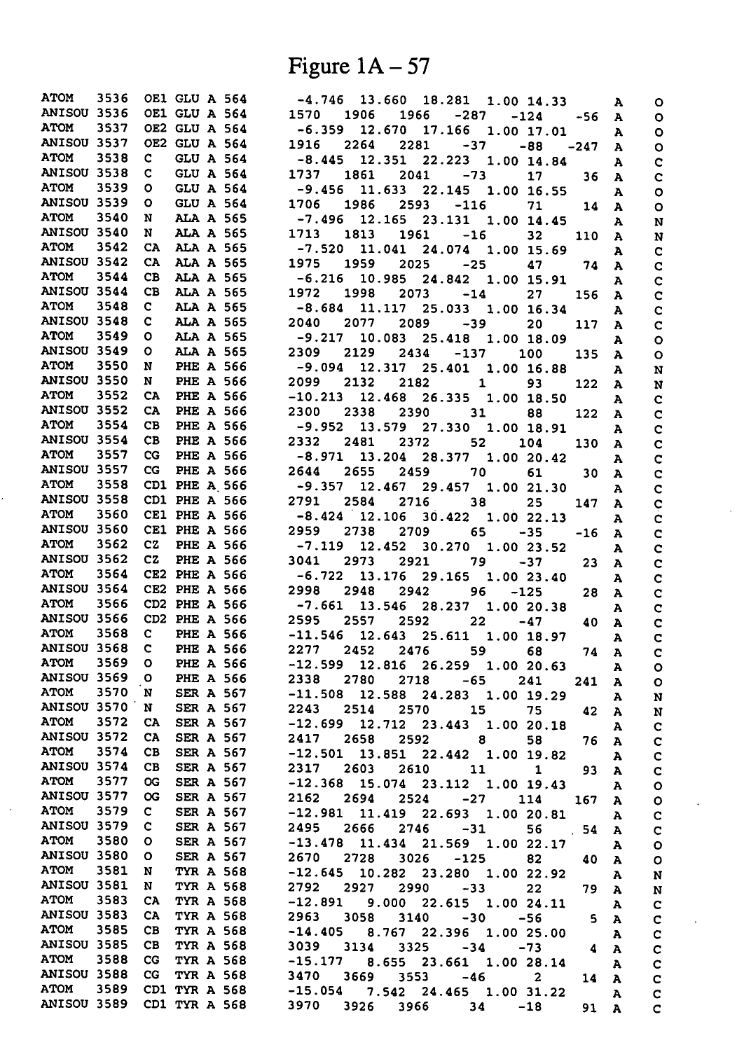

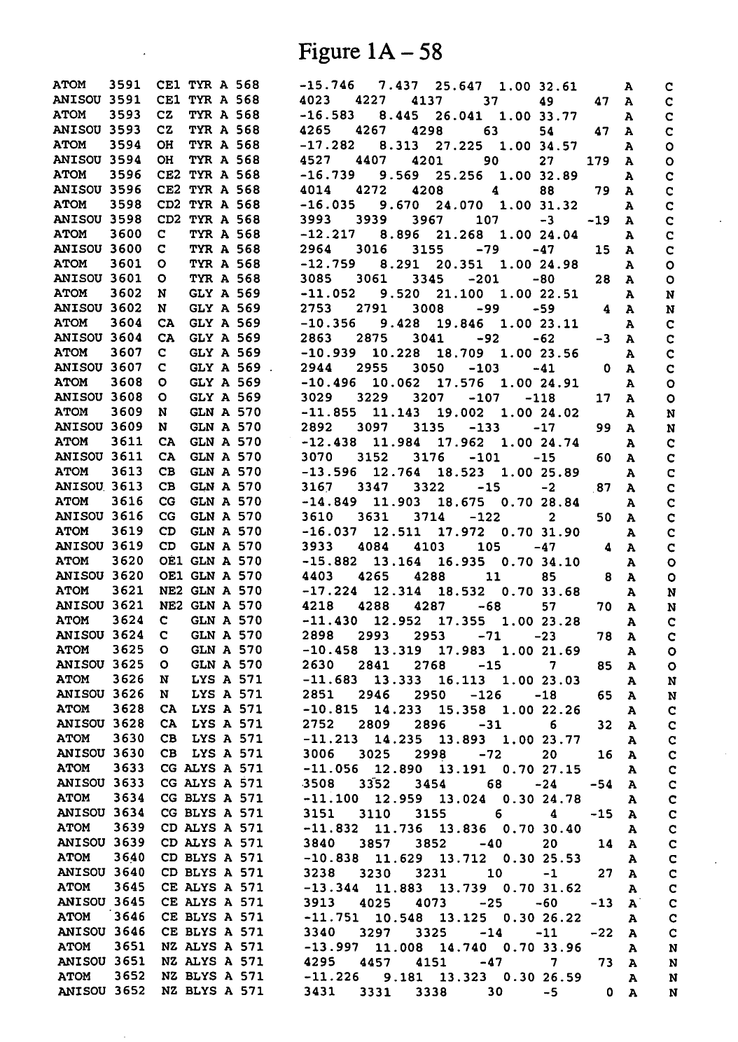

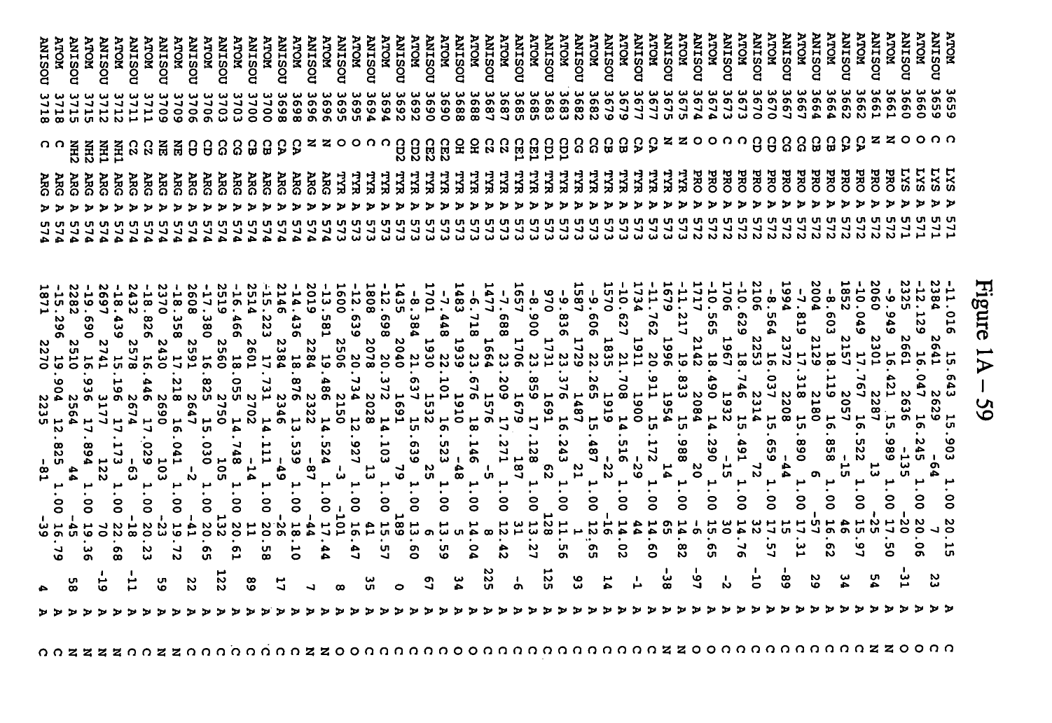

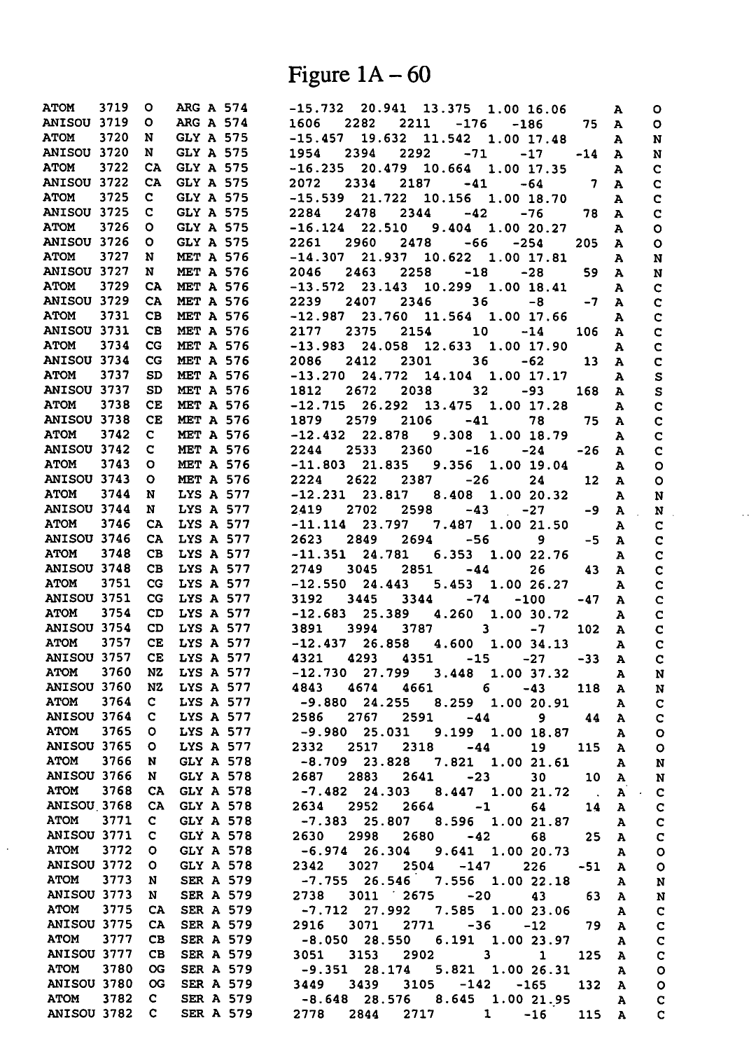

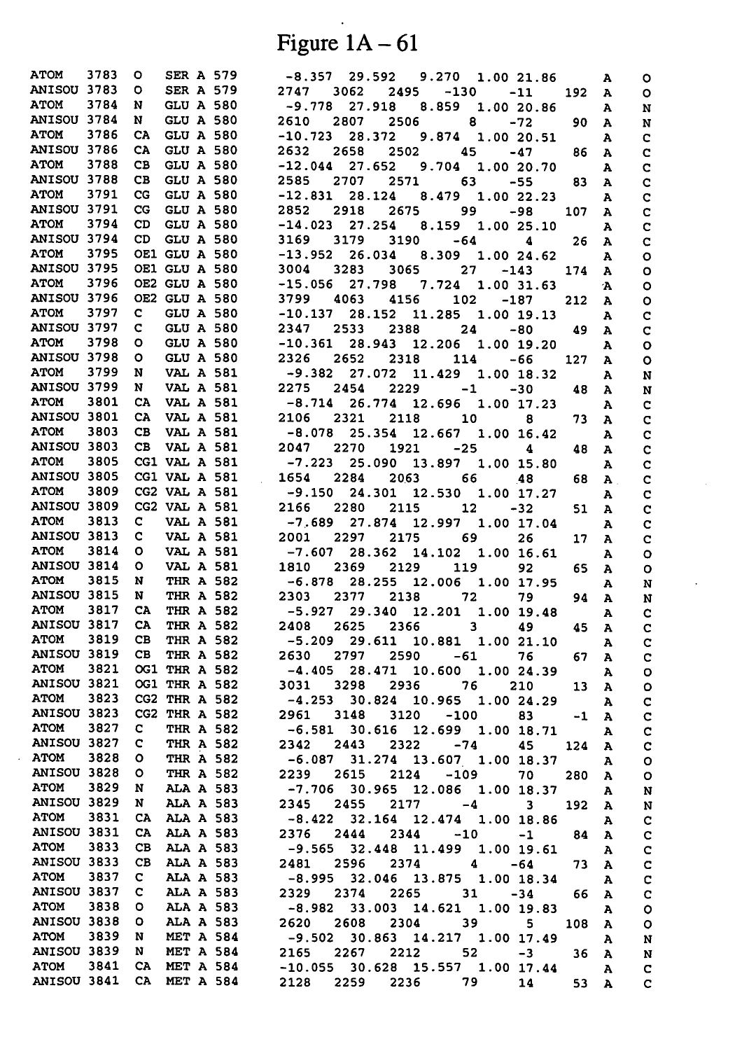

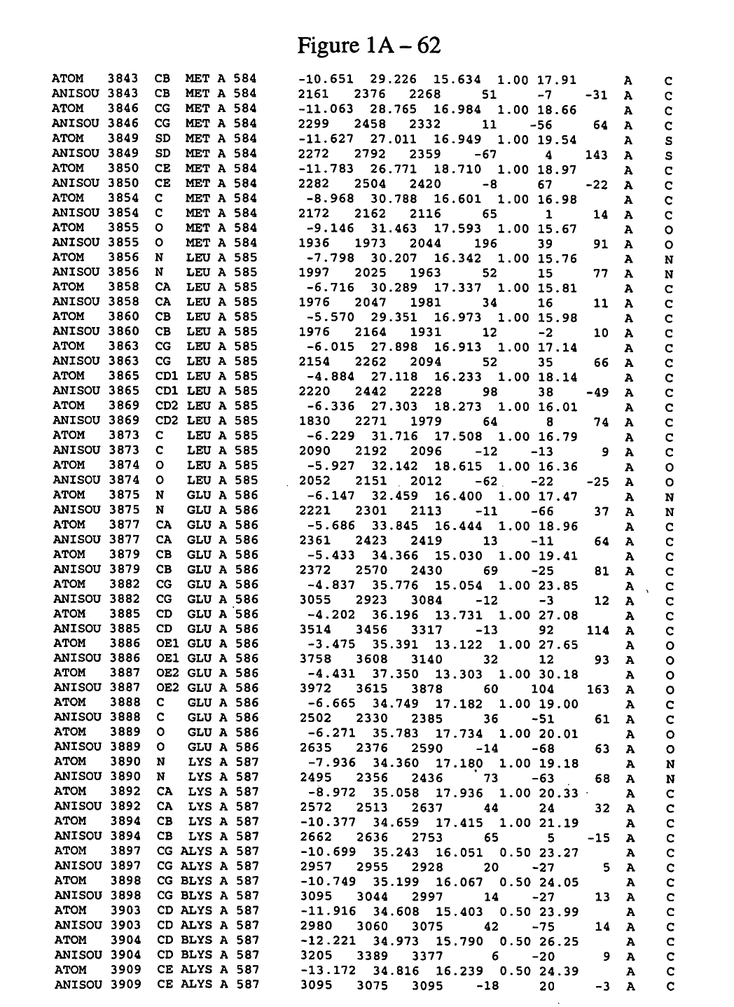

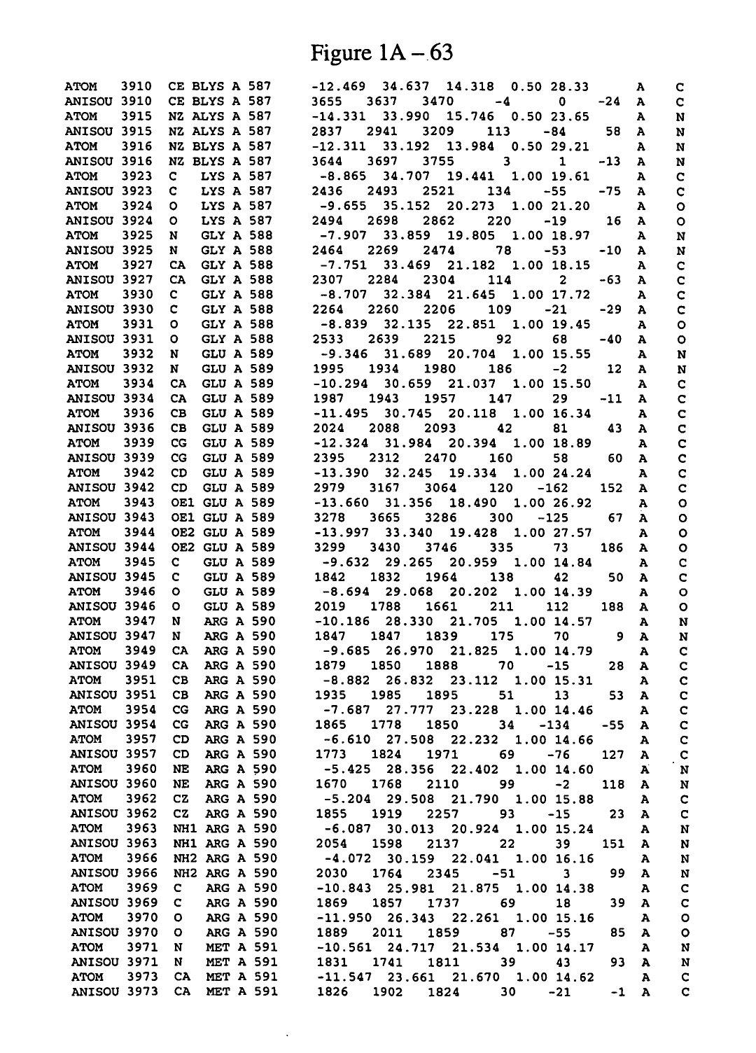

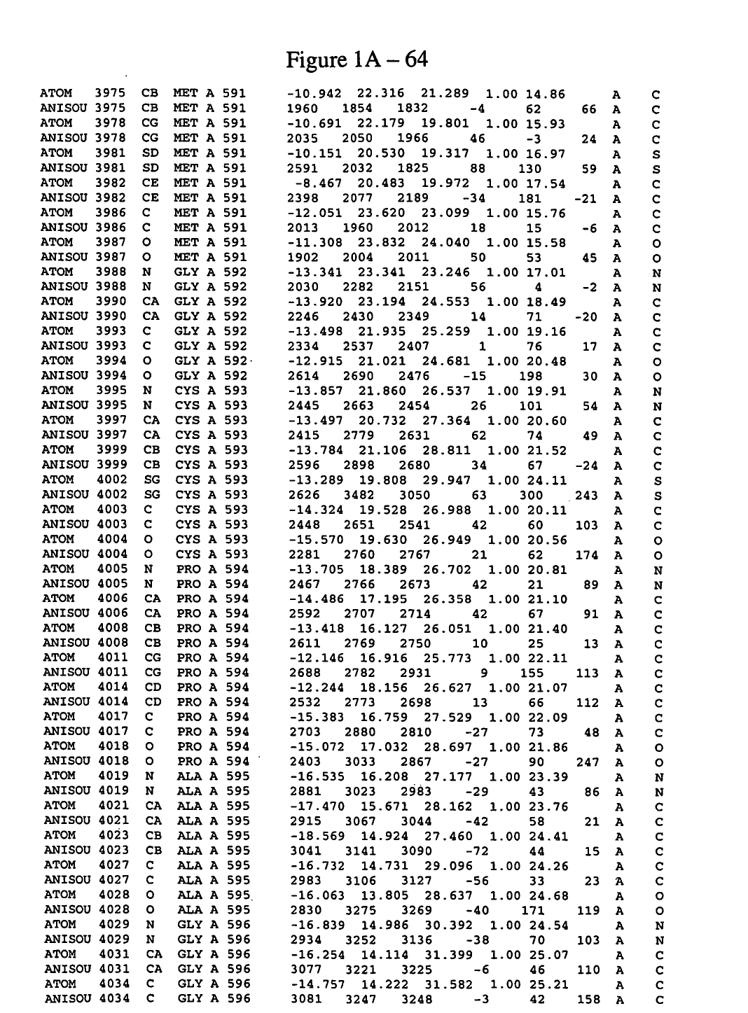

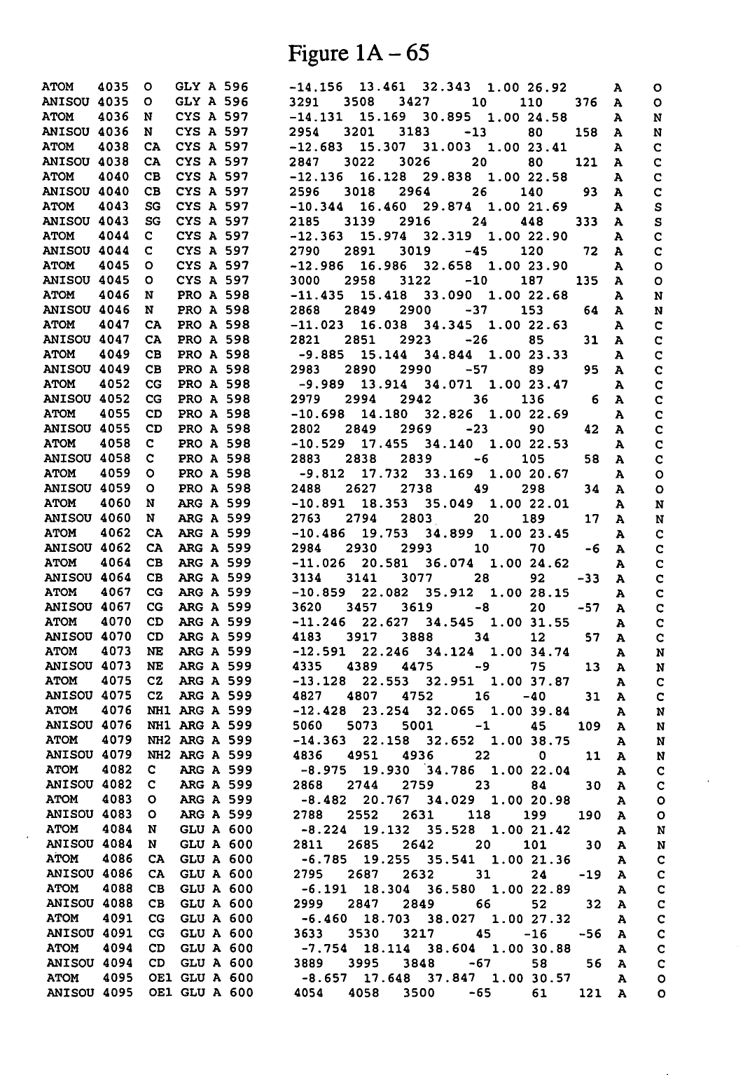

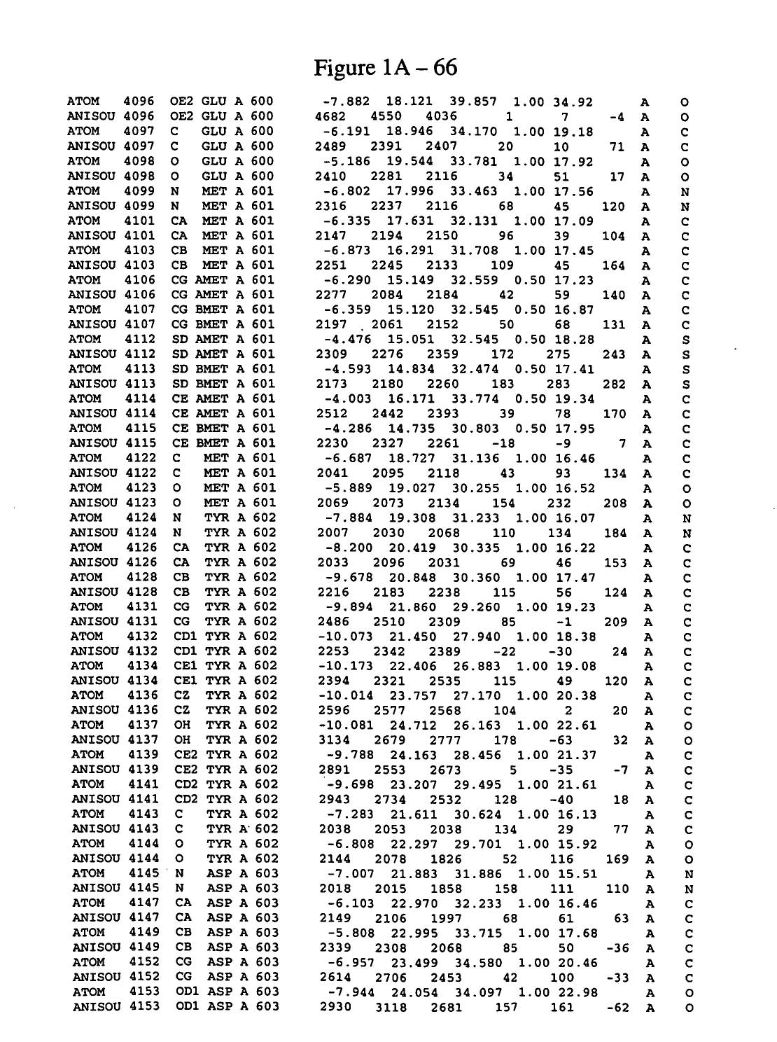

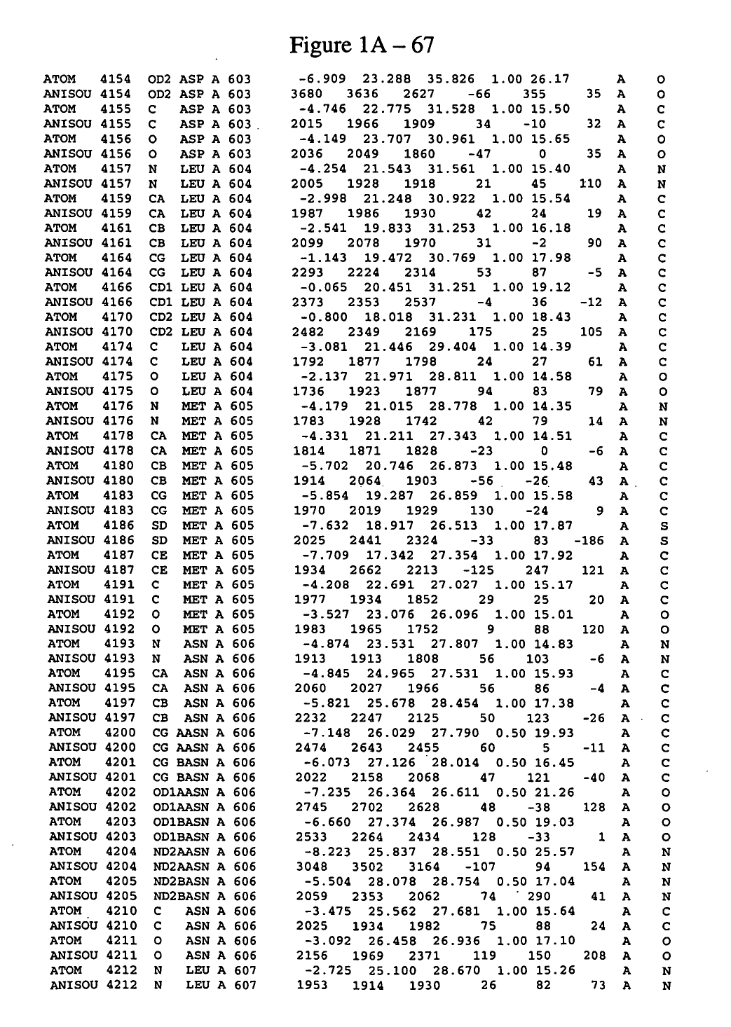

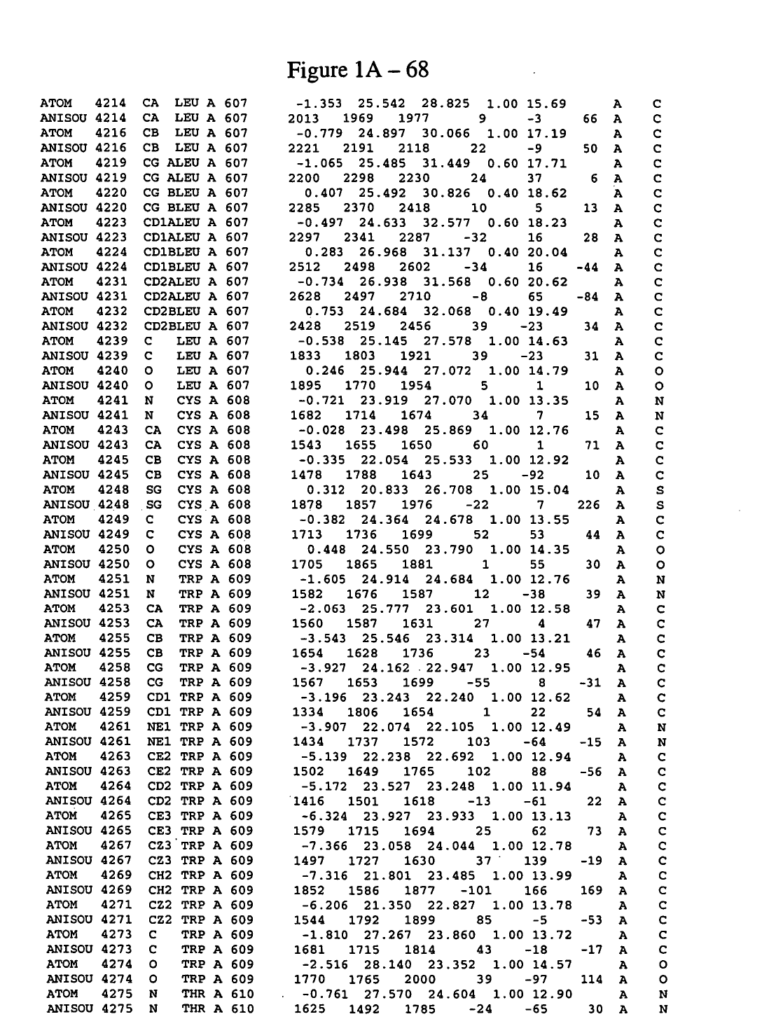

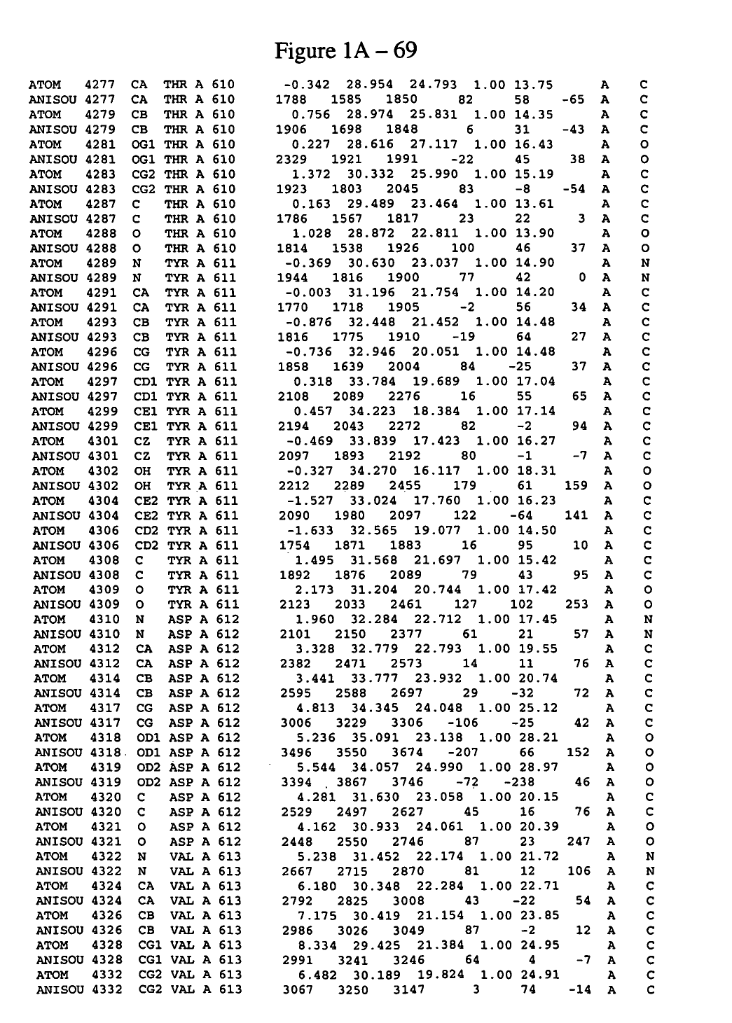

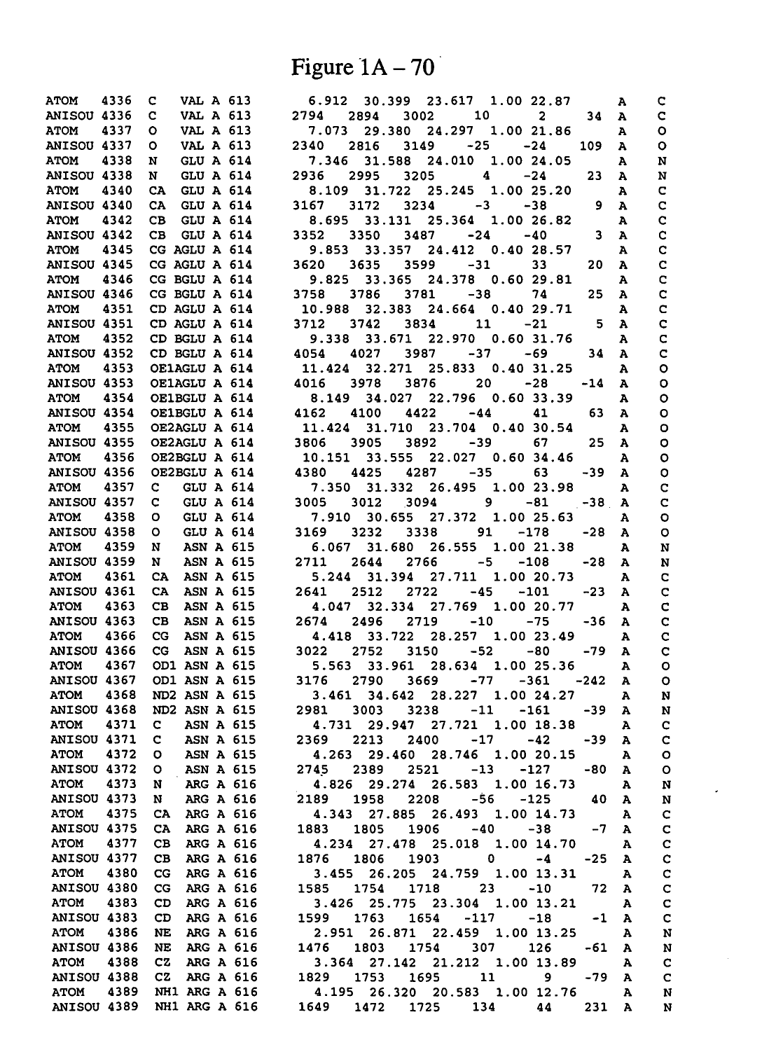

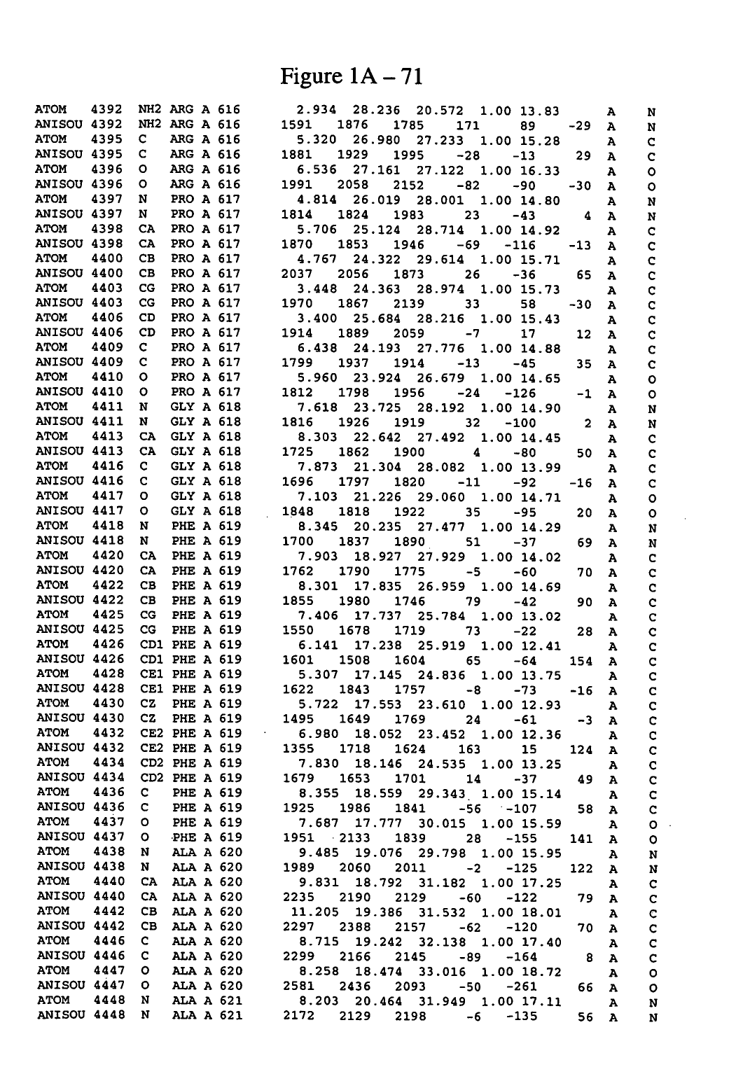

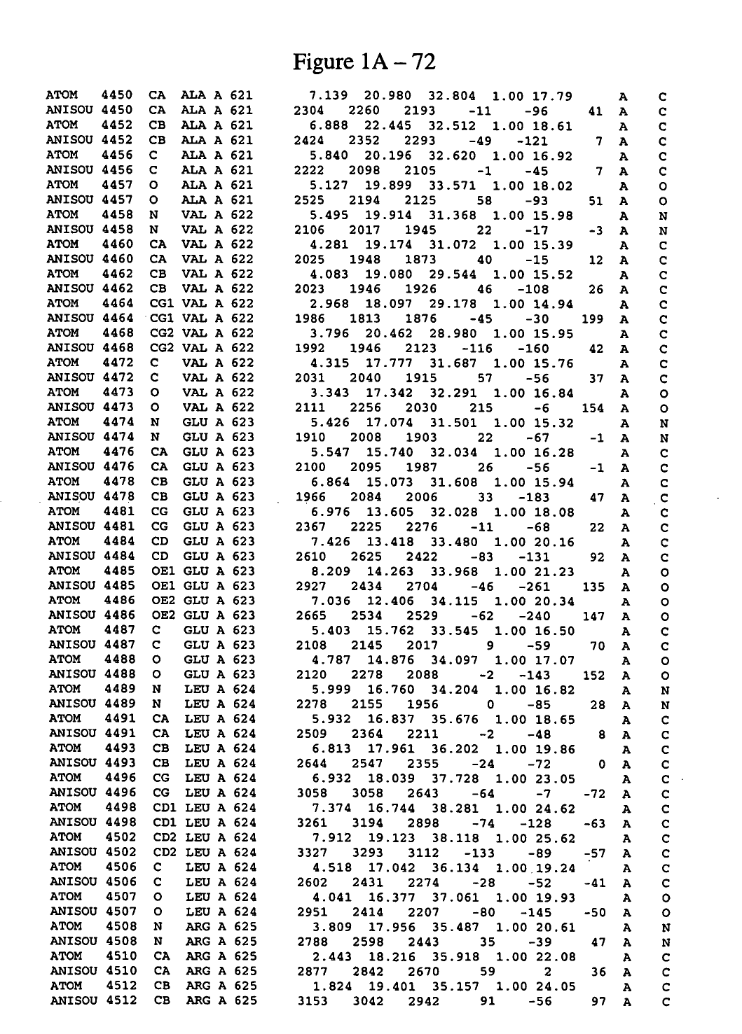

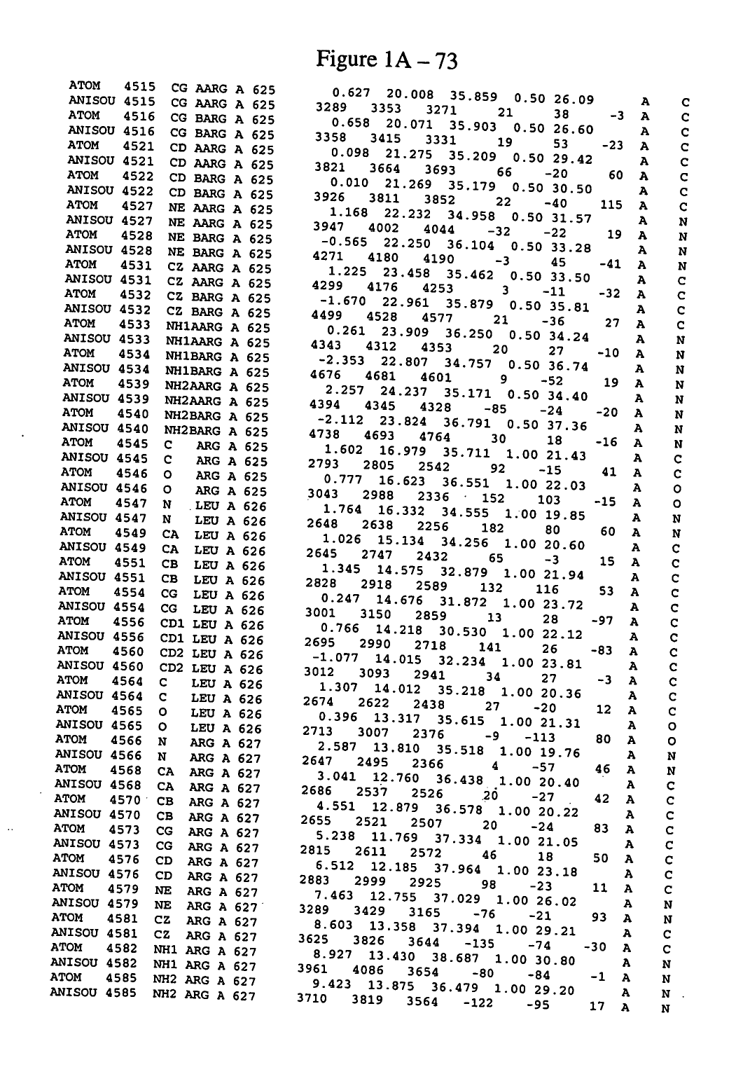

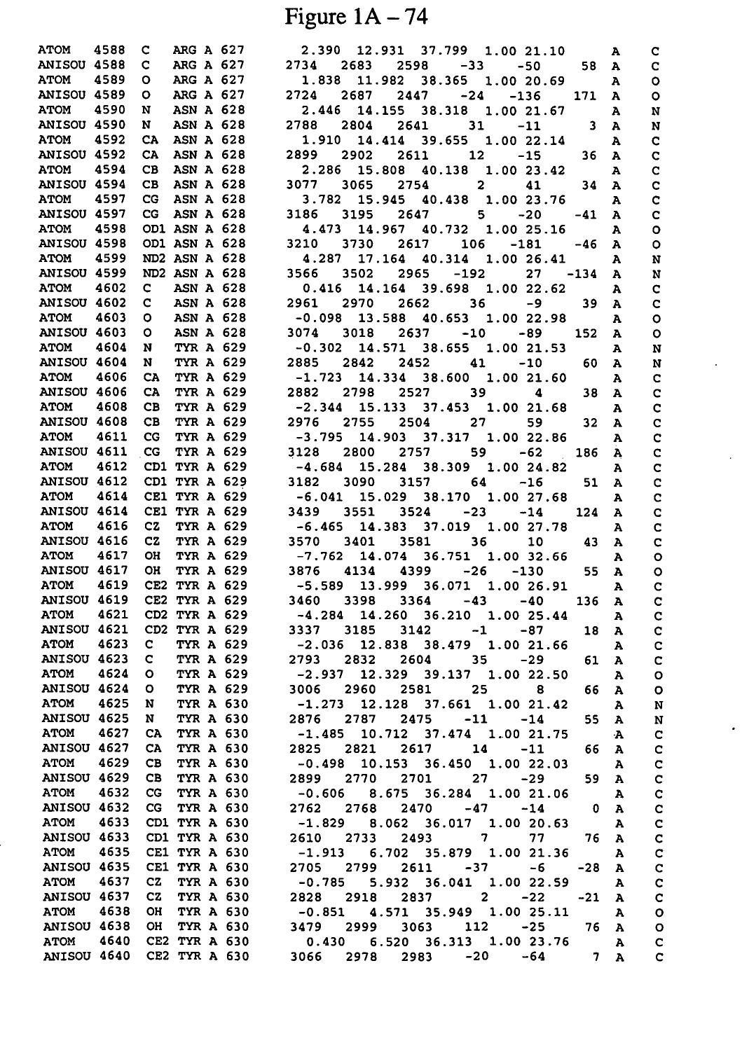

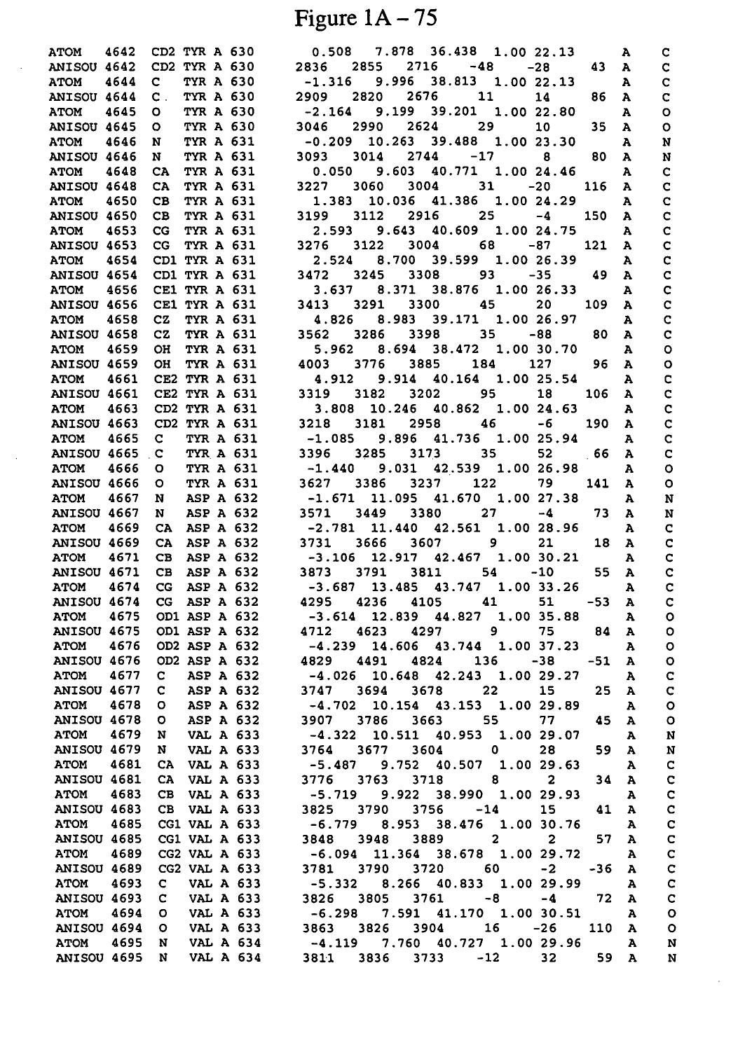

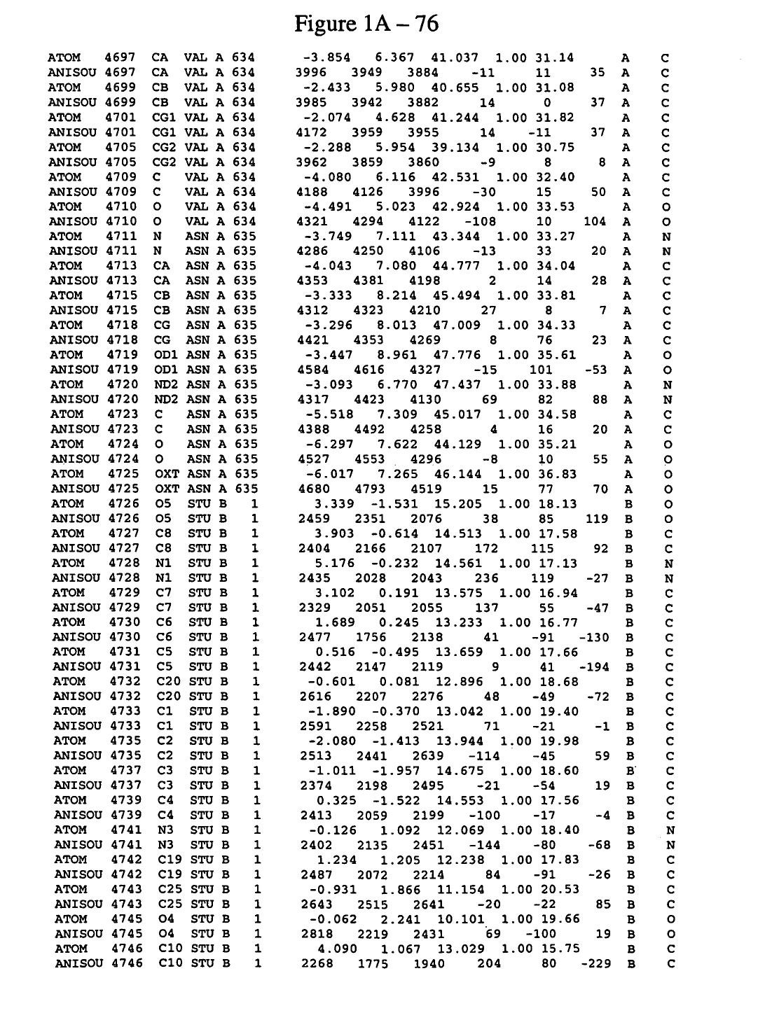

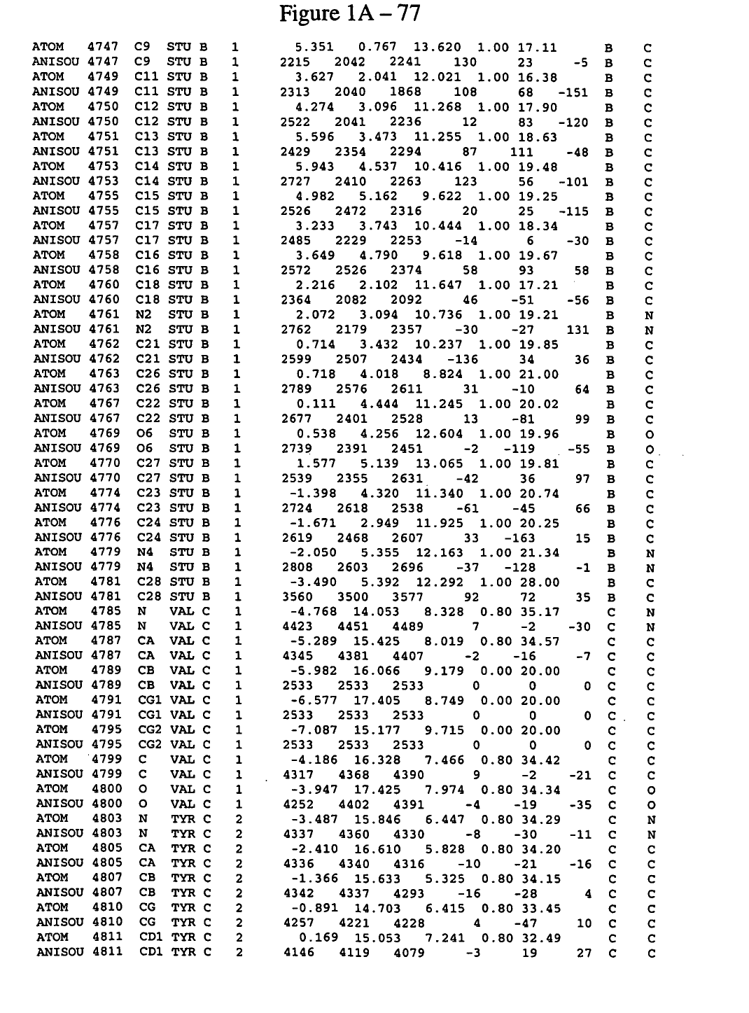

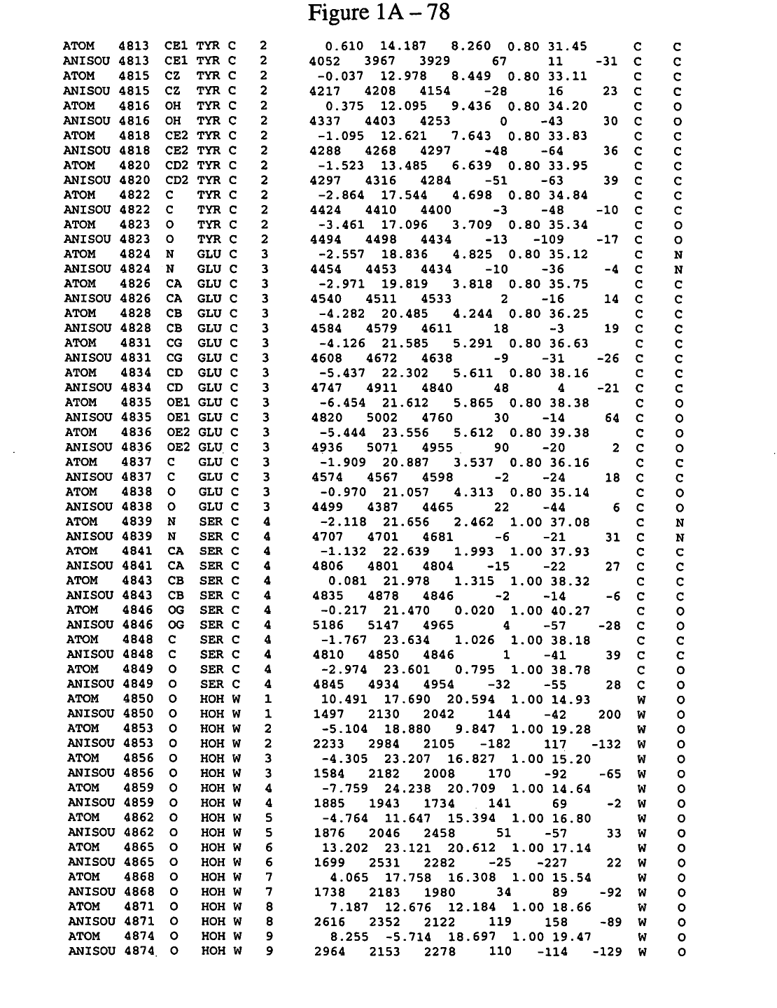

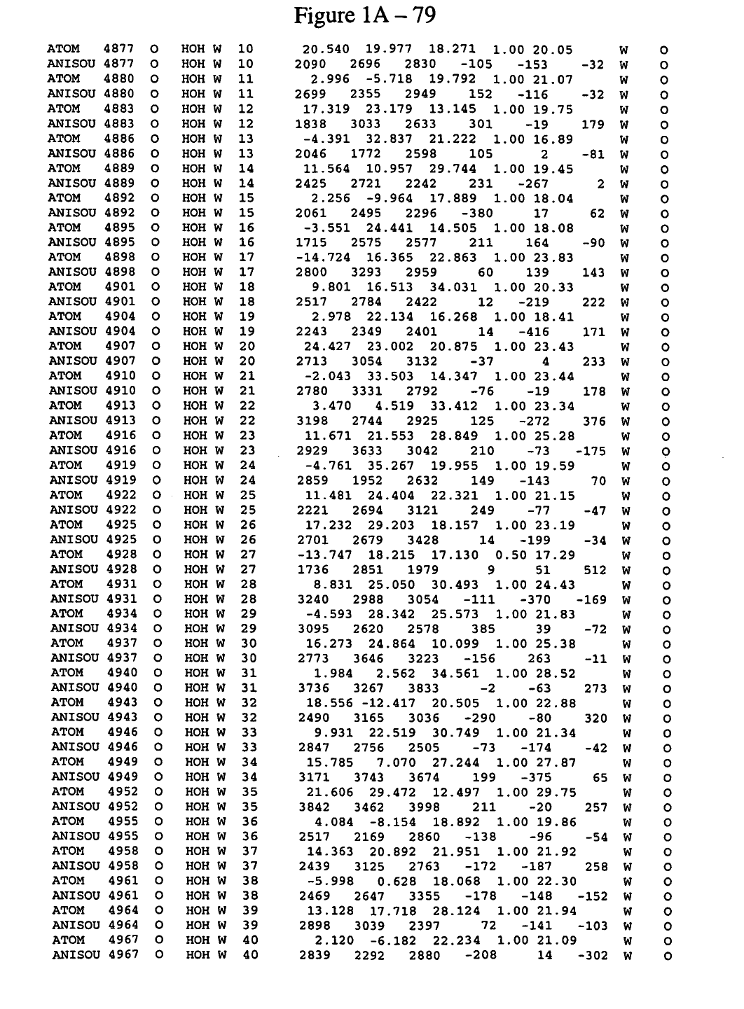

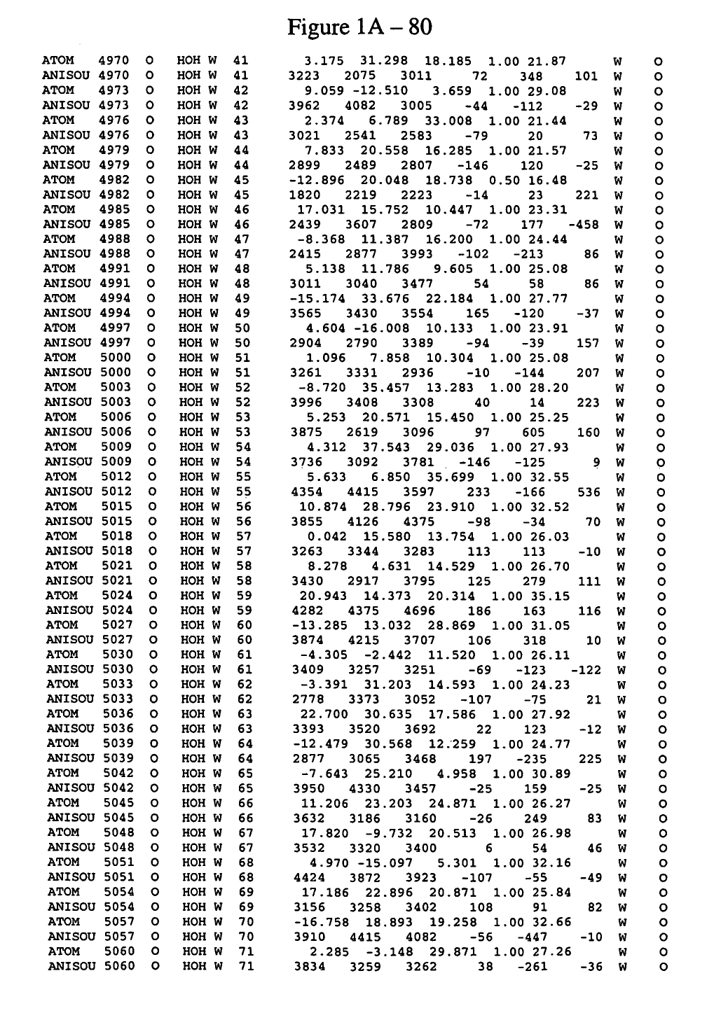

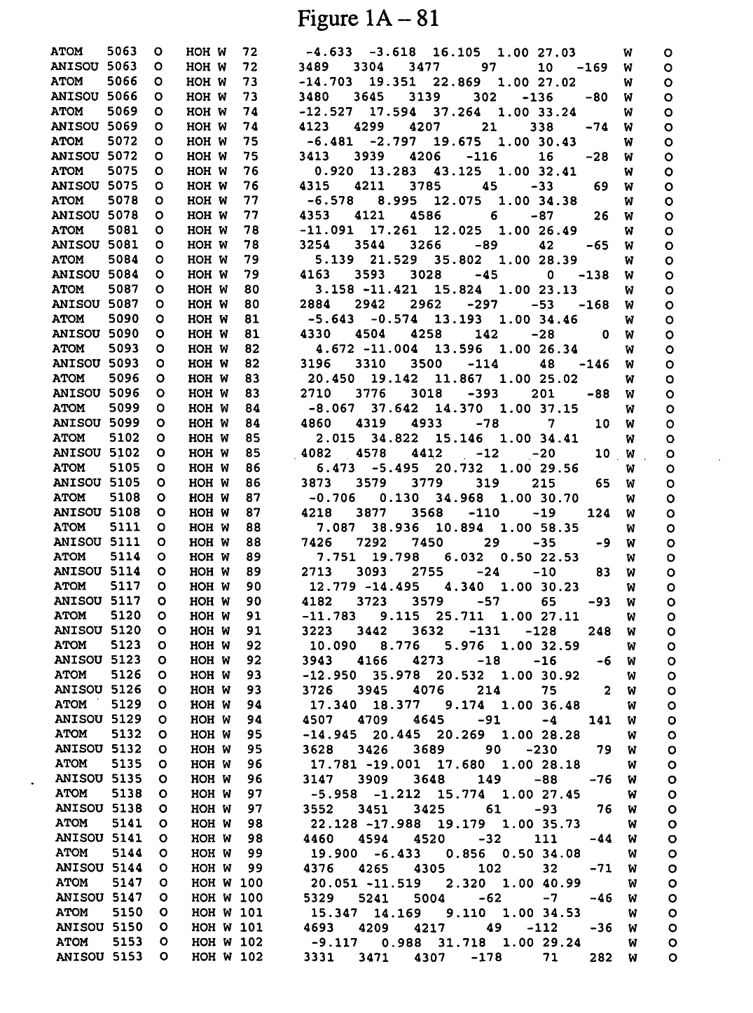

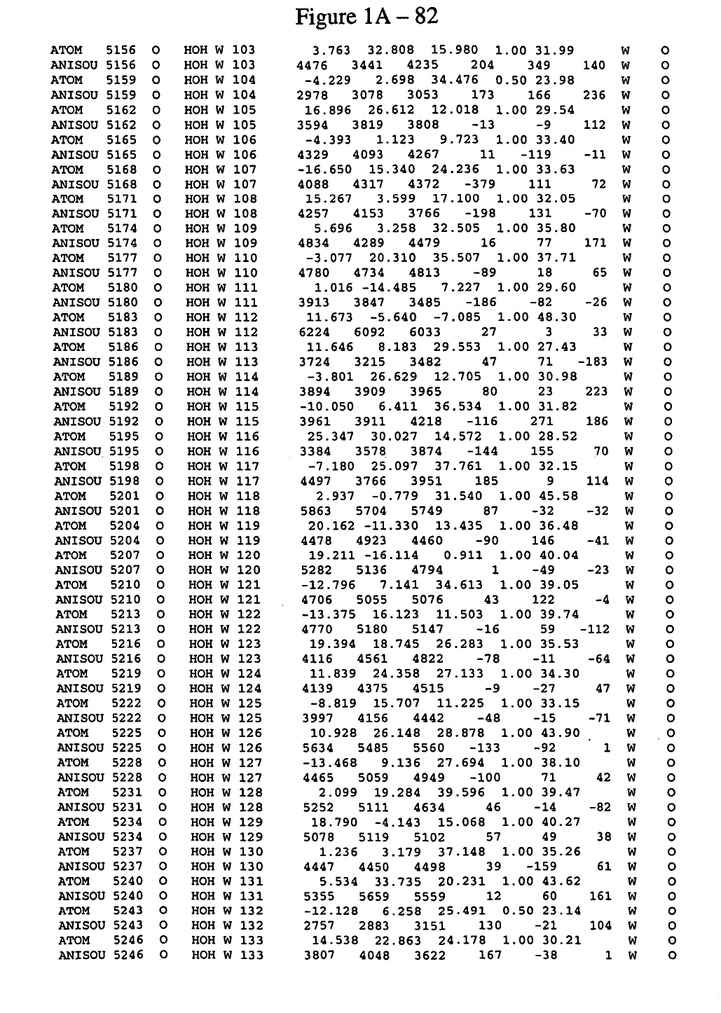

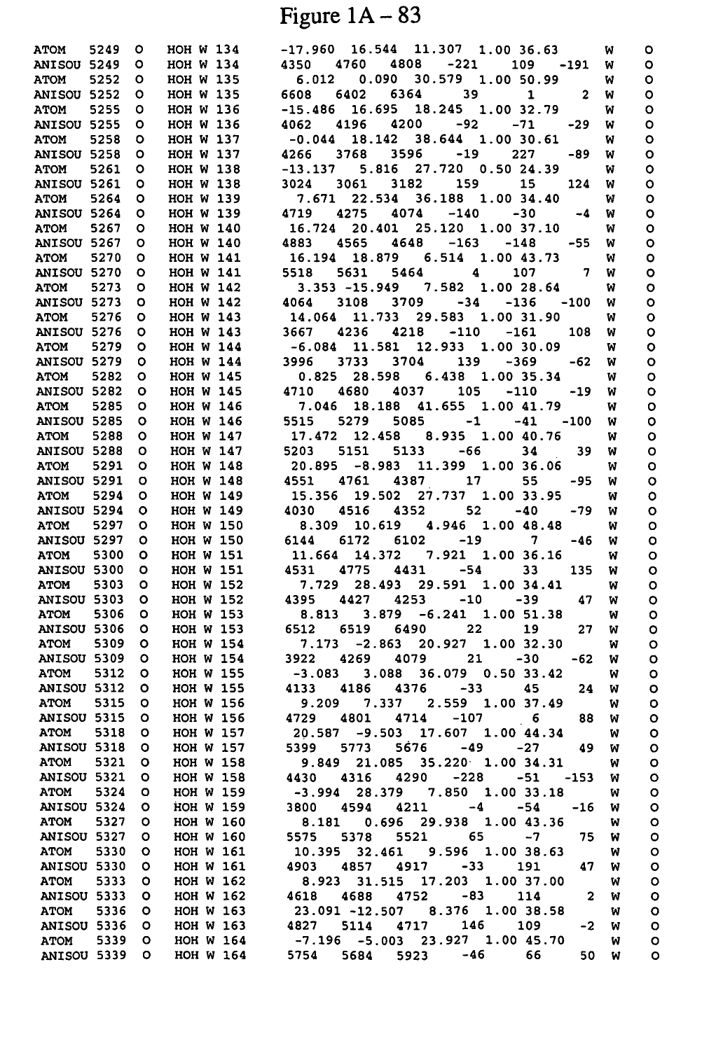

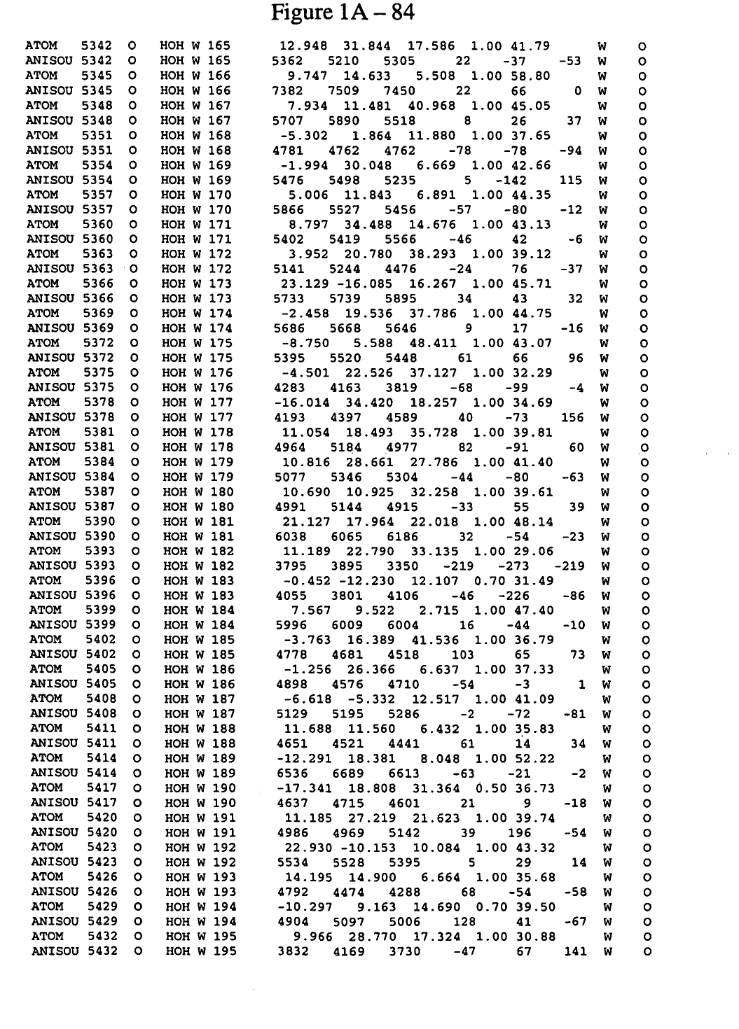

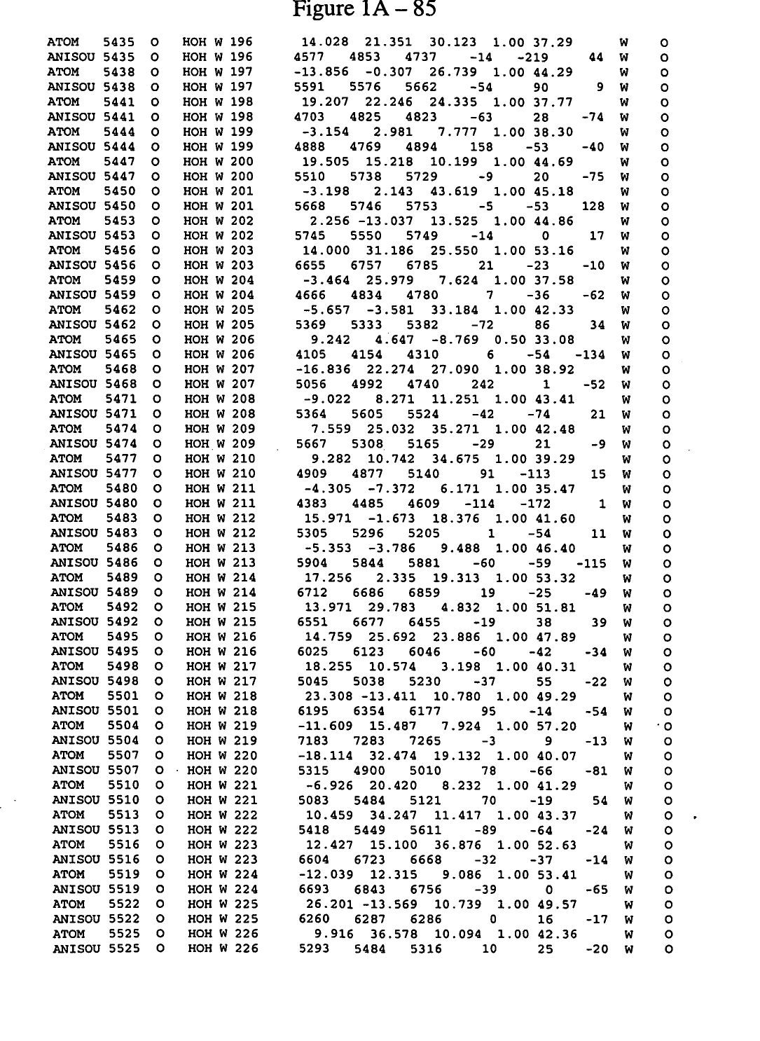

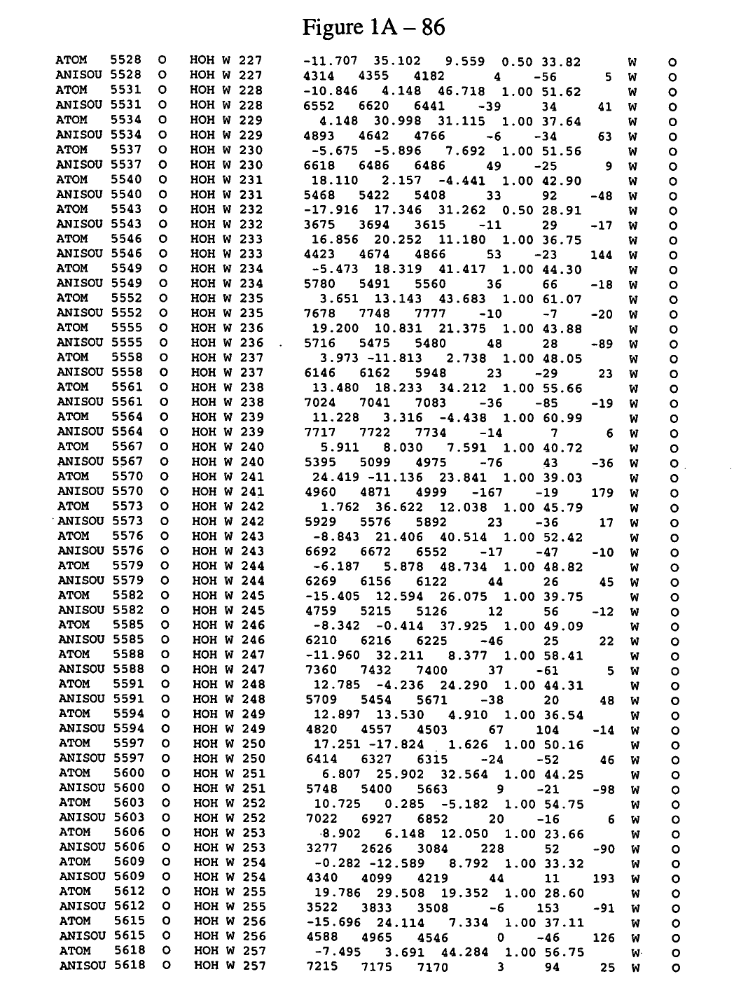

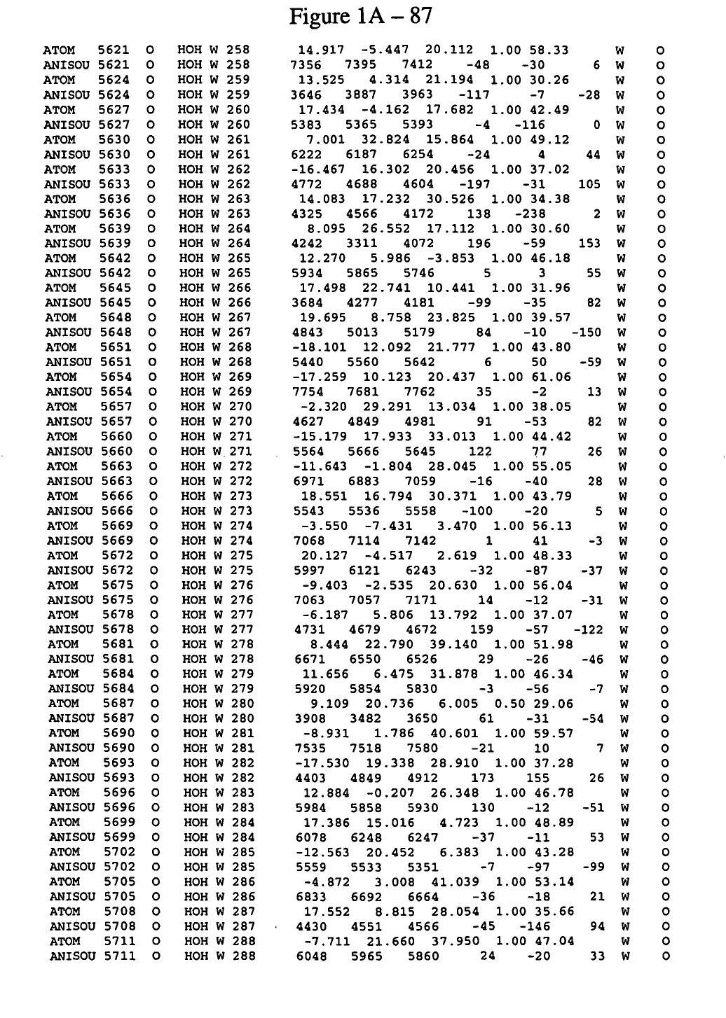

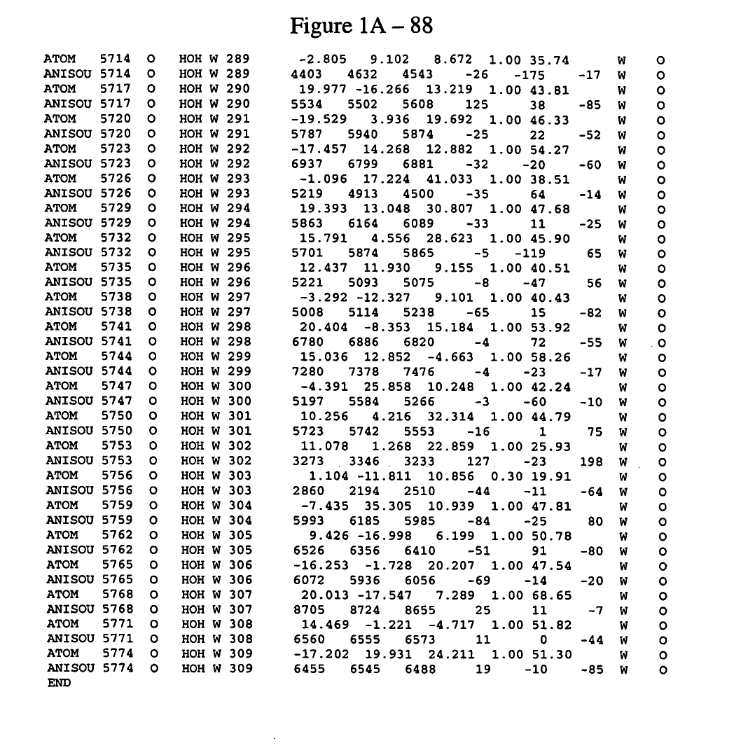

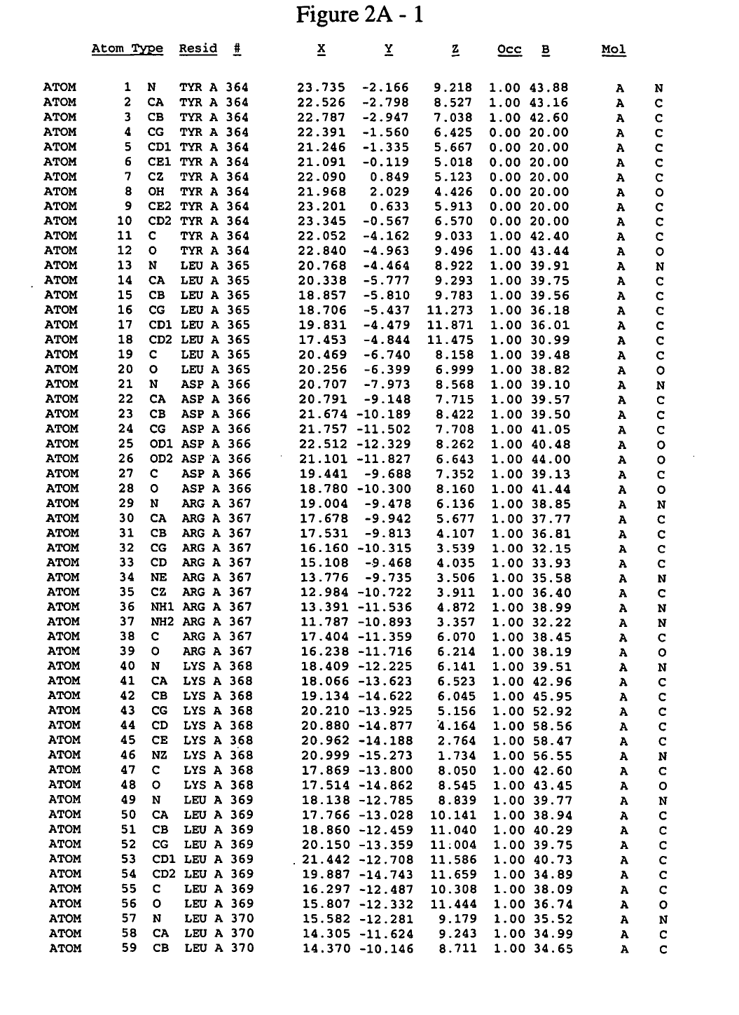

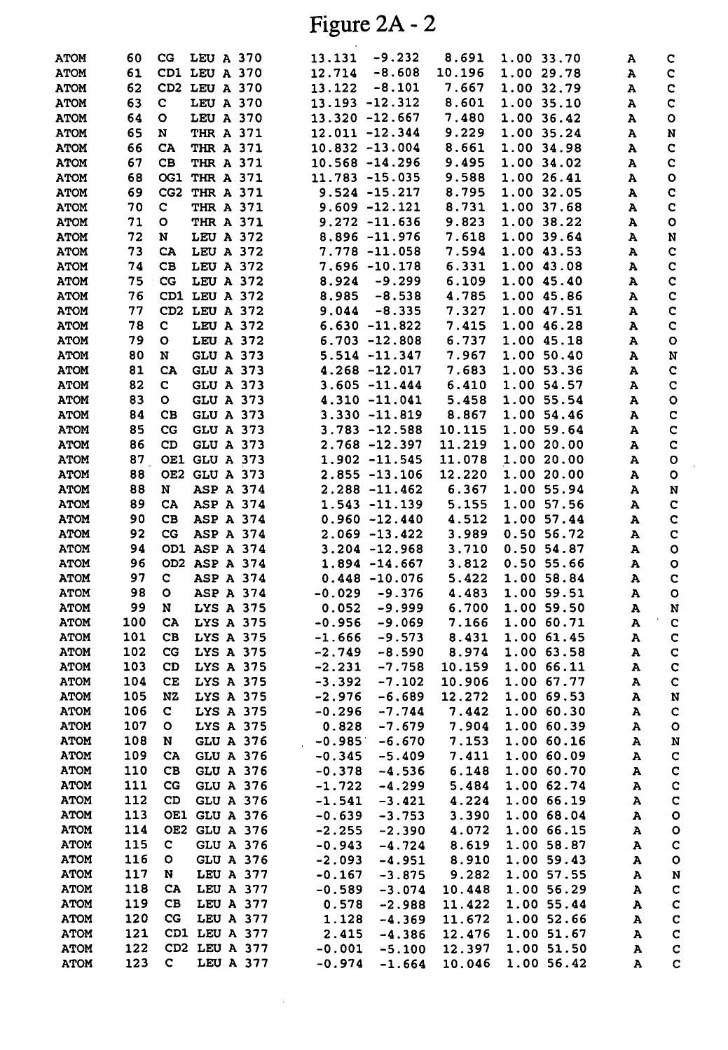

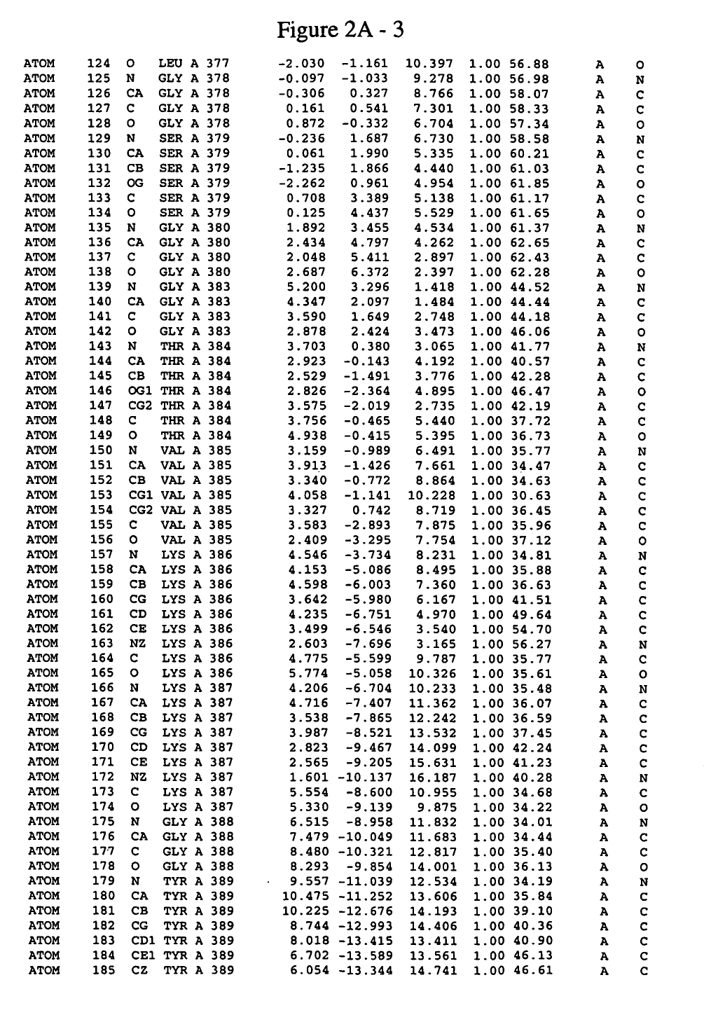

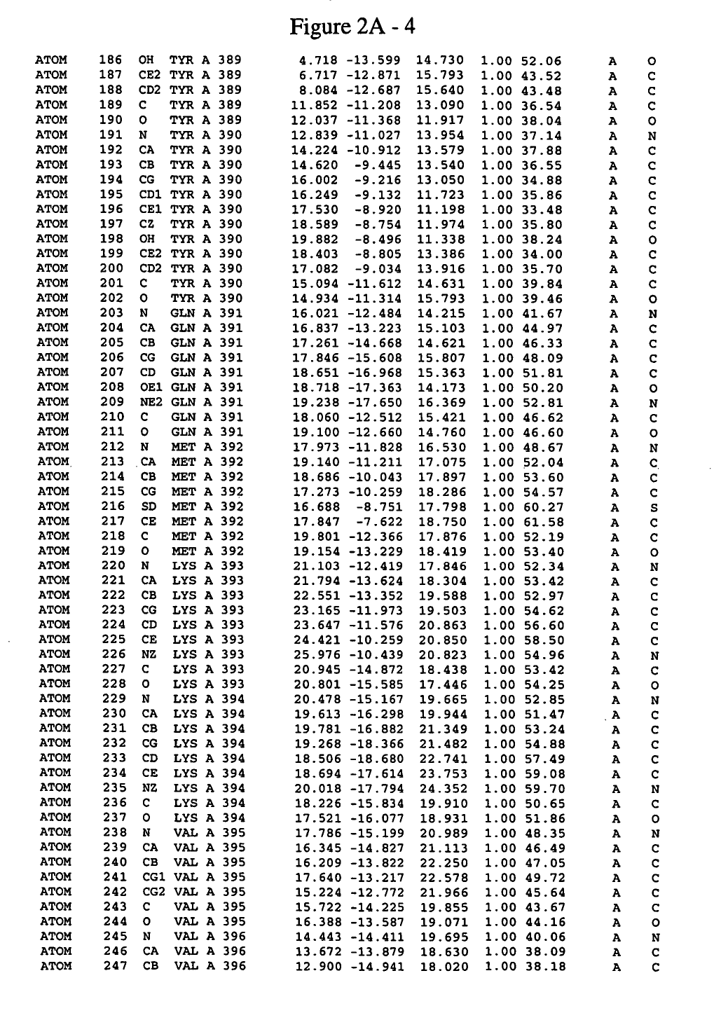

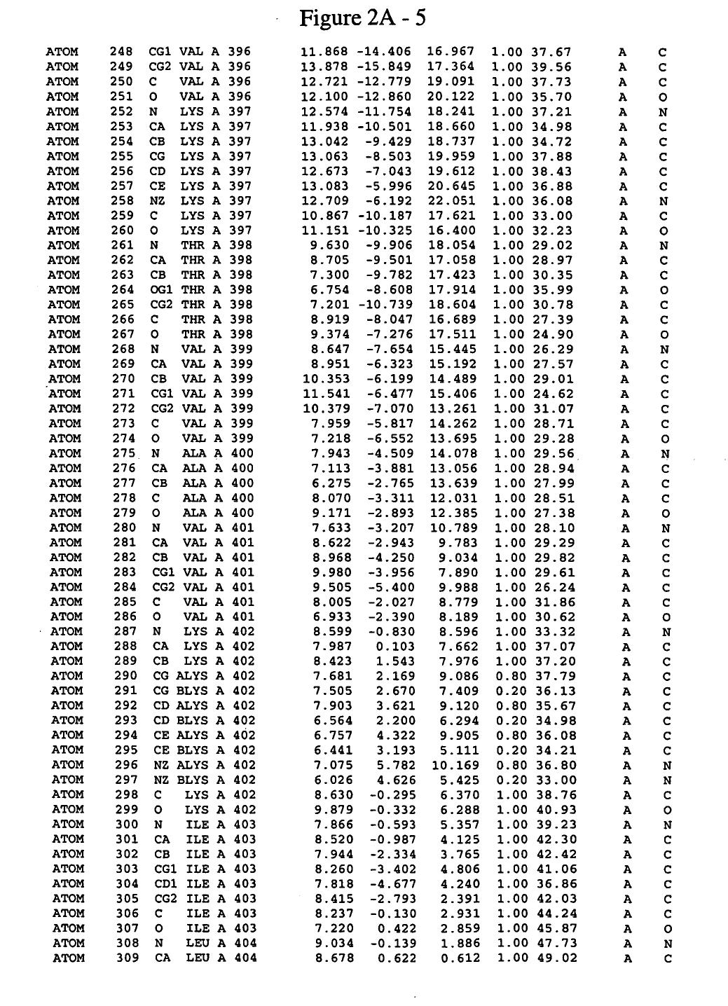

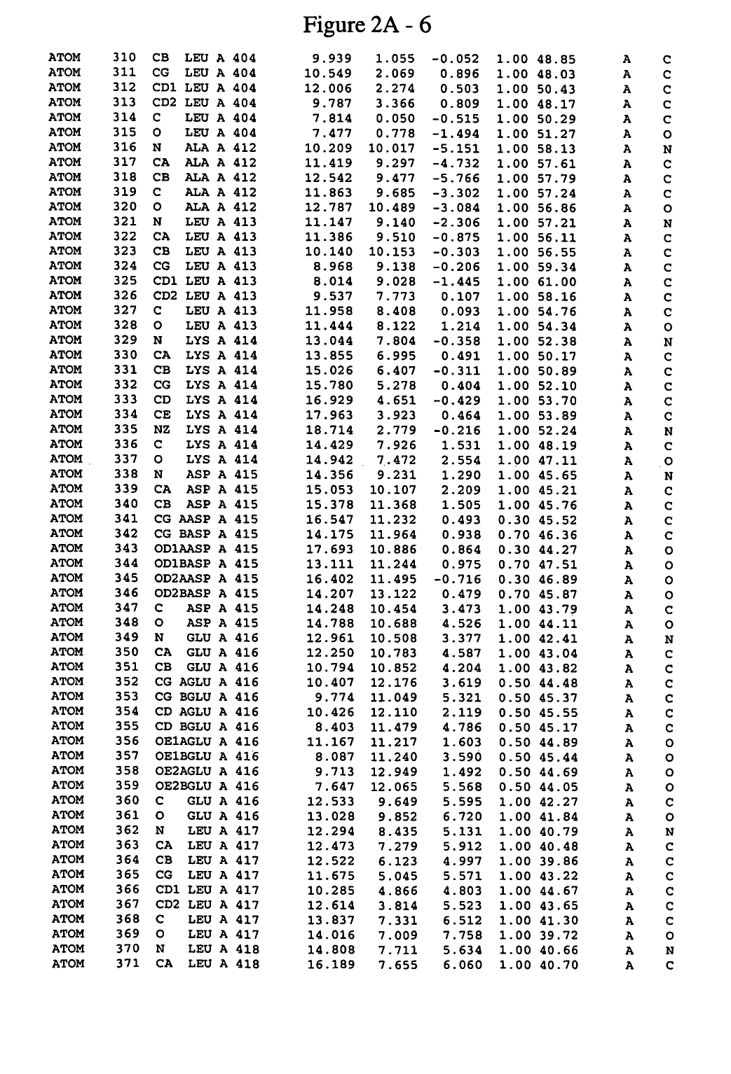

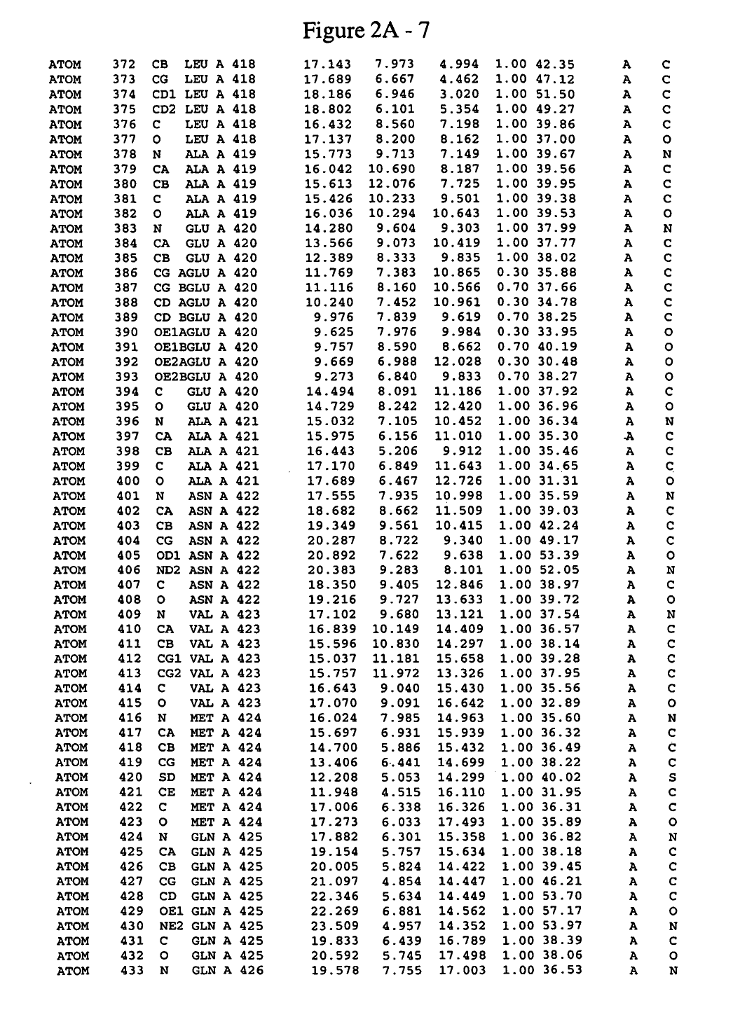

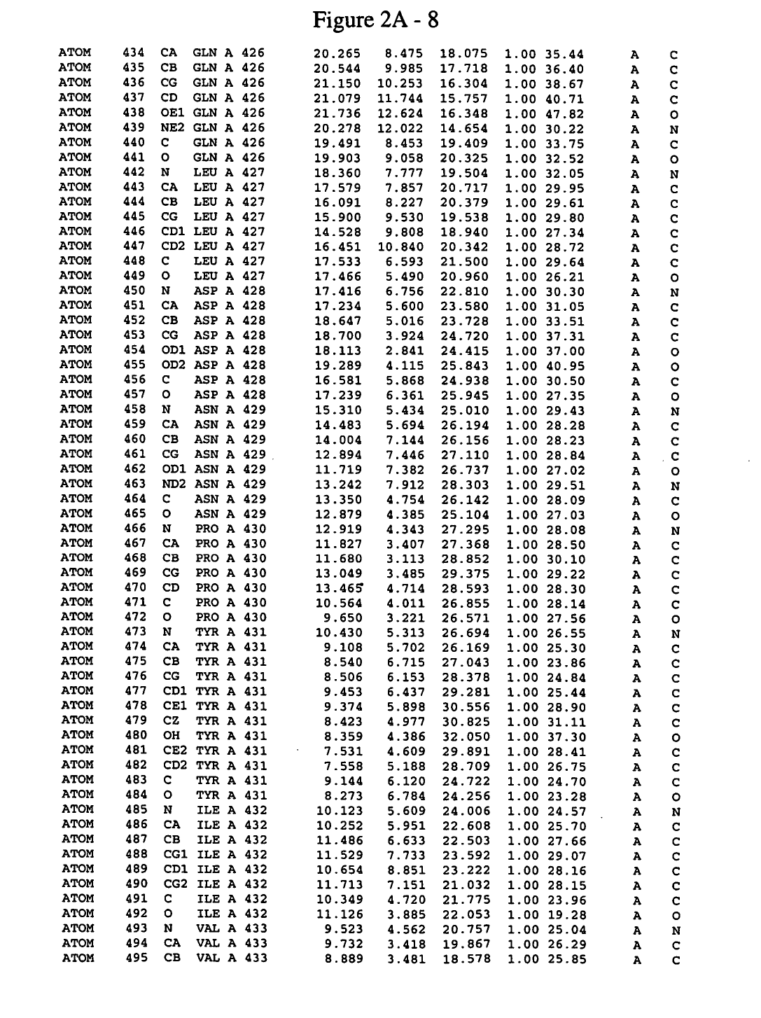

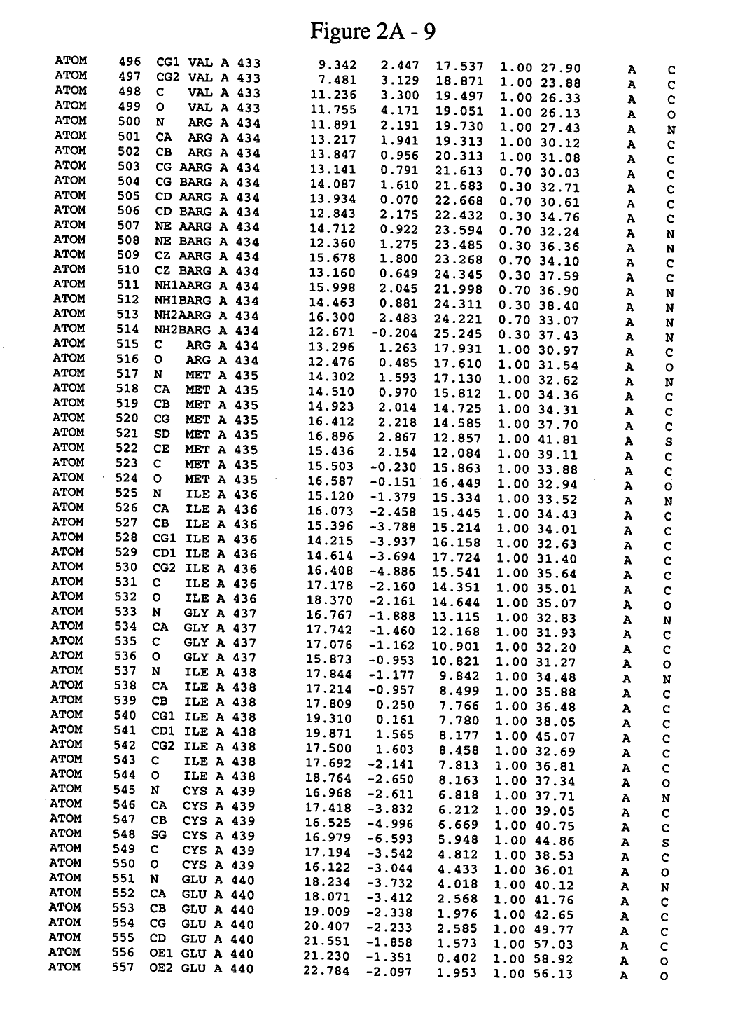

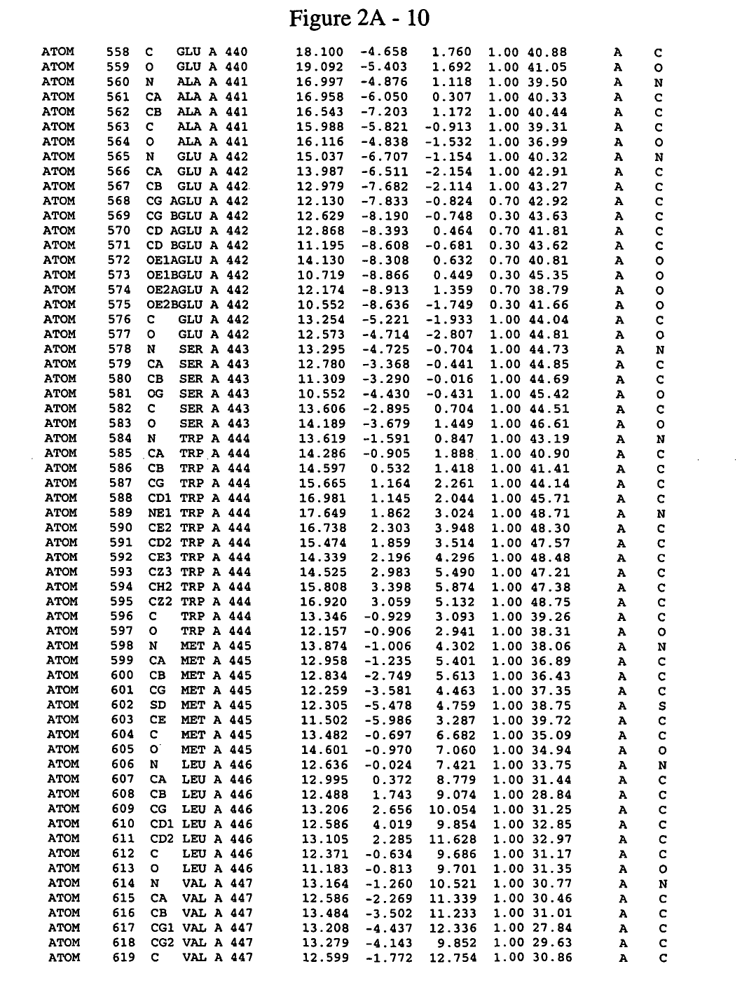

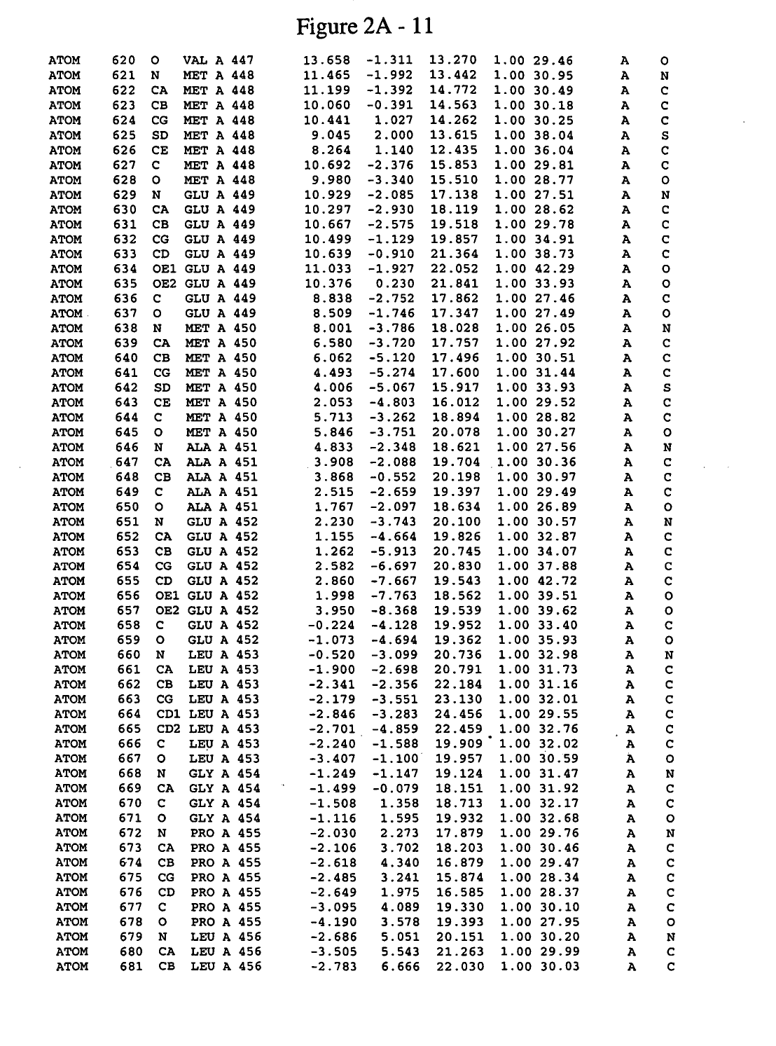

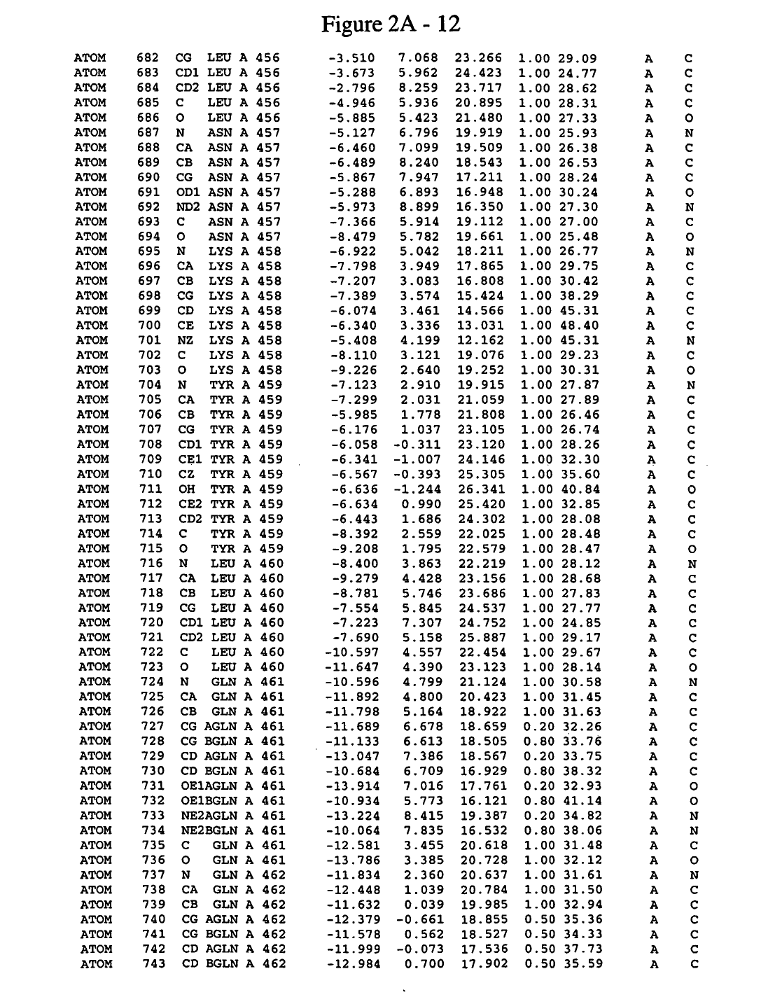

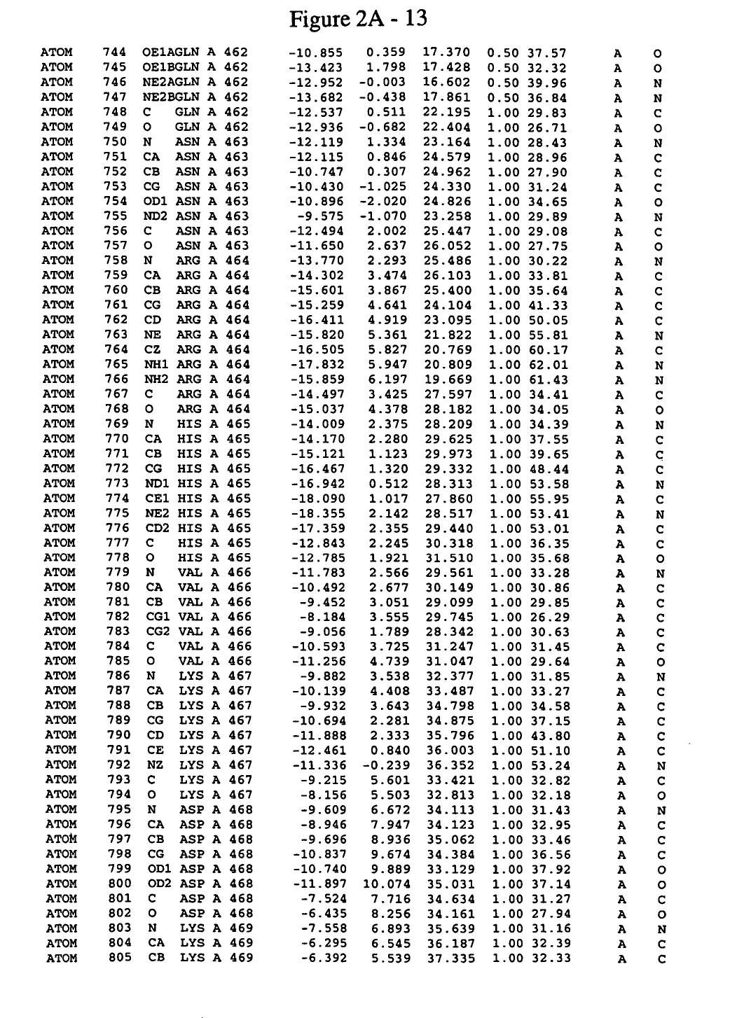

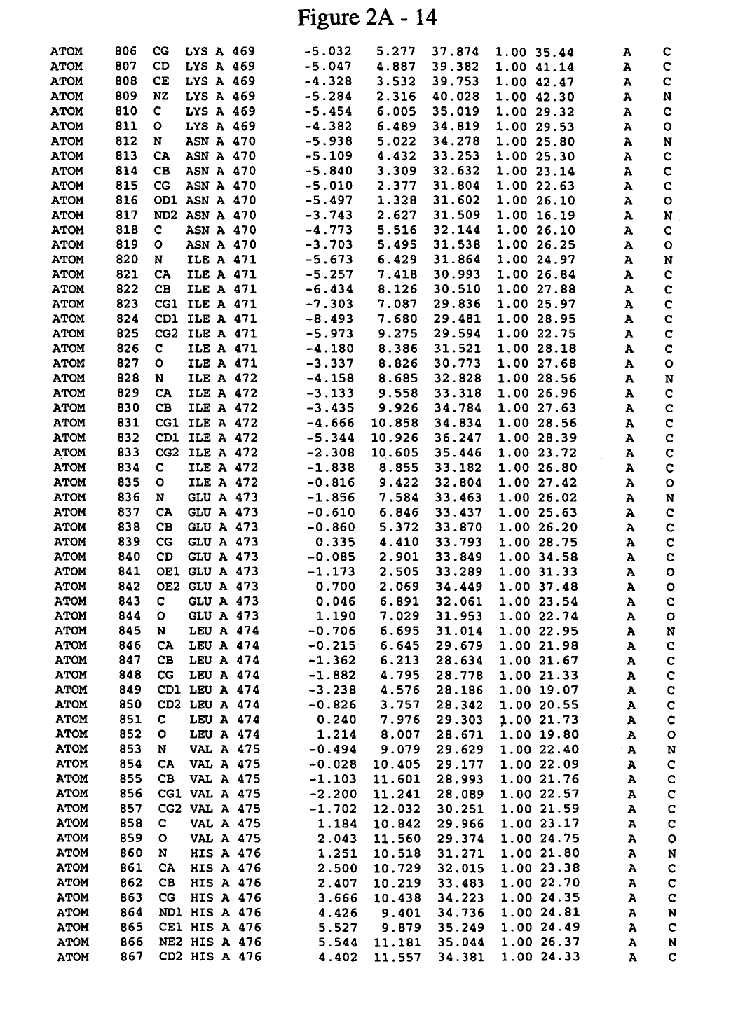

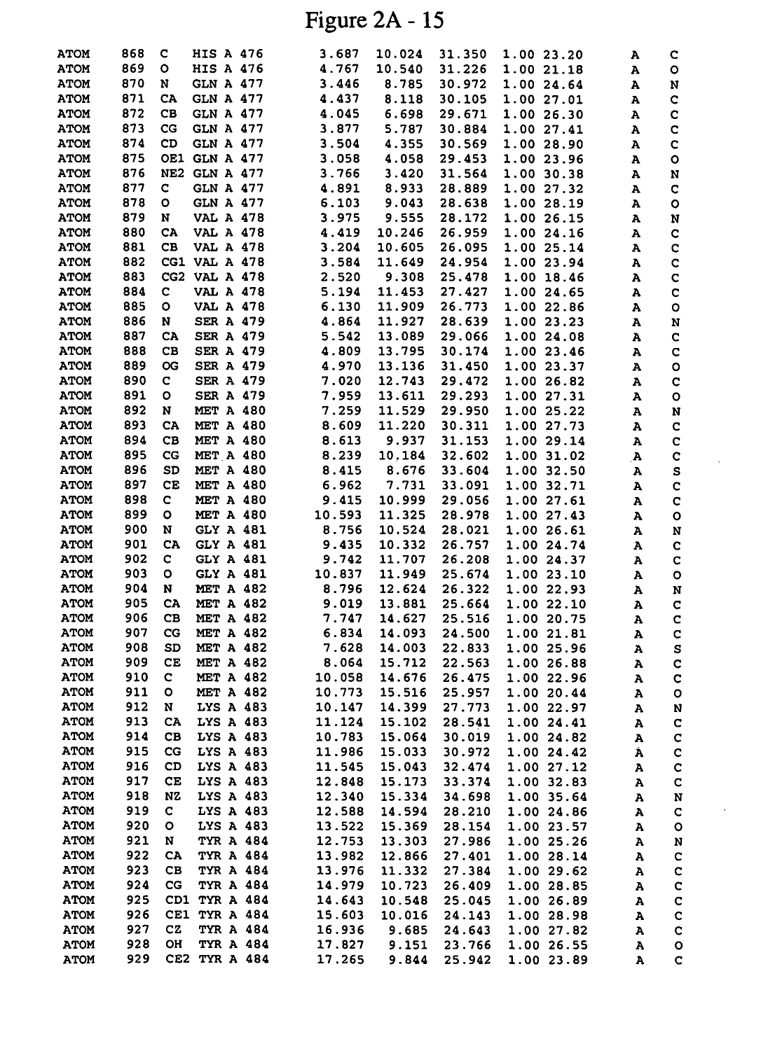

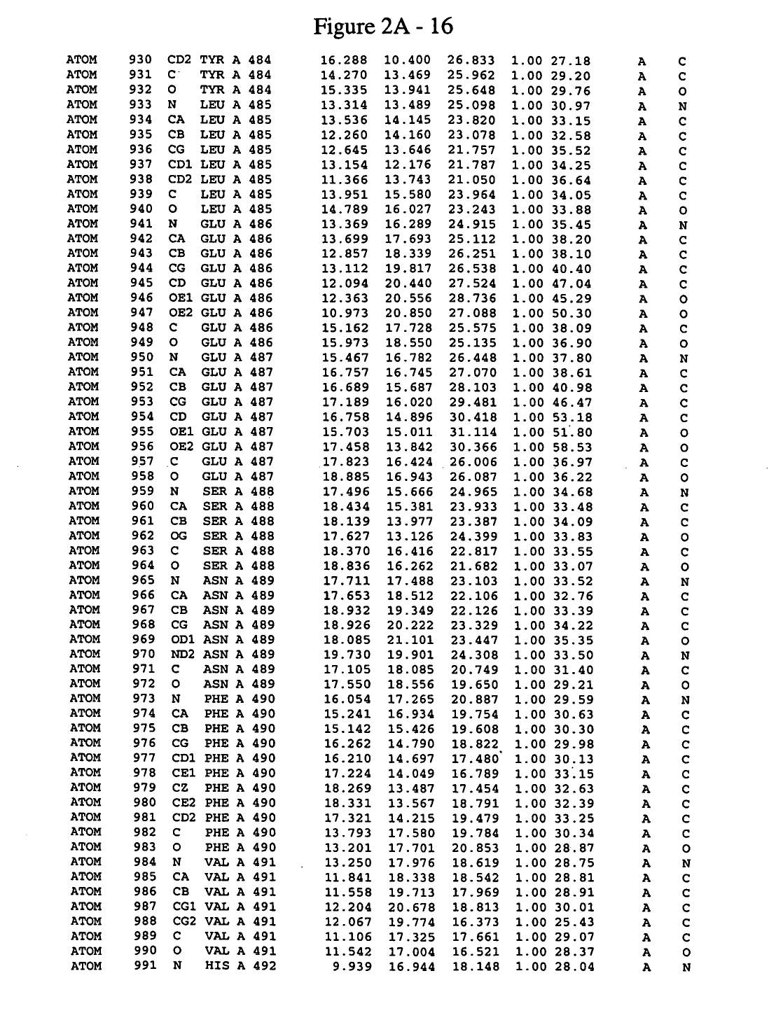

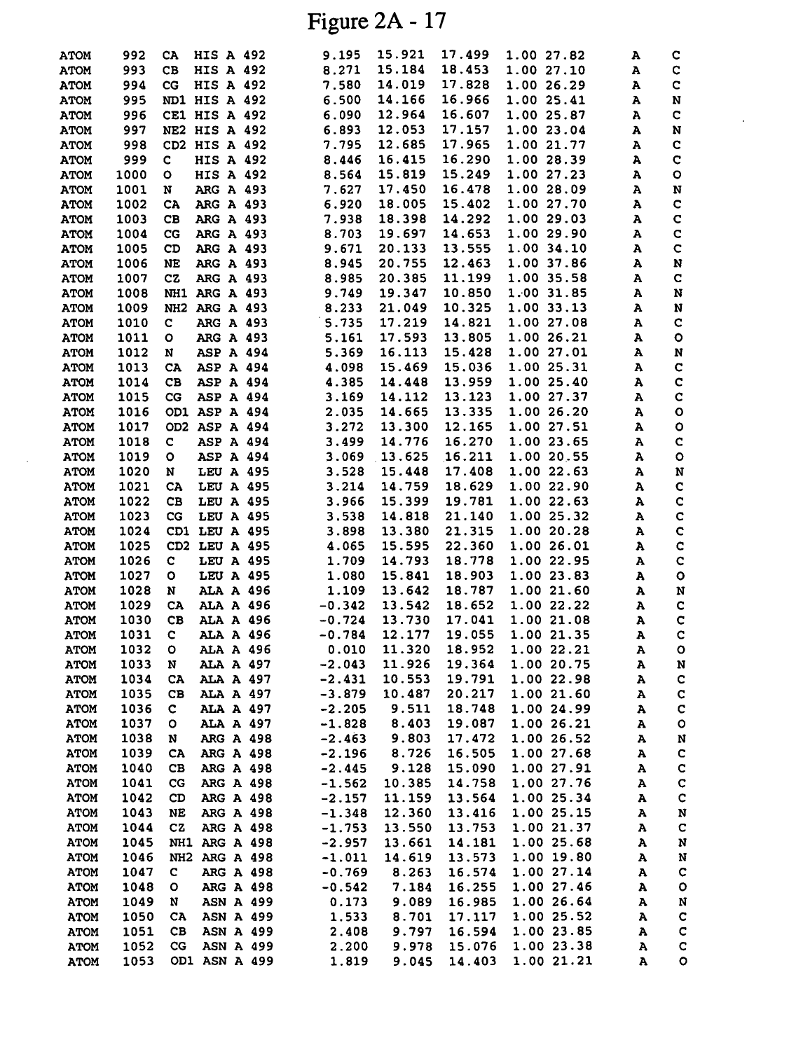

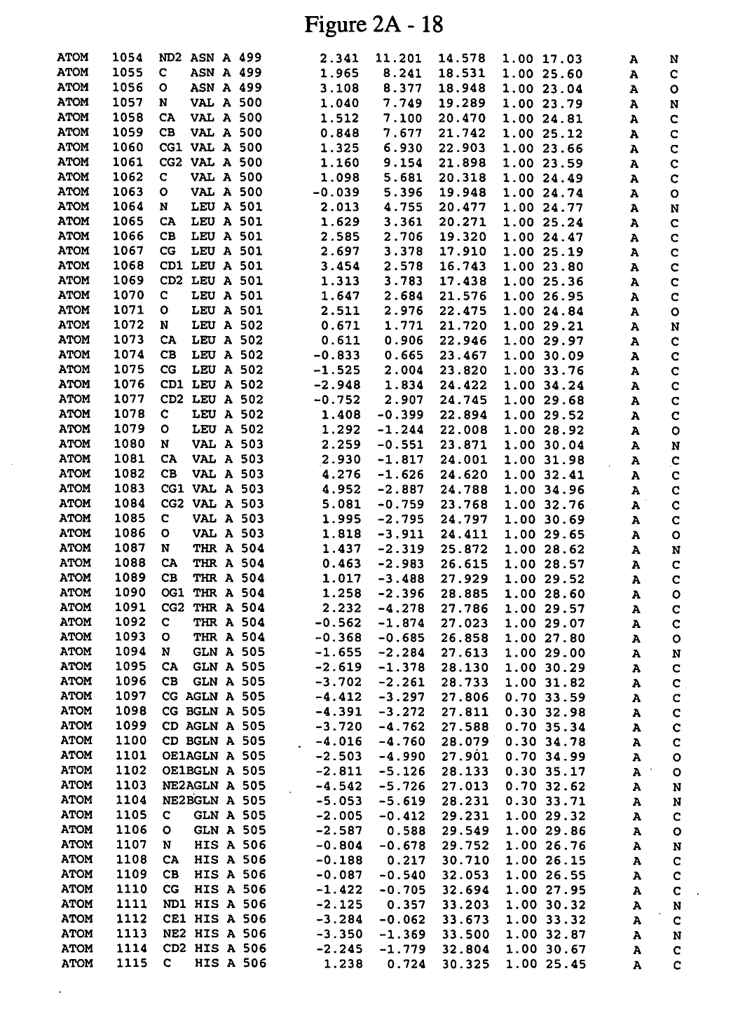

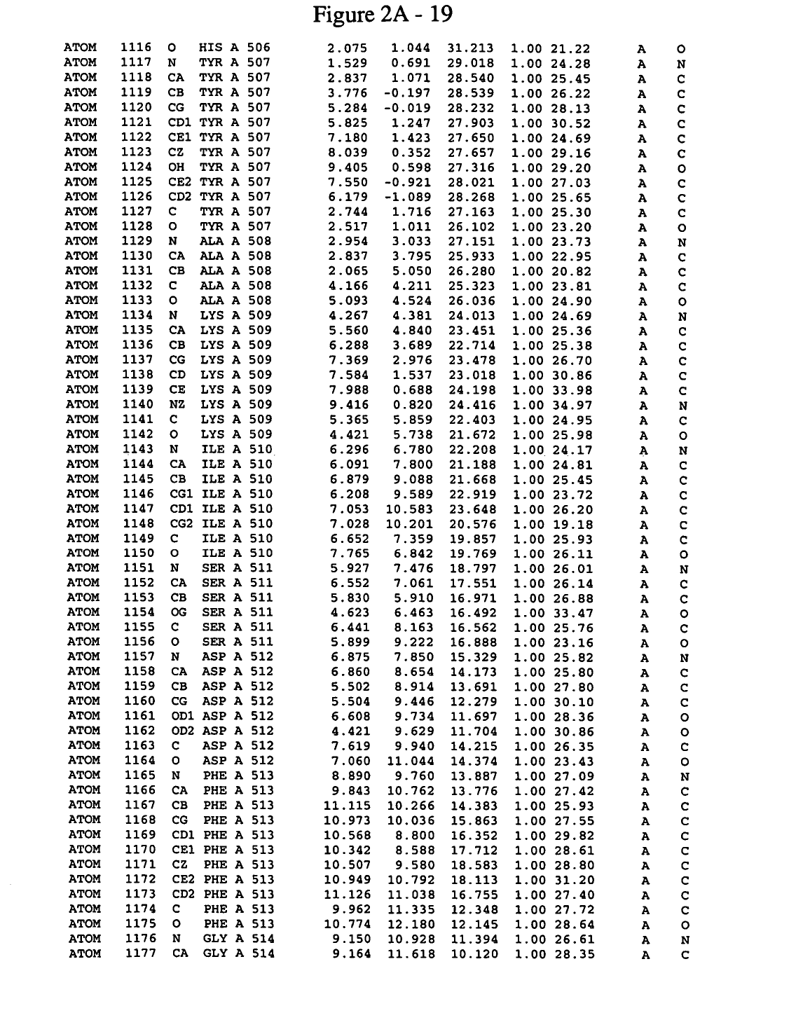

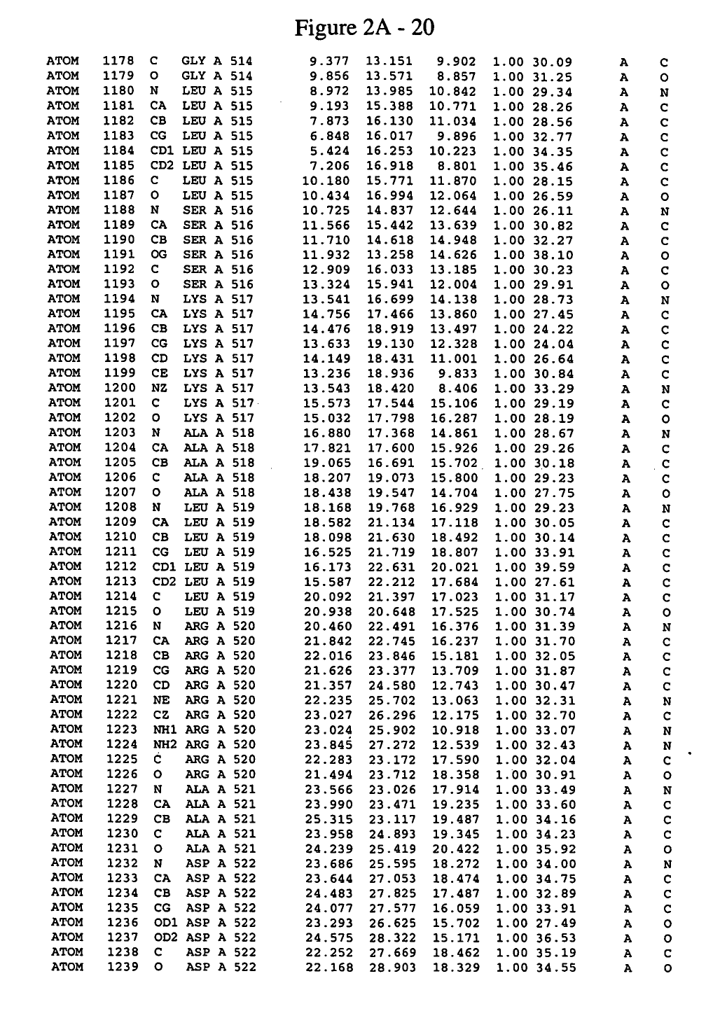

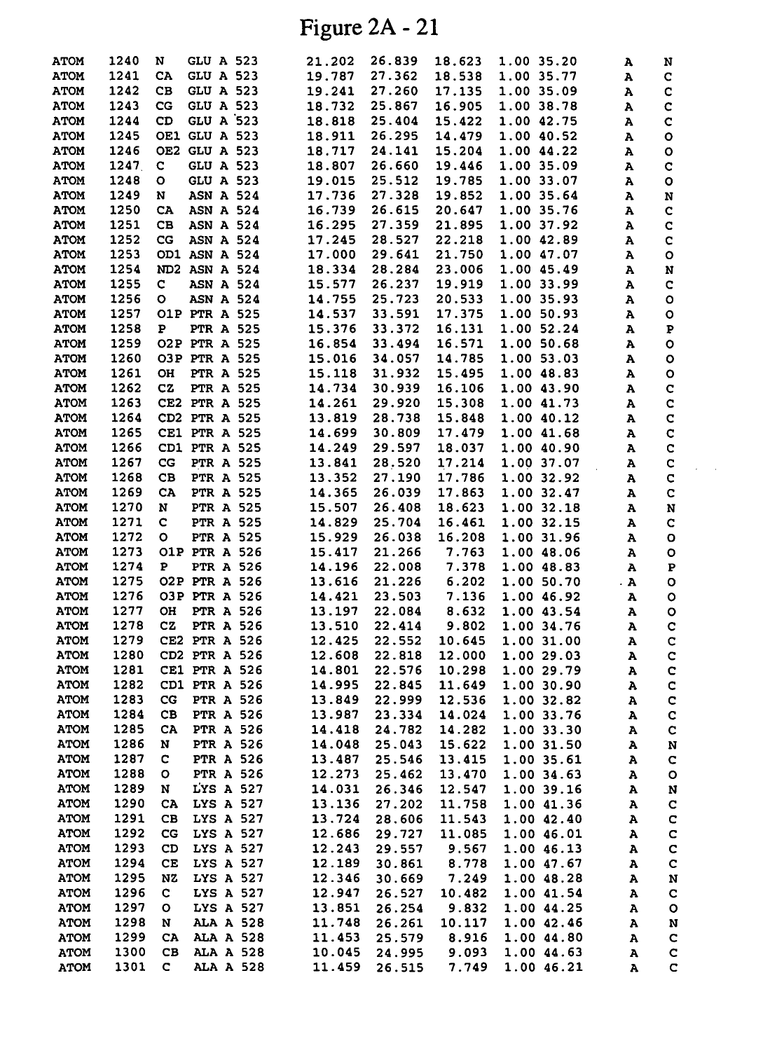

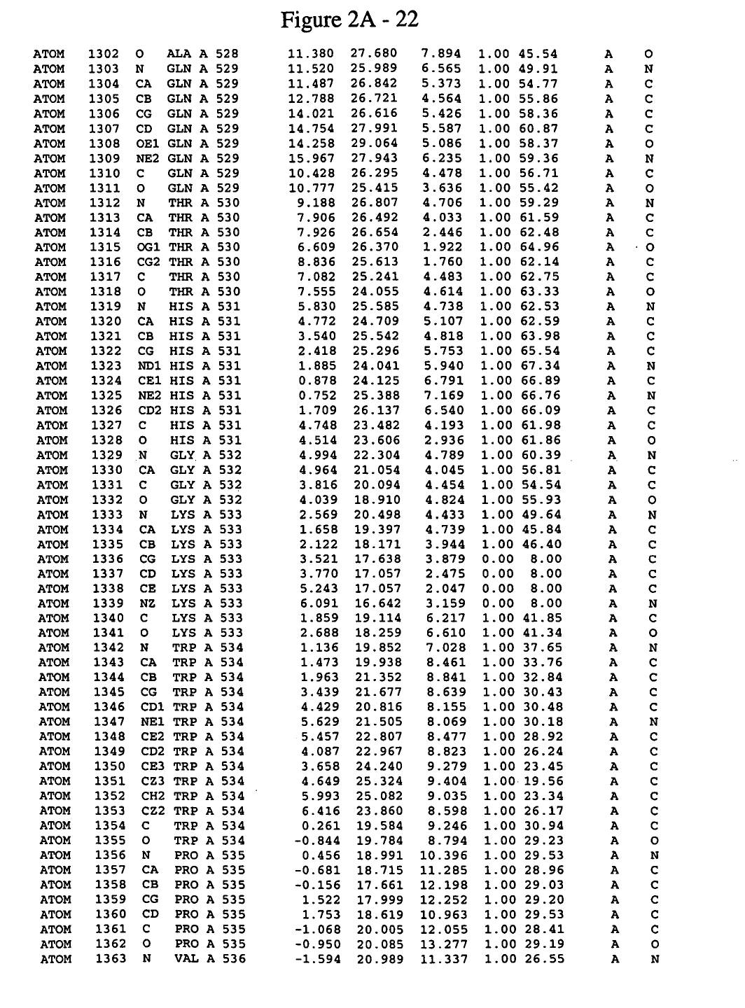

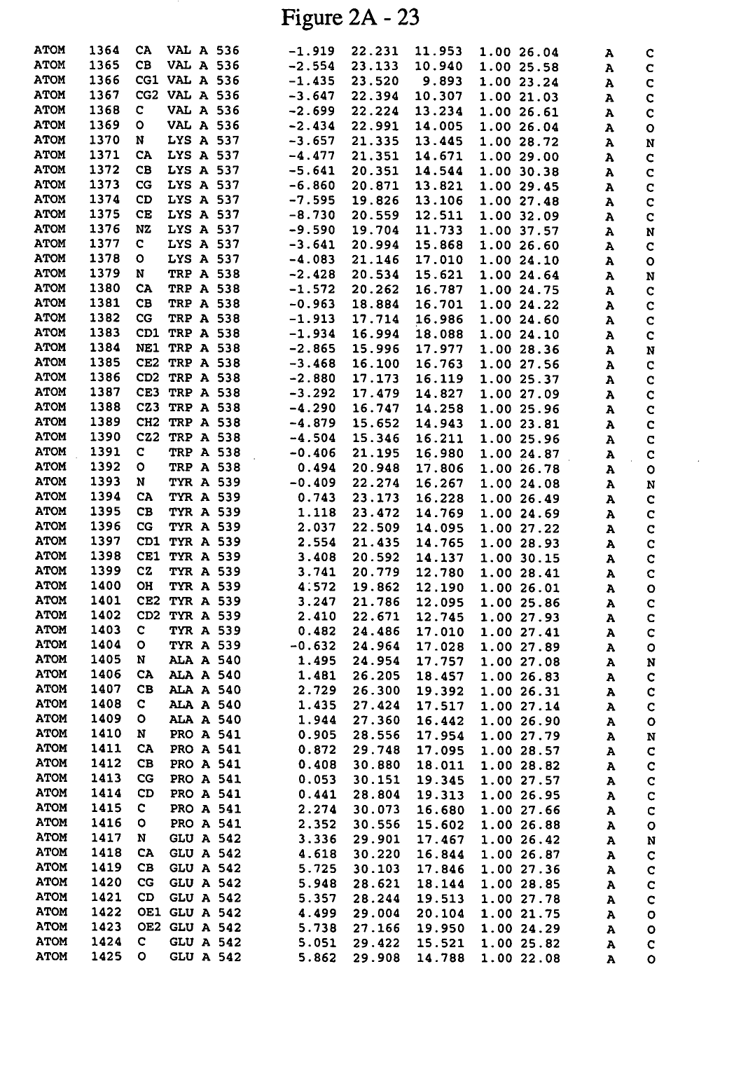

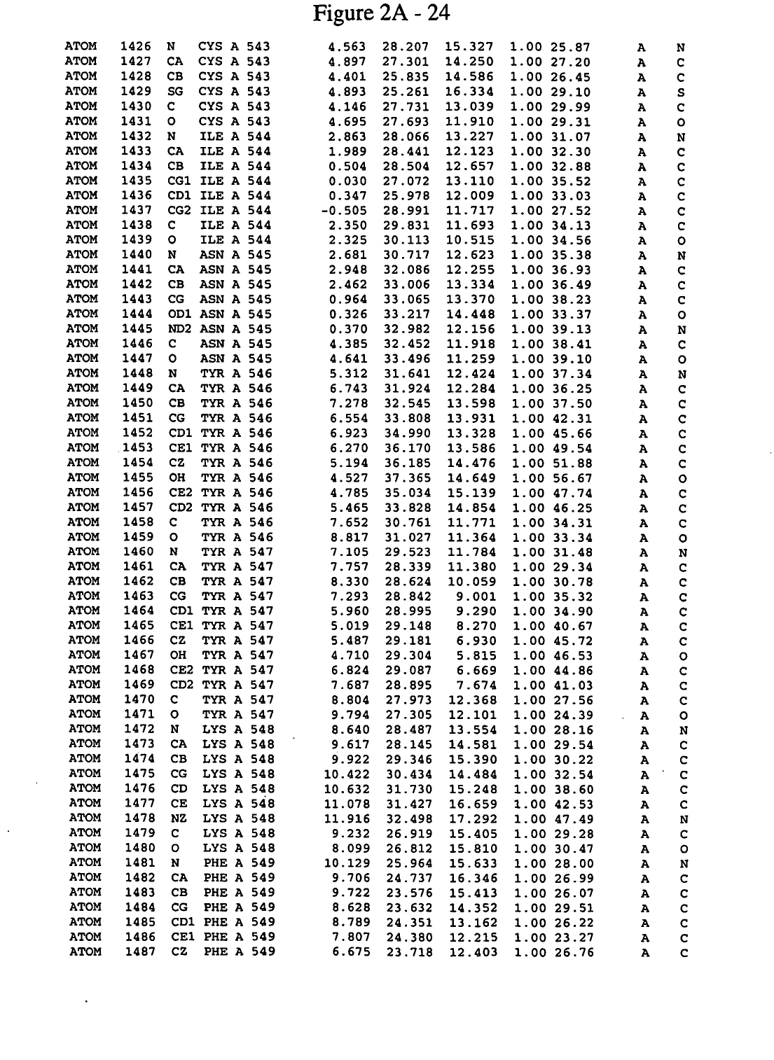

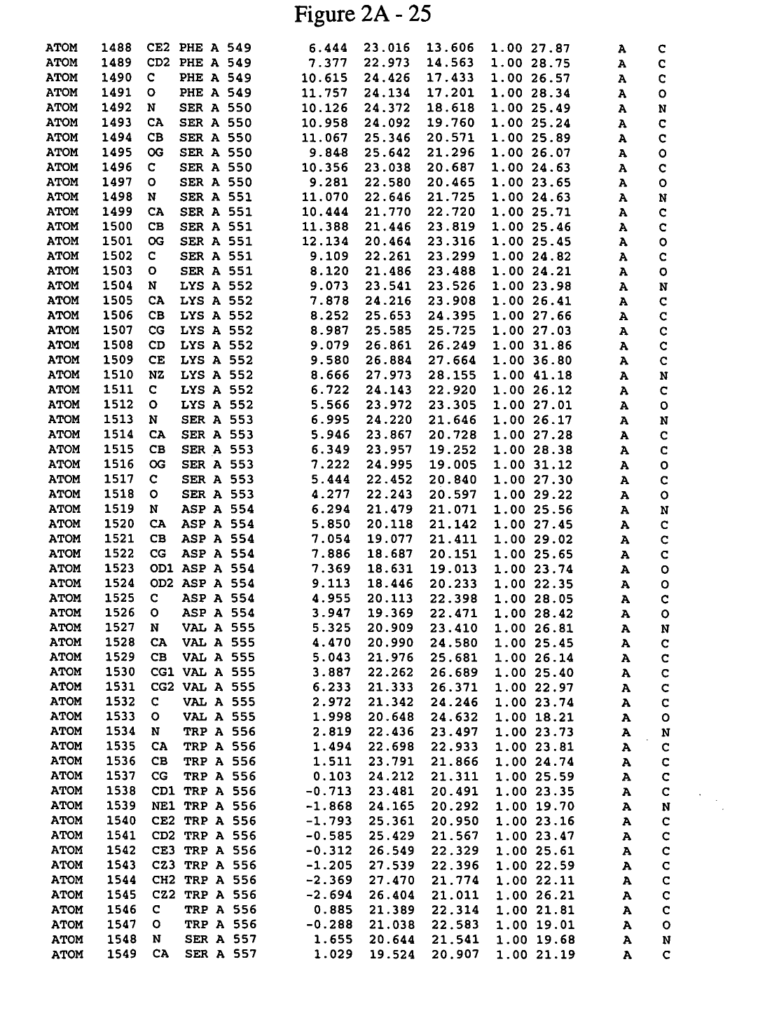

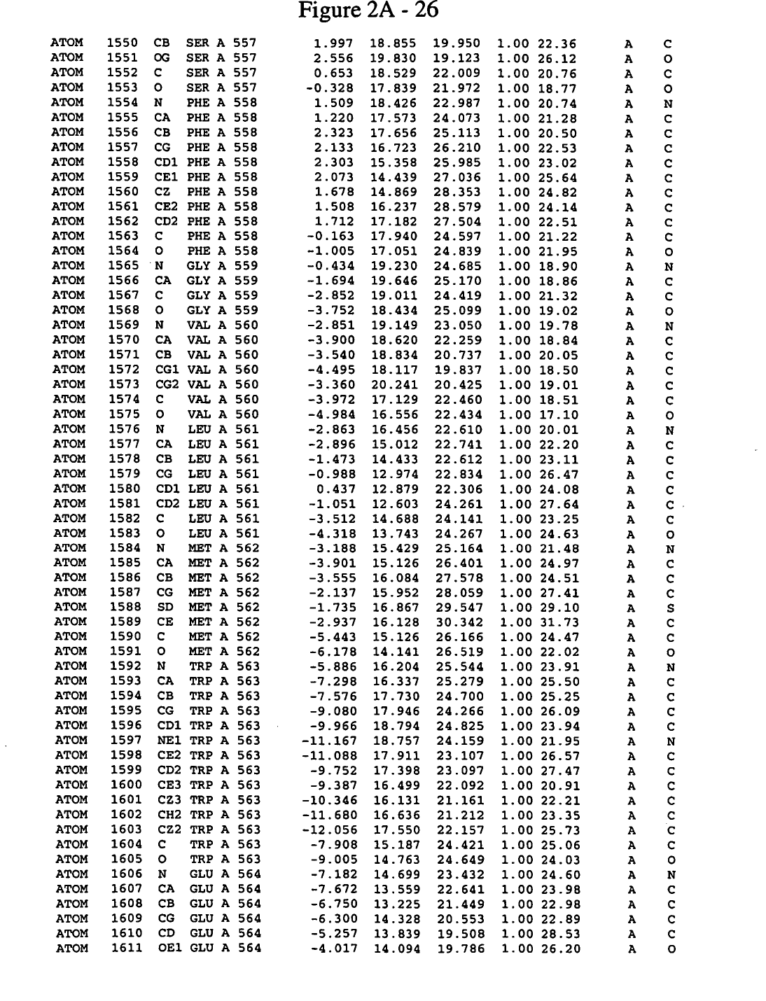

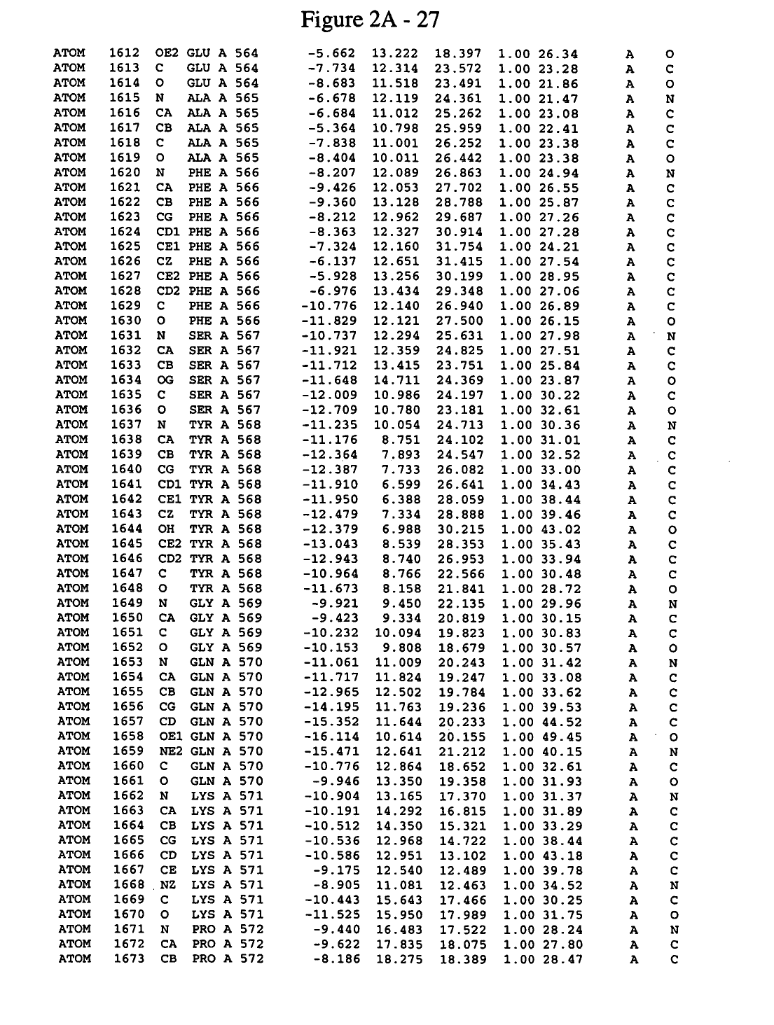

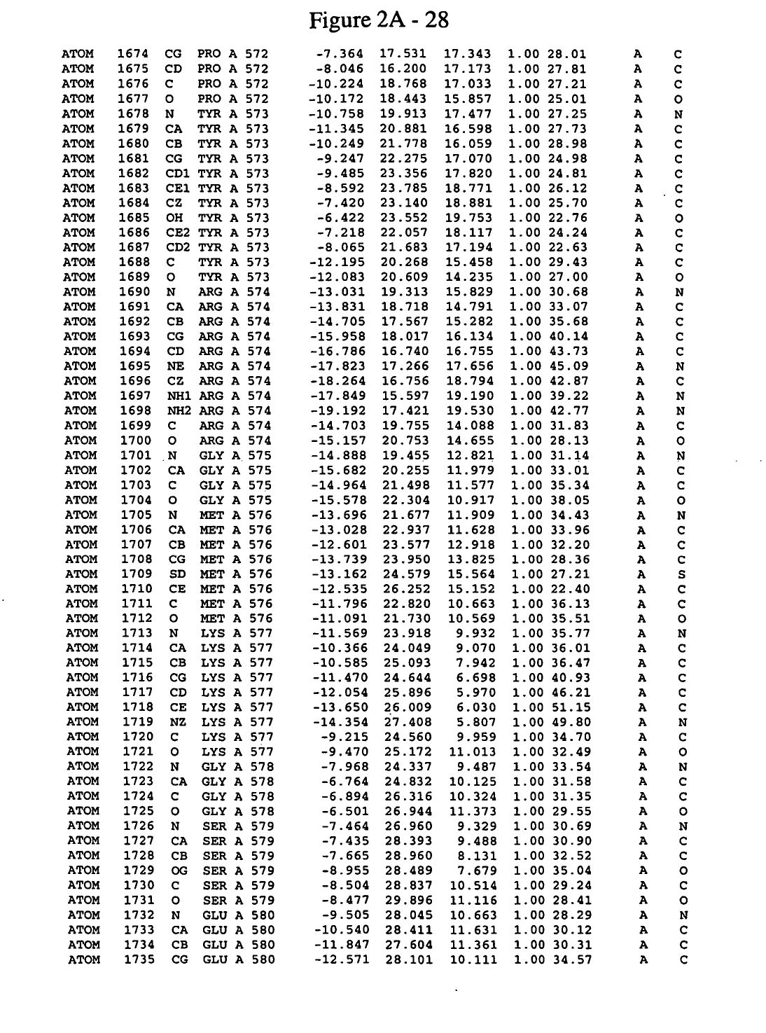

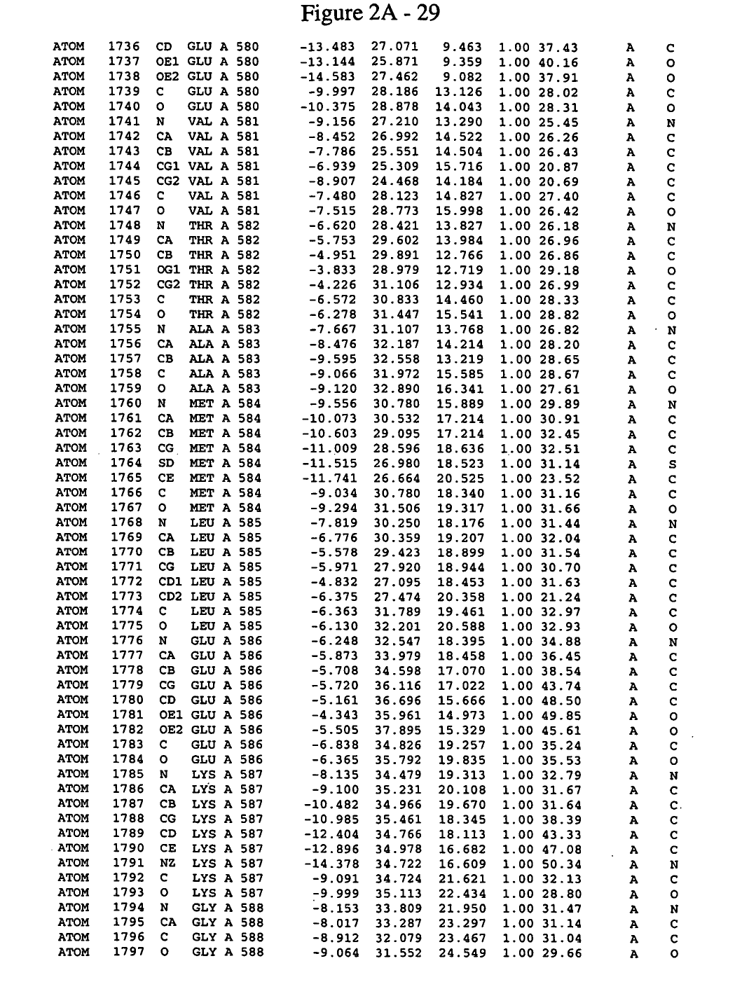

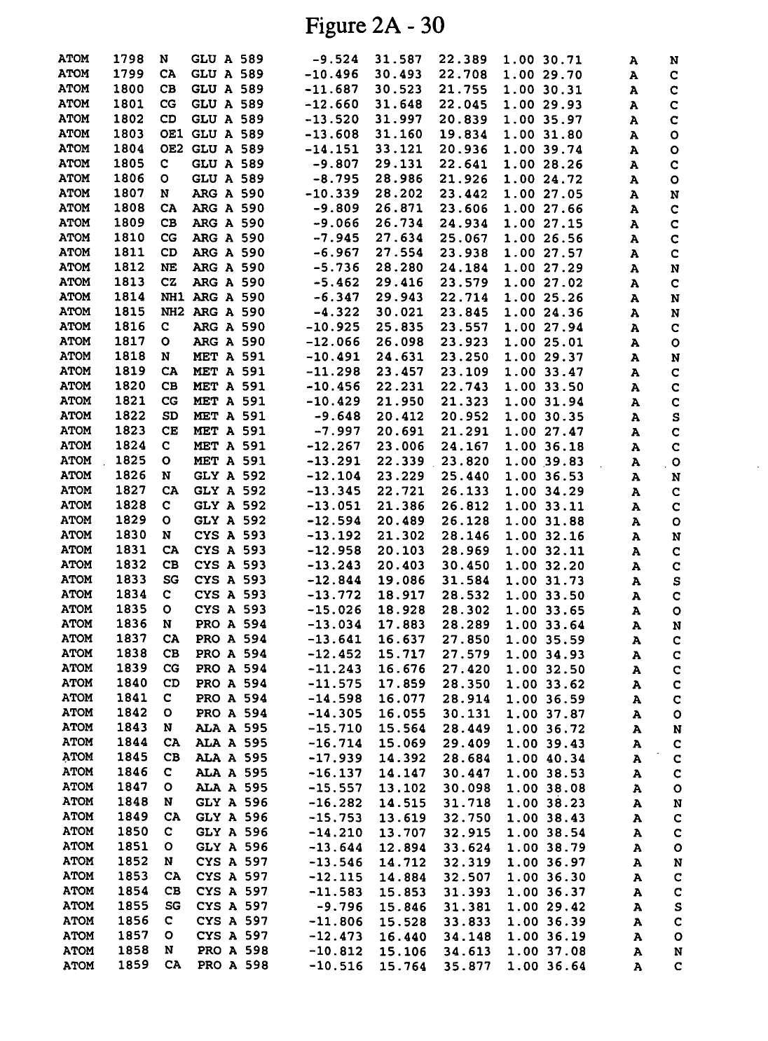

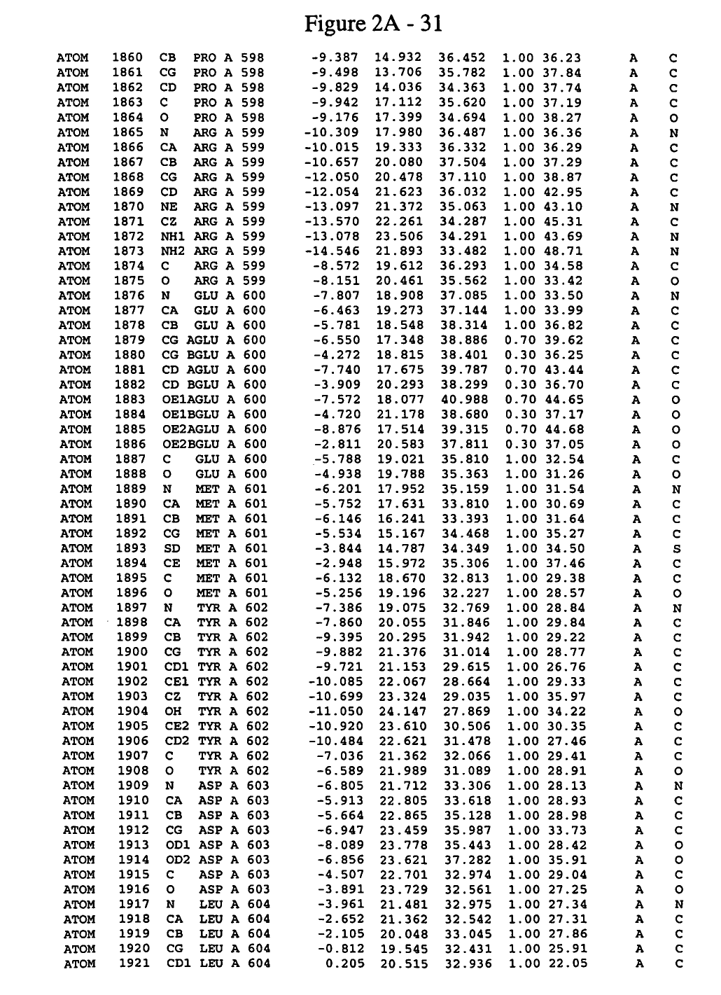

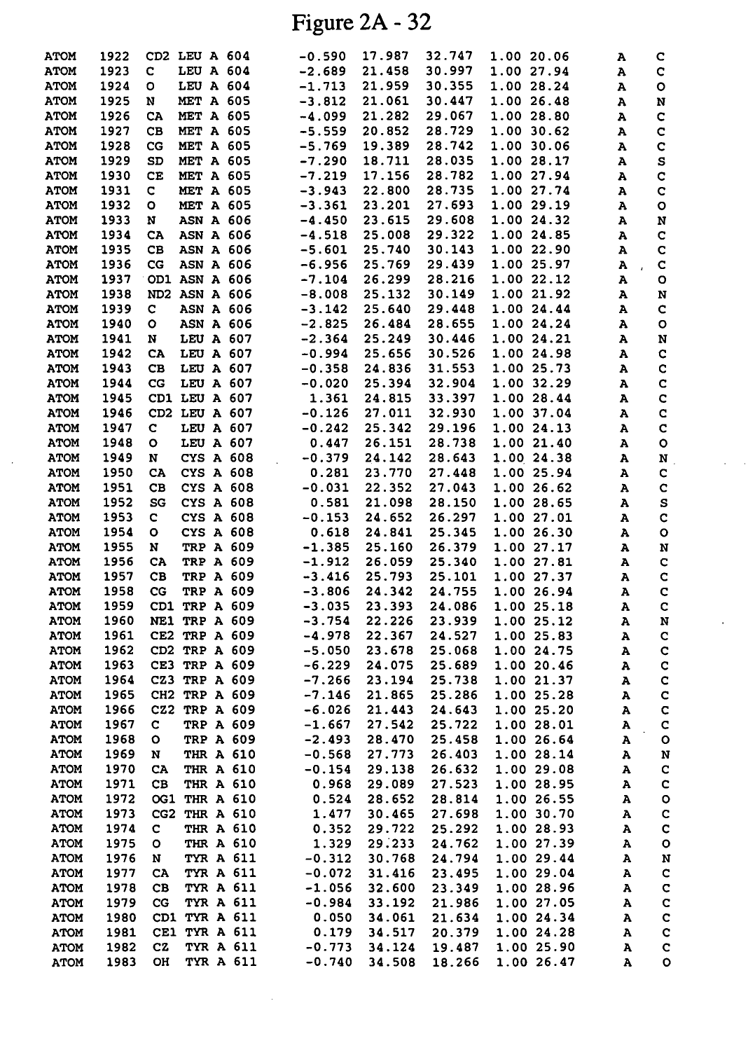

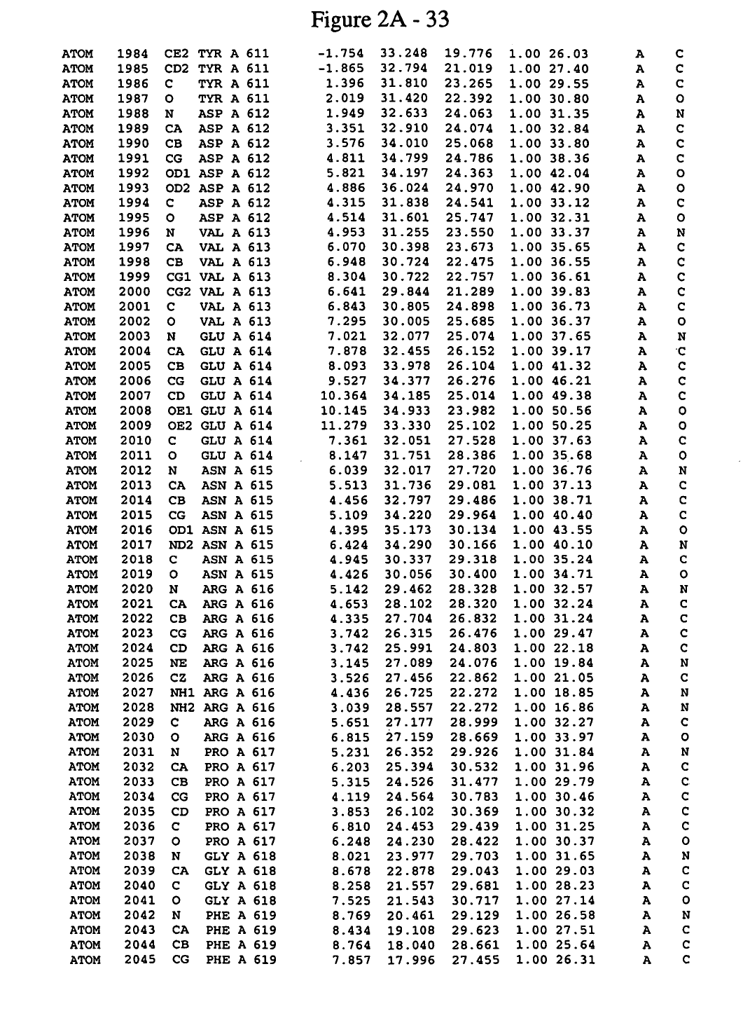

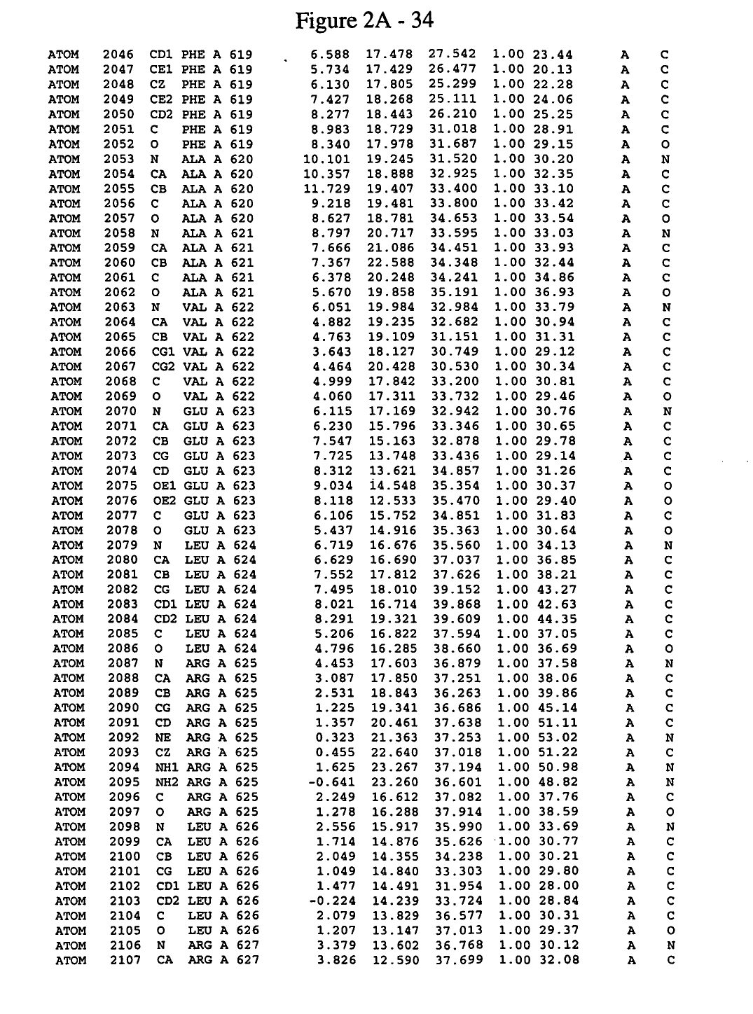

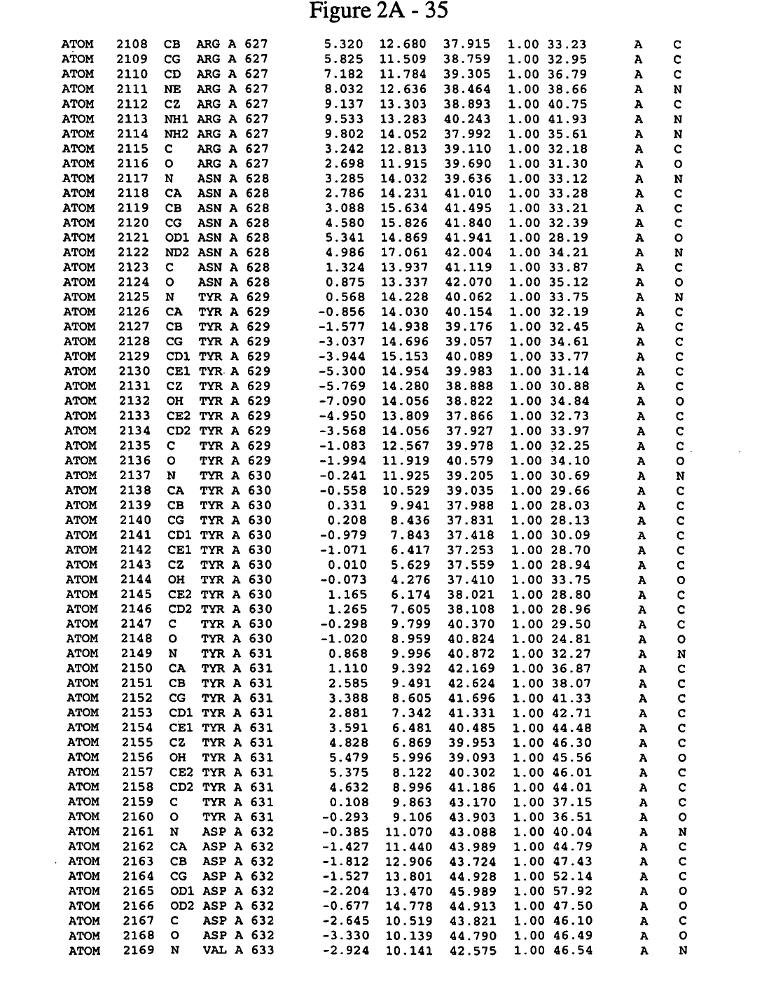

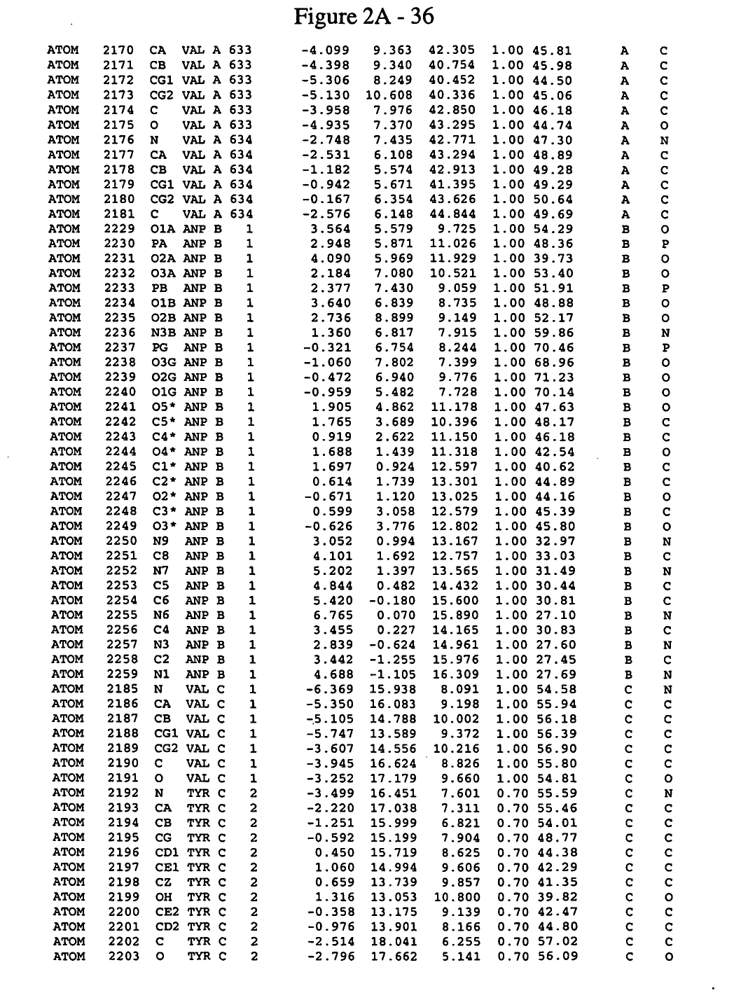

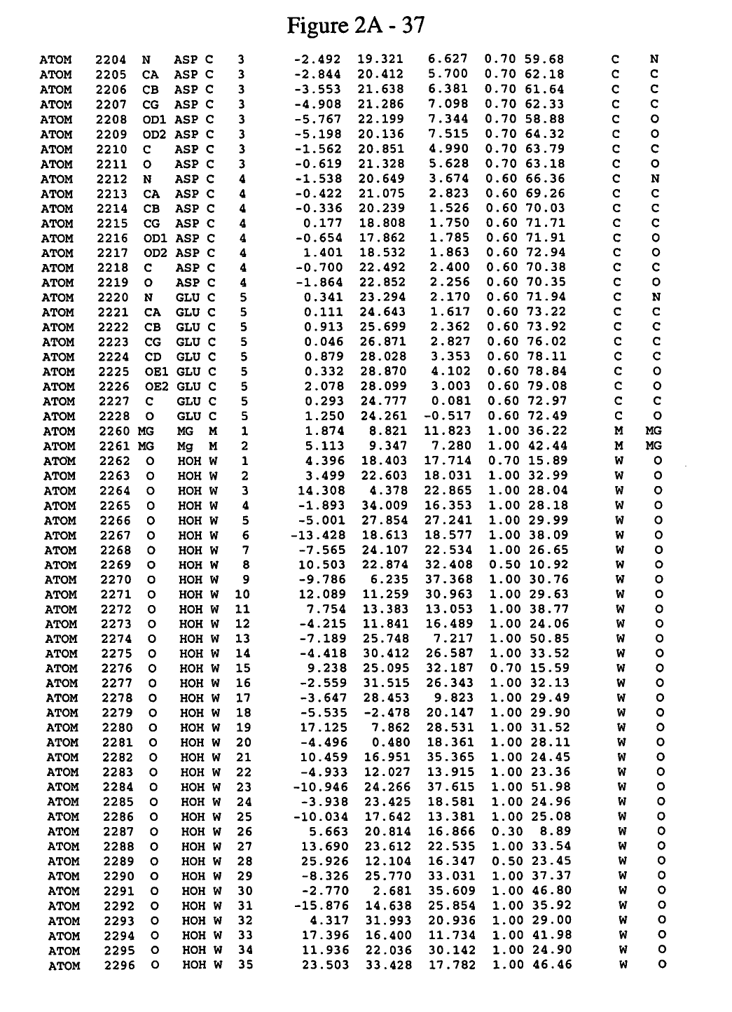

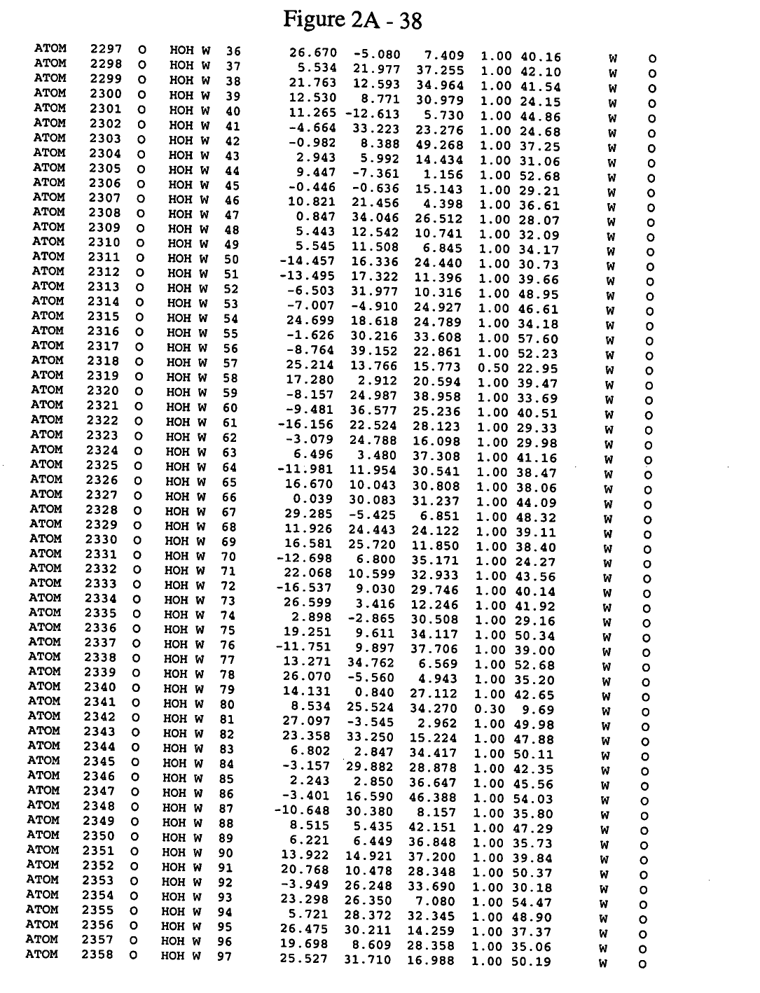

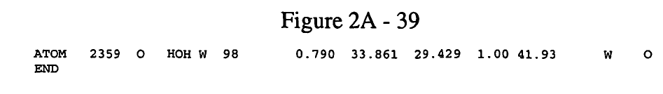

The term “contact score” refers to a measure of shape complementarity between the chemical entity and binding pocket, which is correlated with an RMSD value obtained from a least square superimposition between all or part of the atoms of the chemical entity and all or part of the atoms of the ligand bound (for example, AMP-PNP, staurosporine, PT426) in the binding pocket according to FIG. 1 or 2. The docking process may be facilitated by the contact score or RMSD values. For example, if the chemical entity moves to an orientation with high RMSD, the system will resist the motion. A set of orientations of a chemical entity can be ranked by contact score. A lower RMSD value will give a higher contact score. See Meng et al. J. Comp. Chem., 4, 505-524 (1992).

The term “correspond to” or “corresponding amino acids” when used in the context of amino acid residues that correspond to Syk amino acid residues refers to particular amino acid residues or analogues thereof in a Syk homologue that correspond to amino acid residues in the Syk protein. The corresponding amino acid may be an identical, mutated, chemically modified, conserved, conservatively substituted, functionally equivalent or homologous amino acid when compared to the Syk amino acid residue to which it corresponds.

Methods for identifying a corresponding amino acid are known in the art and are based upon sequence alignment, structural alignment, similarities in biochemical or structural function, or a combination thereof as compared to the Syk protein. For example, corresponding amino acid residues may be identified by superimposing the backbone atoms of the amino acid residues in Syk and the protein using well known software applications, such as QUANTA (Molecular Simulations, Inc., San Diego, Calif. ©2000). The corresponding amino acid residues may also be identified using sequence alignment programs such as the “bestfit” program, available from the Genetics Computer Group which uses the local homology algorithm described by Smith and Waterman in Advances in Applied Mathematics 2, 482 (1981), which is incorporated herein by reference, or CLUSTAL W Alignment Tool, supra.

The term “crystallization solution” refers to a solution that promotes crystallization comprising at least one agent, including a buffer, one or more salts, a precipitating agent, one or more detergents, sugars or organic compounds, lanthanide ions, a poly-ionic compound, and/or a stabilizer.

The term “docking” refers to orienting, rotating, translating a chemical entity in the binding pocket, domain, molecule or molecular complex or portion thereof. Docking may be performed by distance geometry methods that find sets of atoms of a chemical entity that match sets of sphere centers of the binding pocket, domain, molecule or molecular complex or portion thereof. See Meng et al. J. Comp. Chem., 4, 505-524 (1992). Sphere centers are generated by providing an extra radius of given length from the atoms (excluding hydrogen atoms) in the binding pocket, domain, molecule or molecular complex or portion thereof. Real-time interaction energy calculations, energy minimizations or rigid-body minimizations (Gschwend, et al., J. Mol. Recognition, 9:175-186 (1996)) can be performed while orienting the chemical entity to facilitate docking. For example, interactive docking experiments can be designed to follow the path of least resistance thereby simulating an interactive energy minimization. If the user in an interactive docking experiment makes a move to increase the energy, the system will resist that move. However, if that user makes a move to decrease energy, the system will favor that move by increased responsiveness. (Cohen, et al., J. Med. Chem. 33:889-894 (1990)). Docking can also be performed by combining a Monte Carlo search technique with rapid energy evaluation using molecular affinity potentials. See Goodsell and Olson, Proteins: Structure, Function and Genetics 8:195-202 (1990). Software programs that carry out docking functions include but are not limited to MATCHMOL (Cory et al., J. Mol. Graphics, 2, 39 (1984); MOLFIT (Redington, Comput. Chem., 16, 217 (1992)) and DOCK (Meng et al., supra).

The term “domain” refers to a structural unit of the Syk protein or homologue. The domain can comprise a binding pocket, a sequence or structural motif.

The term “full-length Syk” refers to the complete human Syk protein (amino acids residues 1 to 635; GenBank accession number A53596; SEQ ID NO: 1), which includes N-terminal tandem SH2 domains linked to a C-terminal catalytic domain.

The term “generating a three-dimensional structure” or “generating a three-dimensional representation” refers to converting the lists of structure coordinates into structural models or graphical representation in three-dimensional space. This can be achieved through commercially or publicly available software. A model of a three-dimensional structure of a molecule or molecular complex can thus be constructed on a computer screen by a computer that is given the structure coordinates and that comprises the correct software. The three-dimensional structure may be displayed or used to perform computer modeling or fitting operations. In addition, the structure coordinates themselves, without the displayed model, may be used to perform computer-based modeling and fitting operations.

The term “homologue of Syk_{Cat}” or “Syk_{Cat} homologue” or “Syk catalytic domain homologue” refers to a molecule that comprises a domain having at least 70%, 80%, 90%, 95%, 96%, 97%, 98%, 99% or greater than 99% sequence identity to the catalytic domain of Syk. Examples of homologues include but are not limited to human Syk or SykB, Syk, SykB or the catalytic domain thereof from another species, with mutations, conservative substitutions, additions, deletions or a combination thereof. In one embodiment, the homologue comprises a domain having at least 95%, 96%, 97% , 98% or 99% sequence identity to the catalytic domain of Syk, and has conservative mutations as compared to the catalytic domain of Syk. The homologue can be Syk, SykB or the catalytic domain thereof from another animal species. Such animal species include, but are not limited to, mouse, rat, a primate such as monkey or other primates.

The term “homology model” refers to a structural model derived from known three-dimensional structure(s). Generation of the homology model, termed “homology modeling”, can include sequence alignment, residue replacement, residue conformation adjustment through energy minimization, or combination thereof.

The term “interaction energy” refers to the energy determined for the interaction of a chemical entity and a binding pocket, domain, molecule or molecular complex or portion thereof. Interactions include but are not limited to one or more of covalent interactions, non-covalent interactions such as hydrogen bond, electrostatic, hydrophobic, aromatic, van der Waals interactions, and non-complementary electrostatic interactions such as repulsive charge-charge, dipole-dipole and charge-dipole. As interaction energies are measured in negative values, the lower the value the more favorable the interaction.

The term “motif” refers to a group of amino acid residues in the Syk_{Cat} protein or homologue that defines a structural compartment or carries out a function in the protein, for example, catalysis, structural stabilization, or phosphorylation. The motif may be conserved in sequence, structure and function. The motif can be contiguous in primary sequence or three-dimensional space. Examples of a motif include but are not limited to the phosphorylation lip or activation loop, the glycine-rich phosphate anchor loop, the catalytic loop, the DFG or DFGWSxxxxxxxRxTxCGTxDYLPPE loop (see, Xie et al., Structure, 6 pp. 983-991 (1998); Giet and Prigent, J. Cell. Sci., 112, pp. 3591-601 (1991)) and the degradation box.

The term “part of a binding pocket” refers to less than all of the amino acid residues that define the binding pocket. The structure coordinates of residues that constitute part of a binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence. In one embodiment, part of a binding pocket has at least two amino acid residues, preferably at least four, six or eight amino acid residues.

The term “part of a Syk_{Cat} protein” or “part of a Syk_{Cat} homologue” refers to less than all of the amino acid residues of a Syk_{Cat} protein or homologue. In one embodiment, part of a Syk_{Cat} protein or homologue defines the binding pockets, domains, sub-domains, and motifs of the protein or homologue. The structure coordinates of residues that constitute part of a Syk_{Cat} protein or homologue may be specific for defining the chemical environment of the protein, or useful in designing fragments of an inhibitor that may interact with those residues. The portion of residues may also be residues that are spatially related and define a three-dimensional compartment of a binding pocket, motif or domain. The residues may be contiguous or non-contiguous in primary sequence. For example, the portion of residues may be key residues that play a role in ligand or substrate binding, peptide binding, antibody binding, catalysis, structural stabilization or degradation.

The term “root mean square deviation” or “RMSD” means the square root of the arithmetic mean of the squares of the deviations from the mean. It is a way to express the deviation or variation from a trend or object. For purposes of this invention, the “root mean square deviation” defines the variation in the backbone atoms of a protein from the backbone atoms of Syk_{Cat}, a binding pocket, a motif, a domain, or portion thereof, as defined by the structure coordinates of Syk_{Cat} described herein. It would be apparent to the skilled worker that the calculation of RMSD involves a standard error of ±0.1 Å.

The term “soaked” refers to a process in which the crystal is transferred to a solution containing a compound of interest.

The term “structure coordinates” refers to Cartesian coordinates derived from mathematical equations related to the patterns obtained from diffraction of a monochromatic beam of X-rays by the atoms (scattering centers) of a protein or protein complex in crystal form. The diffraction data are used to calculate an electron density map of the repeating unit of the crystal. The electron density maps are then used to establish the positions of the individual atoms of the molecule or molecular complex.

The term “sub-domain” refers to a portion of the domain as defined above in the Syk protein or homologue. The catalytic domain (approximately amino acid residues 343-639) of Syk is a bi-lobal structure consisting of an N-terminal, β-strand sub-domain or lobe and a C-terminal, α-helical sub-domain or lobe.

The term “substantially all of a Syk_{Cat} binding pocket” or “substantially all of a Syk_{Cat} protein” refers to all or almost all of the amino acid residues in the Syk_{Cat} binding pocket or protein. For example, substantially all of a Syk_{Cat} binding pocket can be 100%, 95%, 90%, 80%, or 70% of the residues defining the Syk_{Cat} binding pocket or protein.

The term “substrate binding pocket” refers to the binding pocket for a substrate of Syk_{Cat} or homologue thereof. A substrate is generally defined as the molecule upon which an enzyme performs catalysis. Natural substrates, synthetic substrates or peptides, or mimics of a natural substrates of Syk_{Cat} or homologue thereof may associate with the substrate binding pocket.

The term “sufficiently homologous to Syk_{Cat}” refers to a protein that has a sequence identity of at least 25% compared to Syk_{Cat} protein. In other embodiments, the sequence homology is at least 40%. In other embodiments, the sequence identity is at least 50%, 60%, 70%, 80%, 90%, 95%, 96%, 97%, 98% or 99%.

The term “Syk_{Cat}” or “Syk_{Cat} protein” refers to the catalytic domain of human Syk.

The term “Syk_{Cat}B” or “Syk_{Cat}B protein” refers to the catalytic domain of SykB, a less common form of Syk protein that lacks a twenty-three amino acid residue insert in the linker region present in Syk.

The “Syk_{Cat} ATP-binding pocket” refers to a binding pocket of a molecule or molecular complex defined by the structure coordinates of a certain set of amino acid residues present in the Syk_{Cat} structure, as described below. In general, the ligand for the ATP-binding pocket is a nucleotide such as ATP. This binding pocket is in the catalytic active site of the catalytic domain. In the protein kinase family, the ATP-binding pocket is generally located at the interface of the α-helical and β-strand sub-domains, and is bordered by the glycine rich loop and the hinge (See, Xie et al., Structure, 6, pp. 983-991 (1998), incorporated herein by reference).

The term “Syk_{Cat}-like” refers to all or a portion of a molecule or molecular complex that has a commonality of shape to all or a portion of the Syk_{Cat} protein. For example, in the Syk_{Cat}-like ATP-binding pocket, the commonality of shape is defined by a root mean square deviation of the structure coordinates of the backbone atoms between the amino acid residues in the Syk_{Cat}-like ATP-binding pocket and the amino acid residues in the Syk_{Cat} ATP-binding pocket (as set forth in FIGS. 1 or 2). Compared to an amino acid in the Syk_{Cat} ATP-binding pocket, the corresponding amino acid residues in the Syk_{Cat}-like ATP-binding pocket may or may not be identical. Depending on the Syk_{Cat} amino acid residues that define the Syk_{Cat}-ATP binding pocket, one skilled in the art would be able to locate the corresponding amino acid residues that define a Syk_{Cat}-like-ATP binding pocket in a protein based upon sequence or structural homology.

The term “Syk_{Cat} protein complex” or “Syk_{Cat} homologue complex” refers to a molecular complex formed by associating the Syk_{Cat} protein or Syk_{Cat} homologue with at least a chemical entity, for example, a ligand, a substrate, nucleotide triphosphate, nucleotide diphosphate, phosphate, an agonist or antagonist, inhibitor, antibody, drug or compound. In one embodiment, the chemical entity is selected from the group consisting of ATP, an ATP analogue, a nucleotide triphosphate and ATP-binding pocket inhibitor. In another embodiment, the chemical entity is an ATP analogue such as Mg-AMP-PNP, or adenosine. Mg refers to Mg⁺². In one embodiment, the chemical entity is PT426 or staurosporine.

The term “three-dimensional structural information” refers to information obtained from the structure coordinates. Structural information generated can include the three-dimensional structure or graphical representation of the structure. Structural information can also be generated when subtracting distances between atoms in the structure coordinates, calculating chemical energies for a Syk_{Cat} molecule or molecular complex or homologues thereof, calculating or minimizing energies for an association of a Syk_{Cat} molecule or molecular complex or homologues thereof to a chemical entity.

### Crystallizable Compositions and Crystals of Syk_{Cat} Protein and Protein Complexes

According to one embodiment, the invention provides a crystal or crystal composition comprising a catalytic domain of Syk protein (Syk_{Cat}) or homologue thereof in the presence or absence of a chemical entity. The catalytic domain of Syk protein may be phosphorylated or unphosphorylated. In one embodiment, the chemical entity binds to the active site. In one embodiment, the chemical entity is selected from the group consisting of an ATP analogue, ATP, adenosine, AMP-PNP, nucleotide triphosphate, nucleotide diphosphate, phosphate, staurosporine, an agonist, an antagonist and an active site inhibitor. In one embodiment, the chemical entity is staurosporine. In another embodiment, the chemical entity is AMP-PNP. In one embodiment, the chemical entity binds to the substrate binding pocket. In one embodiment, the chemical entity is selected from the group consisting of NAc-Glu-Glu-Asp-Asp-Tyr-Glu-Ser-Pro-NH₂ (PT426) (SEQ ID NO: 2), Glu-Glu-Asp-Asp-Tyr-Glu-Ser-Pro (SEQ ID NO: 5), a peptide comprising the amino acid sequence Glu-Asp-Asp-Tyr (residues 2-5 of SEQ ID NO: 5), a peptide comprising the amino acid sequence Asp-Asp-Tyr-Glu (residues 3-6 of SEQ ID NO: 5), a peptide comprising the amino acid sequence Asp-Tyr-Glu-Ser (residues 4-7 of SEQ ID NO: 5), a peptide comprising the amino acid sequence Tyr-Glu-Ser-Pro (residues 5-8 of SEQ ID NO: 5), a peptide comprising the amino acid sequence Glu-Glu-Asp-Asp-Tyr (residues 1-5 of SEQ ID NO: 5), a peptide comprising the amino acid sequence Glu-Asp-Asp-Tyr-Glu (residues 2-6 of SEQ ID NO: 5), a peptide comprising the amino acid sequence Asp-Asp-Tyr-Glu-Ser (residues 3-7 of SEQ ID NO: 5), a peptide comprising the amino acid sequence Asp-Tyr-Glu-Ser-Pro (residues 4-8 of SEQ ID NO: 5), a peptide comprising amino acids Asp-Glu-Glu-Asp-Tyr (SEQ ID NO: 6), a peptide comprising amino acids Asp-Glu-Glu-Tyr-Asp (SEQ ID NO: 7), a peptide comprising amino acids Asp-Glu-Tyr-Glu-Asp (SEQ ID NO: 8), a peptide comprising amino acids Asp-Tyr-Glu-Glu-Val (SEQ ID NO: 9), and a peptide comprising amino acids Tyr-Ser-Ile-Ile-Nle (SEQ ID NO: 10). Peptides with SEQ ID NOs 6-10 were screened and found to be preferred substrates for Syk in United States Patent Application Publication No. 2002/0155503, incorporated herein by reference.

In one embodiment, the crystal has unit cell dimensions of a=39.45 Å, b=84.17 Å, c=85.00 Å, α=90°, β=90°, γ=90° and belongs to space group P2₁2₁2₁. Preferably, the crystal comprises the Syk_{Cat}-staurosporine complex. In another embodiment, the crystal has a unit cell dimension of a=39.58 Å, b=84.67 Å, c=90.63 Å, α=90°, β=90°, γ=γ90° and belongs to space group P2₁2₁2₁. In one embodiment, the crystal comprises the Syk_{Cat}-PT426-AMP-PNP complex. It will be readily apparent to those skilled in the art that the unit cells of the crystal compositions may deviate ±1-2 Å from the above cell dimensions depending on the deviation in the unit cell calculations.

As used herein, the Syk_{Cat} protein in the crystal may be amino acid residues 343-635, 358-635 or 364-634 of SEQ ID NO: 1 or fragments of at least 100 of these amino acid residues thereof, or the foregoing with conservative substitutions, deletions or insertions.

The Syk_{Cat} protein or its homologue may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of a natural product. In another embodiment, the protein is produced recombinantly and overexpressed in a baculovirus system.

The invention also provides a method of making a crystal comprising a catalytic domain of Syk protein or a homologue thereof in the presence or absence of a chemical entity. Such methods comprise the steps of:

a. producing and purifying a catalytic domain of Syk protein or homologue thereof;

b. combining said catalytic domain of Syk protein, or a homologue thereof in the presence or absence of a chemical entity with a crystallization solution to produce a crystallizable composition; and

c. subjecting said crystallizable composition to conditions which promote crystallization. In one embodiment, the chemical entity binds to the active site of said Syk protein. In another embodiment, the chemical entity binds to the substrate binding site.

The crystallization solution may include, but is not limited to, polyethylene glycol (PEG) at between 5 to 40% v/v, 50-300 mM acetate, and a buffer that maintains pH at between about 4.0 and 7.0. In one embodiment, the crystallizable composition comprises equal volumes of a solution of Syk_{Cat}, 20 mM diethanolamine (pH 8.6), 500 mM NaCl and 300 mM staurosporine, and a solution of 20% polyethylene glycol with average molecular weight 2000 (PEG 2K), 0.2 ammonium acetate, 0.1 M sodium cacodylate (pH 5.23). In one embodiment, the crystallizable composition comprises equal volumes of a solution of Syk_{Cat} (2-4 mg/mL), 20 mM diethanolamine (pH 8.6), 500 mM NaCl, 2 mM AMP-PNP, 6 mM MgCl₂ and 500 mM of the peptide NAc-Glu-Glu-Asp-Asp-Tyr-Glu-Ser-Pro-NH₂ (SEQ ID NO: 2), and a solution containing 22% PEG 2K, 0.2 M magnesium acetate, 0.1 M sodium cacodylate (pH 5.23). In another embodiment, the Syk protein in the crystallizable composition is at least 95% pure.

In another embodiment, the method of making crystals of Syk_{Cat} proteins, Syk_{Cat} protein complexes, or homologues thereof includes the use of a device for promoting crystallizations. Devices for promoting crystallization include but are not limited to hanging-drop, sitting-drop, sandwich-drop, dialysis, microbatch or microtube batch devices (U.S. Pat. Nos. 4,886,646, 5,096,676, 5,130,105, 5,221,410 and 5,400,741; Pav et al., Proteins: Structure, Function, and Genetics, 20, pp. 98-102 (1994); Chayen, Acta. Cryst., D54, pp. 8-15 (1998), Chayen, Structure, 5, pp. 1269-1274 (1997), D'Arcy et al., J. Cryst. Growth, 168, pp. 175-180 (1996) and Chayen, J. Appl. Cryst., 30, pp. 198-202 (1997), incorporated herein by reference).

The hanging-drop, sitting-drop and some adaptations of the microbatch methods (D'Arcy et al., J. Cryst. Growth, 168, pp. 175-180 (1996) and Chayen, J. Appl. Cryst., 30, pp. 198-202 (1997)) produce crystals by vapor diffusion. The hanging drop and sitting drop containing the crystallizable composition is equilibrated against a reservoir containing a higher or lower concentration of precipitant. As the drop approaches equilibrium with the reservoir, the saturation of protein in the solution leads to the formation of crystals.

Microseeding or seeding may be used to obtain larger, or better quality (i.e., crystals with higher resolution diffraction or single crystals) crystals from initial micro-crystals. Microseeding involves the use of crystalline particles to provide nucleation under controlled crystallization conditions. In this instance, micro-crystals are crushed to yield a stock seed solution. The stock seed solution is diluted in series. Using a needle, glass rod, or strand of hair, a small sample from each diluted solution is added to a set of equilibrated drops containing a protein concentration equal to or less than a concentration needed to create crystals without the presence of seeds. The aim is to end up with a single seed crystal that will act to nucleate crystal growth in the drop.

It would be readily apparent to one of skill in the art following the teachings of the specification to vary the crystallization conditions disclosed herein to identify other crystallization conditions that would produce crystals of Syk_{Cat} protein, Syk_{Cat} protein complex or a homologue thereof. Such variations include, but are not limited to, adjusting pH, protein concentration and/or crystallization temperature, changing the identity or concentration of salt and/or precipitant used, using a different method for crystallization, or introducing additives such as detergents (e.g., TWEEN 20 (monolaurate), LDAO, Brij 30 (4 lauryl ether)), sugars (e.g., glucose, maltose), organic compounds (e.g., dioxane, dimethylformamide), lanthanide ions, or poly-ionic compounds that aid in crystallizations. High throughput crystallization assays may also be used to assist in finding or optimizing the crystallization condition.

### Binding Pockets of the Sykat Protein, Protein Complexes or Homologues Thereof

In order to use the structure coordinates generated for the catalytic domain of Syk, its complexes, one of its binding pockets, or a Syk_{Cat}-like binding pocket thereof, it is often times necessary to convert the coordinates into a three-dimensional shape. This is be achieved through the use of a computer and commercially available software that is capable of generating three-dimensional graphical representations or three-dimensional information of molecules or portions thereof from a set of structure coordinates.

Binding pockets, also referred to as binding sites in the present invention, are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or part of the binding pocket. An understanding of such associations will help lead to the design of drugs having more favorable associations with their target receptor or enzyme, and thus, improved biological effects. Therefore, this information is valuable in designing potential inhibitors of the binding pockets of biologically important targets. The ATP and substrate binding pockets of this invention will be important for drug design.

In one embodiment, the ATP-binding pocket comprises amino acids, L377, M424, V433, M448, A451, L453, G454, L501, and S511 according to the structure of the Syk_{Cat} complexes in FIG. 1 or 2. In another embodiment, the ATP-binding pocket comprises amino acid residues L377, G378, S379, V385, A400, K402, V433, M448, E449, M450, A451, E452, P455, R498, N499, L501, S511 and D512 according to the structures of the Syk_{Cat} complexes in FIG. 1 or 2.

In another embodiment, the ATP-binding pocket comprises amino acid residues L377, G378, S379, F382, V385, A400, K402, E420, V433, M448, E449, M450, A451, E452, L453, G454, P455, N457, R498, N499, L501, S511 and D512 according to the structure of the Syk_{Cat}-staurosporine complex in FIG. 1. In another embodiment, the ATP-binding pocket comprises amino acid residues L377, G378, S379, V385, A400, K402, V433, M448, E449, M450, A451, E452, P455, K458, R498, N499, L501, S511 and D512 according to the structure of the Syk_{Cat}-PT426-AMP-PNP complex in FIG. 2. These amino acid residues are within 5 Å (“5 Å sphere amino acids”) of staurosporine or AMP-PNP bound in the ATP-binding pockets, as identified using the program QUANTA (Accelrys ©2001,2002).

In another embodiment, the ATP-binding pocket comprises amino acid residues K375, E376, L377, G378, S379, G380, N381, F382, G383, T384, V385, K386, K387, T398, V399, A400, V401, K402, E420, M424, V433, R434, L446, V447, M448, E449, M450, A451, E452, L453, G454, P455, L456, N457, K458, D494, A496, A497, R498, N499, V500, L501, L502, V503, K509, I510, S511, D512, F513, and G514 according to the structure of the Syk_{Cat}-staurosporine complex in FIG. 1. In another embodiment, the ATP-binding pocket comprises amino acid residues D376, L377, G378, S379, G380, G383, T384, V385, K386, T398, V399, A400, V401, K402, L417, E420, M424, V433, R434, M435, L446, V447, M448, E449, M450, A451, E452, L453, G454, P455, L456, N457, K458, D494, A497, R498, N499, V500, L501, L502, V503, K509, I510, S511, D512, F513, G514, and L515 according to the structure of Syk_{Cat}-PT426-AMP-PNP complex in FIG. 2. These amino acid residues are within 8 Å (“8 Å sphere amino acids”) of staurosporine or AMP-PNP bound in the ATP-binding pockets, as identified using the program QUANTA (Accelrys ©2001,2002).

In another embodiment, the invention provides a crystalline molecule or molecular complex comprising all or part of a Syk_{Cat} substrate binding pocket defined by structure coordinates of a set of amino acid residues which are identical to Syk amino acid residues Asp494, Gly532, Lys533, Trp534 and Pro535 according to FIG. 2. These Syk_{Cat} residues form hydrogen bonds with the peptide PT426.

It will be readily apparent to those of skill in the art that the numbering of amino acid residues in other homologues of Syk_{Cat} may be different than that set forth for Syk_{Cat}. Corresponding amino acid residues in homologues of Syk_{Cat} may be identified by visual inspection of the amino acid sequences or by using commercially available sequence homology, structural homology or structure superimposition software programs.

Those having skill in the art will understand that a set of structure coordinates for a molecule or a molecular-complex or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

The variations in coordinates discussed above may be generated as a result of mathematical manipulations of the Syk_{Cat} structure coordinates. For example, the structure coordinates set forth in FIG. 1 or 2 may undergo crystallographic permutations of the structure coordinates, fractionalization of the structure coordinates, integer additions or subtractions, inversion or any combination of the above.

Alternatively, modifications in the crystal structure due to mutations, additions, substitutions, and/or deletions of amino acids, or other changes in any of the components that make up the crystal may also account for variations in structure coordinates. If such variations are within a certain RMSD as compared to the original coordinates, the resulting three-dimensional shape is considered encompassed by this invention. Thus, for example, a ligand that binds to the binding pocket of Syk_{Cat} would also be expected to bind to another binding pocket whose structure coordinates define a shape that falls within the acceptable RMSD.

Various computational analyses may be used to determine whether a binding pocket, motif, domain or portion thereof of a molecule or molecular complex is sufficiently similar to the binding pocket, motif, domain or portion thereof of Syk_{Cat}. Such analyses may be carried out using well-known software applications, such as ProFit (A. C. R. Martin, SciTech Software, ProFit version 1.8, University College London, http://www.bioinf.org.uk/software), Swiss-Pdb Viewer (Guex et al., Electrophoresis, 18, pp. 2714-2723 (1997)), the Molecular Similarity application of QUANTA (Accelrys ©2001,2002) and as described in the accompanying User's Guide, all of which are incorporated herein by reference.

The above-identified programs, as well as others known to those of skill in the art, permit comparisons between different structures, different conformations of the same structure, and different parts of the same structure. The procedure used by QUANTA (Accelrys ©2001,2002) and Swiss-Pdb Viewer to compare structures requires four steps: 1) loading the structures to be compared; 2) defining the atom equivalences in these structures; 3) performing a fitting operation on the structures; and 4) analyzing the results.

The procedure used in ProFit to compare structures includes: 1) loading the structures to be compared; 2) specifying selected residues of interest; 3) defining the atom equivalences in the selected residues; 4) performing a fitting operation on the selected residues; and 5) analyzing the results.

Each structure in the comparison is identified by a name. One structure is identified as the target (i.e., the fixed structure); all other structures are loaded in as working structures (i.e., moving structures). Since atom equivalency within the above programs is defined by user input, for the purpose of this invention we will define equivalent atoms as protein backbone atoms (N, Cα, C and 0) for Syk_{Cat} amino acid residues and corresponding amino acid residues in the structures being compared.

The corresponding amino acid residues may be identified by sequence alignment programs such as the “bestfit” program available from the Genetics Computer Group which uses the local homology algorithm described by Smith and Waterman in Advances in Applied Mathematics 2, 482 (1981), which is incorporated herein by reference. A suitable amino acid sequence alignment will require that the proteins being aligned share minimum percentage of identical amino acids. Generally, a first protein being aligned with a second protein should share in excess of about 35% identical amino acid residues (Hanks et al., Science, 241, 42 (1988); Hanks and Quinn, Methods in Enzymology, 200, 38 (1991)). The identification of equivalent residues can also be assisted by secondary structure alignment, for example, aligning the α-helices, β-sheets in the structure. The program Swiss-Pdb Viewer has its own best fit algorithm that is based on secondary sequence alignment.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by the above programs. The Swiss-Pdb Viewer program sets an RMSD cutoff for eliminating pairs of equivalent atoms that have high RMSD values. An RMSD cutoff value can be used to exclude pairs of equivalent atoms with extreme individual RMSD values. In the program ProFit, the RMSD cutoff value can be specified by the user.

For the purpose of this invention, any molecule, molecular complex, binding pocket, motif, domain thereof or portion thereof that is within an RMSD for backbone atoms (N, Cα, C, O) when superimposed on the relevant backbone atoms described by structure coordinates listed in FIG. 1 or 2 are encompassed by this invention. In one embodiment, the amino acid residues that define a binding pocket of Syk protein are identical to the amino acid residues that define the binding pocket of Zap-70 protein.

One embodiment of this invention provides a crystalline molecule or molecular complex comprising a domain defined by structure coordinates of a set of amino acid residues that are identical to Syk amino acid residues according to FIG. 1 or 2, wherein the RMSD of the backbone atoms between said set of amino acid residues and said Syk amino acid residues is not more than about 5.0 Å. In other embodiments, the RMSD between said set and amino acid residues of said Syk amino acid residues is not greater than about 4.0 Å, not greater than about 3.0 Å, not greater than about 2.0 Å, not greater than about 1.5 Å, not greater than about 1.0 Å or not greater than about 0.5 Å.

Another embodiment of this invention provides a crystalline molecule or molecular complex comprising substantially all of a domain defined by structure coordinates of a set of amino acid residues that are identical to Syk amino acid residues according to FIG. 1 or 2, wherein the RMSD of the backbone atoms between said set of amino acid residues of said molecule or molecular complex and said Syk amino acid residues is not more than about 5.0 Å. In other embodiments, the RMSD between said set of amino acid residues of said molecule or molecular complex and said Syk amino acid residues is not greater than about 4.0 Å, not greater than about 3.0 Å, not greater than about 2.0 Å, not greater than about 1.5 Å, not greater than about 1.0 Å or not greater than about 0.5 Å.

Another embodiment of this invention provides a crystalline molecule or molecular complex comprising all or part of a Syk_{Cat} ATP-binding pocket defined by a set of amino acid residues comprising at least four amino acid residues which are identical to Syk amino acid residues L377, M424, V433, M448, A451, G454, L501, and S511 according to FIG. 1 or 2, wherein the root mean square deviation of the backbone atoms between said at least four amino acid residues and said Syk amino acid residues which are identical is not greater than about 3.0 Å. In another embodiment, the RMSD between said set of amino acid residues of said molecule or molecular complex and said Syk amino acid residues is not greater than about 2.0 Å, 1.0 Å or 0.5 Å. In a further embodiment, the binding pocket is defined by at least six amino acid residues or all of the above Syk amino acid residues.

Another embodiment of this invention provides a crystalline molecule or molecular complex comprising all or part of a Syk_{Cat} ATP-binding pocket defined by a set of amino acid residues comprising at least four amino acid residues which are identical to Syk amino acid residues L377, F382, M424, V433, M448, A451, G454, L501, and S511 according to FIG. 1 or 2, wherein the root mean square deviation of the backbone atoms between said at least four amino acid residues and said Syk amino acid residues which are identical is not greater than about 3.0 Å. In another embodiment, the RMSD between said set of amino acid residues of said molecule or molecular complex and said Syk amino acid residues is not greater than about 2.0 Å, 1.0 Å or 0.5 Å. In a further embodiment, the binding pocket is defined by at least six, eight or all of the above Syk amino acid residues.

Another embodiment of this invention provides a crystalline molecule or molecular complex comprising all or part of a Syk_{Cat} ATP-binding pocket defined by a set of amino acid residues comprising at least four amino acid residues which are identical to Syk amino acid residues L377, F382, M424, V433, M448, A451, L453, G454, L501, and S511 according to FIG. 1 or 2, wherein the root mean square deviation of the backbone atoms between said at least four amino acid residues and said Syk amino acid residues which are identical is not greater than about 3 Å. In another embodiment, the RMSD between said set of amino acid residues of said molecule or molecular complex and said Syk amino acid residues is not greater than about 2.0 Å, 1.0 Å or 0.5 Å. In a further embodiment, the binding pocket is defined by at least six, eight or all of the above Syk amino acid residues.

Another embodiment of this invention provides a crystalline molecule or molecular complex comprising all or part of a Syk_{Cat} ATP-binding pocket defined by a set of amino acid residues comprising at least four amino acid residues which are identical to Syk amino acid residues L377, G378, S379, V385, A400, K402, V433, M448, E449, M450, A451, E452, P455, R498, N499, L501, S511 and D512 according to FIG. 1 or 2, wherein the root mean square deviation of the backbone atoms between said at least four amino acid residues and said Syk amino acid residues which are identical is not greater than about 3 Å. In another embodiment, the RMSD between said set of amino acid residues of said molecule or molecular complex and said Syk amino acid residues is not greater than about 2.0 Å, 1.0 Å or 0.5 Å. In a further embodiment, the binding pocket is defined by at least six, eight, ten, twelve, fourteen, sixteen or all of the above Syk amino acid residues.

Another embodiment of this invention provides a crystalline molecule or molecular complex comprising all or part of a Syk_{Cat} ATP-binding pocket defined by a set of amino acid residues comprising at least four amino acid residues which are identical to Syk amino acid residues L377, G378, S379, F382, V385, A400, K402, E420, V433, M448, E449, M450, A451, E452, L453, G454, P455, N457, R498, N499, L501, S511 and D512 according to FIG. 1, wherein the root mean square deviation of the backbone atoms between said at least four amino acid residues and said Syk amino acid residues which are identical is not greater than about 3 Å. In another embodiment, the RMSD between said set of amino acid residues of said molecule or molecular complex and said Syk amino acid residues is not greater than about 2.0 Å, 1.0 Å or 0.5 Å. In a further embodiment, the binding pocket is defined by at least six, eight, ten, twelve, fourteen, sixteen, eighteen, twenty or all of the above Syk amino acid residues.

Another embodiment of this invention provides a crystalline molecule or molecular complex comprising all or part of a Syk_{Cat} ATP-binding pocket defined by a set of amino acid residues comprising at least four amino acid residues which are identical to Syk amino acid residues L377, G378, S379, V385, A400, K402, V433, M448, E449, M450, A451, E452, P455, K458, R498, N499, L501, S511 and D512 according to FIG. 2, wherein the root mean square deviation of the backbone atoms between said at least four amino acid residues and said Syk amino acid residues which are identical is not greater than about 3 Å. In another embodiment, the RMSD between said set of amino acid residues of said molecule or molecular complex and said Syk amino acid residues is not greater than about 2.0 Å, 1.0 Å or 0.5 Å. In a further embodiment, the binding pocket is defined by at least six, eight, ten, twelve, fourteen, sixteen, or all of the above Syk amino acid residues.

Another embodiment of this invention provides a crystalline molecule or molecular complex comprising all or part of a Syk_{Cat} ATP-binding pocket defined by a set of amino acid residues comprising at least four amino acid residues which are identical to Syk amino acid residues K375, E376, L377, G378, S379, G380, N381, F382, G383, T384, V385, K386, K387, T398, V399, A400, V401, K402, E420, M424, V433, R434, L446, V447, M448, E449, M450, A451, E452, L453, G454, P455, L456, N457, K458, D494, A496, A497, R498, N499, V500, L501, L502, V503, K509, I510, S511, D512, F513, and G514 according to FIG. 1, wherein the root mean square deviation of the backbone atoms between said at least four amino acid residues and said Syk amino acid residues which are identical is not greater than about 3 Å. In another embodiment, the RMSD between said set of amino acid residues of said molecule or molecular complex and said Syk amino acid residues is not greater than about 2.0 Å, 1.0 Å or 0.5 Å. In a further embodiment, the binding pocket is defined by at least six, eight, ten, twelve, fourteen, sixteen, eighteen, twenty, twenty-five, thirty, thirty-five, forty, forty-five or all of the above Syk amino acid residues.

Another embodiment of this invention provides a crystalline molecule or molecular complex comprising all or part of a Syk_{Cat} ATP-binding pocket defined a set of amino acid residues comprising at least four amino acid residues which are identical to Syk amino acid residues D376, L377, G378, S379, G380, G383, T384, V385, K386, T398, V399, A400, V401, K402, L417, E420, M424, V433, R434, M435, L446, V447, M448, E449, M450, A451, E452, L453, G454, P455, L456, N457, K458, D494, A497, R498, N499, V500, L501, L502, V503, K509, I510, S511, D512, F513, G514, and L515 according to FIG. 2, wherein the root mean square deviation of the backbone atoms between said at least four amino acid residues and said Syk amino acid residues which are identical is not greater than about 3 Å. In another embodiment, the RMSD between said set of amino acid residues of said molecule or molecular complex and said Syk amino acid residues is not greater than about 2.0 Å, 1.0 Å or 0.5 Å. In a further embodiment, the binding pocket is defined by at least six, eight, ten, twelve, fourteen, sixteen, eighteen, twenty, twenty-five, thirty, thirty-five, forty, forty-five or all of the above Syk amino acid residues.

One embodiment of this invention provides a crystalline molecule or molecular complex comprising all or part of a Syk_{Cat} substrate binding pocket defined by a set of amino acid residues comprising at least four amino acid residues which are identical to Syk amino acid residues Asp494, Gly532, Lys533, Trp534 and Pro535 according to FIG. 2, wherein the root mean square deviation of the backbone atoms between said at least four amino acid residues and said Syk amino acid residues which are identical is not greater than about 3 Å. In another embodiment, the RMSD of the backbone atoms between said set of amino acid residues of said molecule or molecular complex and said Syk amino acid residues is not more than 2.0 Å, 1.0 Å or 0.5 Å.

In another embodiment, the crystalline molecule or molecular complex above is a Syk catalytic domain or a Syk catalytic domain homologue.

Computer Systems

According to another embodiment, this invention provides a machine-readable data storage medium, comprising a data storage material encoded with machine-readable data, wherein said data defines the above-mentioned molecules or molecular complexes. In one embodiment, the data defines the above-mentioned binding pockets by comprising the structure coordinates of said amino acid residues according to FIG. 1 or 2. To use the structure coordinates generated for Syk_{Cat}, homologues thereof, or one of its binding pockets, it is at times necessary to convert them into a three-dimensional shape or to extract three-dimensional structural information from them. This is achieved through the use of commercially or publicly available software that is capable of generating a three-dimensional structure or a three-dimensional representation of molecules or portions thereof from a set of structure coordinates. In one embodiment, the three-dimensional structure or representation may be displayed graphically.

Therefore, according to another embodiment, this invention provides a machine-readable data storage medium comprising a data storage material encoded with machine readable data. In one embodiment, a machine programmed with instructions for using said data, is capable of generating a three-dimensional structure or three-dimensional representation of any of the molecule or molecular complexes, or binding pockets thereof, that are described herein.

This invention also provides a computer comprising:

a) a machine-readable data storage medium, comprising a data storage material encoded with machine-readable data, wherein said data defines any one of the above molecules or molecular complexes;

b) a working memory for storing instructions for processing said machine-readable data;

c) a central processing unit (CPU) coupled to said working memory and to said machine-readable data storage medium for processing said machine readable data and a means for generating three-dimensional structural information of said molecule or molecule complex; and

d) output hardware coupled to said central processing unit for outputting three-dimensional structural information of said molecule or molecular complex, or information produced by using said three-dimensional structural information of said molecule or molecular complex.

In one embodiment, the data defines the binding pocket or domain of the molecule or molecular complex.

Three-dimensional data generation may be provided by an instruction or set of instructions such as a computer program or commands for generating a three-dimensional structure or graphical representation from structure coordinates, or by subtracting distances between atoms, calculating chemical energies for a Syk_{Cat} molecule or molecular complex or homologues thereof, or calculating or minimizing energies for an association of Syk_{Cat} molecule or molecular complex or homologues thereof to a chemical entity. The graphical representation can be generated or displayed by commercially available software programs. Examples of software programs include but are not limited to QUANTA (Accelrys ©2001, 2002), O (Jones et al., Acta Crystallogr. A47, pp. 110-119 (1991)) and RIBBONS (Carson, J. Appl. Crystallogr., 24, pp. 9589-961 (1991)), which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described in the Rational Drug Design section.

Information about said binding pocket or information produced by using said binding pocket can be outputted through a display terminal, touchscreens, facsimile machines, modems, CD-ROMS, printers, a CD or DVD recorder, ZIP™ or JAZ™ drives or disk drives. The information can be in graphical or alphanumeric form.

In one embodiment, the computer is executing an instruction such as a computer program for three dimensional data generation. In another embodiment, the computer further comprises a commercially available software program to display the information as a graphical representation. Examples of software programs include but are not limited to QUANTA (Accelrys ©2001,2002), O (Jones et al., Acta Cryst., A47, pp. 110-119 (1991)) and RIBBONS (Carson, J. Appl. Crystallogr., 24, pp. 9589-961 (1991)), all of which are incorporated herein by reference.

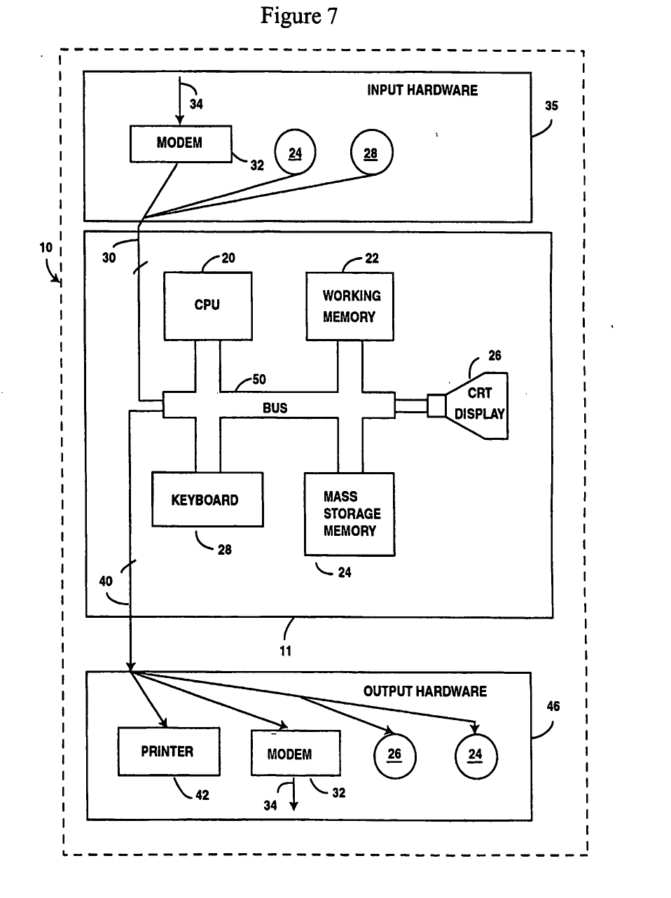

FIG. 7 demonstrates one version of these embodiments. System (10) includes a computer (11) comprising a central processing unit (“CPU”) (20), a working memory (22) which may be, e.g., RAM (random-access memory) or “core” memory, mass storage memory (24) (such as one or more disk drives or CD-ROM drives), one or more cathode-ray tube (“CRT”) display terminals (26), one or more keyboards (28), one or more input lines (30), and one or more output lines (40), all of which are interconnected by a conventional bi-directional system bus (50).

Input hardware (35), coupled to computer (11) by input lines (30), may be implemented in a variety of ways. Machine-readable data of this invention may be inputted via the use of a modem or modems (32) connected by a telephone line or dedicated data line (34). Alternatively or additionally, the input hardware (35) may comprise CD-ROM drives or disk drives (24). In conjunction with display terminal (26), keyboard (28) may also be used as an input device.

Output hardware (46), coupled to computer (11) by output lines (40), may similarly be implemented by conventional devices. By way of example, output hardware (46) may include CRT display terminal (26) for displaying a graphical representation of a binding pocket of this invention using a program such as QUANTA as described herein. Output hardware may also include a printer (42), so that hard copy output may be produced, or a disk drive (24), to store system output for later use. Output hardware may also include a display terminal, touchscreens, facsimile machines, modems, CD-ROMS, printers, a CD or DVD recorder, ZIP™ or JAZ™ drives, disk drives, or other machine-readable data storage device.

In operation, CPU (20) coordinates the use of the various input and output devices (35), (46), coordinates data accesses from mass storage (24) and accesses to and from working memory (22), and determines the sequence of data processing steps. A number of programs may be used to process the machine-readable data of this invention. Such programs are discussed in reference to the computational methods of drug discovery as described herein. Specific references to components of the hardware system (10) are included as appropriate throughout the following description of the data storage medium.

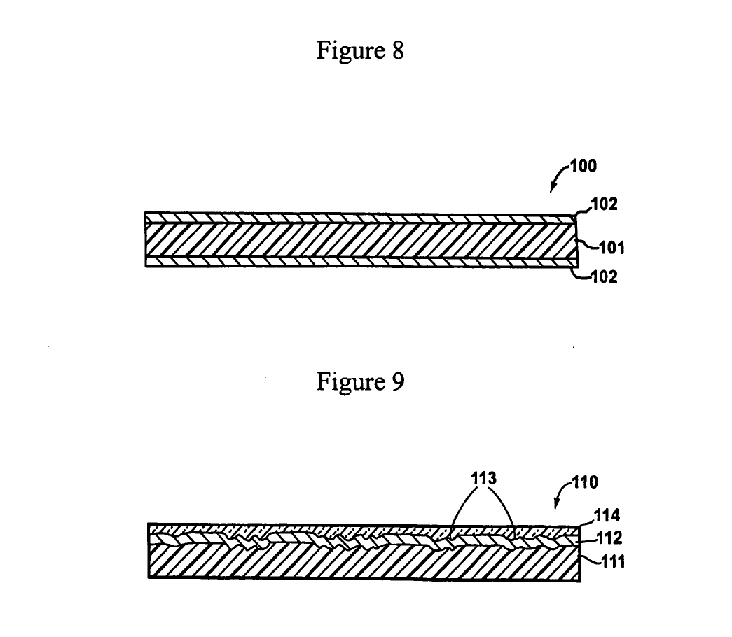

FIG. 8 shows a cross section of a magnetic data storage medium (100) which can be encoded with a machine-readable data that can be carried out by a system such as system (10) of FIG. 7. Medium (100) can be a conventional floppy diskette or hard disk, having a suitable substrate (101), which may be conventional, and a suitable coating (102), which may be conventional, on one or both sides, containing magnetic domains (not visible) whose polarity or orientation can be altered magnetically. Medium (100) may also have an opening (not shown) for receiving the spindle of a disk drive or other data storage device (24).

The magnetic domains of coating (102) of medium (100) are polarized or oriented so as to encode in manner which may be conventional, machine readable data such as that described herein, for execution by a system such as system (10) of FIG. 7.

FIG. 9 shows a cross section of an optically-readable data storage medium (110) which also can be encoded with such a machine-readable data, or set of instructions, which can be carried out by a system such as system (10) of FIG. 7. Medium (110) can be a conventional compact disk read only memory (CD-ROM) or a rewritable medium such as a magneto-optical disk which is optically readable and magneto-optically writable. Medium (100) preferably has a suitable substrate (111), which may be conventional, and a suitable coating (112), which may be conventional, usually on one side of substrate (111).

In the case of CD-ROM, as is well known, the coating (112) is reflective and is impressed with a plurality of pits (113) to encode the machine-readable data. The arrangement of pits is read by reflecting laser light off the surface of the coating (112). A protective coating (114), which preferably is substantially transparent, is provided on top of the coating (112).

In the case of a magneto-optical disk, as is well known, the coating (112) has no pits (113), but has a plurality of magnetic domains whose polarity or orientation can be changed magnetically when heated above a certain temperature, as by a laser (not shown). The orientation of the domains can be read by measuring the polarization of laser light reflected from the coating (112). The arrangement of the domains encodes the data as described above.

In one embodiment, the structure coordinates of said molecules or molecular complexes are produced by homology modeling of at least a portion of the structure coordinates of FIG. 1 or 2. Homology modeling can be used to generate structural models of Syk_{cat} homologues or other homologous proteins based on the known structure of Syk_{cat}. This can be achieved by performing one or more of the following steps: performing sequence alignment between the amino acid sequence of an unknown molecule against the amino acid sequence of Syk_{cat}; identifying conserved and variable regions by sequence or structure; generating structure coordinates for structurally conserved residues of the unknown structure from those of Syk_{cat}; generating conformations for the structurally variable residues in the unknown structure; replacing the non-conserved residues of Syk_{cat} with residues in the unknown structure; building side chain conformations; and refining and/or evaluating the unknown structure.

Software programs that are useful in homology modeling include XALIGN (Wishart, D. S. et al., Comput. Appl. Biosci., 10, pp. 687-88 (1994)) and CLUSTAL W Alignment Tool (Higgins D. G. et al., Methods Enzymol., 266, pp. 383-402 (1996)). See also, U.S. Pat. No. 5,884,230. These references are incorporated herein by reference.

To perform the sequence alignment, programs such as the “bestfit” program available from the Genetics Computer Group (Waterman in Advances in Applied Mathematics 2, 482 (1981), which is incorporated herein by reference] and CLUSTAL W Alignment Tool (Higgins D. G. et al., Methods Enzymol., 266, pp. 383-402 (1996), which is incorporated by reference] can be used. To model the amino acid side chains of another protein, the amino acid residues in Syk_{cat} can be replaced, using a computer graphics program such as “O” (Jones et al, (1991) Acta Cryst. Sect. A, 47: 110-119), by those of the homologous protein, where they differ. The same orientation or a different orientation of the amino acid can be used. Insertions and deletions of amino acid residues may be necessary where gaps occur in the sequence alignment. However, certain portions of the active site of Syk_{cat} and its homologues may be highly conserved with essentially no insertions and deletions.

Homology modeling can be performed using, for example, the computer programs SWISS-MODEL available through Glaxo Wellcome Experimental Research in Geneva, Switzerland; WHATIF available on EMBL servers; Schnare et al., J. Mol. Biol., 256: 701-719 (1996); Blundell et al., Nature 326: 347-352 (1987); Fetrow and Bryant, Bio/Technology 11:479-484 (1993); Greer, Methods in Enzymology 202: 239-252 (1991); and Johnson et al, Crit. Rev. Biochem. Mol. Biol. 29:1-68 (1994). An example of homology modeling can be found, for example, in Szklarz G. D., Life Sci. 61: 2507-2520 (1997). These references are incorporated herein by reference.

Thus, in accordance with the present invention, data capable of generating the three dimensional structure or three-dimensional representation of the above molecules or molecular complexes, or binding pockets or domains thereof, can be stored in a machine-readable storage medium, which is capable of displaying structural information or a graphical three-dimensional representation of the structure. In one embodiment, the means of generating three-dimensional structural information is provided by means for generating a three-dimensional structural representation of the binding pocket or domain of a molecule or molecular complex.

Rational Drug Design

The Syk_{cat} structure coordinates or the three-dimensional graphical representation generated from these coordinates may be used in conjunction with a computer for a variety of purposes, including drug discovery.

For example, the structure encoded by the data may be computationally evaluated for its ability to associate with chemical entities. Chemical entities that associate with Syk_{cat} may inhibit or activate Syk or its homologues, and are potential drug candidates. Alternatively, the structure encoded by the data may be displayed in a graphical three-dimensional representation on a computer screen. This allows visual inspection of the structure, as well as visual inspection of the structure's association with chemical entities.

In one embodiment, the invention provides for a method of using a computer for selecting an orientation of a chemical entity that interacts favorably with a binding pocket or domain comprising the steps of:
(a) providing the structure coordinates of the binding pocket or domain on a computer comprising the means for generating three-dimensional structural information from the structure coordinates;
(b) employing computational means to dock a first chemical entity in the binding pocket or domain;
(c) quantitating the interaction energy between the chemical entity and all or part of the binding pocket or domain for different orientations of the chemical entity; and
(d) selecting the orientation of the chemical entity with the most favorable interaction energy.

In one embodiment, the docking is facilitated by said quantitated interaction energy.

In one embodiment, the above method further comprises the following steps before step (a):
(e) producing a crystal of a molecule or molecular complex comprising Syk_{cat} or homologue thereof;
(f) determining the three-dimensional structure coordinates of the molecule or molecular complex by X-ray diffraction of the crystal; and
(g) identifying all or part of said binding pocket.

Three-dimensional structural information in step (a) may be generated by instructions such as a computer program or commands that can generate a three-dimensional representation; subtract distances between atoms; calculate chemical energies for a Syk_{Cat} molecule, molecular complex or homologues thereof; or calculate or minimize the chemical energies of an association of Syk molecule, molecular complex or homologues thereof to a chemical entity. These types of computer programs are known in the art. The graphical representation can be generated or displayed by commercially available software programs. Examples of software programs include but are not limited to QUANTA (Accelrys ©2001, 2002), O (Jones et al., Acta Crystallogr. A47, pp. 110-119 (1991)) and RIBBONS (Carson, J. Appl. Crystallogr., 24, pp. 9589-961 (1991)), which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described below.

The above method may further comprise the following step after step (d): outputting said quantified interaction energy to a suitable output hardware, such as a CRT display terminal, a CD or DVD recorder, ZIP™ or JAZ™ drive, a disk drive, or other machine-readable data storage device, as described previously. The method may further comprise generating a three-dimensional structure, graphical representation thereof, or both, of the molecule or molecular complex prior to step (b).

One embodiment of this invention provides for the above method, wherein energy minimization with or without molecular dynamics simulations are performed simultaneously with or following step (b).

The above method may further comprise the steps of:
(e) repeating steps (b) through (d) with a second chemical entity; and
(f) selecting at least one of said first or second chemical entity that interacts more favorably with said binding pocket or domain based on said quantitated interaction energy of said first or second chemical entity.

In one embodiment, the invention provides for a method of using a computer for selecting an orientation of a chemical entity with a favorable shape complementarity in a binding pocket comprising the steps of:
(a) providing the structure coordinates of said binding pocket and ligand bound therein on a computer comprising the means for generating three-dimensional structural information from said structure coordinates;
(b) employing computational means to dock a first chemical entity in the binding pocket;
(c) quantitating the contact score of said chemical entity in different orientions; and
(d) selecting an orientation with the highest contact score.

In one embodiment, the docking is facilitated by the contact score.

The method above may further comprise the step of generating a three-dimensional graphical representation of the binding pocket and ligand bound therein prior to step (b).

The method above may further comprise the steps of:
(e) repeating steps (b) through (d) with a second chemical entity; and
(f) selecting at least one of said first or second chemical entity that has a higher contact score based on said quantitated contact score of said first or second chemical entity.

In another embodiment, the invention provides a method for screening a plurality of chemical entities to associate at a deformation energy of binding of less than −7 kcal/mol with said binding pocket:
(a) employing computational means, which utilize said structure coordinates to dock one of said chemical entities from the plurality of chemical entities in said binding pocket;
(b) quantifying the deformation energy of binding between the chemical entity and the binding pocket;
(c) repeating steps (a) and (b) for each remaining chemical entity; and
(d) outputting a set of chemical entities that associate with the binding pocket at a deformation energy of binding of less than −7 kcal/mol to a suitable output hardware.

In another embodiment, the method comprises the steps of:
(a) constructing a computer model of a binding pocket of the molecule or molecular complex;
(b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of a Syk protein or homologue thereof;
(c) employing computational means to dock said chemical entity to be evaluated in said binding pocket in order to provide an energy-minimized configuration of said chemical entity in the binding pocket; and
(d) evaluating the results of said docking to quantify the interaction energy between said chemical entity and the binding pocket.

Alternatively, the structure coordinates of the Syk_{Cat} binding pockets may be utilized in a method for identifying a candidate inhibitor of a molecule comprising a binding pocket of Syk_{Cat}. This method comprises the steps of:

(a) using a three-dimensional structure of the binding pocket or domain to design, select or optimize a plurality of chemical entities;

(b) contacting each chemical entity with the molecule or the molecular complex;

(c) monitoring the inhibition to the catalytic activity of the molecule or molecular complex by the chemical entity; and

(d) selecting a chemical entity based on the inhibitory effect of the chemical entity on the catalytic activity of the molecule or molecular complex.

In one embodiment, step (a) is performed using a three-dimensional structure of the binding pocket or domain or portion thereof of the molecule or molecular complex. In another embodiment, the three-dimensional structure is displayed as a graphical representation.

In another embodiment, the method comprises the steps of:
(a) constructing a computer model of a binding pocket of the molecule or molecular complex;
(b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of a Syk protein or homologue thereof;
(c) employing computational means to dock said chemical entity to be evaluated in said binding pocket in order to provide an energy-minimized configuration of said chemical entity in the binding pocket;
(d) evaluating the results of said docking to quantify the interaction energy between said chemical entity and the binding pocket;
(e) synthesizing said chemical entity; and
(f) contacting said chemical entity with said molecule or molecular complex to determine the ability of said compound to activate or inhibit said molecule.

In one embodiment, the invention provides a method of designing a compound or complex that interacts with all or part of the binding pocket comprising the steps of:
(a) providing the structure coordinates of said binding pocket or domain on a computer comprising the means for generating three-dimensional structural information from said structure coordinates;
(b) using the computer to dock a first chemical entity in part of the binding pocket or domain;
(c) docking at least a second chemical entity in another part of the binding pocket or domain;
(d) quantifying the interaction energy between the first or second chemical entity and part of the binding pocket or domain;
(e) repeating steps (b) to (d) with another first and second chemical entity, selecting a first and a second chemical entity based on said quantified interaction energy of of all of said first and second chemical entity;
(f) optionally, visually inspecting the relationship of the first and second chemical entity to each other in relation to the binding pocket or domain on a computer screen using the three-dimensional graphical representation of the binding pocket or domain and said first and second chemical entity; and
(g) assembling the first and second chemical entity into a compound or complex that interacts with said binding pocket or domain by model building.

For the first time, the present invention permits the use of molecular design techniques to identify, select and design chemical entities, including inhibitory compounds, capable of binding to Syk_{Cat} or Syk_{Cat}-like binding pockets, motifs and domains.

Applicants' elucidation of binding pockets on Syk_{Cat} provides the necessary information for designing new chemical entities and compounds that may interact with Syk_{Cat} substrate or ATP-binding pockets or Syk_{Cat}-like substrate or ATP-binding pockets, in whole or in part. Due to the homology in the kinase core between Syk_{Cat} and ZAP-70, compounds that inhibit Syk_{Cat} are also expected to inhibit ZAP-70, especially those compounds that bind the ATP-binding pocket. Throughout this section, discussions about the ability of a chemical entity to bind to, associate with, or inhibit Syk_{Cat} binding pockets refer to features of the entity alone.

The design of compounds that bind to or inhibit Syk_{Cat} binding pockets according to this invention generally involves consideration of two factors. First, the chemical entity must be capable of physically and structurally associating with parts or all of the Syk_{Cat} binding pockets. Non-covalent molecular interactions important in this association include hydrogen bonding, van der Waals interactions, hydrophobic interactions and electrostatic interactions.

Second, the chemical entity must be able to assume a conformation that allows it to associate with the Syk_{Cat} binding pockets directly. Although certain portions of the chemical entity will not directly participate in these associations, those portions of the chemical entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of a chemical entity comprising several chemical entities that directly interact with the Syk_{Cat} or Syk_{Cat}-like binding pockets.

The potential inhibitory or binding effect of a chemical entity on Syk_{Cat} binding pockets may be analyzed prior to its actual synthesis and testing by the use of computer modeling techniques. If the theoretical structure of the given entity suggests insufficient interaction and association between it and the Syk_{Cat} binding pockets, testing of the entity is obviated. However, if computer modeling indicates a strong interaction, the molecule may then be synthesized and tested for its ability to bind to a Syk_{Cat} binding pocket. This may be achieved by testing the ability of the molecule to inhibit the catalytic domain of Syk by methods disclosed in International PCT Applications WO 01/09134, WO 01/47922 and U.S. Pat. No. 6,114,333, all of which are specifically incorporated herein by reference. In this manner, synthesis of inoperative compounds may be avoided.

A potential inhibitor that binds to a binding pocket may be computationally evaluated by means of a series of steps in which chemical entities or fragments are screened and selected for their ability to associate with the above binding pockets.

One skilled in the art may use one of several methods to screen chemical entities or fragments or moieties thereof for their ability to associate with the binding pockets described herein. This process may begin by visual inspection of, for example, any of the binding pockets on the computer screen based on the Syk structure coordinates in FIG. 1 or 2 or other coordinates that define a similar shape generated from the machine-readable storage medium. Selected chemical entities, or fragments or moieties thereof may then be positioned in a variety of orientations, or docked, within that binding pocket as defined supra. Docking may be accomplished using software such as QUANTA and Sybyl (Tripos Associates, St. Louis, Mo.), followed by, or performed simultaneously with, energy minimization, rigid-body minimization (Gshwend, supra) and molecular dynamics with standard molecular mechanics force fields, such as CHARMM and AMBER.

Specialized computer programs may also assist in the process of selecting fragments or chemical entities or fragments or moieties thereof. These include:
1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK.
2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif.
3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.
4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of Syk_{Cat}. This would be followed by manual model building using software such as QUANTA or Sybyl.

Useful programs to aid one of skill in the art in connecting the individual chemical entities or fragments include:
1. CAVEAT (P. A. Bartlett et al., “CAVEAT: A Program to Facilitate the Structure-Derived Design of Biologically Active Molecules”, in “Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des. , 8, pp. 51-66 (1994)). CAVEAT is available from the University of California, Berkeley, Calif.
2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992).
3. HOOK (M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994). HOOK is available from Molecular Simulations, San Diego, Calif.

Instead of proceeding to build an agonist or an inhibitor of any of the above binding pockets in a step-wise fashion, one fragment or chemical entity at a time as described above, inhibitory or other Syk binding compounds may be designed as a whole or “de novo” using either an empty binding pocket or optionally including some portion(s) of a known inhibitor(s). There are many de novo ligand design methods including:
1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.
2. LEGEND (Y. Nishibata et al., Tetrahedron, 47, p. 8985 (1991)). LEGEND is available from Molecular Simulations Incorporated, San Diego, Calif.
3. LeapFrog (available from Tripos Associates, St. Louis, Mo.).
4. SPROUT (V. Gillet et al., “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)).

Once a chemical entity has been designed or selected by the above methods, the efficiency with which that entity may bind to any of the above binding pockets may be tested and optimized by computational evaluation. For example, an effective binding pocket inhibitor must preferably demonstrate a relatively small difference in energy between its bound and free states (i.e., a small deformation energy of binding). Thus, the most efficient binding pocket inhibitors should preferably be designed with a deformation energy of binding of not greater than about 10 kcal/mole, more preferably, not greater than 7 kcal/mole. Binding pocket inhibitors may interact with the binding pocket in more than one conformation that is similar in overall binding energy. In those cases, the deformation energy of binding is taken to be the difference between the energy of the free entity and the average energy of the conformations observed when the inhibitor binds to the protein.

A chemical entity designed or selected as binding to any one of the above binding pockets may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. ©1995); AMBER, version 4.1 (P. A. Kollman, University of California at San Francisco, ©1995); QUANTA/CHARMM (Molecular Simulations, Inc., San Diego, Calif. ©1998); Insight II/Discover (Molecular Simulations, Inc., San Diego, Calif. ©1998); DelPhi (Molecular Simulations, Inc., San Diego, Calif. ©1998); and AMSOL (Quantum Chemistry Program Exchange, Indiana University). These programs may be implemented, for instance, using a Silicon Graphics workstation such as an Indigo2 with “IMPACT” graphics. Other hardware systems and software packages will be known to those skilled in the art.

Another approach enabled by this invention, is the computational screening of small molecule databases for chemical entities or compounds that can bind in whole, or in part, to any of the above binding pockets. In this screening, the quality of fit of such entities to the binding pocket may be judged either by shape complementarity or by estimated interaction energy (E. C. Meng et al., J. Comp. Chem., 13, pp. 505-524 (1992)).

Another particularly useful drug design technique enabled by this invention is iterative drug design. Iterative drug design is a method for optimizing associations between a protein and a compound by determining and evaluating the three-dimensional structures of successive sets of protein/compound complexes.

According to another embodiment, the invention provides chemical entities which associate with a Syk_{cat} binding pocket produced or identified by the method set forth above.

Another particularly useful drug design technique enabled by this invention is iterative drug design. Iterative drug design is a method for optimizing associations between a protein and a chemical entity by determining and evaluating the three-dimensional structures of successive sets of protein/chemical entity complexes.

In iterative drug design, crystals of a series of protein or protein complexes are obtained and then the three-dimensional structures of each crystal is solved. Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. High throughput crystallization assays may be used to find a new crystallization condition or to optimize the original protein or complex crystallization condition for the new complex. Alternatively, a pre-formed protein crystal may be soaked in the presence of an inhibitor, thereby forming a protein/compound complex and obviating the need to crystallize each individual protein/compound complex.

Structure Determination of Other Molecules

The structure coordinates set forth in FIG. 1 or 2 can also be used to aid in obtaining structural information about other crystallized molecules or molecular complexes. This may be achieved by any of a number of well-known techniques, including molecular replacement.

In one embodiment, the structure coordinates of said molecules or molecular complexes are produced by homology modeling of the coordinates of FIG. 1 or 2. Homology modeling can be used to generate structural models of Syk_{Cat} homologues or other homologous proteins based on the known structure of Syk_{Cat}. This can be achieved by performing one or more of the following steps: performing sequence alignment between the amino acid sequence of an unknown molecule against the amino acid of Syk; identifying conserved and variable regions by sequence or structure; generating structure coordinates for structurally conserved residues of the unknown structure from those of Syk; generating conformations for the structurally variable residues in the unknown structure; replacing the non-conserved residues of Syk with residues in the unknown structure; building side chain conformations; and refining and/or evaluating the unknown structure.

For example, since the protein sequence of the catalytic domains of Syk and ZAP-70 can be aligned relative to each other, it is possible to construct models of the structures of ZAP-70, particularly in the regions of the active site, using the Syk_{Cat} structure. Software programs that are useful in homology modeling include XALIGN (Wishart, D. S. et al., Comput. Appl. Biosci., 10, pp. 687-88 (1994)) and CLUSTAL W Alignment Tool (Higgins D. G. et al., Methods Enzymol, 266, pp. 383-402 (1996)). See also, U.S. Pat. No. 5,884,230. These references are incorporated herein by reference.

To perform the sequence alignment, programs such as the “bestfit” program available from the Genetics Computer Group (Waterman in Advances in Applied Mathematics 2, 482 (1981), which is incorporated herein by reference) and CLUSTAL W Alignment Tool (Higgins D. G. et al., Methods Enzymol, 266, pp. 383-402 (1996), which is incorporated by reference) can be used. To model the amino acid side chains of ZAP-70, the amino acid residues in Syk can be replaced, using a computer graphics program such as “O” (Jones et al, (1991) Acta Cryst. Sect. A, 47: 110-119), by those of the homologous protein, where they differ. The same orientation or a different orientation of the amino acid can be used. Insertions and deletions of amino acid residues may be necessary where gaps occur in the sequence alignment. However, certain portions of the active site of Syk and its homologues are highly conserved with essentially no insertions and deletions.

Homology modeling can be performed using, for example, the computer programs SWISS-MODEL available through Glaxo Wellcome Experimental Research in Geneva, Switzerland; WHATIF available on EMBL servers; Schnare et al. (1996) J. Mol. Biol, 256: 701-719; Blundell et al. (1987) Nature 326: 347-352; Fetrow and Bryant (1993) Bio/Technology 11:479-484; Greer (1991) Methods in Enzymology 202: 239-252; and Johnson et al (1994) Crit. Rev. Biochem. Mol Biol. 29:1-68. An example of homology modeling can be found, for example, in Szklarz G. D (1997) Life Sci. 61: 2507-2520. These references are incorporated herein by reference.

According to an alternate embodiment, the machine-readable data storage medium comprises a data storage material encoded with a first set of machine readable data that comprises the Fourier transform of at least a portion of the structure coordinates set forth in FIG. 1 or 2 or homology model thereof, and which, when using a machine programmed with instructions for using said data, can be combined with a second set of machine readable data comprising the X-ray diffraction pattern of a molecule or molecular complex to determine at least a portion of the structure coordinates corresponding to the second set of machine readable data.

In another embodiment, the invention provides a computer for determining at least a portion of the structure coordinates corresponding to X-ray diffraction data obtained from a molecule or molecular complex having an unknown structure, wherein said computer comprises:
(a) a machine-readable data storage medium comprising a data storage material encoded with machine-readable data, wherein said data comprises at least a portion of the structure coordinates of Syk_{Cat} according to FIG. 1 or 2 or homology model thereof;
(b) a machine-readable data storage medium comprising a data storage material encoded with machine-readable data, wherein said data comprises X-ray diffraction data obtained from said molecule or molecular complex having an unknown structure; and
(c) instructions for performing a Fourier transform of the machine-readable data of (a) and for processing said machine-readable data of (b) into structure coordinates.

For example, the Fourier transform of at least a portion of the structure coordinates set forth in FIG. 1 or 2 or homology model thereof may be used to determine at least a portion of the structure coordinates of Syk protein, Syk_{Cat} protein homologues, or proteins sufficiently homologous to Syk_{Cat}. In one embodiment, the molecule is a Syk_{Cat} homologue. In another embodiment, the molecular complex is selected from the group consisting of Syk_{Cat} complex and Syk_{Cat} homologue complex.

Therefore, in another embodiment this invention provides a method of utilizing molecular replacement to obtain structural information about a molecule or a molecular complex of unknown structure wherein the molecule or molecular complex is sufficiently homologous to Syk_{Cat}, comprising the steps of:
(a) crystallizing said molecule or molecular complex of unknown structure;
(b) generating a X-ray diffraction pattern from said crystallized molecule or molecular complex;
(c) applying at least a portion of the Syk_{Cat} structure coordinates set forth in one of FIG. 1 or 2 or in a homology model thereof to the X-ray diffraction pattern to generate a three-dimensional electron density map of at least a portion of the molecule or molecular complex whose structure is unknown; and
(d) generating a structural model of the molecule or molecular complex from the three-dimensional electron density map.

In one embodiment, the method is performed using a computer. In another embodiment, the molecule is selected from the group consisting of a Syk catalytic domain protein, a Syk catalytic domain homologue, and a Syk protein. In another embodiment, the molecule is selected from the group consisting of a Syk catalytic domain protein complex, a Syk catalytic domain homologue complex, and a Syk protein complex.

By using molecular replacement, all or part of the structure coordinates of the Syk_{Cat} as provided by this invention (and set forth in FIG. 1 or 2) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that can not be determined directly. Obtaining accurate values for the phases, by methods other than molecular replacement, can be a time-consuming process that involves iterative cycles of approximations and refinements and greatly hinders the solution of crystal structures. However, when the crystal structure of a protein containing at least a homologous portion has been solved, the phases from the known structure may provide a satisfactory estimate of the phases for the unknown structure.

Thus, this method involves generating a preliminary model of a molecule or molecular complex whose structure coordinates are unknown, by orienting and positioning the relevant portion of the Syk_{Cat} according to FIG. 1 or 2 or homology model thereof within the unit cell of the crystal of the unknown molecule or molecular complex so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex (E. Lattman, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)).

The structure of any portion of any crystallized molecule or molecular complex that is sufficiently homologous to any portion of the Syk_{Cat} can be resolved by this method.

In one embodiment, the method of molecular replacement is utilized to obtain structural information about a Syk_{Cat} homologue. The structure coordinates of Syk_{Cat} as provided by this invention are particularly useful in solving the structure of Syk_{Cat} complexes that are bound by ligands, substrates and inhibitors.

Furthermore, the structure coordinates of Syk_{Cat} as provided by this invention are useful in solving the structure of Syk_{Cat} proteins that have amino acid substitutions, additions and/or deletions (referred to collectively as “Syk_{Cat} mutants”, as compared to naturally occurring Syk_{Cat}). These Syk_{Cat} mutants may optionally be crystallized in co-complex with a chemical entity, such as a non-hydrolyzable ATP analogue or a suicide substrate. The crystal structures of a series of such complexes may then be solved by molecular replacement and compared with that of wild-type Syk_{Cat}. Potential sites for modification within the various binding pockets of the enzyme may thus be identified. This information provides an additional tool for determining the most efficient binding interactions, for example, increased hydrophobic interactions, between Syk_{Cat} and a chemical entity or compound.

The structure coordinates are also particularly useful in solving the structure of crystals of Syk_{Cat} or Syk_{Cat} homologues co-complexed with a variety of chemical entities. This approach enables the determination of the optimal sites for interaction between chemical entities, including candidate Syk_{Cat} inhibitors. For example, high resolution X-ray diffraction data collected from crystals exposed to different types of solvent allows the determination of where each type of solvent molecule resides. Small molecules that bind tightly to those sites can then be designed and synthesized and tested for their Syk_{Cat} inhibition activity.

All of the complexes referred to above may be studied using well-known X-ray diffraction techniques and may be refined using 1.2-3.4 Å resolution X-ray data to an R value of about 0.30 or less using computer software, such as X-PLOR (Yale University, ©1992, distributed by Molecular Simulations, Inc.; see, e.g., Blundell & Johnson, supra; Meth. Enzymol., vol. 114 & 115, H. W. Wyckoff et al., eds., Academic Press (1985)) or CNS (Brunger et al., Acta Cryst., D54, pp. 905-921, (1998)).

In order that this invention be more fully understood, the following examples are set forth. These examples are for the purpose of illustration only and are not to be construed as limiting the scope of the invention in any way.

### EXAMPLE 1

### Expression and Purification of Syk_{Cat}

Residues 343-635 of human Syk (accession number A53596) were cloned. PCR was carried out on the basophil-like leukemic cell line KU 812 (Kishi, K., Leuk. Res. 9, pp. 381-390 (1985)) using the following primers in order to clone Human Syk kinase domain cDNA:

| | |
|---|---|
| 5′-CGCGGATCCGCCACCATGGACACAGAGGTGTAC | (SEQ ID NO: 3) |
| GAGAGC-3′ | |
| and | |
| 5′-CGGCGGATCCTTAATGATGATGATGATGATGGT | (SEQ ID NO: 4) |
| TCACCACGTCATAGTAGTAATTGCG-3′. | |

The PCR amplicon was cloned directly into pFastBac1 (Life Technologies) using BamH1 to make the recombinant baculoviral shuttle vector pFB-CatSyk. The DNA sequence was verified using an Applied Biosystems sequencer. The PCR primer sequences introduced an optimal Kozak translational initiation sequence (Kozak, M., Nuc. Acids Res 12, pp. 857-872 (1984)) at the 5′ end of the sequence coding for the catalytic domain of Syk (residues 343-635 of full-length SEQ ID NO: 1; GenBank accession number A53596) and a hexahistidine purification tag at the carboxyl-terminal end of Syk_{Cat}. SEQ ID NO: 1 (GenBank accession number A53596) below shows the amino acid sequence of human Syk. Amino acid residue numbers in the text and FIGS. 1 and 2 follow the amino acid residue numbering system of the full-length Syk protein.

SEQ ID NO: 1:

| | |
|---|---|
| 1 | M A S S G M A D S A N H L P F F F G N I |
| 21 | T R E E A E D Y L V Q G G M S D G L Y L |
| 41 | L R Q S R N Y L G G F A L S V A H G R K |
| 61 | A H H Y T I E R E L N G T Y A I A G G R |
| 81 | T H A S P A D L C H Y H S Q E S D G L V |
| 101 | C L L K K P F N R P Q G V Q P K T G P F |
| 121 | E D L K E N L I R E Y V K Q T W N L Q G |
| 141 | Q A L E Q A I I S Q K P Q L E K L I A T |
| 161 | T A H E K M P W F H G K I S R E E S E Q |
| 181 | I V L I G S K T N G K F L I R A R D N N |
| 201 | G S Y A L C L L H E G K V L H Y R I D K |
| 221 | D K T G K L S I P E G K K F D T L W Q L |
| 241 | V E H Y S Y K A D G L L R V L T V P C Q |
| 261 | K I G T Q G N V N F G G R P Q L P G S H |
| 281 | P A T W S A G G I I S R I K S Y S F P K |
| 301 | P G H R K S S P A Q G N R Q E S T V S F |
| 321 | N P Y E P E L A P W A A D K G P Q R E A |
| 341 | L P M D T E V Y E S P Y A D P E E I R P |
| 361 | K E V Y L D R K L L T L E D K E L G S G |
| 381 | N F G T V K K G Y Y Q M K K V V K T V A |
| 401 | V K I L K N E A N D P A L K D E L L A E |
| 421 | A N V M Q Q L D N P Y I V R M I G I C E |
| 441 | A E S W M L V M E M A E L G P L N K Y L |
| 461 | Q Q N R H V K D K N I I E L V H Q V S M |
| 481 | G M K Y L E E S N F V H R D L A A R N V |
| 501 | L L V T Q H Y A K I S D F G L S K A L R |
| 521 | A D E N Y Y K A Q T H G K W P V K W Y A |
| 541 | P E C I N Y Y K F S S K S D V W S F G V |
| 561 | L M W E A F S Y G Q K P Y R G M K G S E |
| 581 | V T A M L E K G E R M G C P A G C P R E |
| 601 | M Y D L M N L C W T Y D V E N R P G F A |
| 621 | A V E L R L R N Y Y Y D V V N |

Transposition to generate Syk baculoviral DNA was performed in DH10Bac E.coli (Life Technologies). Lipofection was then used to transform Sf9 insect cells with baculoviral DNA in order to generate seed baculoviral stocks. The baculoviral stocks were amplified four times in insect-free media (Insect Xpress, Biowhittaker) using shaker flasks at 27° C. and 100 rpm. The fourth amplification viral stocks were used at a multiplicity of infection of 10 in 3 day infections of 2×10⁶ cells/ml shaker cultures for protein production.

Protein yields of 2-5 mg/l intracellular protein were obtained for Syk_{Cat} after cobalt chelation chromatography/FPLC purification. The buffer was exchanged to 20 mM diethanolamine (pH 8.6), 500 mM NaCl, and the protein was concentrated using microfiltration. The protein used in crystallizations of Syk_{Cat} complexed with PT426 and adenylyl imidodiphosphate (AMP-PNP) was further purified using a Mono Q column (Pharmacia).

### EXAMPLE 2

### Formation of Syk_{Cat}-inhibitor or Syk_{Cat}-Peptide-AMP-PNP Complex for Crystallization

Syk_{Cat} in Complex with Staurosporine

Staurosporine, a microbial alkaloid from Streptomyces sp. (Omuru et al., J. Antibiot., 48, pp. 275-282 (1977)), is a potent broad-range kinase inhibitor. Syk_{Cat} protein (2-4 mg/mL) in 20 mM Diethanolamine at pH 8.6 and 0.5 M NaCl was combined with 300 μM staurosporine.

Syk_{Cat} in Complex with Peptide, PT426

Syk_{Cat} protein (2-4 mg/mL) in diethylamine hydrochloride at pH 8.6 and 0.5 M NaCl was combined with 500 μM NAc-Glu-Glu-Asp-Asp-Tyr-Glu-Ser-Pro-NH₂ peptide (NAc-EEDDYESP-NH₂, or PT426; SEQ ID NO: 2), 2 mM AMP-PNP and 6 mM MgCl₂.

### EXAMPLE 3

### Crystallization of Syk_{Cat} and Syk_{Cat}-inhibitor Complexes Thereof Syk Catalytic Domain in Complex with Staurosporine

Syk_{Cat}-staurosporine complex was crystallized by the hanging-drop vapor diffusion method. Equal volumes of 2 mg/ml protein solution in 20 mM diethanolamine (pH 8.6), 500 mM NaCl with 300 μM staurosporine and a reservoir solution containing 20% PEG 2K, 0.2 ammonium acetate, 0.1 M sodium cacodylate (pH 5.23) were combined and placed over the reservoir containing reservoir solution. Plate like crystals began to form 24 hours later, and grew to a maximum size of 0.3×0.3×0.1 mm³ after 10 days.

Syk_{Cat} in Complex with Peptide, PT426

Syk_{Cat}-PT426-AMP-PNP complex was also crystallized by hanging-drop vapor diffusion method. Equal volumes of 3 mg/ml protein solution in 20 mM Diethanolamine (pH 8.6), 500 mM NaCl with 2 mM AMP-PNP, 6 mM MgCl₂ and 500 mM of the peptide PT426 and reservoir solution containing 22% PEG 2K, 0.2 M magnesium acetate, 0.1 M sodium cacodylate (pH 5.23) were combined and placed over the reservoir. Plate-like crystals started to form after 1 day and grew to 0.3×0.3×0.1 mm³ after seven days.

### EXAMPLE 4

### X-Ray Data Collection and Structure Determination

X-ray diffraction data was collected from Syk_{Cat} complex crystals at 100 K at European Synchrotron Radiation facility (ESRF). Crystals were flash-frozen from cryosolution containing reservoir or well solution.

Crystals of Syk_{Cat}-staurosporine were “annealed” before data collection. The term “anneal” refers to allowing a previously cryogenically-cooled sample to warm in temperature (as seen by visible signs of thawing) before flash-cooling or flash-freezing the sample again. This annealing process may be performed once or repeated multiple times. Annealing is performed by blocking a cryocooling stream from a crystal sample mounted on a goniostat for several seconds before allowing the crystal to be exposed to the stream again. Annealing is used to reduce mosaicity and increase the limit of resolution (Yeh and Hol, Acta Cryst. D54, pp. 479-480 (1998); Harp et al., Acta Cryst. D55, pp. 1129-1334 (1999); Harp et al, Acta Cryst., D54, pp. 622-628 (1998)). The annealing step may also reduce the static disorder of the crystal (Garman, Acta Cryst. D55, pp. 1641-1653 (1999)).

The data sets were processed with DENZO and SCALEPACK (Otwinowski, Z. & Minor, W., Methods in Enzymol. 276, pp. 307-326 (1997)). Detailed information about data statistics is provided in Table 1 and Table 2 for Syk_{Cat}-staurosporine and Syk_{Cat}-PT426-AMP-PNP complexes, respectively. All crystallographic calculations were performed using the CCP4 program package CCP4 (The CCP4 suite: Programs for protein crystallography. Acta. Cryst. D50, pp. 760-763 (1994)).

TABLE 1
Summary of data collection for the Syk_{Cat}-
staurosporine complex.

| | Syk_{Cat}-Staurosporine |
|---|---|
| Crystal data | |
| Space group | P2₁2₁2₁ |
| Unit cell parameters (Å) | a = 39.45 |
| | b = 84.17 |
| | c = 85.00 |
| Monomers per | 1 |
| asymmetric unit | |
| Solvent content (%) | 38.5 |
| Data collection | |
| X-ray source | ID14-4 (ESRF) |
| Image plate system | Q4 CCD (ADSC) |
| Wavelength (Å) | 1.0055 |
| Data | |
| Resolution range (Å) | 35-1.65 (1.69-1.65) |
| (overall/outer shell) | |
| Total reflections | 679,250 |
| Unique reflections | 42,026 |
| Completeness (%) | 99.0 (90.0) |
| (overall/outer shell) | |
| I/σ (I) | 24.6 (4.2) |
| (overall/outer shell) | |
| R merge (%)* | 4.2 (18.2) |
| (overall/outer shell) | |

*R_{merge} = 100 × ΣₕΣᵢ @Iₕᵢ − <Iₕ> @/ΣₕΣᵢIₕᵢ.

TABLE 2
Summary of data collection for the Syk_{Cat}-PT426-
AMP-PNP complex.

| | Syk_{Cat}-PT426-AMP-PNP |
|---|---|
| Crystal data | |
| Space group | P2₁2₁2₁ |
| Unit cell parameters | a = 39.58 |
| (Å) | b = 84.67 |
| | c = 90.63 |
| Monomers per | 1 |
| asymmetric unit | |
| Solvent content (%) | 43.1 |
| Data collection | |
| X-ray source | ID14-4 (ESRF) |
| Image plate system | Q4 CCD (ADSC) |
| Wavelength (Å) | 0.95 |
| Data | |
| Resolution range (Å) | 40-2.4 (2.44-2.40) |
| (overall/outer shell) | |
| Total reflections | 314 575 |
| Unique reflections | 12 559 |
| Completeness (%) | 99.4 (90.9) |
| (overall/outer shell) | |
| I/σ (I) | 32.2 (9.5) |
| (overall/outer shell) | |
| R merge (%)* | 9.1 (2.5) |
| (overall/outer shell) | |

*R_{merge} = 100 × ΣₕΣᵢ @Iₕᵢ − <Iₕ> @/ΣₕΣᵢIₕᵢ.

Syk_{Cat} In Complex with Staurosporine

The Syk_{Cat}-staurosporine structure was solved by molecular replacement. Molecular replacement calculations were performed with the program AMoRe as implemented in the CCP4 suite (Navaza, Acta. Cryst. D50, pp. 157-163 (1993)). A homology model of the catalytic domain of Syk (residues Lys368 to Asn635) was used as a search model in molecular replacement. This Syk_{Cat} homology model was built predominantly using the structure of the Lck (LymphoCyte-specific Kinase) protein kinase structure (PDB accession code 1QPJ) with a program Modeler™ (Accelrys). A rotation search and a subsequent translation search (resolution ranges 10.0-2.8 Å) produced a single top solution (α=9.10, β=53.83, γ=304.71, x=0.09, y=0.132 and z=0.211) with a correlation coefficient of 76.2% and an R factor of 35.9%.

The initial molecular replacement solution featured only amino acid residues present in the original search model. The automated refinement program ARP/wARP (Lamzin & Wilson, Acta Cryst. , D49, pp. 129-147 (1993); Lamzin & Wilson, Meth. Enzym., 277, pp. 269-305 (1997)) was used to improve the electron density maps and build in more amino acid residues and solvent molecules.

The output model from ARP/wARP was then further refined using the maximum likelihood approach implemented in RefMac 5.0 (Murshudov, et al., “Application of Maximum Likelihood Refinement” in the Refinement of Protein structures, Proceedings of Daresbury Study Weekend (1996); Murshudov, et al., Acta Cryst. , D53, pp. 240-255(1997); Murshudov, et al., Acta Cryst. , D55 pp. 247-255 (1999)). After five cycles of restrained isotropic refinement, the resulting R factor was 19.4% and R_{free} was 23%.

Electron density maps built after RefMac cycles were viewed using QUANTA (Accelrys ©2001,2002) and model building was performed using a QUANTA module X-AUTOFIT (Oldfield, Proceedings from the 1996 Meeting of the International Union of Crystallography Macromolecular Computing School; see http://www.sdsc.edu/Xtal/IUCr/CC/School96/; Accelrys ©2001,2002). Graphical remodeling steps included mutating amino acid side chains of the Syk_{Cat} homology model to the Syk_{Cat} residue side chains, and rebuilding the loop and disordered regions. Syk_{Cat} amino acid residues Asn406 to Pro411 were not built into the model because the electron density was weak in these regions.

Electron density maps showed the presence of extra electron density in the shape of a flat, multi-cyclic ligand located in the cleft between the N-terminal and C-terminal domains, where the putative ATP-binding site is located. Staurosporine was built into the model using geometric restraints inferred by modeling staurosporine into the Syk_{Cat} homology model used earlier for molecular replacement, and restraints of the crystal structure of Csk (C-terminal Src Kinase) complexed with staurosporine (PDB accession code 1BYG).

After minimal model building, the ligand fit well with the extra electron density present in the ATP-binding site. Final refinement was done by using the program RefMac interspersed with manual model building using X-AUTOFIT in QUANTA (Oldfield, supra).

For the Syk_{Cat}-staurosporine complex, the final R_{working} and R_{free} was 16.4% and 19.5%, respectively. 5.0% of data was used for the test set in the calculation of R_{free}. The final model contains all amino acid residues of Syk_{Cat} except the first 15 N-terminal amino acid residues and amino acid residues Asn406-Asp410, 374 water molecules, four amino acid residues from the N-terminal end of a neighboring Syk_{Cat} molecule, and one staurosporine. No tyrosines amino acid residues were phosphorylated in the final model.

The final model was validated using programs PROCHECK (Laskowski et al., J. Appl. Cryst., 26, pp. 283-291 (1993); Morris et al., Proteins, 12, pp. 345-364 (1992)) and SQUID VALIDATE (Molecular Simulations, Inc., San Diego, Calif. ©1998, 2000; Accelrys ©2001,2002). A Ramachandran plot for the resulting Syk_{Cat}-staurosporine complex showed 92.5% of all non-Gly, non-Pro residues in the “core” or most favorable regions, 7.1% in “allowed” regions, and one residue in the “generally allowed” regions.

Suk_{Cat} In Complex with Peptide, PT426

The Syk_{Cat}-PT426-AMP-PNP structure was solved by molecular replacement using the structure of the Syk_{Cat}-staurosporine complex described above, without ligand or solvent molecules, as the search model. A rotation search followed by a translation search (resolution ranges 10.0-3.0 Å) produced a single top solution with a correlation coefficient of 63% and an R-factor of 42.4% after refinement in AmoRe (Navaza, J., Acta. Cryst. D50, pp. 157-163 (1993)). The molecular replacement solution corresponds to the rotation and translation function α=9.00, β=50.80, γ=306.77, x=0.110, y=0.134 and z=0.212

Refinement was performed using the maximum likelihood approached implemented in RefMac (Murshudov, et al., “Application of Maximum Likelihood Refinement” in the Refinement of Protein structures, Proceedings of Daresbury Study Weekend (1996); Murshudov, et al., Acta Cryst. , D53, pp. 240-255(1997); Murshudov, et al., Acta Cryst. , D55 pp. 247-255 (1999)). Resulting electron density maps were viewed in QUANTA (Accelrys ©2001,2002) and model building was carried out using X-AUTOFIT (Oldfield, supra). Iterative cycles of model building and refinement were used to improve electron density maps. No model was built for Syk_{Cat} amino acid residues Lys405-Pro411 because of weak electron density.

Difference density maps showed tetrahedral bulges of density around the end of tyrosine residues Tyr525 and Tyr526, suggesting that these amino acid residues were phosphorylated. Phosphotyrosine residues (PTyr525 and PTyr526) were modeled into these positions. An AMP-PNP molecule was built into a region of electron density in the ATP-binding site.

Additional electron density was apparent in the region around amino acid residues His531 to Pro535. These residues are located in the substrate-binding site. Density for a tyrosine amino acid side chain was visible. Amino acid residues E, D, D, and Y were built into the additional density. The remaining density was weak and difficult to fit. The weak density may result from a flexible or disordered region in the molecule or less than 100% occupancy of the peptide in the binding pocket.

Final refinement was done by using the program RefMac interspersed with manual model building using X-AUTOFIT in QUANTA (Oldfield, supra). The final model of the Syk_{Cat}-PT426-AMP-PNP complex has a final R_{working} and R_{free} of 19.8% and 28.8%, respectively. 4.8% of data was used for the test set in calculating the R_{free}. The final model contained the catalytic domain of Syk including 271 water molecules, PT426, AMP-PNP, and two Mg+² atoms. The first 21 N-terminal residues, the last C-terminal residue, the loop residues Asn381, Phe382, and Lys405-Pro411 of Syk_{Cat} are not present in the model. These regions have weak density and can not be modeled. Tyr525 and Tyr526 were phosphorylated in the final model.

The final model was validated using programs PROCHECK (Laskowski et al., J. Appl. Cryst., 26, pp. 283-291 (1993); Morris et al., Proteins, 12, pp. 345-364 (1992)) and SQUID VALIDATE (Molecular Simulations, Inc., San Diego, Calif. ©1998, 2000; Accelrys ©2001,2002). A Ramachandran plot for the resulting Syk_{Cat}-PT426-AMP-PNP complex showed 87.9% of all non-Gly, non-Pro residues in the “core” or most favorable regions, 10.4% in “allowed” regions, 13% in the “generally allowed” regions and 0.4% (1 residue) in disallowed regions.

### EXAMPLE 5

### Overall Structure of Syk Catalytic Domain

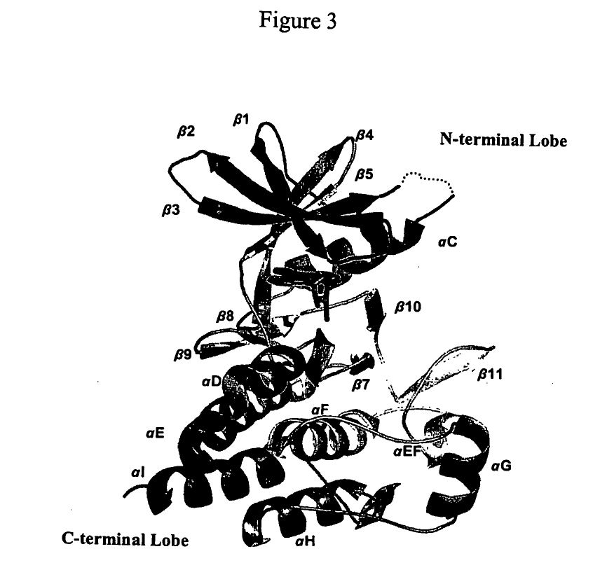

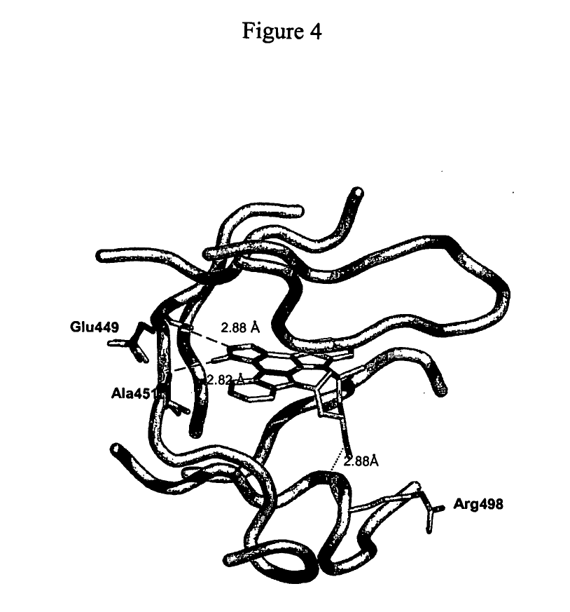

Syk family tyrosine kinases contain a C-terminal catalytic domain and tandem N-terminal SH2 domains. The present invention provides for the crystal structure of the C-terminal catalytic domain of Syk (Syk_{Cat}) (FIGS. 3 and 4). The conventional nomenclature for PK secondary structural elements are used in FIGS. 3 and 4 (Knighton et al., (1991); Hubbard et al. (1994)). The N-terminal lobe or sub-domain of the catalytic domain contains a curled β-sheet of five anti-parallel β-strands (β1-β5) and one α-helix (αC) positioned between the β3 and β4 strands. The C-terminal lobe or sub-domain comprises four β-strands (β7, β8, β9, and β10) and eight helices (αD, αE, αEF, αF, αG, αH, αHI, and αI).

The two phosphorylated tyrosine residues in the Syk_{Cat}-PT426-AMP-PNP structure, PTyr525 and PTyr526, are found in the activation loop (amino acid residues L515 to Y539), which is present in the kinase domains of most PTKs. These tyrosine residues have been previously identified as sites of autophosphorylation within the activation loop of murine Syk (Furlong et al., Biochim. Biophys. Acta, 1355, pp. 177-190 (1997); Zhang et al., J. Biol. Chem., 275, pp. 35442-35447, (2000)). Lys517, a highly conserved residue in the PTK family, is in close proximity of PTyr526. PTyr525 is in close proximity of Lys548.

The structures of the two Syk_{Cat} complexes in the present invention have highly similar structures. When the two structures are superimposed, the regions of highest RMSDs occur in specific regions within the catalytic domains including strand 3 and helix C in the N-terminal lobe, helices F and H of the C-terminal lobe, the sides of the nucleotide binding site, and the substrate binding site and activation loop.

### EXAMPLE 6

### Catalytic Active Site of Syk_{Cat} Complexes

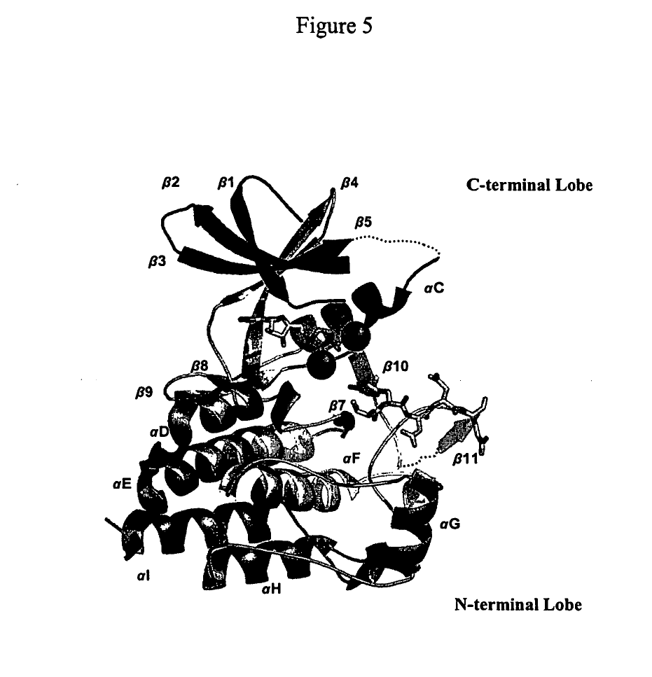

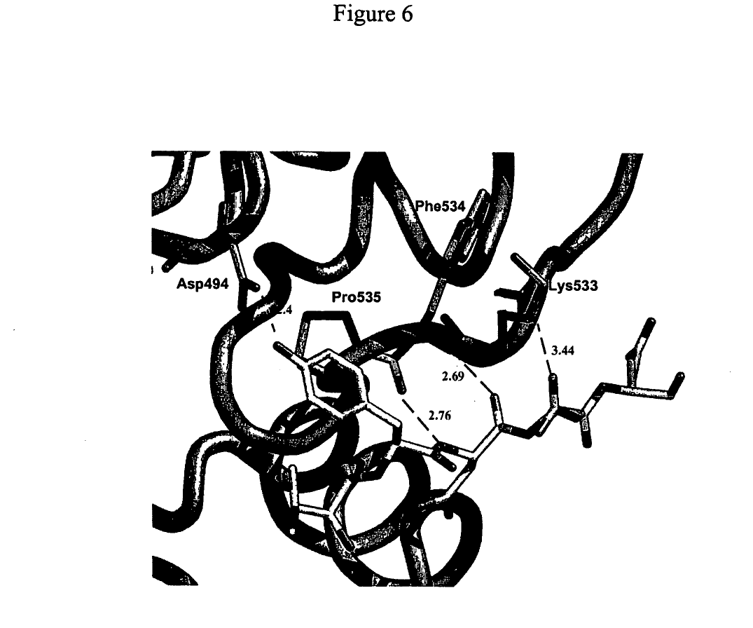

The inhibitor staurosporine binds in a hydrophobic cleft between the N- and C-terminal lobes of the Syk_{Cat} structure (FIGS. 3 and 5). Staurosporine forms hydrogen bond interactions with Glu449, Ala451, and Arg498. Staurosporine also forms hydrophobic interactions with cleft residue side chains.

In the Syk_{Cat}-PT426-AMP-PNP complex structure, AMP-PNP binds in the nucleotide-binding site that is situated between the N- and C-terminal lobes (FIG. 4). Interactions between AMP-PNP and Syk_{Cat} protein include hydrogen bonds with residues Ser379, Glu449, Asp512 and hydrophobic interactions with cleft residue side chains. Additionally, some interaction exists between AMP-PNP and Lys402. The two divalent magnesium ions are coordinated with negatively charged residues, α- and β-phosphate groups and/or water molecules. Mg1 from FIG. 2 makes contacts to the α- and β-phosphate groups and residues Asn499 and Asp512. Mg2 from FIG. 2 coordinates to the β-phosphate group of AMP-PNP, residue Glu420 and water molecule 121, which forms a bridge to residue Glu416. In turn, the α- and β-phosphate groups of AMP-PNP interact with the magnesium ions, Syk_{Cat} amino acid residues Lys402, Asn499, Asp512, and a water molecule.

### EXAMPLE 7

### Substrate-Binding Site of Syk_{Cat} Complexes

In the Syk_{Cat}-PT426-AMP-PNP complex, supporting electron density was seen for the backbone of only four of the eight PT426 residues. These amino acid residues were initially built in the PT426 structural model as E, D, D, Y. The exact identity of these residues can not be known for certain since the electron density for all the side chains in the electron density was weak in the peptide region. In addition, valine was inadvertently left as the first amino acid residue in the PT426 model as a remnant of peptide model building. Although valine was given in the structure coordinates listed in FIG. 2, it is not part of the PT426 peptide.

These factors are not expected to affect the scope or utility of the present invention because electron density is visible for the backbone of four amino acid residues in the peptide. The visible PT426 tyrosine residue is the acceptor tyrosine residue for phosphorylation. The PT426 peptide forms various hydrogen bonds with amino acid residues Gly532, Lys533, Trp534 and Pro535 at the end of the activation loop. The hydroxyl group of the tyrosine in the peptide forms a hydrogen bond with the carboxylate group of Asp494.

In the structure of the Syk_{Cat}-staurosporine complex, unexpected electron density appeared at the substrate binding site. No substrate was added to the Syk_{Cat}-staurosporine complex crystallization, but the N-terminal end of Syk_{Cat} could be built into this unexpected density. The N-terminal region of the Syk catalytic domain mimics the sequence of PT426:

| | | |
|---|---|---|
| N-term | E E D D Y E S P | unmodified PT426 |
| | | (SEQ ID NO: 5) |
| N-term | D T E V Y E S P | Syk (amino acid residues |
| | | 344-351 of SEQ ID NO: 1) |

The N-terminal region of Syk_{Cat} also contains Tyr348, one of the proposed major sites of autophosphorylation of Syk (Furlong et al., Biochim. Biophys. Acta, 1355, pp. 177-190 (1997)). In the crystal, the N-terminal end of the Syk_{Cat} packs against the substrate binding pocket of the neighboring Syk_{Cat} molecule, and it extends within a very close distance (˜20 Å) to the peptide moiety in the substrate binding pocket of the Syk_{Cat}-PT426 complex structure. This distance could easily be bridged by eight residues between the visible N-terminus of the Syk_{Cat}-Staurosporine complex (Ile358)and the first visible residue of the bound peptide (Ser350, if it were an extension of the N-terminus). The presence of the N-terminal end of the Syk_{Cat} domain of Syk bound in the substrate binding site either may be the structure of the Syk molecule at the time of autophosphorylation or a result of crystal packing that forces the N-terminal end of Syk_{Cat} into the substrate binding site of the Syk_{Cat}-staurosporine complex.

We claim:

1. A crystal comprising a catalytic domain of Syk protein or homologue thereof.

2. The crystal according to claim 1, further comprising a chemical entity, wherein said chemical entity binds to the catalytic domain of Syk protein or homologue thereof.

3. The crystal according to claim 2, wherein said chemical entity binds to an active site on the catalytic domain of Syk protein or homologue thereof.

4. The crystal according to claim 3, wherein the chemical entity is selected from the group consisting of staurosporine, adenosine, ATP, an ATP analogue, a nucleotide triphosphate, a nucleotide diphosphate, phosphate and active site inhibitor.

5. The crystal according to claim 3, wherein the chemical entity is selected from the group consisting of staurosporine and AMP-PNP.

6. The crystal according to claim 2, wherein the chemical entity binds to a substrate binding site on the catalytic domain of Syk protein or homologue thereof.

7. The crystal according to claim 6, wherein the chemical entity is selected from the group consisting of NAc-Glu-Glu-Asp-Asp-Tyr-Glu-Ser-Pro-NH₂ (SEQ ID NO: 2), Glu-Glu-Asp-Asp-Tyr-Glu-Ser-Pro (SEQ ID NO: 5), a peptide comprising the amino acid sequence Glu-Asp-Asp-Tyr (residues 2-5 of SEQ ID NO: 5), a peptide comprising the amino acid sequence Asp-Asp-Tyr-Glu (residues 3-6 of SEQ ID NO: 5), a peptide comprising the amino acid sequence Asp-Tyr-Glu-Ser (residues 4-7 of SEQ ID NO: 5), a peptide comprising the amino acid sequence Tyr-Glu-Ser-Pro (residues 5-8 of SEQ ID NO: 5), a peptide comprising the amino acid sequence Glu-Glu-Asp-Asp-Tyr (residues 1-5 of SEQ ID NO: 5), a peptide comprising the amino acid sequence Glu-Asp-Asp-Tyr-Glu (residues 2-6 of SEQ ID NO: 5), a peptide comprising the amino acid sequence Asp-Asp-Tyr-Glu-Ser (residues 3-7 of SEQ ID NO: 5), a peptide comprising the amino acid sequence Asp-Tyr-Glu-Ser-Pro (residues 4-8 of SEQ ID NO: 5), a peptide comprising amino acids Asp-Glu-Glu-Asp-Tyr (SEQ ID NO: 6), a peptide comprising amino acids Asp-Glu-Glu-Tyr-Asp (SEQ ID NO: 7), a peptide comprising amino acids Asp-Glu-Tyr-Glu-Asp (SEQ ID NO: 8), a peptide comprising amino acids Asp-Tyr-Glu-Glu-Val (SEQ ID NO: 9), and a peptide comprising amino acids Tyr-Ser-Ile-Ile-Nle (SEQ ID NO: 10).

8. The crystal of claim 1 or 2, wherein said catalytic domain of Syk protein or homologue thereof is phosphorylated.

9. The crystal according to claim 1 or 2, wherein said catalytic domain of Syk protein is selected from the group consisting of amino acid residues 343-635, amino acid residues 358-635 and amino acid residues 364-634 of SEQ ID NO: 1.

10. The crystal according to claim 1 or 2, wherein said catalytic domain of Syk protein comprises amino acid residues 343-635 of SEQ ID NO: 1.

11. A crystallizable composition comprising a catalytic domain of Syk protein or homologue thereof.

12. The crystallizable composition according to claim 11, further comprising a chemical entity, wherein said chemical entity binds to the catalytic domain of Syk protein or homologue thereof.

13. The crystallizable composition according to claim 12, wherein said chemical entity binds to an active site on the catalytic domain of Syk protein or homologue thereof.

14. The crystallizable composition according to claim 13, wherein the chemical entity is selected from the group consisting of staurosporine, adenosine, ATP, an ATP analogue, a nucleotide triphosphate, a nucleotide diphosphate, phosphate and active site inhibitor.

15. The crystallizable composition according to claim 13, wherein the chemical entity is selected from the group consisting of staurosporine and AMP-PNP.

16. The crystallizable composition according to claim 12, wherein the chemical entity binds to a substrate binding site on the catalytic domain of Syk protein or homologue thereof.

17. The crystallizable composition according to claim 16, wherein the chemical entity is selected from the group consisting of NAc-Glu-Glu-Asp-Asp-Tyr-Glu-Ser-Pro-NH₂ (SEQ ID NO: 2), Glu-Glu-Asp-Asp-Tyr-Glu-Ser-Pro (SEQ ID NO: 5), a peptide comprising the amino acid sequence Glu-Asp-Asp-Tyr (amino acids 2-5 of SEQ ID NO: 5) a peptide comprising the amino acid sequence Asp-Asp-Tyr-Glu (residues 3-6 of SEQ ID NO: 5), a peptide comprising the amino acid sequence Asp-Tyr-Glu-Ser (residues 4-7 of SEQ ID NO: 5), a peptide comprising the amino acid sequence Tyr-Glu-Ser-Pro (residues 5-8 of SEQ ID NO: 5), a peptide comprising the amino acid sequence Glu-Glu-Asp-Asp-Tyr (residues 1-5 of SEQ ID NO: 5), a peptide comprising the amino acid sequence Glu-Asp-Asp-Tyr-Glu (residues 2-6 of SEQ ID NO: 5), a peptide comprising the amino acid sequence Asp-Asp-Tyr-Glu-Ser (residues 3-7 of SEQ ID NO: 5), a peptide comprising the amino acid sequence Asp-Tyr-Glu-Ser-Pro (residues 4-8 of SEQ ID NO: 5), a peptide comprising amino acids Asp-Glu-Glu-Asp-Tyr (SEQ ID NO: 6), a peptide comprising amino acids Asp-Glu-Glu-Tyr-Asp (SEQ ID NO: 7), a peptide comprising amino acids Asp-Glu-Tyr-Glu-Asp (SEQ ID NO: 8), a peptide comprising amino acids Asp-Tyr-Glu-Glu-Val (SEQ ID NO: 9), and a peptide comprising amino acids Tyr-Ser-Ile-Ile-Nle (SEQ ID NO: 10).

18. The crystallizable composition of claim 11 or 12, wherein said catalytic domain of Syk protein or homologue thereof is phosphorylated.

19. The crystallizable composition according to claim 11 or 12, wherein said catalytic domain of Syk protein is selected from the group consisting of amino acid residues 343-635, amino acid residues 358-635 and amino acid residues 364-634 of SEQ ID NO: 1.

20. The crystallizable composition according to claim 11 or 12, wherein said catalytic domain of Syk protein comprises amino acid residues 343-635 of SEQ ID NO: 1.

21. A computer comprising:
(a) a machine-readable data storage medium, comprising a data storage material encoded with machine-readable data, wherein said data defines the binding pocket or domain selected from the group consisting of:
(i) a set of amino acid residues comprising at least four amino acid residues which are identical to Syk amino acid residues Asp494, Gly532, Lys533, Trp534 and Pro535 according to FIG. 2, wherein the root mean square deviation of the backbone atoms between said at least four amino acid residues and said Syk amino acid residues which are identical is not greater than about 3 Å;
(ii) a set of amino acid residues comprising at least four amino acid residues which are identical to Syk amino acid residues L377, M424, V433, M448, A451, G454, L501 and S511 according to FIG. 1 or 2, wherein the root mean square deviation of the backbone atoms between said at least four amino acid residues and said Syk amino acid residues which are identical is not greater than about 3 Å;
(iii) a set of amino acid residues comprising at least four amino acid residues which are identical to Syk amino acid residues L377, G378, S379, V385, A400, K402, V433, M448, E449, M450, A451, E452, P455, R498, N499, L501, S511 and D512 according to FIG. 1 or 2, wherein the root mean square deviation of the backbone atoms between said at least four amino acid residues and said Syk amino acid residues which are identical is not greater than about 3 Å;
(iv) a set of amino acid residues comprising at least four amino acid residues which are identical to Syk amino acid residues K375, E376, G380, N381, G383, T384, K386, K387, T398, V399, V401, M424, R434, L446, V447, L456, K458, D494, A496, A497, V500, L502, V503, K509, I510, F513 and G514 according to FIG. 1, wherein the root mean square deviation of the backbone atoms between said at least four amino acid residues and said Syk amino acid residues which are identical is not greater than about 3 Å;
(v) a set of amino acid residues comprising at least four amino acid residues which are identical to Syk amino acid residues D376, G380, G383, T384, K386, T398, V399, V401, L417, E420, M424, R434, M435, L446, V447, L453, G454, L456, N457, D494, A497, V500, L502, V503, K509, I510, F513, G514 and L515 according to FIG. 2, wherein the root mean square deviation of the backbone atoms between said at least four amino acid residues and said Syk amino acid residues which are identical is not greater than about 3 Å; and
(vi) a set of amino acid residues which are identical to Syk amino acid residues according to FIG. 1 or 2, wherein the root mean square deviation of the backbone atoms between said amino acid residues and said Syk amino acid residues which are identical is not greater than about 5 Å;
(b) a working memory for storing instructions for processing said machine-readable data;
(c) a central processing unit coupled to said working memory and to said machine-readable data storage medium for processing said machine-readable data and a means for generating three-dimensional structural information of said binding pocket or domain; and
(d) output hardware coupled to said central processing unit for outputting three-dimensional structural information of said binding pocket or domain, or information produced using said three-dimensional structural information of said binding pocket or domain.

22. The computer according to claim 21, wherein the binding pocket is produced by homology modeling of the structure coordinates of said Syk amino acid residues according to FIG. 1 or 2.

23. The computer according to claim 21, wherein said means for generating three-dimensional structural information is provided by means for generating a three-dimensional structural representation of said binding pocket or domain.

24. The computer according to claim 21, wherein said output hardware is a display terminal, a printer, CD or DVD recorder, ZIP™ or JAZ™ drive, a disk drive, or other machine-readable data storage device.

25. A method of using a computer for selecting an orientation of a chemical entity that interacts favorably with a binding pocket or domain selected from the group consisting of:
(i) a set of amino acid residues comprising at least four amino acid residues which are identical to Syk amino acid residues Asp494, Gly532, Lys533, Trp534 and Pro535 according to FIG. 2, wherein the root mean square deviation of the backbone atoms between said at least four amino acid residues and said Syk amino acid residues which are identical is not greater than about 3 Å;
(ii) a set of amino acid residues comprising at least four amino acid residues which are identical to Syk amino acid residues L377, M424, V433, M448, A451, G454, L501 and S511 according to FIG. 1 or 2, wherein the root mean square deviation of the backbone atoms between said at least four amino acid residues and said Syk amino acid residues which are identical is not greater than about 3 Å;
(iii) a set of amino acid residues comprising at least four amino acid residues which are identical to Syk amino acid residues L377, G378, S379, V385, A400, K402, V433, M448, E449, M450, A451, E452, P455, R498, N499, L501, S511 and D512 according to FIG. 1 or 2, wherein the root mean square deviation of the backbone atoms between said at least four amino acid residues and said Syk amino acid residues which are identical is not greater than about 3 Å;
(iv) a set of amino acid residues comprising at least four amino acid residues which are identical to Syk amino acid residues K375, E376, G380, N381, G383, T384, K386, K387, T398, V399, V401, M424, R434, L446, V447, L456, K458, D494, A496, A497, V500, L502, V503, K509, I510, F513 and G514 according to FIG. 1, wherein the root mean square deviation of the backbone atoms between said at least four amino acid residues and said Syk amino acid residues which are identical is not greater than about 3 Å;
(v) a set of amino acid residues comprising at least four amino acid residues which are identical to Syk amino acid residues D376, G380, G383, T384, K386, T398, V399, V401, L417, E420, M424, R434, M435, L446, V447, L453, G454, L456, N457, D494, A497, V500, L502, V503, K509, I510, F513, G514 and L515 according to FIG. 2, wherein the root mean square deviation of the backbone atoms between said at least four amino acid residues and said Syk amino acid residues which are identical is not greater than about 3 Å; and
(vi) a set of amino acid residues which are identical to Syk amino acid residues according to FIG. 1 or 2, wherein the root mean square deviation of the backbone atoms between said amino acid residues and said Syk amino acid residues which are identical is not greater than about 5 Å;
said method comprising the steps of:
(a) providing the structure coordinates of said binding pocket or domain on a computer comprising the means for generating three-dimensional structural information from said structure coordinates;
(b) employing computational means to dock a first chemical entity in the binding pocket or domain;
(c) quantitating the interaction energy between said chemical entity and all or part of the binding pocket or domain for different orientations of the chemical entity; and
(d) selecting the orientation of the chemical entity with the most favorable interaction energy.

26. The method according to claim 25, further comprising generating a three-dimensional graphical representation of the binding pocket or domain prior to step (b).

27. The method of claim 25, wherein energy minimization with or without molecular dynamics simulations or rigid-body minimizations are performed simultaneously with or following step (b).

28. The method according to claim 25, further comprising the steps of:
(e) repeating steps (b) through (d) with a second chemical entity; and
(f) selecting at least one of said first or second chemical entity that interacts more favorably with said binding pocket or domain based on said quantitated interaction energy of said first or second chemical entity.

29. A method of using a computer for selecting an orientation of a chemical entity with a favorable shape complementarity in a binding pocket selected from the group consisting of:
(i) a set of amino acid residues comprising at least four amino acid residues which are identical to Syk amino acid residues Asp494, Gly532, Lys533, Trp534 and Pro535 according to FIG. 2, wherein the root mean square deviation of the backbone atoms between said at least four amino acid residues and said Syk amino acid residues which are identical is not greater than about 3 Å;
(ii) a set of amino acid residues comprising at least four amino acid residues which are identical to Syk amino acid residues L377, M424, V433, M448, A451, G454, L501 and S511 according to FIG. 1 or 2, wherein the root mean square deviation of the backbone atoms between said at least four amino acid residues and said Syk amino acid residues which are identical is not greater than about 3 Å;
(iii) a set of amino acid residues comprising at least four amino acid residues which are identical to Syk amino acid residues L377, G378, S379, V385, A400, K402, V433, M448, E449, M450, A451, E452, P455, R498, N499, L501, S511 and D512 according to FIG. 1 or 2, wherein the root mean square deviation of the backbone atoms between said at least four amino acid residues and said Syk amino acid residues which are identical is not greater than about 3 Å;
(iv) a set of amino acid residues comprising at least four amino acid residues which are identical to Syk amino acid residues K375, E376, G380, N381, G383, T384, K386, K387, T398, V399, V401, M424, R434, L446, V447, L456, K458, D494, A496, A497, V500, L502, V503, K509, I510, F513 and G514 according to FIG. 1, wherein the root mean square deviation of the backbone atoms between said at least four amino acid residues and said Syk amino acid residues which are identical is not greater than about 3 Å;
(v) a set of amino acid residues comprising at least four amino acid residues which are identical to Syk amino acid residues D376, G380, G383, T384, K386, T398, V399, V401, L417, E420, M424, R434, M435, L446, V447, L453, G454, L456, N457, D494, A497, V500, L502, V503, K509, I510, F513, G514 and L515 according to FIG. 2, wherein the root mean square deviation of the backbone atoms between said at least four amino acid residues and said Syk amino acid residues which are identical is not greater than about 3 A; and
(vi) a set of amino acid residues which are identical to Syk amino acid residues according to FIG. 1 or 2, wherein the root mean square deviation of the backbone atoms between said amino acid residues and said Syk amino acid residues which are identical is not greater than about 5 Å;
said method comprising the steps of:
(a) providing the structure coordinates of said binding pocket and ligand bound therein on a computer comprising the means for generating three-dimensional structural information from said structure coordinates;
(b) employing computational means to dock a first chemical entity in the binding pocket;
(c) quantitating the contact score of said chemical entity in different orientions; and
(d) selecting an orientation with the highest contact score.

30. The method according to claim 29, further comprising generating a three-dimensional graphical representation of the binding pocket and ligand bound therein prior to step (b).

31. The method according to claim 29, further comprising the steps of:
(e) repeating steps (b) through (d) with a second chemical entity; and
(f) selecting at least one of said first or second chemical entity that has a higher contact score based on said quantitated contact score of said first or second chemical entity.

32. A method for identifying a candidate inhibitor of a molecule or molecular complex comprising a binding pocket or domain selected from the group consisting of:
(i) a set of amino acid residues comprising at least four amino acid residues which are identical to Syk amino acid residues Asp494, Gly532, Lys533, Trp534 and Pro535 according to FIG. 2, wherein the root mean square deviation of the backbone atoms between said at least four amino acid residues and said Syk amino acid residues which are identical is not greater than about 3 Å;
(ii) a set of amino acid residues comprising at least four amino acid residues which are identical to Syk amino acid residues L377, M424, V433, M448, A451, G454, L501 and S511 according to FIG. 1 or 2, wherein the root mean square deviation of the backbone atoms between said at least four amino acid residues and said Syk amino acid residues which are identical is not greater than about 3 Å;
(iii) a set of amino acid residues comprising at least four amino acid residues which are identical to Syk amino acid residues L377, G378, S379, V385, A400, K402, V433, M448, E449, M450, A451, E452, P455, R498, N499, L501, S511 and D512 according to FIG. 1 or 2, wherein the root mean square deviation of the backbone atoms between said at least four amino acid residues and said Syk amino acid residues which are identical is not greater than about 3 Å;
(iv) a set of amino acid residues comprising at least four amino acid residues which are identical to Syk amino acid residues K375, E376, G380, N381, G383, T384, K386, K387, T398, V399, V401, M424, R434, L446, V447, L456, K458, D494, A496, A497, V500, L502, V503, K509, I510, F513 and G514 according to FIG. 1, wherein the root mean square deviation of the backbone atoms between said at least four amino acid residues and said Syk amino acid residues which are identical is not greater than about 3 Å;
(v) a set of amino acid residues comprising at least four amino acid residues which are identical to Syk amino acid residues D376, G380, G383, T384, K386, T398, V399, V401, L417, E420, M424, R434, M435, L446, V447, L453, G454, L456, N457, D494, A497, V500, L502, V503, K509, I510, F513, G514 and L515 according to FIG. 2, wherein the root mean square deviation of the backbone atoms between said at least four amino acid residues and said Syk amino acid residues which are identical is not greater than about 3 Å; and
(vi) a set of amino acid residues which are identical to Syk amino acid residues according to FIG. 1 or 2, wherein the root mean square deviation of the backbone atoms between said amino acid residues and said Syk amino acid residues which are identical is not greater than about 5 Å;
comprising the steps of:
(a) using a three-dimensional structure of the binding pocket or domain to design, select or optimize a plurality of chemical entities;
(b) contacting each chemical entity with the molecule or the molecular complex;
(c) monitoring the inhibition to the catalytic activity of the molecule or molecular complex by each chemical entity; and
(d) selecting a chemical entity based on the inhibitory effect of the chemical entity on the catalytic activity of the molecule or molecular complex.

33. A method of designing a compound or complex that interacts with a binding pocket or domain selected from the group consisting of:
(i) a set of amino acid residues comprising at least four amino acid residues which are identical to Syk amino acid residues Asp494, Gly532, Lys533, Trp534 and Pro535 according to FIG. 2, wherein the root mean square deviation of the backbone atoms between said at least four amino acid residues and said Syk amino acid residues which are identical is not greater than about 3 Å;
(ii) a set of amino acid residues comprising at least four amino acid residues which are identical to Syk amino acid residues L377, M424, V433, M448, A451, G454, L501 and S511 according to FIG. 1 or 2, wherein the root mean square deviation of the backbone atoms between said at least four amino acid residues and said Syk amino acid residues which are identical is not greater than about 3 Å;
(iii) a set of amino acid residues comprising at least four amino acid residues which are identical to Syk amino acid residues L377, G378, S379, V385, A400, K402, V433, M448, E449, M450, A451, E452, P455, R498, N499, L501, S511 and D512 according to FIG. 1 or 2, wherein the root mean square deviation of the backbone atoms between said at least four amino acid residues and said Syk amino acid residues which are identical is not greater than about 3 Å;
(iv) a set of amino acid residues comprising at least four amino acid residues which are identical to Syk amino acid residues K375, E376, G380, N381, G383, T384, K386, K387, T398, V399, V401, M424, R434, L446, V447, L456, K458, D494, A496, A497, V500, L502, V503, K509, I510, F513 and G514 according to FIG. 1, wherein the root mean square deviation of the backbone atoms between said at least four amino acid residues and said Syk amino acid residues which are identical is not greater than about 3 Å;
(v) a set of amino acid residues comprising at least four amino acid residues which are identical to Syk amino acid residues D376, G380, G383, T384, K386, T398, V399, V401, L417, E420, M424, R434, M435, L446, V447, L453, G454, L456, N457, D494, A497, V500, L502, V503, K509, I510, F513, G514 and L515 according to FIG. 2, wherein the root mean square deviation of the backbone atoms between said at least four amino acid residues and said Syk amino acid residues which are identical is not greater than about 3 Å; and
(vi) a set of amino acid residues which are identical to Syk amino acid residues according to FIG. 1 or 2, wherein the root mean square deviation of the backbone atoms between said amino acid residues and said Syk amino acid residues which are identical is not greater than about 5 Å;
comprising the steps of:
(a) providing the structure coordinates of said binding pocket or domain on a computer comprising the means for generating three-dimensional structural information from said structure coordinates;
(b) using the computer to dock a first chemical entity in part of the binding pocket or domain;
(c) docking at least a second chemical entity in another part of the binding pocket or domain;
(d) quantifying the interaction energy between the first or second chemical entity and part of the binding pocket or domain;
(e) repeating steps (b) to (d) with another first and second chemical entity, selecting a first and a second chemical entity based on said quantified interaction energy of of all of said first and second chemical entity;
(f) optionally, visually inspecting the relationship of the first and second chemical entity to each other in relation to the binding pocket or domain on a computer screen using the three-dimensional graphical representation of the binding pocket or domain and said first and second chemical entity; and
(g) assembling the first and second chemical entity into a compound or complex that interacts with said binding pocket or domain by model building.

34. A method of utilizing molecular replacement to obtain a structural model of a molecule or a molecular complex of unknown structure, comprising the steps of:
(a) crystallizing said molecule or molecular complex;
(b) generating an X-ray diffraction pattern from said crystallized molecule or molecular complex;
(c) applying at least a portion of the structure coordinates set forth in FIGS. 1, 2 or a homology model thereof to the X-ray diffraction pattern to generate a three-dimensional electron density map of at least a portion of the molecule or molecular complex whose structure is unknown; and
(d) generating a structural model of the molecule or molecular complex from the three-dimensional electron density map.

35. The method according to claim 34, wherein the molecule is selected from the group consisting of a Syk catalytic domain protein and a Syk catalytic domain homologue.

36. The method according to claim 34, wherein the molecular complex is selected from the group consisting of a Syk catalytic domain protein complex and a Syk catalytic domain homologue complex.
